(19) United States
(12) Patent Application Publication
(10) Pub. No.: US 2025/0295800 A1
(43) Pub. Date: Sep. 25, 2025

(54) CONJUGATE OF A SINGLE DOMAIN ANTIBODY, A SAPONIN AND AN EFFECTOR MOLECULE, PHARMACEUTICAL COMPOSITION COMPRISING THE SAME, THERAPEUTIC USE OF SAID PHARMACEUTICAL COMPOSITION

(71) Applicant: SAPREME TECHNOLOGIES B.V., Bilthoven (NL)

(72) Inventors: Ruben POSTEL, Bilthoven (NL); Guy HERMANS, Bilthoven (NL)

(21) Appl. No.: 18/723,191

(22) PCT Filed: Dec. 20, 2022
(86) PCT No.: PCT/NL2022/050737

§ 371 Date: Jun. 21, 2024

(30) Foreign Application Priority Data
Dec. 22, 2021 (NL) 2030227

(51) Int. Cl. A61K 47/68; C07K 14/415; C07K 16/28; C07K 16/32

(52) U.S. Cl. CPC A61K 47/6825; A61K 47/6849; A61K 47/6889; C07K 14/415; C07K 16/2863; C07K 16/2881; C07K 16/32; C07K 2317/569

(57) ABSTRACT

The invention relates to a conjugate for transferring an effector molecule from outside a cell into said cell, the conjugate comprising at least one effector molecule to be transferred into the cell, at least one saponin of the mono-desmosidic triterpene glycoside type or the bi-desmosidic triterpene glycoside type, and at least one single-domain antibody (sdAb), covalently bound to each other, wherein the sdAb is capable of binding to a cell-surface molecule of said cell. The invention also relates to a pharmaceutical composition comprising the conjugate of the invention. Furthermore, the invention relates to a pharmaceutical composition of the invention, for use as a medicament. In addition, the invention relates to a pharmaceutical composition of the invention, for use in the treatment or the prophylaxis of any one or more of: a cancer, an auto-immune disease such as rheumatoid arthritis, an enzyme deficiency, a disease related to an enzyme deficiency, a gene defect, a disease relating to a gene defect, an infection such as a viral infection, hypercholesterolemia, primary hyperoxaluria, haemophilia A, haemophilia B, alpha-1 antitrypsin related liver disease, acute hepatic porphyria, an amyloidosis and transthyretin-mediated amyloidosis. The invention also relates to an in vitro or ex vivo method for transferring the conjugate from outside a cell to inside said cell or for transferring the effector molecule comprised by the conjugate of the invention from outside a cell to inside said cell, preferably to the cytosol of said cell.

### TECHNOLOGICAL FIELD

The invention relates to a conjugate for delivering an effector molecule from outside a cell into said cell, preferably into the cytosol and/or nucleus of said cell, the conjugate comprising at least one effector molecule to be transferred into the cell, preferably into the cytosol and/or nucleus of the cell, at least one saponin of the mono-desmosidic triterpene glycoside type or the bi-desmosidic triterpene glycoside type, and at least one single-domain antibody (sdAb), preferably at least one multivalent nanobody, preferably a bivalent sdAb tandem, covalently bound to each other, wherein the sdAb(s) is/are capable of binding to a cell-surface molecule of said cell, wherein the cell-surface molecule preferably is an endocytic cell-surface molecule such as a receptor on the cell. The effector molecule is typically an oligonucleotide. The invention also relates to a pharmaceutical composition comprising the conjugate of the invention. Furthermore, the invention relates to the pharmaceutical composition of the invention, for use as a medicament. In addition, the invention relates to a pharmaceutical composition of the invention, for use in the treatment or the prophylaxis of any one or more of: a cancer, an auto-immune disease such as rheumatoid arthritis, an enzyme deficiency, a disease related to an enzyme deficiency, a gene defect, a disease relating to a gene defect, an infection such as a viral infection, hypercholesterolemia, primary hyperoxaluria, haemophilia A, haemophilia B, alpha-1 antitrypsin related liver disease, acute hepatic porphyria, an amyloidosis and transthyretin-mediated amyloidosis. The invention also relates to an in vitro or ex vivo method for transferring the conjugate from outside a cell to inside said cell or for transferring the effector molecule comprised by the conjugate of the invention from outside a cell to inside said cell, preferably to the cytosol and/or nucleus of said cell.

### BACKGROUND

Molecules with a therapeutic biological activity are in many occasions in theory suitable for application as an effective therapeutic drug for the treatment of a disease such as a cancer in human patients in need thereof. A typical example are small-molecule biologically active moieties. However, many if not all potential drug-like molecules and therapeutics currently used in the clinic suffer from at least one of a plethora of shortcomings and drawbacks. When administered to a human body, therapeutically active molecules may exert off-target effects, in addition to the desired biological activity which is directed to the treatment of a disease or health problem. Such off-target effects are undesired and bear a risk for induction of health- or even life-threatening side effects of the administered molecule. It is the occurrence of such adverse events that cause many drug-like compounds and therapeutic moieties to fail phase Ill clinical trials or even phase IV clinical trials (post-authorisation surveillance). Therefore, there is a strong desire to provide drug molecules, wherein the therapeutic effect of the drug molecule should, e.g., (1) be highly specific for a biological factor or biological process driving the disease, (2) be sufficiently safe, (3) be sufficiently efficacious, (4) be sufficiently directed to the diseased cell with little to no off-target activity on non-diseased cells, (5) have a sufficiently timely mode of action (e.g. the administered drug molecule should reach the targeted site in the human patient within a certain time frame and should remain at the targeted site for a certain time frame), and/or (6) have sufficiently long lasting therapeutic activity in the patient's body, amongst others. Unfortunately, to date, ‘ideal’ therapeutics with many or even all of the beneficial characteristics (1)-(6) here above outlined, are not available to the patients, despite already long-lasting and intensive research and despite the progress made in several areas of the individually addressed encountered difficulties and drawbacks.

Chemotherapy is one of the most important therapeutic options for cancer treatment. However, it is often associated with a small therapeutic window because it has no specificity towards cancer cells over dividing cells in healthy tissue. The invention of monoclonal antibodies offered the possibility of exploiting their specific binding properties as a mechanism for the targeted delivery of cytotoxic agents to cancer cells, while sparing normal cells. This can be achieved by chemical conjugation of cytotoxic effectors (also known as effector molecules, effector moieties, payloads or warheads) to antibodies, to create antibody-drug conjugates (ADCs). Typically, very potent payloads such as emtansine (DM1) are used which have a limited therapeutic index (a ratio that compares toxic dose to efficacious dose) in their unconjugated forms. The conjugation of DM1 to trastuzumab (ado-trastuzumab emtansine), also known as Kadcycla, enhances the tolerable dose of DM1 at least two-fold in monkeys. In the past few decades tremendous efforts and investments have been made to develop therapeutic ADCs. However, it remains challenging to bring ADCs into the clinic, despite promising preclinical data. The first ADC approved for clinical use was gemtuzumab ozogamicin (Mylotarg, CD33 targeted, Pfizer/Wyeth) for relapsed acute myelogenous leukemia (AML) in 2000. Mylotarg was however, withdrawn from the market at the request of the Federal Drug Administration (FDA) due to a number of concerns including its safety profile. Patients treated with Mylotarg were more often found to die than patients treated with conventional chemotherapy. Mylotarg was admitted to the market again in 2017 with a lower recommended dose, a different schedule in combination with chemotherapy or on its own, and a new patient population. To date, only a few ADCs have been approved for clinical use, and meanwhile clinical development of several tens of ADCs has been halted. However, interest remains high and little less than 100 ADCs are still in clinical development in about five-hundred clinical trials.

Despite the potential to use toxic payloads that are normally not tolerated by patients, a low therapeutic index is a major problem accounting for the discontinuance of many ADCs in clinical development, which can be caused by several mechanisms such as off-target toxicity on normal cells, development of resistance against the cytotoxic agents and premature release of drugs in the circulation. A systematic review by the FDA of ADCs found that the toxicity profiles of most ADCs could be categorized according to the payload used, but not the antibody used, suggesting that toxicity is mostly determined by premature release of the payload. Of the ADCs that were discontinued, it is estimated that at least twenty-three were due to a poor therapeutic index. For example, development of a trastuzumab tesirine conjugate (ADCT-502, HER-2 targeted, ADC therapeutics) was discontinued due to a low therapeutic index, possibly due to an on-target, off-tissue effect in pulmonary tissue which expresses considerable levels of HER2. In addition, several ADCs in phase 3 trials have been discontinued due to missing primary endpoint. For example, phase 3 trials of a depatuxizumab mafodotin conjugate (ABT-414, EGFR targeted, AbbVie) tested in patients with newly diagnosed glioblastoma, and a mirvetuximab soravtansine conjugate (IMGN853, folate receptor alpha (FRα) targeted, ImmunoGen) tested in patients with platinum-resistant ovarian cancer, were stopped, showing no survival benefit. It is important to note that the clinically usable dose of some ADCs may not be sufficient for its full anticancer activity. For example, ado-trastuzumab emtansine has an MTD of 3.6 mg/kg in humans. In preclinical models of breast cancer, ado-trastuzumab emtansine induced tumor regression at dose levels at or above 3 mg/kg, but more potent efficacy was observed at 15 mg/kg. This suggests that at the clinically administered dose, ado-trastuzumab emtansine may not exert its maximal potential anti-tumor effect.

ADCs are mainly composed of an antibody, a cytotoxic moiety such as a payload, and a linker. Several novel strategies have been proposed and carried out in the design and development of new ADCs to overcome the existing problems, targeting each of the components of ADCs. For example, by identification and validation of adequate antigenic targets for the antibody component, by selecting antigens which have high expression levels in tumor and little or no expression in normal tissues, antigens which are present on the cell surface to be accessible to the circulating ADCs, and antigens which allows internalizing of ADCs into the cell after binding; and alternative mechanisms of activity; design and optimize linkers which enhance the solubility and the drug-to-antibody ratio (DAR) of ADCs and overcome resistance induced by proteins that can transport the chemotherapeutic agent out of the cells; enhance the DAR ratio by inclusion of more payloads, select and optimize antibodies to improve antibody homogeneity and developability. In addition to the technological development of ADCs, new clinical and translational strategies are also being deployed to maximize the therapeutic index, such as, change dosing schedules through fractionated dosing; perform biodistribution studies; include biomarkers to optimize patient selection, to capture response signals early and monitor the duration and depth of response, and to apply combination studies.

An example of ADCs with clinical potential are those ADCs such as brentuximab vedotin, inotuzumab ozogamicin, moxetumomab pasudotox, and polatuzumab vedotin, which are evaluated as a treatment option for lymphoid malignancies and multiple myeloma. Polatuzumab vedotin, binding to CD79b on (malignant) B-cells, and pinatuzumab vedotin, binding to CD22, are tested in clinical trials wherein the ADCs each were combined with co-administered rituximab, a monoclonal antibody binding to CD20 and not provided with a payload [B. Yu and D. Liu, Antibody-drug conjugates in clinical trials for lymphoid malignancies and multiple myeloma; Journal of Hematology & Oncology (2019) 12.94]. Combinations of monoclonal antibodies such as these examples are yet a further approach and attempt to arrive at the ‘magic bullet’ which combines many or even all of the aforementioned desired characteristics of ADCs.

Meanwhile in the past few decades, nucleic acid-based therapeutics are under development. Therapeutic nucleic acids can be based on deoxyribonucleic acid (DNA) or ribonucleic acid (RNA), Anti-sense oligonucleotides (ASOs, AONs), and short interfering RNAs (siRNAs), MicroRNAs, and DNA and RNA aptamers, for example, for approaches such as gene therapy, RNA interference (RNAi). Many of them share the same fundamental basis of action by inhibition of either DNA or RNA expression, thereby preventing expression of disease-related abnormal proteins. The largest number of clinical trials is being carried out in the field of gene therapy, with almost 2600 ongoing or completed clinical trials worldwide but with only about 4% entering phase 3. This is followed by clinical trials with ASOs. Similarly to ADCs, despite the large number of techniques being explored, therapeutic nucleic acids share two major issues during clinical development: delivery into target cells, more specifically for example into the cytosol of target (diseased) cells, and off-target effects. For instance, ASOs such as peptide nucleic acid (PNA), phosphoramidate morpholino oligomer (PMO), locked nucleic acid (LNA) and bridged nucleic acid (BNA), are being investigated as an attractive strategy to inhibit specifically target genes and especially those genes that are difficult to target with small molecules inhibitors or neutralizing antibodies. The efficacy of different ASOs is being studied in many neurodegenerative diseases such as Huntington's disease, Parkinson's disease, Alzheimer's disease, and amyotrophic lateral sclerosis and also in several cancer stages. The application of ASOs as potential therapeutic agents requires safe and effective methods for their delivery to the cytoplasm and/or nucleus of the target cells and tissues. Although the clinical relevance of ASOs has been demonstrated, inefficient cellular uptake, both in vitro and in vivo, limit the efficacy of ASOs and has been a barrier to therapeutic development. Cellular uptake can be <2% of the dose resulting in too low ASO concentration at the active site for an effective and sustained outcome. This consequently requires an increase of the administered dose which induces off-target effects. Most common side-effects are activation of the complement cascade, the inhibition of the clotting cascade and toll-like receptor mediated stimulation of the immune system.

Chemotherapeutics are most commonly small molecules, however, their efficacy is hampered by the severe off-target side toxicity, as well as their poor solubility, rapid clearance and limited tumor exposure. Scaffold-small-molecule drug conjugates such as polymer-drug conjugates (PDCs) are macromolecular constructs with pharmacologically activity, which comprises one or more molecules of a small-molecule drug bound to a carrier scaffold (e.g. polyethylene glycol (PEG)).

Such conjugate principle has attracted much attention and has been under investigation for several decades. The majority of conjugates of small-molecule drugs under pre-clinical or clinical development are for oncological indications. However, up-to-date only one drug not related to cancer has been approved (Movantik, a PEG oligomer conjugate of opioid antagonist naloxone, AstraZeneca) for opioid-induced constipation in patients with chronic pain in 2014, which is a non-oncology indication. Translating application of drug-scaffold conjugates into treatment of human subjects provides little clinical success so far. For example, PK1 (N-(2-hydroxypropyl)methacrylamide (HPMA) copolymer doxorubicin; development by Pharmacia, Pfizer) showed great anti-cancer activity in both solid tumors and leukaemia in murine models, and was under clinical investigation for oncological indications. Despite that it demonstrated significant reduction of nonspecific toxicity and improved pharmacokinetics in man, improvements in anticancer efficacy turned out to be marginal in patients, and as a consequence further development of PK1 was discontinued.

The failure of scaffold-small-molecule drug conjugates is at least partially attributed to its poor accumulation at the tumor site. For example, while in murine models PK1 showed 45-250 times higher accumulation in the tumor than in healthy tissues (liver, kidney, lung, spleen, and heart), accumulation in tumor was only observed in a small subset of patients in the clinical trial.

A potential solution to the aforementioned problems is application of nanoparticle systems for drug delivery such as liposomes, a technology sometimes referred to as ‘nanoplexing’. Liposomes are sphere-shaped vesicles consisting of one or more phospholipid bilayers, which are spontaneously formed when phospholipids are dispersed in water. The amphiphilicity characteristics of the phospholipids provide it with the properties of self-assembly, emulsifying and wetting characteristics, and these properties can be employed in the design of new drugs and new drug delivery systems. Drug encapsulation in a liposomal delivery system may convey several advantages over a direct administration of the drug, such as an improvement of and control over pharmacokinetics and pharmacodynamics, tissue targeting property, decreased toxicity and enhanced drug activity. An example of such success is liposome-encapsulated form of a small molecule chemotherapy agent doxorubicin (Doxil: a pegylated liposome-encapsulated form of doxorubicin; Myocet: a non-pegylated liposomal doxorubicin), which have been approved for clinical use.

A new field of drug discovery and drug development technology related to ADC and AOC was opened with the discovery of single domain antibodies, in particular the V_{HH} domains derived from camelid heavy-chain only antibodies. Application of a single domain antibody in development of for example cancer therapeutics is considered as a next-generation of antibody-derived tool. Compared to application of immunoglobulins such as IgG in design of ADCs and AOCs, single domain antibodies are recognized for their improved tissue penetration and for example tumor penetration when administered to the body. Also the beneficially higher solubility of a single domain antibody compared to IgG's is appreciated.

A solution still needs to be found that allows for drug therapies such as anti-tumor therapies, applicable for non-systemic use when desired, wherein the drug has for example an acceptable safety profile, little off-target activity, sufficient efficacy, sufficiently low clearance rate from the patient's body, sufficiently wide therapeutic window, etc.

In European patent EP1623715B1, a composition comprising a pharmacologically active agent coupled to a target-cell specific binding molecule such as an antibody or a fragment thereof, combined with a free saponin, has been described. The pharmacologically active agent is for example a toxin.

### SUMMARY

For an embodiment of the present invention, it is a first goal to provide an improved biologically active compound or composition comprising such improved biologically active compound.

As outlined here above in the Background, application of a single domain antibody in development of for example cancer therapeutics is considered as a next-generation of antibody-derived tool. However, drawbacks for single domain antibody-based ADCs are recognized: rapid (renal) clearance from the body compared with conventional IgG-based ADCs was apparent. Approaches to prolong the in vivo circulation are for example multimerization of single domain antibodies, fusion of single domain antibody to albumin or albumin binding domain (preferably serum albumin or preferably albumin binding protein capable of binding to serum albumin), fusion to an Fc domain or to IgG, or the conjugation of single domain antibody to a poly-ethylene glycol polymer. Furthermore, for the single-domain antibody based ADCs efficacy is hampered by the delicate balance between affinity for target cell epitopes and extend of (cancerous) tissue penetration. Too high affinity may hamper sufficient tissue penetration. Once the problem of too short blood circulation time and sufficient target (tumor) tissue penetration have been overcome, the single-domain antibody based ADC or AOC suffers from the aspect in common with IgG-based ADC and AOC: lysosomal degradation once the conjugate is taken up by the target cell. For the single-domain antibody based ADC, several approaches may be tested in order to improve intracellular efficacy of a payload conjugated with a single domain antibody in an ADC or AOC. For improving the delivery of a payload conjugated to a single domain antibody, an approach could be the conjugation of the single domain antibody with a cell-penetrating peptide. Furthermore, conjugating single domain antibody with (serum) albumin or with an albumin binding protein, may result in improved intracellular delivery of the single domain antibody with payload bound thereto since (serum) albumin has the ability to accumulate in tumors and in inflamed tissue, and in addition has the ability to escape from catabolism after cellular uptake. Furthermore, efficacy of a single-domain antibody based ADC or AOC may be hampered due to presence of lysosome-sensitive sites, resulting in lysosomal degradation after uptake of the conjugate by the target cell. Improving efficacy of the payload in a single-domain antibody based ADC or AOC may therefore rely on addressing the lysosome-sensitive sites by mutations in the amino-acid sequence. However, although the single domain antibody-based ADC and AOC field is assessing such approaches to attempt to improve efficacy, successful single domain antibody-based ADC or AOC on the market are limited up till now. It is therefore one of several objectives of embodiments of the current invention to provide a solution to the problem of current drugs being less efficacious than desired, when administered to human patients in need thereof.

It is one of several objectives of embodiments of the current invention to provide a solution to the problem of non-specificity, encountered when administering therapeutically active compounds to a human patient in need thereof. It is one of several objectives of embodiments of the current invention to provide a solution to the problem of drugs with non-optimal specificity for a biological factor or biological process driving a disease. It is one of several objectives of embodiments of the current invention to provide a solution to the problem of insufficient safety characteristics of current drugs, when administered to human patients in need thereof. It is one of several objectives of embodiments of the current invention to provide a solution to the problem of current drugs being not sufficiently directed to the diseased cell with little to no off-target activity on non-diseased cells, when administered to human patients in need thereof. It is one of several objectives of embodiments of the current invention to provide a solution to the problem that current drugs do not have a sufficiently timely mode of action (e.g. the administered drug molecule should reach the targeted site in the human patient within a certain time frame and should remain at the targeted site for a certain time frame), when administered to human patients in need thereof. It is one of several objectives of embodiments of the current invention to provide a solution to the problem that current drugs have not sufficiently long lasting therapeutic activity in the patient's body, when administered to human patients in need thereof.

At least one of the above objectives of embodiments of the invention is achieved by providing an antibody-drug conjugate (ADC) or an antibody-oligonucleotide (AOC) such as an antibody-BNA covalent complex, of the invention, comprising a cell-targeting moiety which is at least one, preferably at least two (bivalent), single-domain antibody/antibodies (sdAb(s)) such as a V_{HH} or a bivalent V_{HH}-V_{HH} tandem, and at least one saponin and at least one effector moiety such as a proteinaceous toxin (therewith providing an ADC) and/or a polynucleotide such as a BNA (therewith providing an AOC), the ADC provided with a covalently linked saponin and/or the AOC provided with a covalently linked saponin also suitable for use as a medicament, according to the invention. Driven by the presence of the covalently linked at least one saponin in the conjugate, the delivery of the effector moiety comprised by the conjugate, from outside the cell into said target cell and subsequently out of the endosome and/or lysosome and into the cytoplasm (cytosol) and/or into the nucleus, is enhanced and improved. Thereby, the effective amount of the effector moiety at the side of its disease-related target in the diseased target cell is increased. For example, the effective amount is an amount of a gene-silencing polynucleotide delivered in the cytoplasm of a target cell such as a tumor cell, sufficient for silencing the target gene in the tumor cell.

The present invention will be described with respect to particular embodiments but the invention is not limited thereto but only by the claims. The embodiments of the invention described herein can operate in combination and cooperation, unless specified otherwise.

An aspect of the invention relates to a conjugate for delivery of an effector molecule from outside a cell into said cell, preferably into the endosome and/or lysosome of said cell and preferably (subsequently) into the cytosol and/or into the nucleus of said cell, the conjugate comprising at least one saponin, at least one effector molecule and at least one, preferably at least two, single domain antibody (sdAb), preferably at least one multivalent nanobody, preferably at least one bivalent nanobody comprising two single domain antibodies (sdAbs), wherein the saponin, the effector molecule and the at least one sdAb are covalently bound together, wherein the at least one saponin is a triterpenoid saponin of the 12,13-dehydrooleanane type,

and wherein the at least one effector molecule is selected from: a pharmaceutically active substance (drug molecule), a toxin, an oligonucleotide, a peptide and a protein,
and wherein the at least one sdAb, preferably the at least one multivalent, preferably bivalent nanobody, targets a cell surface molecule that is present on the cell, preferably targets an endocytic receptor that is present on the cell.

An aspect of the invention relates to a conjugate for delivery of an effector molecule from outside a cell into said cell, preferably into the endosome and/or lysosome of said cell and preferably (subsequently) into the cytosol and/or into the nucleus of said cell, the conjugate comprising at least one saponin, at least one effector molecule and at least one, preferably at least two, single domain antibody (sdAb), preferably at least one multivalent nanobody, preferably at least one bivalent nanobody comprising two single domain antibodies (sdAbs), wherein the saponin, effector molecule and at least one sdAb are covalently bound together, wherein the at least one saponin is a triterpenoid saponin of 12,13-dehydrooleanane type, preferably with an aldehyde group at position C-23 of the aglycone core structure of the saponin, and preferably comprising an aglycone core structure selected from:
2alpha-hydroxy oleanolic acid;
16alpha-hydroxy oleanolic acid;
hederagenin (23-hydroxy oleanolic acid);
16alpha,23-dihydroxy oleanolic acid;
gypsogenin;
quillaic acid;
protoaescigenin-21(2-methylbut-2-enoate)-22-acetate;
23-oxo-barringtogenol C-21,22-bis(2-methylbut-2-enoate);
23-oxo-barringtogenol C-21(2-methylbut-2-enoate)-16,22-diacetate;
3,16,28-trihydroxy oleanan-12-en;
gypsogenic acid,
   and wherein the at least one effector molecule is selected from: a pharmaceutically active substance (drug molecule), a toxin, an oligonucleotide, a peptide and a protein,
and wherein the at least one sdAb, preferably the at least one multivalent, preferably bivalent nanobody, targets a cell surface molecule that is present on the cell, preferably targets an endocytic receptor that is present on the cell.

An aspect of the invention relates to a conjugate for delivery of an effector molecule from outside a cell into the cytosol and/or into the nucleus of said cell, the conjugate comprising at least one saponin, at least one effector molecule and at least one, preferably at least two, single domain antibody (sdAb), preferably at least one multivalent nanobody, preferably at least one bivalent nanobody comprising two single domain antibodies (sdAbs), wherein the saponin, effector molecule and sdAb or multivalent or bivalent nanobody are covalently bound together, wherein the at least one saponin is a triterpenoid saponin of 12,13-dehydrooleanane type comprising an aglycone core structure selected from:
2alpha-hydroxy oleanolic acid;
16alpha-hydroxy oleanolic acid;
hederagenin (23-hydroxy oleanolic acid);
16alpha,23-dihydroxy oleanolic acid;
gypsogenin;
quillaic acid;
protoaescigenin-21(2-methylbut-2-enoate)-22-acetate;
23-oxo-barringtogenol C-21,22-bis(2-methylbut-2-enoate);
23-oxo-barringtogenol C-21(2-methylbut-2-enoate)-16,22-diacetate;
3,16,28-trihydroxy oleanan-12-en;
gypsogenic acid,
   and wherein the at least one effector molecule is selected from: a pharmaceutically active substance (drug molecule), a toxin, an oligonucleotide, a peptide and a protein,
and wherein the at least one multivalent nanobody, preferably at least one bivalent nanobody or the at least one, preferably at least two, sdAb target a cell surface molecule that is present on the cell, preferably an endocytic receptor.

An aspect of the invention relates to a conjugate for delivery of an effector molecule from outside a cell into the cytosol of said cell, the conjugate comprising at least one saponin, at least one effector molecule and at least one multivalent nanobody, preferably at least one bivalent nanobody comprising two single domain antibodies (sdAbs), wherein the saponin, effector molecule and at least one sdAb are covalently bound together, wherein the at least one saponin is a triterpenoid saponin of 12,13-dehydrooleanane type comprising an aglycone core structure selected from:
2alpha-hydroxy oleanolic acid;
16alpha-hydroxy oleanolic acid;
hederagenin (23-hydroxy oleanolic acid);
16alpha,23-dihydroxy oleanolic acid;
gypsogenin;
quillaic acid;
protoaescigenin-21(2-methylbut-2-enoate)-22-acetate;
23-oxo-barringtogenol C-21,22-bis(2-methylbut-2-enoate);
23-oxo-barringtogenol C-21(2-methylbut-2-enoate)-16,22-diacetate;
3,16,28-trihydroxy oleanan-12-en;
gypsogenic acid,
   and wherein the at least one effector molecule is selected from: a pharmaceutically active substance, a toxin, an oligonucleotide, a peptide and a protein,
   and wherein the at least one multivalent, preferably the bivalent nanobody comprising two single domain antibodies (sdAbs) targets a cell surface molecule that is an endocytic cell-surface receptor present on the cell.

An aspect of the invention relates to a conjugate for delivery of an effector molecule from outside a first cell into the cytosol of said first cell, the conjugate comprising at least one saponin, at least one effector molecule and at least one single domain antibody (sdAb), wherein the saponin, the effector molecule and the at least one sdAb are covalently bound together, wherein the at least one saponin is a triterpenoid saponin of 12,13-dehydrooleanane type comprising an aglycone core structure selected from:
2alpha-hydroxy oleanolic acid;
16alpha-hydroxy oleanolic acid;
hederagenin (23-hydroxy oleanolic acid);
16alpha,23-dihydroxy oleanolic acid;
gypsogenin;
quillaic acid;
protoaescigenin-21(2-methylbut-2-enoate)-22-acetate;
23-oxo-barringtogenol C-21,22-bis(2-methylbut-2-enoate);
23-oxo-barringtogenol C-21(2-methylbut-2-enoate)-16,22-diacetate;
3,16,28-trihydroxy oleanan-12-en;
gypsogenic acid,
   and wherein the at least one effector molecule is selected from: a pharmaceutically active substance, a toxin, an oligonucleotide, a peptide and a protein,
   and wherein the at least one sdAb targets a first cell surface molecule that is present on the first cell,
for example, the conjugate comprises at least one multivalent nanobody, preferably at least one bivalent nanobody comprising two single domain antibodies (sdAbs).

In an embodiment, a conjugate as defined herein is provided wherein the conjugate comprises a further sdAb, which is different from the at least one sdAb, the further sdAb for binding to albumin, such as any one or more of the further sdAbs with an amino-acid sequence of SEQ ID NO: 33, 34 and 35, preferably the further sdAb is a V_{HH}, more preferably a camelid V_{H}. In an embodiment, a conjugate as defined herein is provided wherein the conjugate comprises at least two different sdAbs, such as a first sdAb as defined below, and a further sdAb for binding to (serum) albumin, such as any one or more of the further sdAbs with an amino-acid sequence of SEQ ID NO: 33, 34 and 35, preferably the further sdAb is a V_{HH}, more preferably a camelid V_{H}. Without wishing to be bound by any theory, it is believed that said further sdAb may extend the half-life of the conjugate. Hence, in an embodiment a conjugate as defined herein is provided wherein the conjugate further comprises an sdAb, which is different from the at least one sdAb, and which is capable of extending the half-life of the conjugate. In addition or alternatively, the conjugate may comprise albumin such as covalently bound albumin, and/or a (covalently linked) albumin binding protein. Alternatively, or in addition, the conjugate is provided with a half-life extending moiety different from albumin, an sdAb specific for binding to albumin or an albumin binding protein. Preferably, the albumin is serum albumin.

Preferably, in the conjugate the effector molecule is a single copy or multiple copies of an oligonucleotide, such as a single copy of an oligonucleotide. The cell-surface molecule preferably is an endocytic receptor on the target cell surface. The at least one sdAb are for example 1-10 sdAbs, comprising at least one multivalent nanobody, preferably at least one, more preferably one bivalent nanobody tandem capable of binding to a single type of cell surface molecule such as an endocytic receptor, preferably 1-8 or 1-6 or 2-4 or 3 sdAbs comprised by the conjugate. Typically, multiple sdAbs such as the two sdAbs of a bivalent nanobody in the conjugate are covalently linked together through peptide linkers, i.e. via peptide bonds.

An aspect of the invention relates to a conjugate for transferring an effector molecule from outside a cell into said cell, the conjugate comprising at least one effector molecule to be transferred into the cell, at least one single-domain antibody (sdAb) and at least one saponin, covalently bound to each other, directly or via at least one linker, wherein the at least one saponin is a mono-desmosidic triterpene glycoside or is a bi-desmosidic triterpene glycoside, and wherein the sdAb is capable of binding to a cell-surface molecule of said cell. If the conjugate comprises more than one sdAb, these sdAb's either bind to the same cell-surface molecule present on the same cell, that is to say to the same molecule or to different copies of the same type of cell-surface molecule, or these sdAb's bind to a first cell-surface molecule and to a second cell-surface molecule which is present at the same cell as the first cell-surface molecule. The cell-surface molecule(s) is/are typically (a) cell-surface receptor such as an endocytic receptor.

An aspect of the invention relates to a pharmaceutical composition comprising the conjugate of the invention, and optionally a pharmaceutically acceptable excipient and/or pharmaceutically acceptable diluent.

An aspect of the invention relates to a pharmaceutical composition of the invention, for use as a medicament.

An aspect of the invention relates to a pharmaceutical composition of the invention, for use in the treatment or the prophylaxis of any one or more of: a cancer, an auto-immune disease such as rheumatoid arthritis, an enzyme deficiency, a disease related to an enzyme deficiency, a gene defect, a disease relating to a gene defect, an infection such as a viral infection, hypercholesterolemia, primary hyperoxaluria, haemophilia A, haemophilia B, alpha-1 antitrypsin related liver disease, acute hepatic porphyria, an amyloidosis and transthyretin-mediated amyloidosis. An aspect of the invention relates to a pharmaceutical composition of the invention, for use in the treatment or the prophylaxis of any one or more of: a cancer, an auto-immune disease such as rheumatoid arthritis, an enzyme deficiency, a disease related to an enzyme deficiency, a gene defect, a disease relating to a gene defect, an infection such as a viral infection, hypercholesterolemia, primary hyperoxaluria, haemophilia A, haemophilia B, alpha-1 antitrypsin related liver disease, acute hepatic porphyria, an amyloidosis and transthyretin-mediated amyloidosis, preferably a cancer such as bladder cancer such as metastatic bladder cancer, urothelial carcinoma, cancer of the urinary tract, urologic neoplasms, prostate cancer such as castration resistant prostate cancer, breast cancer, malignant neoplasm of pancreas, ovary cancer, lung cancer such as non-squamous non-small cell lung cancer and squamous cell carcinoma of lung. The conjugate preferably comprises a bivalent nanobody or 1-4, such as 3 or 4 sdAbs, optionally comprising a bivalent nanobody. The conjugate preferably comprises at least one oligonucleotide. Preferably, the saponin is a saponin isolated from Saponaria Officinalis, such as SO1861, SO1832.

An aspect of the invention relates to an in vitro or ex vivo method for transferring the effector molecule of the invention (the effector molecule comprised by the conjugate of the invention) from outside a cell to inside said cell, preferably to the cytosol of said cell, comprising the steps of:
a) providing a cell which expresses on its cell surface the binding site for the at least one sdAb comprised by the conjugate of the invention, said binding site preferably being present on a cell-surface molecule of the cell, as described herein, said cell preferably being selected from a liver cell, an aberrant cell such as a virally infected cell, an auto-immune cell, a cell comprising a gene defect, a cell comprising an enzyme deficiency and a tumor cell;
b) providing the conjugate of the invention, said conjugate comprising the effector molecule to be transferred into the cell provided in step a); and
c) contacting the cell of step a) in vitro or ex vivo with the conjugate of step b),
   therewith effecting the transfer of said conjugate comprising the effector molecule from outside the cell to inside said cell, and by effecting the transfer of said conjugate effecting the transfer of the effector molecule from outside the cell to inside said cell, preferably into the cytosol of said cell.

An aspect of the invention relates to an in vitro or ex vivo method for transferring the conjugate of the invention from outside a cell to inside said cell, comprising the steps of:
a) providing a cell which expresses on its cell surface the binding site for the at least one sdAb comprised by the conjugate of the invention, said binding site preferably being present on a cell-surface molecule of the cell, as described herein, said cell preferably being selected from a liver cell, an aberrant cell such as a virally infected cell, an auto-immune cell, a cell comprising a gene defect, a cell comprising an enzyme deficiency and a tumor cell;
b) providing the conjugate of any one of the invention; and
c) contacting the cell of step a) in vitro or ex vivo with the conjugate of step b), therewith effecting the transfer of the conjugate from outside the cell to inside said cell.

An aspect of the invention relates to a kit of parts, comprising the conjugate of the invention or the pharmaceutical composition of the invention, and instructions for use of said conjugate or said pharmaceutical composition in the use for treatment or prophylaxis of any one or more of: a cancer, an auto-immune disease such as rheumatoid arthritis, an enzyme deficiency, a disease related to an enzyme deficiency, a gene defect, a disease relating to a gene defect, an infection such as a viral infection, hypercholesterolemia, primary hyperoxaluria, haemophilia A, haemophilia B, alpha-1 antitrypsin related liver disease, acute hepatic porphyria, an amyloidosis and transthyretin-mediated amyloidosis, preferably a cancer such as bladder cancer such as metastatic bladder cancer, urothelial carcinoma, cancer of the urinary tract, urologic neoplasms, prostate cancer such as castration resistant prostate cancer, breast cancer, malignant neoplasm of pancreas, ovary cancer, lung cancer such as non-squamous non-small cell lung cancer and squamous cell carcinoma of lung, or instructions for application of the in vitro or ex vivo methods according to the invention.

An aspect of the invention relates to a conjugate such as an ADC or an AOC, or to a semi-finished ADC conjugate or a semi-finished AOC conjugate, comprising a cell-surface molecule targeting molecule comprising at least an sdAb and preferably at least a bivalent sdAb, and comprising at least one effector moiety of the invention and/or comprising at least one saponin of the invention, of Structure C:

A(-S)_{b}(-E)_{c} (Structure C)

wherein A is the cell-surface molecule targeting molecule i.e. the one or more sdAb, preferably at least one bivalent sdAb (sdAb-sdAb tandem);
S is the saponin;
E is the effector moiety;
b=0-64, preferably 0, 1, 2, 3, 4, 8, 16, 32, 64 or any whole number (or fraction) therein between, preferably 1-8, more preferably 1, 2, 4 or 8, most preferably 1, 4 or 8 saponin moieties;
c=0-8, preferably 0, 1, 2, 3, 4, 6, 8 or any whole number (or fraction) therein between, preferably 1 or 2 copies of the same effector moiety or different effector moieties, more preferably a single copy of the effector moiety,
wherein S is coupled to A and/or to E, E is coupled to A and/or to S, preferably S is coupled to A and E is coupled to A, more preferably, S and E are coupled covalently to a trifunctional linker, wherein preferably the trifunctional linker is coupled to A. Optionally, more than one trifunctional linker each with the covalently bound one or more S and with the covalently bound E, are covalently bound to A, for example 1-4 of such trifunctional linkers which are functionalized with coupled A and E moieties, preferable 1-2, for example (on average) 1, 1.1, 1.2, 1.3, 1.4, 1.5, 1.6, 1.7, 1.8, 1.9 or 2 of such trifunctional linkers. Preferably, the A is at least a tandem of sdAbs, e.g. a bivalent sdAb such as a biparatopic sdAb. Typically, the conjugate comprises 1, 4 or 8 saponin moieties, or a multiple thereof when more than one (trifunctional) linker to which the saponin(s) are bound, are linked to the sdAb(s). For example, when on average 1.6 of such (trifunctional) linkers are bound to for example a bivalent sdAb, the number of saponin moieties in the conjugate would be 1.6, 6.4 and 12.8 when the (trifunctional) linker contains 1, 4 or 8 bound saponin moieties, respectively. Typically, the conjugate comprises a single copy of the effector moiety, or a multiple thereof when more than one (trifunctional) linker to which the effector moiety is bound, are linked to the sdAb(s). For example, when on average 1.6 of such (trifunctional) linkers are bound to for example a bivalent sdAb, the average number of effector moieties in the conjugate would be 1.6. It is to be understood that in the conjugate such bivalent sdAbs have for example a single linker or two linkers bound, these linkers each comprising the bound at least one saponin and the bound at least one effector moiety. The linker is typically a trifunctional linker. The at least one saponin is a saponin as claimed, preferably SO1861. The at least one effector moiety is an effector moiety as claimed, preferably an oligonucleotide. The at least one sdAb is preferably a bivalent sdAb or a string of 3-6 sdAb's preferably comprising at least one bivalent antibody. The binding partner for the at least one sdAb in the conjugate is for example an endocytic receptor present on the target cell, such as a tumor-cell specific receptor such as for example CD71 and EGFR, or is another receptor as claimed. For example, the (endocytic) receptor is CD63 (also known as LAMP-3).

### Definitions

The term “proteinaceous” has its regular scientific meaning and here refers to a molecule that is protein-like, meaning that the molecule possesses, to some degree, the physicochemical properties characteristic of a protein, is of protein, relating to protein, containing protein, pertaining to protein, consisting of protein, resembling protein, or being a protein. The term “proteinaceous” as used in for example ‘proteinaceous molecule’ refers to the presence of at least a part of the molecule that resembles or is a protein, wherein ‘protein’ is to be understood to include a chain of amino-acid residues at least two residues long, thus including a peptide, a polypeptide and a protein and an assembly of proteins or protein domains. In the proteinaceous molecule, the at least two amino-acid residues are for example bound via (an) amide bond(s), such as (a) peptide bond(s). In the proteinaceous molecule, the amino-acid residues are natural amino-acid residues and/or artificial amino-acid residues such as modified natural amino-acid residues. In a preferred embodiment, a proteinaceous molecule is a molecule comprising at least two amino-acid residues, preferably between two and about 2.000 amino-acid residues. In one embodiment, a proteinaceous molecule is a molecule comprising from 2 to 20 (typical for a peptide) amino acids. In one embodiment, a proteinaceous molecule is a molecule comprising from 21 to 1.000 (typical for a polypeptide, a protein, a protein domain, such as an antibody, a Fab, an scFv, a ligand for a receptor such as EGF) amino acids. Preferably, the amino-acid residues are (typically) bound via (a) peptide bond(s). According to the invention, said amino-acid residues are or comprise (modified) (non-)natural amino acid residues.

The term “effector molecule”, or “effector moiety” when referring to the effector molecule as part of e.g. a covalent conjugate, has its regular scientific meaning and here refers to a molecule that can selectively bind to for example any one or more of the target molecules: a protein, a peptide, a carbohydrate, a saccharide such as a glycan, a (phospho)lipid, a nucleic acid such as DNA, RNA, an enzyme, and that regulates the biological activity of such one or more target molecule(s). In the conjugate of the invention the effector moiety for example exerts its effect in the cytosol (cytoplasm) and/or in the cell nucleus, and/or is delivered intracellularly in the endosome and/or lysosome and/or is active after exiting or escaping the endosomal-lysosomal pathway (therewith entering the cytoplasm). The effector molecule is for example a molecule selected from any one or more of a small molecule such as a drug molecule, a toxin such as a protein toxin, an polynucleotide such as a BNA, a xeno nucleic acid or an siRNA, an enzyme, a peptide, a protein, or an active fragment or active domain thereof, or any combination thereof. Thus, for example, an effector molecule or an effector moiety is a molecule or moiety selected from any one or more of a small molecule such as a drug molecule, a toxin such as a protein toxin, an polynucleotide such as a BNA, a xeno nucleic acid or an siRNA, an enzyme, a peptide, a protein, or any combination thereof, that can selectively bind to any one or more of the target molecules: a protein, a peptide, a carbohydrate, a saccharide such as a glycan, a (phospho)lipid, a nucleic acid such as DNA, RNA, an enzyme, and that upon binding to the target molecule regulates the biological activity of such one or more target molecule(s). For example, an effector moiety is a toxin or an active toxic fragment thereof or an active toxic derivative or an active toxic domain thereof. Typically, an effector molecule can exert a biological effect inside a cell such as a mammalian cell such as a human cell, such as in the cytosol of said cell or in the nucleus of said cell. An effector molecule or moiety of the invention is thus any substance that affects the metabolism of a cell by interaction with an intracellular effector molecule target, wherein this effector molecule target is any molecule or structure inside cells excluding the lumen of compartments and vesicles of the endocytic and recycling pathway but including the membranes of these compartments and vesicles. Said structures inside cells thus include the nucleus, mitochondria, chloroplasts, endoplasmic reticulum, Golgi apparatus, other transport vesicles, the inner part of the plasma membrane and the cytosol. Typical effector molecules are thus drug molecules, an enzyme, plasmid DNA, toxins such as toxins comprised by antibody-drug conjugates (ADCs), polynucleotides such as siRNA, BNA, nucleic acids comprised by an antibody-polynucleotide conjugate (AOC). For example, an effector molecule/moiety is a molecule which can act as a ligand that can increase or decrease (intracellular) enzyme activity, gene expression (e.g. gene silencing), or cell signalling. Typically, an effector moiety comprised by the conjugate exerts its therapeutic (for example toxic, enzymatic, inhibitory, gene silencing, etc.) effect in the cytosol and/or in the cell nucleus. Typically, the effector moiety is delivered intracellularly in the endosome and/or in the lysosome, and typically the effector moiety is active after exiting or escaping the endosomal-lysosomal pathway.

The term “saponin” has its regular scientific meaning and here refers to a group of amphiphatic glycosides which comprise one or more hydrophilic glycone moieties combined with a lipophilic aglycone core which is a sapogenin. The saponin may be naturally occurring or synthetic (i.e. non-naturally occurring). The term “saponin” includes naturally-occurring saponins, functional derivatives of naturally-occurring saponins as well as saponins synthesized de novo through chemical and/or biotechnological synthesis routes. Saponin according to the conjugate of the invention has a triterpene backbone, which is a pentacyclic C30 terpene skeleton, also referred to as sapogenin or aglycone. Within the conjugate of the invention saponin is not considered an effector molecule nor an effector moiety in the conjugates according to the invention. Thus, in the conjugates comprising a saponin and an effector moiety, the effector moiety is a different molecule than the conjugated saponin. In the context of the conjugate of the invention, the term saponin refers to those saponins which exert an endosomal/lysosomal escape enhancing activity, when present in the endosome and/or lysosome of a mammalian cell such as a human cell, towards an effector moiety comprised by the conjugate of the invention and present in said endosome/lysosome together with the saponin.

The term “saponin derivative” (also known as “modified saponin”) has its regular scientific meaning and here refers to a compound corresponding to a naturally-occurring saponin (with endosomal/lysosomal escape enhancing activity towards an effector molecule, when present together in the endosome or lysosome of a mammalian cell) which has been derivatised by one or more chemical modifications, such as the oxidation of a functional group, the reduction of a functional group and/or the formation of a covalent bond with another molecule (also referred to as “conjugation” or as “covalent conjugation”). Preferred modifications include derivatisation of an aldehyde group of the aglycone core; of a carboxyl group of a saccharide chain or of an acetoxy group of a saccharide chain. Typically, the saponin derivative does not have a natural counterpart, i.e. the saponin derivative is not produced naturally by e.g. plants or trees. The term “saponin derivative” includes derivatives obtained by derivatisation of naturally-occurring saponins as well as derivatives synthesized de novo through chemical and/or biotechnological synthesis routes resulting in a compound corresponding to a naturally-occurring saponin which has been derivatised by one or more chemical modifications. A saponin derivative in the context of the invention should be understood as a saponin functional derivative. “Functional” in the context of the saponin derivative is understood as the capacity or activity of the saponin or the saponin derivative to enhance the endosomal escape of an effector molecule which is contacted with a cell together with the saponin or the saponin derivative.

The term “aglycone core structure” has its regular scientific meaning and here refers to the aglycone core of a saponin without the one or two carbohydrate antenna or saccharide chains (glycans) bound thereto. For example, quillaic acid is the aglycone core structure for SO1861, QS-7 and QS21. Typically, the glycans of a saponin are mono-saccharides or oligo-saccharides, such as linear or branched glycans.

The term “saccharide chain” has its regular scientific meaning and here refers to any of a glycan, a carbohydrate antenna, a single saccharide moiety (mono-saccharide) or a chain comprising multiple saccharide moieties (oligosaccharide, polysaccharide). The saccharide chain can consist of only saccharide moieties or may also comprise further moieties such as any one of 4E-Methoxycinnamic acid, 4Z-Methoxycinnamic acid, and 5-O-[5-O-Ara/Api-3,5-dihydroxy-6-methyl-octanoyl]-3,5-dihydroxy-6-methyl-octanoic acid), such as for example present in QS-21.

The term “Api/Xyl-” or “Api- or Xyl-” in the context of the name of a saccharide chain has its regular scientific meaning and here refers to the saccharide chain either comprising an apiose (Api) moiety, or comprising a xylose (Xyl) moiety.

As used herein, the terms “nucleic acid”, “oligonucleotide” and “polynucleotide” are synonymous to one another and are to be construed as encompassing any polymeric molecule made of units, wherein a unit comprises a nucleobase (or simply “base” e.g. being a canonical nucleobase like adenine (A), cytosine (C), guanine (G), thymine (T), or uracil (U), or any known non-canonical, modified, or synthetic nucleobase like 5-methylcytosine, 5-hydroxymethylcytosine, xanthine, hypoxanthine, 7-methylguanine; 5,6-dihydrouracil etc.) or a functional equivalent thereof, which renders said polymeric molecule capable of engaging in hydrogen bond-based nucleobase pairing (such as Watson-Crick base pairing) under appropriate hybridisation conditions with naturally-occurring nucleic acids such as deoxyribonucleic acid (DNA) or ribonucleic acid (RNA), which naturally-occurring nucleic acids are to be understood being polymeric molecules made of units being nucleotides.

Hence, from a chemistry perspective, the term nucleic acid under the present definition can be construed as encompassing polymeric molecules that chemically are DNA or RNA, as well as polymeric molecules that are nucleic acid analogues, also known as xeno nucleic acids (XNA) or artificial nucleic acids, which are polymeric molecules wherein one or more (or all) of the units are modified nucleotides or are functional equivalents of nucleotides. Nucleic acid analogues are well known in the art and due to various properties, such as improved specificity and/or affinity, higher binding strength to their target and/or increased stability in vivo, they are extensively used in research and medicine. Typical examples of nucleic acid analogues include but are not limited to locked nucleic acid (LNA) (that is also known as bridged nucleic acid (BNA)), phosphorodiamidate morpholino oligomer (PMO also known as Morpholino), peptide nucleic acid (PNA), glycol nucleic acid (GNA), threose nucleic acid (TNA), hexitol nucleic acid (HNA), 2′-deoxy-2′-fluoroarabinonucleic acid (FANA or FNA), 2′-deoxy-2′-fluororibonucleic acid (2′-F RNA or FRNA); altritol nucleic acids (ANA), cyclohexene nucleic acids (CeNA) etc.

In accordance with the cannon, length of a nucleic acid is expressed herein the number of units from which a single strand of a nucleic acid is build. Because each unit corresponds to exactly one nucleobase capable of engaging in one base pairing event, the length is frequently expressed in so called “base pairs” or “bp” regardless whether the nucleic acid in question is a single stranded (ss) or double stranded (ds) nucleic acid. In naturally-occurring nucleic acids 1 bp corresponds to 1 nucleotide, abbreviated to 1 nt. For example, a single stranded nucleic acid made of 1000 nucleotides (or a double stranded nucleic acid made of two complementary strands each of which is made of 1000 nucleotides) is described as having a length of 1000 base pairs or 1000 bp, which length can also be expressed as 1000 nt or as 1 kilobase that is abbreviated to 1 kb. 2 kilobases or 2 kb are equal to the length of 2000 base pair which equates 2000 nucleotides of a single stranded RNA or DNA. To avoid confusion however, in view of the fact the nucleic acids as defined herein may comprise or consist of units not only chemically being nucleotides but also being functional equivalents thereof, the length of nucleic acids will preferentially be expressed herein in “bp” or “kb” rather than in the equally common in the art denotation “nt”.

In advantageous embodiments, the nucleic acid as disclosed herein are no longer than 1 kb, preferably no longer than 500 bp, most preferably no longer than 250 bp.

In particularly advantageous embodiments, the nucleic acid is an oligonucleotide (or simply an oligo) defined as nucleic acid being no longer than 100 bp, i.e. in accordance with the above provided definition, being any polymeric molecule made of no more than 100 units, wherein each unit comprises a nucleobase or a functional equivalent thereof, which renders said oligonucleotide capable of engaging in hydrogen bond-based nucleobase pairing under appropriate hybridisation conditions with DNA or RNA. Within the ambit of said definition, it will immediately be appreciated that the disclosed herein oligonucleotides can comprise or consist of units not only being nucleotides but also being synthetic equivalents thereof. In other words, from a chemistry perspective, as used herein the term oligonucleotide will be construed as possibly comprising or consisting of RNA, DNA, or a nucleic acid analogue such as but not limited to LNA (BNA), PMO (Morpholino), PNA, GNA, TNA, HNA, FANA, FRNA, ANA, CeNA and/or the like. For example, PMO is preferred or for example, PNA is preferred. For example, PS-ASO is preferred. Preferably, the oligonucleotide is any of PMO, PNA, PS-ASO, more preferred is PMO.

The term “antibody-drug conjugate” or “ADC” has its regular scientific meaning and here refers to any conjugate of an antibody such as an IgG, a Fab, an scFv, an immunoglobulin, an immunoglobulin fragment, one or multiple V_{H} domains, single-domain antibodies, a V_{HH}, a camelid V_{H}, etc., and any molecule that can exert a therapeutic effect when contacted with cells of a subject such as a human patient, such as an active pharmaceutical ingredient, a toxin, an oligonucleotide, an enzyme, a small molecule drug compound, etc., in general referred to as an effector moiety.

The term “antibody-oligonucleotide conjugate” or “AOC” has its regular scientific meaning and here refers to any conjugate of an antibody such as an IgG, a Fab, an scFv, an immunoglobulin, an immunoglobulin fragment, one or multiple V_{H} domains, single-domain antibodies, a V_{HH}, a camelid V_{H}, etc., and any polynucleotide (oligonucleotide) molecule that can exert a therapeutic effect when contacted with cells of a subject such as a human patient, such as an oligonucleotide selected from a natural or synthetic string of nucleic acids encompassing DNA, modified DNA, RNA, mRNA, modified RNA, synthetic nucleic acids, presented as a single-stranded molecule or a double-stranded molecule, such as a BNA, an antisense oligonucleotide (ASO, AON), a short or small interfering RNA (siRNA; silencing RNA), an anti-sense DNA, anti-sense RNA, etc.

The term “bridged nucleic acid”, or “BNA” in short, or “locked nucleic acid” or “LNA” in short or 2′-O,4-C-aminoethylene or 2′-O,4′-C-aminomethylene bridged nucleic acid (BNA^{NC}), has its regular scientific meaning and here refers to a modified RNA nucleotide. A BNA is also referred to as ‘constrained RNA molecule’ or ‘inaccessible RNA molecule’. A BNA monomer can contain a five-membered, six-membered or even a seven-membered bridged structure with a “fixed” C₃′-endo sugar puckering. The bridge is synthetically incorporated at the 2′, 4′-position of the ribose to afford a 2′, 4′-BNA monomer. A BNA monomer can be incorporated into an oligonucleotide polymeric structure using standard phosphoramidite chemistry known in the art. A BNA is a structurally rigid oligonucleotide with increased binding affinity and stability.

The term “single domain antibody”, or “sdAb”, in short, or ‘nanobody’, has its regular scientific meaning and here refers to an antibody fragment consisting of a single monomeric variable antibody domain, unless referred to as more than one monomeric variable antibody domain such as for example in the context of a bivalent sdAb, which comprises two of such monomeric variable antibody domains in tandem. In the conjugates of the invention, more than one sdAb can be present, which sdAb's can be the same (multivalent and mono-specific) or can be different (multivalent and/or for example multi-paratope, bi-paratope, multi-specific, bi-specific). In addition, for example the more than two sdAb's are for example a combination of mono-specific and multivalent sdAb's and at least one further sdAb that binds to a different epitope (e.g. multispecific or biparatope). Additionally, for example at least one multivalent nanobody comprises multiple sdAbs which multiple sdAbs comprise at least two different sdAbs.

The term “compete” here refers to the binding of a first “single domain antibody”, or “sdAb”, in short, or ‘nanobody’, to the same epitope or to overlapping epitopes on for example a cell-surface molecule, to which for example a second sdAb or an immunoglobulin also can bind, such that either the first sdAb will be bound to the cell-surface molecule or the second sdAb or immunoglobulin will be bound to the cell-surface molecule, when the combination of the first and second sdAb or the combination of the first sdAb and the immunoglobulin are contacted with the cell-surface molecule.

A bivalent nanobody is a molecule comprising two single domain antibodies targeting epitopes on molecules present at the extracellular side of a cell, such as epitopes on the extracellular domain of a cell surface molecule that is present on the cell. Preferably the cell-surface molecule is a cell-surface receptor. A bivalent nanobody is also named a bivalent single domain antibody. Preferably the two different single domain antibodies are directly covalently bound or covalently bound through an intermediate molecule that is covalently bound to the two different single domain antibodies. Preferably the intermediate molecule of the bivalent nanobody has a molecular weight of less than 10,000 Dalton, more preferably less than 5000 Dalton, even more preferably less than 2000 Dalton, most preferably less than 1500 Dalton.

Preferably the two single domain antibodies of the bivalent nanobody do not bind to the same copy of the cell surface molecule present on a cell but bind to different copies of that cell surface molecule present on the same cell. It is believed that binding of the bivalent nanobody to different copies further enhances the uptake (endocytosis) of the nanobody in the cell, or when comprised in a conjugate, it is believed that binding of the bivalent nanobody to different copies on the same cell further enhances the uptake of the conjugate in the cell. This further enhancement may be due to the cross-linking of two cell surface molecules by the bivalent nanobody, which cross-linking is believed to stimulate the uptake. Furthermore, it is believed that improved uptake and internalization enhances endosomal and lysosomal delivery of the bivalent nanobody or the conjugate comprising the bivalent nanobody.

In a different embodiment preferably the two different single domain antibodies of the hetero-bivalent nanobody bind to the same copy of the cell surface molecule present on a cell.

A homo-bivalent nanobody is a bivalent nanobody wherein each of the two single domain antibodies target the same epitope on the extracellular cell-surface molecule, such the extracellular domain of a cell surface molecule that is present on a cell. A homo-bivalent nanobody is also named a homo-bivalent single domain antibody.

A hetero-bivalent nanobody, here also named a biparatopic nanobody, is a bivalent nanobody wherein the two single domain antibodies target different, non-overlapping epitopes on the extracellular domain of a cell surface molecule that is present on a cell. A hetero-bivalent nanobody is also named a hetero-bivalent single domain antibody and is also named a biparatopic single domain antibody or biparatopic nanobody. Preferably, the (first and/or second) cell surface molecule is an endocytic cell-surface receptor. Preferably, the non-overlapping epitopes are located on the same copy of the (first) cell surface molecule.

A conjugate is a combination of two or more different molecules that have been and are covalently bound. The different molecules of the conjugate for this invention comprise one or more saponins, one or more effector molecules, one or more (bivalent) nanobodies, preferably a single bivalent nanobody molecule comprising two single domain antibodies, more preferably a bi-paratopic sdAb, and optionally though preferably one or more intermediate molecules such as linkers linking the two or more different molecules covalently together, such as for example via linking to a central further linker. In a conjugate, not all of the two or more, such as three, different molecules need to be directly covalently bound to each other. Different molecules in the conjugate may also be covalently bound by being both covalently bound to the same intermediate molecule such as a linker or each by being covalently bound to an intermediate molecule such as a further linker wherein these two intermediate molecules such as two (different) linkers, are covalently bound to each other. According to this definition even more intermediate molecules, such as linkers, may be present between the two different molecules in the conjugate as long as there is a chain of covalently bound atoms in between.

The term ‘S’ as used in the context such as in an antibody-saponin conjugate comprising a linker, represents ‘stable linker’ which remains intact in the endosome and in the lysosome of mammalian cells, such as human cells, such as a human tumor cell, thus under slightly acid conditions (pH<6.6, such as pH 4.0-5.5).

The term ‘L’ as used in the context such as in an antibody-saponin conjugate comprising a linker, represents ‘labile linker’ which is cleaved under slightly acid conditions (pH<6.6, such as pH 4.0-5.5) in the endosome and in the lysosome of mammalian cells, such as human cells, such as a human tumor cell.

The terms first, second, third and the like in the description and in the claims, are used for distinguishing between for example similar elements, compositions, constituents in a composition, or separate method steps, and not necessarily for describing a sequential or chronological order. The terms are interchangeable under appropriate circumstances and the embodiments of the invention can operate in other sequences than described or illustrated herein, unless specified otherwise.

The embodiments of the invention described herein can operate in combination and cooperation, unless specified otherwise.

Furthermore, the various embodiments, although referred to as “preferred” or “e.g.” or “for example” or “in particular” and the like are to be construed as exemplary manners in which the invention may be implemented rather than as limiting the scope of the invention.

The term “comprising”, used in the claims, should not be interpreted as being restricted to for example the elements or the method steps or the constituents of a compositions listed thereafter, it does not exclude other elements or method steps or constituents in a certain composition. It needs to be interpreted as specifying the presence of the stated features, integers, (method) steps or components as referred to, but does not preclude the presence or addition of one or more other features, integers, steps or components, or groups thereof. Thus, the scope of the expression “a method comprising steps A and B” should not be limited to a method consisting only of steps A and B, rather with respect to the present invention, the only enumerated steps of the method are A and B, and further the claim should be interpreted as including equivalents of those method steps. Thus, the scope of the expression “a composition comprising components A and B” should not be limited to a composition consisting only of components A and B, rather with respect to the present invention, the only enumerated components of the composition are A and B, and further the claim should be interpreted as including equivalents of those components.

In addition, reference to an element or a component by the indefinite article “a” or “an” does not exclude the possibility that more than one of the element or component are present, unless the context clearly requires that there is one and only one of the elements or components. The indefinite article “a” or “an” thus usually means “at least one”.

The term “Saponinum album” has its normal meaning and here refers to a mixture of saponins produced by Merck KGaA (Darmstadt, Germany) containing saponins from Gypsophila paniculata and Gypsophila arostii, containing SA1657 and mainly SA1641.

The term “Quillaja saponin” has its normal meaning and here refers to the saponin fraction of Quillaja saponaria and thus the source for all other QS saponins, mainly containing QS-18 and QS-21.

“QS-21” or “QS21” has its regular scientific meaning and here refers to a mixture of QS-21A-apio (˜63%), QS-21A-xylo (˜32%), QS-21 B-apio (˜3.3%), and QS-21 B-xylo (˜1.7%).

Similarly, “QS-21A” has its regular scientific meaning and here refers to a mixture of QS-21A-apio (˜65%) and QS-21A-xylo (˜35%).

Similarly, “QS-21 B” has its regular scientific meaning and here refers to a mixture of QS-21 B-apio (˜65%) and QS-21 B-xylo (˜35%).

The term “Quil-A” refers to a commercially available semi-purified extract from Quillaja saponaria and contains variable quantities of more than 50 distinct saponins, many of which incorporate the triterpene-trisaccharide substructure Gal-(1→2)-[Xyl-(1→3)]-GlcA- at the C-3beta-OH group found in QS-7, QS-17, QS18, and QS-21. The saponins found in Quil-A are listed in van Setten (1995), Table 2 [Dirk C. van Setten, Gerrit van de Werken, Gijsbert Zomer and Gideon F. A. Kersten, Glycosyl Compositions and Structural Characteristics of the Potential Immuno-adjuvant Active Saponins in the Quillaja saponaria Molina Extract Quil A, RAPID COMMUNICATIONS IN MASS SPECTROMETRY, VOL. 9,660-666 (1995)]. Quil-A and also Quillaja saponin are fractions of saponins from Quillaja saponaria and both contain a large variety of different saponins with largely overlapping content. The two fractions differ in their specific composition as the two fractions are gained by different purification procedures.

The term “QS1861” and the term “QS1862” refer to QS-7 and QS-7 api. QS1861 has a molecular mass of 1861 Dalton, QS1862 has a molecular mass of 1862 Dalton. QS1862 is described in Fleck et al. (2019) in Table 1, row no. 28 [Juliane Deise Fleck, Andresa Heemann Betti, Francini Pereira da Silva, Eduardo Artur Troian, Cristina Olivaro, Fernando Ferreira and Simone Gasparin Verza, Saponins from Quillaja saponaria and Quillaja brasiliensis: Particular Chemical Characteristics and Biological Activities, Molecules 2019, 24, 171; doi:10.3390/molecules24010171]. The described structure is the api-variant QS1862 of QS-7. The molecular mass is 1862 Dalton as this mass is the formal mass including proton at the glucuronic acid. At neutral pH, the molecule is deprotonated. When measuring in mass spectrometry in negative ion mode, the measured mass is 1861 Dalton.

The terms “SO1861” and “SO1862” refer to the same saponin of Saponaria officinalis, though in deprotonated form or api form, respectively. The molecular mass is 1862 Dalton as this mass is the formal mass including a proton at the glucuronic acid. At neutral pH, the molecule is deprotonated. When measuring the mass using mass spectrometry in negative ion mode, the measured mass is 1861 Dalton.

### BRIEF DESCRIPTION OF THE DRAWINGS

FIG. 1A (FIG. 1A): The targeted 2-component approach (1 target). SO1861 and toxin (e.g. ribosomal inactivating protein) are each, separately, conjugated to a V_{HH} or antibody (mAb) for delivery and internalization into target cells. 1) mAb-toxin and V_{HH}-SO1861 bind to the cell surface receptor, 2) receptor-mediated endocytosis of both conjugates occurs (binding of conjugates to the receptor is followed by internalization of the conjugate/receptor complex), 3) at low endolysosomal pH and appropriate concentration, SO1861 becomes active to enable endolysosomal escape, 4) release of toxin into cytoplasm occurs and 5) toxin induces cell death.

FIG. 1B (FIG. 1B): The targeted 2-component approach (2 targets). SO1861 and toxin (ribosomal inactivating protein) are each, separately, conjugated to a V_{HH} or antibody (mAb) for delivery and internalization into target cells. 1) mAb-toxin and V_{HH}-SO1861 bind to their corresponding cell surface receptor, 2) receptor-mediated endocytosis of both conjugates occurs (binding of conjugates to the receptor is followed by internalization of the conjugate/receptor complex), 3) at low endolysosomal pH and appropriate concentration, SO1861 becomes active to enable endolysosomal escape, 4) release of toxin into cytoplasm occurs and 5) toxin induces cell death.

FIG. 1C (FIG. 1C): The targeted 2-component approach (2 targets). SO1861 and toxin (ribosomal inactivating protein) are each, separately, conjugated to a V_{HH} for delivery and internalization into target cells. 1) V_{HH}-toxin and V_{HH}-SO1861 bind to their corresponding cell surface receptor, 2) receptor-mediated endocytosis of both conjugates occurs (binding of conjugates to the receptor is followed by internalization of the conjugate/receptor complex), 3) at low endolysosomal pH and appropriate concentration, SO1861 becomes active to enable endolysosomal escape, 4) release of toxin into cytoplasm occurs and 5) toxin induces cell death.

FIG. 1D (FIG. 1D): The targeted 2-component approach (2 targets). SO1861 and toxin (ribosomal inactivating protein) are each, separately, conjugated to a V_{HH} or mAb for delivery and internalization into target cells. 1) V_{HH}-toxin and mAb-SO1861 bind to their corresponding cell surface receptor, 2) receptor-mediated endocytosis of both conjugates occurs (binding of conjugates to the receptor is followed by internalization of the conjugate/receptor complex), 3) at low endolysosomal pH and appropriate concentration, SO1861 becomes active to enable endolysosomal escape, 4) release of toxin into cytoplasm occurs and 5) toxin induces cell death.

FIG. 1E (FIG. 1E): The targeted 2-component approach (1 target). SO1861 and toxin (ribosomal inactivating protein) are each, separately, conjugated to a V_{HH} or antibody (mAb) for delivery and internalization into target cells. 1) V_{HH}-toxin and mAb-SO1861 bind to the cell surface receptor, 2) receptor-mediated endocytosis of both conjugates occurs (binding of conjugates to the receptor is followed by internalization of the conjugate/receptor complex), 3) at low endolysosomal pH and appropriate concentration, SO1861 becomes active to enable endolysosomal escape, 4) release of toxin into cytoplasm occurs and 5) toxin induces cell death.

FIG. 1F (FIG. 1F): Cartoon displaying an exemplifying molecule and conjugate of the present invention. Shown is an IgG antibody covalently conjugated with four saponin molecules ‘S’, bound to the light chains of the antibody, and with for effector molecules ‘E’ that are covalently bound to the constant domains of the heavy chain of the antibody.

FIG. 1G (FIG. 1G): Cartoon displaying an exemplifying molecule and conjugate of the present invention. Shown is an IgG antibody covalently conjugated with four trivalent linkers, wherein each linker is covalently bound to a saponin and is covalently bound to an effector molecule. The trivalent linkers are covalently bound to the antibody light chains.

FIG. 1H (FIG. 1H): Cartoon displaying an exemplifying molecule and conjugate of the present invention. Shown is a single domain antibody covalently conjugated with two trivalent linkers, wherein each linker is covalently bound to a saponin and is covalently bound to an effector molecule.

FIG. 1I (FIG. 1I): Cartoon displaying an exemplifying molecule and conjugate of the present invention. Shown is a single domain antibody covalently conjugated with a trivalent linker, wherein the trivalent linker is covalently bound to a saponin and is covalently bound to an effector molecule.

FIG. 1J (FIG. 1J): (S)n-(L)(E) concept: mAb-(SO1861)ⁿ(protein toxin)^{m}. Both, SO1861 covalently linked at the cysteine residues (Cys) and protein toxin (ribosomal inactivating protein) at the lysine residues are conjugated to the same antibody (mAb) for delivery and internalization into the target cells. 1) mAb-(Cys-L-SO1861)⁴(Lys-protein toxin)² bind to its corresponding cell surface receptor, 2) receptor-mediated endocytosis the conjugate occurs (binding of conjugates to the receptor is followed by internalization of the conjugate/receptor complex), 3) at low endolysosomal pH and appropriate concentration, SO1861 becomes active to enable endolysosomal escape, 4) release of toxin into cytoplasm occurs and 5) toxin induces cell death

FIG. 1K (FIG. 1K): (S)n-(L)(E) concept: mAb-(SO1861)ⁿ(antisense BNA oligo)m. Both, SO1861, bound to the cysteine residues (Cys) and the antisense BNA oligonucleotide bound to the lysine residues are conjugated to the same antibody (mAb) for delivery and internalization into the target cells. 1) mAb-(Cys-SO1861)⁴(Lys-BNAoligo)² binds to its corresponding cell surface receptor, 2) receptor-mediated endocytosis of both conjugates occurs (binding of conjugates to the receptor is followed by internalization of the conjugate/receptor complex), 3) at low endosomal, lysosomal and endolysosomal pH and appropriate concentration, SO1861 becomes active to enable endosomal, lysosomal and endolysosomal escape, 4) release of BNA oligo into cytoplasm occurs and 5) target gene silencing is induced.

FIG. 1L (FIG. 1L): (S)n-(L)(E) concept: mAb-(SO1861-scaffold-antisense BNA oligo)ⁿ. the (SO1861-trifunctional linker-BNAoligo)ⁿ is conjugated to an antibody (mAb) for delivery and internalization into the target cells. The antibody is for example an IgG, or an sdAb such as a V_{HH}. 1) mAb-(SO1861-trifunctional linker-BNAoligo)⁴ binds to its corresponding cell surface receptor, 2) receptor-mediated endocytosis of both conjugates occurs (binding of conjugates to the receptor is followed by internalization of the conjugate/receptor complex), 3) at low endolysosomal pH and appropriate concentration, SO1861 becomes active to enable endolysosomal escape, 4) release of BNA oligo into cytoplasm occurs and 5) target gene silencing is induced. The term “scaffold” in the context of the conjugates of the invention is to be understood as an oligomeric molecule or polymeric molecule bearing one or multiple chemical groups for covalent binding to one or multiple further molecule(s) such as saponin molecules and/or effector molecules such as a protein toxin or an oligonucleotide, and bearing at least one chemical group for covalent coupling to a protein such as an antibody, such as an IgG or an sdAb.

FIG. 1M (FIG. 1M): Single component or 1-component Bivalent V_{HH}-trifunctional linker-dendron(saponin)⁴(oligonucleotide)¹ concept. Four SO1861 moieties are covalently bound to a G2 dendron having four binding sites for saponin moieties, and the dendron is covalently bound to an arm of the central trifunctional linker. A single copy of the BNA is covalently coupled to another arm of the trifunctional linker. Yet, the third arm of the trifunctional linker is covalently bound to the bivalent V_{HH} tandem. Herewith, the 1-component conjugate is provided.

FIG. 2 (FIG. 2): Bivalent V_{HH}_EGFR-TFL-dendron(L-SO1861)4(L-HSP27BNA) (DAR1), combination therapies and controls were tested for cell viability and enhanced HSP27 gene silencing in A431 cells (EGFR⁺⁺) and A2058 (EGFR⁻) cells. This revealed in A431 cells (EGFR⁺⁺) that the bivalent V_{HH}_EGFR-TFL-dendron(L-SO1861)4(L-HSP27BNA) and combi therapies showed no effect on cell viability up to 100 nM (FIG. 2A), whereas V_{HH}_EGFR-TFL-(block)(L-HSP27BNA) showed toxicity at higher concentrations (IC50=1000 nM; FIG. 2A). V_{HH}_EGFR-TFL(block)(L-HSP27BNA)+4000 nM SO1861-EMCH showed strong cell viability reduction at low concentrations conjugate (IC50=5 nM; FIG. 2A). In A2058 (EGFR⁻) cells no significant decrease in cell viability was observed up to 100 nM for all the conjugates or combinations (FIG. 2B).

FIG. 3 (FIG. 3): BNA-mediated gene silencing of HSP27 was determined and this revealed in A431 cells (EGFR⁺⁺) that the bivalent V_{HH}_EGFR-TFL-dendron(L-SO1861)4(L-HSP27BNA) efficiently induces HSP27 gene silencing in A431 cells (IC50=8 nM; FIG. 3A) compared to bivalent V_{HH}_EGFR-TFL-(block)(L-HSP27BNA) alone (IC50=1000 nM; FIG. 3A) that also showed cell viability reduction at the same concentration range (see FIG. 2A). In A2058 cells (EGFR⁻) no gene silencing activity can be observed at low concentrations V_{HH}_EGFR-TFL-dendron(L-SO1861)4(L-HSP27BNA). At concentrations with more than 100 nM V_{HH}_EGFR-TFL-dendron(L-SO1861)4(L-HSP27BNA) HSP27 gene silencing is observed (FIG. 3B), however with similar concentrations also cell viability reduction is observed (see FIG. 2B).

FIG. 4 (FIG. 4): Bivalent V_{HH}_EGFR-TFL-dendron(L-SO1861)4 was tested. The bivalent V_{HH}_EGFR binds with one arm the binding site of cetuximab and with the other arm the binding site of matuzumab on the EGFR receptor. Bivalent V_{HH}_EGFR-TFL-dendron(L-SO1861)4 was titrated on a fixed concentration of 10 pM CD71mab-saporin and targeted protein toxin-mediated cell killing on A431 (EGFR⁺⁺/CD71⁺) (FIG. 4A) and A2058 (EGFR⁻/CD71⁺) (FIG. 4B) cells was determined.

FIG. 5 (FIG. 5): The 1 target 2-components system (1T2C) (competing and non-competing) is the combination treatment of bivalent V_{HH}-SO1861 and mAb-protein toxin, as illustrated in FIG. 1A. Bivalent V_{HH}_EGFR-TFL-dendron(L-SO1861)4 was tested. The bivalent V_{HH}_EGFR binds with one arm the binding site of cetuximab and with the other arm the binding site of matuzumab on the EGFR receptor. Bivalent V_{HH}_EGFR-TFL-dendron(L-SO1861)4 was titrated on a fixed concentration of 10 pM cetuximab-saporin, and targeted protein toxin-mediated cell killing on A431 (EGFR⁺⁺) (FIG. 5A) and A2058 (EGFR⁻) (FIG. 5B) was determined.

FIG. 6 (FIG. 6): The 1 target 2-components system (1T2C) (competing and non-competing) is the combination treatment of bivalent V_{HH}-SO1861 and mAb-protein toxin, as illustrated in FIG. 1A. Bivalent V_{HH}_EGFR-TFL-dendron-(L-SO1861)4 was titrated on a fixed concentration of 10 pM matuzumab-dianthin and targeted protein toxin-mediated cell killing on A431 (EGFR⁺⁺) (FIG. 6A) and A2058 (EGFR⁻) (FIG. 6B) cells was determined.

FIG. 7 (FIG. 7): The 1 target 2-components system (1T2C) (competing) is the combination treatment of bivalent V_{HH}-SO1861 and V_{HH}-protein toxin. Bivalent V_{HH}_EGFR-TFL-dendron(L-SO1861)4 was titrated on a fixed concentration of 5 pM bivalent V_{HH}-EGFR-dianthin and targeted protein toxin-mediated cell killing on A431 (EGFR⁺⁺) (FIG. 7A) and A2058 (EGFR⁻) (FIG. 7B) cells was determined.

FIG. 8 (FIG. 8): Cell killing (MTS) assay) with the combination treatment according to the invention of V_{HH}(HER2)-SO1861 (DAR1)+50 pM CD71V_{HH}-dianthin (DAR1) on SK-BR-3 cells (HER2⁺⁺/CD71⁺) (A) and MD-MB-468 cells (HER2⁻/CD71⁺) (B). The legend to FIG. 2B, depicted next to the graphs, also applies to FIG. 2A.

FIG. 9 (FIG. 9): Cell killing (MTS) assay) with the combination treatment according to the invention of CD71V_{HH}-dianthin (DAR1)+900 nM HER2V_{HH}-SO1861 (DAR1) on SK-BR-3 cells (HER2⁺⁺/CD71⁺) (A) and MD-MB-468 cells (HER2⁻/CD71⁺) (B). The legend to FIG. 3B, depicted next to the graphs, also applies to FIG. 3A.

FIG. 10 (FIG. 10): Cell killing (MTS) assay) with the combination treatment according to the invention of CD71V_{HH}-dianthin (DAR1)+cetuximab-SO1861 (DAR4) or HER2V_{HH}-dianthin (DAR1)+cetuximab-SO1861 (DAR4) or EGFRV_{HH}-dianthin (DAR1)+cetuximab-SO1861 (DAR4) on A431 cells (EGFR⁺⁼/HER^{+/-}/CD71⁺) (A) and A2058 cells (EGFR⁻/HER^{+/-}/CD71⁺) (B).

FIG. 11 (FIG. 11): Cell killing (MTS) assay) with the combination treatment according to the invention of CD71V_{HH}-dianthin (DAR1)+77 nM cetuximab-SO1861 (DAR4) or HER2V_{HH}-dianthin (DAR1)+77 nM cetuximab-SO1861 (DAR4) or EGFRV_{HH}-dianthin (DAR1)+77 nM cetuximab-SO1861 (DAR4) on SK-BR-3 cells (HER2⁺⁺/EGFR⁺/CD71⁺) (A) and MDA-MB-468 cells (HER2⁻/EGFR⁺⁺/CD71⁺) (B).

FIG. 12: Cell killing assay with SO1861+VHH-toxin

FIG. 13: Cell killing assay with SO1861+VHH-toxin

FIG. 14: Cell killing (MTS) assay with bivalentVHHEGFR-dianthin+SO1861-EMCH, bivalentVHHEGFR-dianthin+76.9 nM cetuximab-SO1861 (DAR4), cetuximab-saporin+SO1861-EMCH orcetuximab-saporin+cetuximab-SO1861 (DAR4) on A) MDA-MB-468 cells (EGFR⁺⁺) and B) A431 cells (EGFR⁺⁺).

FIG. 15: Cell killing (MTS) assay with SO1861+1 or 5 pM bivalentVHHEGFR-dianthin or cetuximab-SO1861 (DAR4)+1 or 5 pM bivalentVHHEGFR-dianthin A) MDA-MB-468 cells (EGFR⁺⁺) and B) A2058 cells (EGFR⁻)

FIG. 16: Cell killing (MTS) assay with the combination treatment according to the invention of cetuximab-SO1861 (DAR4)+50 pM bivalent VHHEGFR-dianthin (recombinant fusion protein) or trastuzumab-SO1861 (DAR4)+50 pM bivalent VHHEGFR-dianthin (recombinant fusion protein) on A431 cells (EGFR⁺⁺/HER^{+/-}), A2058 cells (EGFR⁻/HER^{+/-}), SK-BR-3 cells (EGFR⁺/HER2⁺⁺) and MDA-MB-468 cells (EGFR⁺⁺/HER2⁻).

FIG. 17: V_{HH}-EGFR-dianthin (conjugate) was titrated alone or on a fixed concentration of 4000 nM SO1861-EMCH or 76.9 nM cetuximab-SO1861 (DAR4) and targeted protein toxin (dianthin) mediated cell killing was determined on A431 (EGFR⁺⁺) cells. “SPT001” is saponin SO1861.

FIG. 18: synthesis of VHH-L-BNA.

FIG. 19: synthesis of Trifunctional linker-(L-SO1861)-(L-BNA)-(VHH), intermediate 2, 3.

FIG. 20: synthesis of Trifunctional linker-(L-SO1861)-(L-BNA)-(VHH), intermediate 4.

FIG. 21: synthesis of Trifunctional linker-(dendron-(L-SO1861)4)-(L-BNA)-(VHH), intermediate 5, 6

FIG. 22: synthesis of Trifunctional linker-(dendron-(L-SO1861)4)-(L-BNA)-(VHH)

FIG. 23. Structure and two simplified drawings of molecule 6, i.e. Trifunctional linker-(DBCO)-(TCO)-(Maleimide) (also abbreviated as ‘TFL’).

FIG. 24. Molecular structure of 12,13-dehydrooleanane type triterpenoid saponin SO1861 comprising a quillaic acid aglycone core structure with a carbohydrate chain linked to the C-3 and a PEG4 azide linker conjugated to the aglycone core by forming a hydrazone bond with the aldehyde group at the C-23 of the aglycone, present in the natural, free form of the saponin, as well as a simplified drawing of the SO1861 with a covalently linked linker with an azide group, referred to as molecule 7, i.e. SO1861-L-azide, with ‘L’ referring to the acid lability of the hydrazone bond.

FIG. 25. Molecular structure of the conjugate of molecule 6 coupled to a single copy of the saponin SO1861-L-azide (molecule 7), i.e. molecule 8 or Trifunctional linker-(L-SO1861)-(TCO)-(Maleimide). The schematic representation is shown on the second page of FIG. 25.

FIG. 26. Molecular structure and a simplified drawing are depicted of molecule 1, i.e. BNA-disulfide, and molecule 10, i.e. Hydrazone containing Heterobifunctional methyl-tetrazine maleimide linker (A); and of molecule 11, i.e. methyltetrazine-L-ApoB BNA (B).

FIG. 27. Molecular structure and simplified representation of the 1-component single V_{HH} domain conjugate referred to as molecule 12, comprising a single copy of the saponin SO1861 and a single copy of the oligonucleotide moiety BNA, the conjugate also referred to as Trifunctional linker-(L-SO1861)-(L-BNA)-(V_{HH}). The schematic representation is shown on the second page of FIG. 27.

FIG. 28. Molecular structure and simplified representation of a G2 dendron with four copies of the saponin SO1861 covalently bound to it via hydrazone bonds, the dendron provided with a PEG4 linker with an terminal azide group, termed dendron-(SO1861)4-azide and molecule 13.

FIG. 29. Molecular structure and simplified representation of molecule 14, consisting of a G2 dendron with four copies of the saponin SO1861 covalently bound to it via hydrazone bonds, the dendron provided with a PEG4 linker, through which the dendron is covalently bound to the trifunctional linker referred to as molecule 6, the molecule 14 also referred to as trifunctional linker-(dendron(-L-SO1861)4)-(TCO)-(Maleimide).

FIG. 30. Molecular structure and simplified representation of molecule 16, referred to as 1-component covalent conjugate trifunctional linker-(dendron-(L-SO1861)4)-(L-BNA)-(V_{HH}), comprising four copies of the saponin SO1861, a single copy of the BNA oligonucleotide and a single V_{HH} domain, here for binding to EGFR on target cells. The saponin moieties are coupled to the G2 dendron according to FIG. 28, via hydrazone bonds. The schematic representation is shown on the second page of FIG. 30.

FIG. 31. Synthesis of Trifunctional linker-(dendron(L-SO1861)4)-(L-BNA oligo)-(Maleimide) (molecule 17) from Methyltetrazine-BNA oligo (molecule 11) and trifunctional linker-(dendron-(L-SO1861)4)-(TCO)-(Maleimide) (molecule 14).

FIG. 32. Synthesis of Trifunctional linker-(dendron(L-SO1861)4)-(L-BNA oligo)-(bivalent VHH) (molecule 19) by reacting bivalent VHH-SH (molecule 18) with TFL-dendron-(L-SO1861)4)-(L-BNA)-(Maleimide) (molecule 17).

FIG. 33. Synthesis of Trifunctional linker-(dendron(L-SO1861)4)-(blocked TCO)-(Maleimide) (molecule 21) by coupling together trifunctional linker-(dendron-(L-SO1861)4)-(TCO)-(Maleimide) (molecule 14) and N-(2-hydroxyethyl)-2-(4-(6-methyl-1,2,4,5-tetrazin-3-yl)phenyl)acetamide (molecule 20).

FIG. 34. Synthesis of Trifunctional linker-(dendron(L-SO1861)4)-(blocked TCO)-(bivalent VHH) (molecule 22) by linking bivalent VHH-SH (molecule 18) and TFL-(dendron(L-SO1861)4)-(blocked TCO)-(Maleimide) (molecule 21) together.

FIG. 35. Synthesis of the Intermediate 9, Trifunctional linker-(blocked DBCO)-(TCO)-(Maleimide) (molecule 24) by covalently conjugating 1-azido-3,6,9-trioxaundecane-11-ol (molecule 23) and TFL-(DBCO)-(TCO)-(Maleimide) (molecule 6) together.

FIG. 36. Synthesis of the Intermediate 10, Trifunctional linker-(blocked DBCO)-(L-BNA oligo)-(Maleimide) (molecule 25) by covalently conjugating methyltetrazine-BNA oligo (molecule 11) and trifunctional linker-(blocked DBCO)-(TCO)-(Maleimide) (molecule 24).

FIG. 37. Synthesis of Trifunctional linker-(blocked DBCO)-(L-BNA oligo)-(bivalent VHH) (molecule 26) by covalently conjugating bivalent VHH-SH (molecule 18) and TFL-(blocked DBCO)-(L-BNA oligo)-(Maleimide) (molecule 25).

FIG. 38A. Synthesis of G3 dendron with 8 copies of the saponin SO1861(-L-)EMCH covalently bound to it (SO1861 is linked to the linker via an acid-labile hydrazone bond).

FIG. 38B. Molecular structure and simplified representation of saponin SO1861 with coupled EMCH though a hydrazone bond formed from the aldehyde group of the saponin aglycone.

FIG. 38C. Providing the dendron(-L-SO1861)8 conjugate of FIG. 38A with an azide group through coupling as depicted, therewith providing dendron(-L-SO1861)8-azide.

FIG. 39. Conjugation product Trifunctional linker-(dendron(-L-SO1861)8)-(TCO)-(Maleimide) of the dendron(-L-SO1861)8-azide (FIG. 38C) with the trifunctional linker (molecule 6), also referred to as TFL, is displayed.

FIG. 40. Reaction product Trifunctional linker-(dendron(-L-SO1861)⁸)-(L-BNA)-(V_{HH}) of the conjugation of Trifunctional linker-(dendron(-L-SO1861)8)-(TCO)-(Maleimide) (FIG. 39) with the oligonucleotide as displayed in FIG. 36 and the V_{HH} domain which is coupled via a thiol group of a cysteine residue in the amino-acid sequence of the domain (see also FIG. 41A), is displayed.

FIG. 41A. Bivalent V_{HH} with a C-terminal linker sequence comprising a tetra-Cys repeat (SEQ ID NO: 77: Amino-acid sequence of tetra-Cys artificial linker) for covalent coupling is displayed. Molecule 18 is the Bivalent V_{HH} with free thiol groups for covalent coupling to for example TFL depicted as molecule 6, or the TFL conjugated with for example 1, 4 or 8 saponin moieties and/or conjugated with at least a single copy of an oligonucleotide. The disulphide bonds are reduced in molecule 18 as depicted, providing the bivalent V_{HH} molecule with the thiol groups available for covalent bonding, for example with the maleimide group of the trifunctional linker displayed as molecule 6.

FIG. 41B. The 1-component bivalent V_{HH} conjugate Trifunctional linker-(dendron(L-SO1861)4)-(L-BNA oligo)-(bivalent V_{HH}) comprising four saponin moieties and a single oligonucleotide moiety is displayed (referred to as molecule 19).

FIG. 42A-C: Representation of Trifunctional linker-(L-hydrazone-saponin)-(L-oligonucleotide)-(Trivalent-GalNAc) and Trifunctional linker-(L-semicarbazone-saponin)-(L-oligonucleotide)-(Trivalent-GalNAc) (A); the saponin is SO1861, the oligonucleotide is ApoB BNA. For synthesis of the 1-component conjugate, the saponin is covalently linked to the trifunctional linker via either a hydrazone bond formed upon binding of 1-azido-3,6,9,12-tetraoxapentadecane-15-hydrazide linker to the aldehyde group of the saponin (here SO1861) (B), or a semicarbazone bond formed upon binding of linker 4-(6-azidohexanoyl)piperazine-1-carbohydrazide to the aldehyde group of the saponin (C).

FIG. 43A-C: Representation of Trifunctional linker-(dendron(-L-hydrazone-saponin)4)-(L-oligonucleotide)-(Trivalent-GalNAc) and Trifunctional linker-(dendron(-L-semicarbazone-saponin)4)-(L-oligonucleotide)-(Trivalent-GalNAc) (A); the saponin is SO1861, the oligonucleotide is ApoB BNA. For synthesis of the 1-component conjugate, the four copies of the saponin are covalently linked to the G2 dendron (which in turn is covalently coupled to the trifunctional linker) via either a hydrazone bond formed upon binding of EMCH linker to the aldehyde group of the saponin (here SO1861) (B), or a semicarbazone bond formed upon binding of linker tert-butyl 2-(4-(6-(2,5-dioxo-2,5-dihydro-1H-pyrrol-1-yl)hexanoyl)piperazine-1-carbonyl)hydrazine-1-carboxylate to the aldehyde group of the saponin (C).

FIG. 44: Representation of Trifunctional linker-(dendron(-L-hydrazone-saponin)8)-(oligonucleotide)-(Trivalent-GalNAc) and Trifunctional linker-(dendron(-L-semicarbazone-saponin)8)-(oligonucleotide)-(Trivalent-GalNAc) (A); the saponin is SO1861, the oligonucleotide is ApoB BNA. The eight copies of the saponin are covalently linked to the G3 dendron as described for the conjugates of FIG. 43 (See FIG. 43B, C).

FIG. 45: ApoB expression analysis in liver tissue of C57BL/6J mice.

FIG. 46: Serum ApoB protein analysis in C57BL/6J mice.

FIG. 47: Release kinetic assay of SO1861-EMCH (saponin covalently coupled to the linker through an acid-labile hydrazone bond) (A) and SO1861-SC-Mal (blocked) (saponin covalently coupled to the linker through an acid-labile semicarbazone (sc) bond) (B), at various indicated pH values.

### DETAILED DESCRIPTION

In order for a bioactive molecule (e.g. an effector molecule) to work, the molecule must be able to engage with its target, e.g. in the blood serum, on the outside of the cell surface or inside a cell or an organelle. The active moiety of almost all protein-based targeted toxins, e.g., must enter the cytosol of the target cell to mediate its target modulatory effect. In many constellations the toxin remains ineffective since (1) the targeting moiety is poorly internalized and remains bound to the outside of the cells, (2) is recycled back to the cell surface after internalization or (3) transported to the endolysosomes where it is degraded. Although these fundamental issues are known for decades and more than 500 targeted toxins have been investigated in the past decades, the problems have not been solved yet and only a couple of antibody-targeted protein toxin have been admitted to the market, albeit with warning labels for severe toxicity. Moxetumomab pasudotox-tdfk (LUMOXITI®, AstraZeneca Pharmaceuticals LP), has been approved for relapsed or refractory hairy cell leukemia by the FDA to date. Other of such approved ADCs are Elzonris, Ontak.

To overcome these problems, many strategies have been described including approaches to redirect the toxins to endogenous cellular membrane transport complexes of the biosynthetic pathway in the endoplasmic reticulum and techniques to disrupt or weaken the membrane integrity of endosomes, i.e. the compartments of the endocytic pathway in a cell, and thus facilitating the endosomal escape. This comprises the use of lysosomotropic amines, carboxylic ionophores, calcium channel antagonists, various cell-penetrating peptides of viral, bacterial, plant, animal, human and synthetic origin, other organic molecules and light-induced techniques. Although the efficacy of the targeted toxins was typically augmented in cell culture hundred- or thousand-fold, in exceptional cases more than million-fold, the requirement to co-administer endosomal escape enhancers with other substances harbors new problems including additional side effects, loss of target specificity, difficulties to determine the therapeutic window and cell type-dependent variations.

All strategies, including physicochemical techniques, require enhancer molecules that interact more or less directly with membranes and comprise essentially small chemical molecules, secondary metabolites, peptides and proteins. A common feature of all these substances is that they are per se not target cell-specific and distribute with other kinetics than the targeted toxins. This is one major drawback of the current approaches.

It is a first goal of the present invention to provide improved ADCs and AOCs with an increased therapeutic window, and to provide improved ADCs and AOCs for delivery of an effective amount or dose of an effector molecule, when for example the delivery from outside a target cell into said cell, is considered, or more in particular when the delivery of the effector molecule in the cytosol of said target cell is considered. It is a second goal of the present invention to provide an improved method of treatment of a (human) patient suffering from a disease to be treated with a conjugate comprising an effector molecule and a ligand, such as Her2 targeting V_{HH}, EGFR targeting V_{HH}, etc, or a binding derivative or binding fragment thereof, preferably an sdAb or multiple sdAb's, such as one or more V_{HH}'s, preferably a bivalent V_{HH}, for e.g. a target tumor cell, i.e. to improve the therapeutic window of the ADC or the AOC comprising the effector molecule to be delivered in the cytosol of e.g. target tumor cells. The multiple sdAb's either can bind to the same type of cell-surface molecule, such as the same copy of the cell-surface molecule or to different copies of the same type of cell-surface molecule present on the same cell, or for example the multiple sdAb's such as two sdAb's can bind to a first cell-surface molecule and to a different second cell-surface molecule present on the same cell, or present on two different cells. Such one or more kinds of cell-surface molecules are typically endocytic receptors. The multiple sdAb's can be multivalent for the same binding site on a cell-surface molecule, multiparatopic such as biparatopic, and/or multi-specific such as bi-specific for a first cell-surface molecule and a second cell-surface molecule present at the same cell, or present at two different cells. The multiple sdAb's such as two sdAb's (a first sdAb and a second sdAb) are selected for their binding capacity towards the same binding site or different binding sites present at a single cell selected for binding of the conjugate of the invention or different binding sites present at two different cells selected for binding of the conjugate. Binding sites for the at least two sdAb's are present at the same cell or are present at two different cells. Typically, binding sites are epitopes in cell-surface receptors, such as tumor-cell specific cell-surface receptors, preferably endocytic receptors.

It is an objective of the current invention to provide a conjugate which is a combination of an effector-molecule activity enhancing molecule and an ADC or an AOC, for use in therapy such as anti-cancer therapy. By provision of such conjugate of the invention, the therapeutic window of the effector molecule, which is part of the conjugate, such as an ADC or an AOC, is effectively widened. Typically, the effector molecule is an oligonucleotide.

At least one of the above objectives is achieved by providing improved ADCs and improved AOCs, which are conjugates further comprising an effector-molecule activity enhancing molecule.

The present invention will be described with respect to particular embodiments but the invention is not limited thereto but only by the claims. While the invention has been described in terms of several embodiments, it is contemplated that alternatives, modifications, permutations and equivalents thereof will become apparent to one having ordinary skill in the art upon reading the specification and upon study of the drawings and graphs. The invention is not limited in any way to the illustrated embodiments. Changes can be made without departing from the scope which is defined by the appended claims.

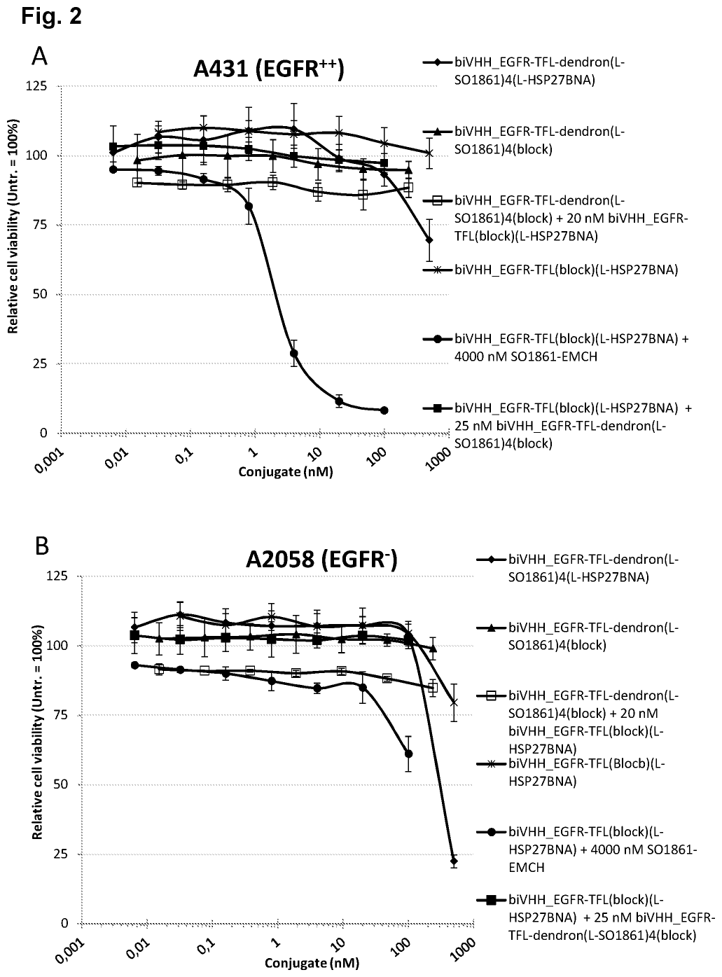

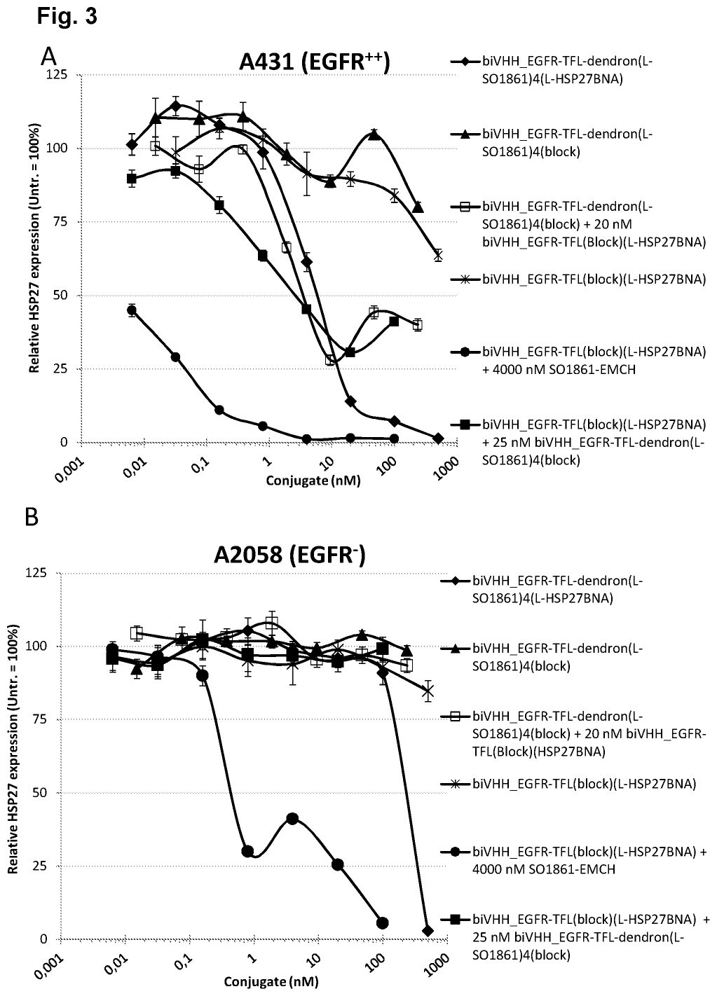

The therapeutic window of a conjugate such as an antibody drug conjugate or an antibody-oligonucleotide conjugate, according to the invention, increases when administered to a tumor-bearing mammal (human) to whom the conjugate is administered, when said conjugate comprises at least one covalently bound saponin. The saponin is conjugated with at least one sdAb such as a bivalent sdAb tandem, and an effector molecule such as protein toxins and an oligonucleotide such as a BNA. The inventors were the first who established and determined that conjugating a saponin of the invention with a ligand for binding to a cell-surface molecule, such as an antibody such as a full-length intact IgG, or such as an sdAb such as a (bivalent) V_{HH}, provides a conjugate for cell-specific delivery of the saponin at the cell surface of a target cell exposing the cell-surface molecule at its cell surface, and subsequent delivery of the saponin inside the cell, such as the cell endosome, endolysosome, lysosome and ultimately in the cell cytosol. Examples of such cell-targeting saponin conjugates are for example provided in FIGS. 2 and 3 for saponin-V_{HH} conjugates further comprising an effector moiety, here an oligonucleotide, as further detailed and outlined in the Examples section here below. Saponin (and the effector moiety) is conjugated to Her2 targeting V_{HH} or bivalent V_{HH}, EGFR targeting V_{HH} or bivalent V_{HH}, EGFR binding V_{HH} 7D12 with amino-acid sequence as depicted as SEQ ID NO: 75, EGFR binding V_{HH} 9G8 with amino acid sequence as depicted as SEQ ID NO: 76, EGFR binding covalently linked tandem of biparatopic V_{HH}'s 7D12-9G8 with amino-acid sequence as depicted as SEQ ID NO: 73, etc., in the conjugates comprising at least one V_{HH} for binding to a cell-surface molecule such as an endocytic receptor, such as a bivalent V_{HH}. The V_{HH}'s are typically V_{HH}'S of camelid origin such as V_{HH}'s derived from camelid heavy-chain only antibodies. Thus, in the conjugate preferably, the at least one sdAb, such as a bivalent nanobody, consist(s) of V_{HH}'s, preferably camelid V_{HH}'s.

A first aspect of the invention relates to a conjugate for delivery of an effector molecule from outside a cell into said cell, preferably into the endosome and/or lysosome of said cell and preferably (subsequently) into the cytosol and/or into the nucleus of said cell, the conjugate comprising at least one saponin, at least one effector molecule and at least one, preferably at least two single domain antibody (sdAb), preferably at least one multivalent nanobody, preferably at least one bivalent nanobody comprising two single domain antibodies (sdAbs), wherein in said conjugate the saponin, effector molecule and at least one sdAb are covalently bound together, wherein the at least one saponin is a triterpenoid saponin of 12,13-dehydrooleanane type, preferably with an aldehyde group at position C-23 of the aglycone core structure of the saponin, and preferably comprising an aglycone core structure selected from:
2alpha-hydroxy oleanolic acid;
16alpha-hydroxy oleanolic acid;
hederagenin (23-hydroxy oleanolic acid);
16alpha,23-dihydroxy oleanolic acid;
gypsogenin;
quillaic acid;
protoaescigenin-21(2-methylbut-2-enoate)-22-acetate;
23-oxo-barringtogenol C-21,22-bis(2-methylbut-2-enoate);
23-oxo-barringtogenol C-21(2-methylbut-2-enoate)-16,22-diacetate;
3,16,28-trihydroxy oleanan-12-en;
gypsogenic acid,
   and wherein the at least one effector molecule is selected from: a drug molecule (a pharmaceutically active substance), a toxin, an oligonucleotide, a peptide and a protein,
and wherein the at least one sdAb, preferably the at least one multivalent nanobody, preferably at least one bivalent nanobody, targets a cell surface molecule that is present on the cell, preferably targets an endocytic receptor that is present on the cell.

An aspect of the invention relates to a conjugate for delivery of an effector molecule from outside a cell into said cell, preferably into the endosome and/or lysosome of said cell and preferably (subsequently) into the cytosol and/or into the nucleus of said cell, the conjugate comprising at least one saponin, at least one effector molecule and at least one, preferably at least two, single domain antibody (sdAb), preferably at least one multivalent nanobody, preferably at least one bivalent nanobody comprising two single domain antibodies (sdAbs), wherein the saponin, the effector molecule and the at least one sdAb are covalently bound together, wherein the at least one saponin is a triterpenoid saponin of 12,13-dehydrooleanane type,

and wherein the at least one effector molecule is selected from: a pharmaceutically active substance (drug molecule), a toxin, an oligonucleotide, a peptide and a protein,
and wherein the at least one sdAb, preferably the at least one multivalent, preferably bivalent nanobody, targets a cell surface molecule that is present on the cell, preferably targets an endocytic receptor that is present on the cell.

An aspect of the invention relates to a conjugate for delivery of an effector molecule from outside a first cell into the cytosol of said first cell, the conjugate comprising at least one saponin, at least one effector molecule and at least one single domain antibody (sdAb), wherein the saponin, the effector molecule and the at least one sdAb are covalently bound together, wherein the at least one saponin is a triterpenoid saponin of 12,13-dehydrooleanane type comprising an aglycone core structure selected from:
2alpha-hydroxy oleanolic acid;
16alpha-hydroxy oleanolic acid;
hederagenin (23-hydroxy oleanolic acid);
16alpha,23-dihydroxy oleanolic acid;
gypsogenin;
quillaic acid;
protoaescigenin-21(2-methylbut-2-enoate)-22-acetate;
23-oxo-barringtogenol C-21,22-bis(2-methylbut-2-enoate);
23-oxo-barringtogenol C-21(2-methylbut-2-enoate)-16,22-diacetate;
3,16,28-trihydroxy oleanan-12-en;
gypsogenic acid,
   and wherein the at least one effector molecule is selected from: a pharmaceutically active substance, a toxin, an oligonucleotide, a peptide and a protein,
   and wherein the at least one sdAb targets a first cell surface molecule that is present on the first cell.

For example, the conjugate comprises at least one multivalent nanobody, preferably at least one bivalent nanobody comprising two single domain antibodies (sdAbs).

An aspect of the invention relates to a conjugate for transferring an effector molecule from outside a cell into said cell, the conjugate comprising at least one effector molecule to be transferred into the cell, at least one single-domain antibody (sdAb) and at least one saponin, covalently bound to each other, directly or via at least one linker, wherein the at least one saponin is a mono-desmosidic triterpene glycoside or is a bi-desmosidic triterpene glycoside, and wherein the sdAb(s) is/are capable of binding to a cell-surface molecule of said cell. The cell-surface molecule typically is an endocytic receptor. Binding of at least one sdAb of the at least sdAb in the conjugate to said cell-surface molecule (endocytic receptor) results in endocytosis of the conjugate and delivery of the conjugate into the endosome and/or lysosome of the cell. Without wishing to be bound by any theory, under influence of the saponin comprised by the conjugate, the effector molecule comprised by the conjugate is subsequently delivered in the cytosol of the cell. The saponin is preferably a mono-desmosidic or bi-desmosidic triterpenoid saponin with an aglycone of the 12,13-dehydrooleanane type and preferably with an aldehyde group at the C-23 position of the aglycone core structure and optionally a glucuronic acid group in a carbohydrate antenna linked to the aglycone.

An aspect of the invention relates to a conjugate for delivery of an effector molecule from outside a cell into the cytosol of said cell, the conjugate comprising at least one saponin, at least one effector molecule and at least one single domain antibody (sdAb), preferably at least one multivalent nanobody, preferably at least one bivalent nanobody comprising two single domain antibodies (sdAbs), wherein in said conjugate the saponin, effector molecule and at least one sdAb are covalently bound together, wherein the at least one saponin is a triterpenoid saponin of 12,13-dehydrooleanane type, and wherein the at least one effector molecule is selected from: a pharmaceutically active substance, a toxin, an oligonucleotide, a peptide and a protein, preferably an oligonucleotide, and wherein the at least one, preferably at least two, sdAb(s), preferably the at least one multivalent, preferably bivalent nanobody target(s) a cell surface molecule that is present on the cell.

An aspect of the invention relates to a conjugate for delivery of an effector molecule from outside a cell into the cytosol of said cell, the conjugate comprising at least one saponin, at least one effector molecule and at least one single domain antibody (sdAb), preferably at least one multivalent nanobody, preferably at least one bivalent nanobody comprising two single domain antibodies (sdAbs), wherein the saponin, effector molecule and at least one sdAb are covalently bound together, wherein the at least one saponin is a triterpenoid saponin of 12,13-dehydrooleanane type, preferably comprising an aldehyde group at the C-23 position of the aglycone core structure of the saponin, more preferably comprising an aglycone core structure selected from:
2alpha-hydroxy oleanolic acid;
16alpha-hydroxy oleanolic acid;
hederagenin (23-hydroxy oleanolic acid);
16alpha,23-dihydroxy oleanolic acid;
gypsogenin;
quillaic acid;
protoaescigenin-21(2-methylbut-2-enoate)-22-acetate;
23-oxo-barringtogenol C-21,22-bis(2-methylbut-2-enoate);
23-oxo-barringtogenol C-21(2-methylbut-2-enoate)-16,22-diacetate;
3,16,28-trihydroxy oleanan-12-en;
gypsogenic acid,
and wherein the at least one effector molecule is selected from: a pharmaceutically active substance (a drug molecule), a toxin, an oligonucleotide, a peptide and a protein, preferably an oligonucleotide,
and wherein the at least one, preferably at least two, sdAb(s), preferably the at least one multivalent, preferably bivalent nanobody targets a cell surface molecule that is present on the cell.

An aspect of the invention relates to a conjugate for delivery of an effector molecule from outside a cell into the cytosol of said cell, the conjugate comprising at least one saponin, at least one effector molecule and at least one multivalent nanobody, preferably at least one bivalent nanobody comprising two single domain antibodies (sdAbs), wherein (in said conjugate) the saponin, effector molecule and at least one sdAb are covalently bound together, wherein the at least one saponin is a triterpenoid saponin of 12,13-dehydrooleanane type comprising an aglycone core structure selected from:
2alpha-hydroxy oleanolic acid;
16alpha-hydroxy oleanolic acid;
hederagenin (23-hydroxy oleanolic acid);
16alpha,23-dihydroxy oleanolic acid;
gypsogenin;
quillaic acid;
protoaescigenin-21(2-methylbut-2-enoate)-22-acetate;
23-oxo-barringtogenol C-21,22-bis(2-methylbut-2-enoate);
23-oxo-barringtogenol C-21(2-methylbut-2-enoate)-16,22-diacetate;
3,16,28-trihydroxy oleanan-12-en;
gypsogenic acid,
and wherein the at least one effector molecule is selected from: a pharmaceutically active substance, a toxin, an oligonucleotide, a peptide and a protein,
   and wherein the at least one multivalent, preferably the bivalent nanobody comprising two single domain antibodies (sdAbs) targets a cell surface molecule that is an endocytic cell-surface receptor present on the cell. Preferably, the saponin is selected from a saponin comprising gypsogenin or quillaic acid.

In exemplary and preferred embodiments of the invention, a conjugate is provided as defined herein, wherein the saponin comprises an aldehyde group at position C-23 of the saponin's aglycone core structure, or a covalent bond at position C-23 of the saponin's aglycone core structure (preferably quillaic acid or gypsogenin), such as a covalent bond that is the reaction/condensation product of an aldehyde, typically the C-23 aldehyde of the saponin, and another functional/reactive group, typically a group that is reactive towards aldehydes, the covalent bond covalently linking the saponin within the conjugate, preferably wherein the covalent bond at position C-23 is a cleavable bond that is subject to cleavage under conditions present in endosomes or lysosomes,

more preferably, wherein the cleavable covalent bond at position C-23 is adapted to restore aldehyde group at position C-23 upon cleavage.

In particularly preferred embodiments, the saponin used for preparing the conjugate, in the unconjugated state, e.g. prior to being covalently linked within the disclosed herein conjugates and/or in its natural form as existing or extracted from its source plant material, comprises an aldehyde group at position C-23 of the saponin's aglycone core structure.

Such saponins can be covalently linked to the effector molecule and/or to the sdAb(s) by any functional group present in said saponin as suitable for conjugation as known in the art, or can be covalently linked by reacting said aldehyde group at position C-23 of the saponin's aglycone core structure, which reacting results in a conversion of the aldehyde group at position C-23 into a covalent bond at position C-23 wherein said covalent bond at position C-23 is covalently linking the saponin within the conjugate.

Without wishing to be bound by any theory, it has been observed that such free aldehyde group is beneficial for a triterpenoid 12,13-dehydrooleanane-type saponin for its endosomal escape-stimulating properties, which is likely related to enhanced destabilisation of the vesicular membrane.

Consequently, for potentiating even further the endosomal escape-enhancing properties of the saponin, and thus for further improving the escaping of the effector molecule such as an oligonucleotide and/or pharmaceutically active substance, provided therewith in the endosomal compartment as part of the disclosed pharmaceutical compositions, the covalent bond at position C-23 can be selected such that upon its cleavage (e.g. in response to conditions present in mammalian endosomes or lysosomes), the aldehyde group at position C-23 of the saponin's aglycone core structure is restored.

Examples of suitable bond types that can be designed for this aldehyde-group restoration purpose include one or more of: a semicarbazone bond, a hydrazone bond, an imine bond, an acetal bond including a 1,3-dioxolane bond, and/or an oxime bond. As shown herein below in examples, such functional design of a conjugate as disclosed herein has successfully been achieved with a saponin whereby the aldehyde group naturally present at position C-23 of the saponin, was converted into a semicarbazone covalent bond or a hydrazone covalent bond at position C-23 of the saponin's aglycone core structure and linking the saponin within the conjugate. In response to acidic conditions, these bonds at position C-23 efficiently released the saponin from the conjugate, whereby the released saponin had the aldehyde group restored at position C-23.

In advantageous embodiments, such cleavable covalent bond can be selected from a semicarbazone bond, a hydrazone bond, and an imine bond, preferably from a semicarbazone bond and a hydrazone bond.

Particularly suitable for the conjugates, saponins of the 12,13-dehydrooleanane-type which naturally comprise the aldehyde group in position C-23 in their native or unconjugated form are saponins which aglycone core structure is either quillaic acid or gypsogenin. In line with this, it was observed that particularly suitable saponins for the conjugates are 12,13-dehydrooleanane-type saponins comprising a quillaic acid aglycone core structure or a gypsogenin aglycone core structure, or if the C-23 aldehyde group of these aglycone core structures was used for conjugation, derivatives of said saponins wherein the aldehyde group at position C-23 of both of these aglycones has been converted to a covalent bond at the position C-23.

For example, the covalent linking of the saponin with the effector molecule and/or with the sdAb(s) is made via a linker to which the saponin is covalently bound; preferably wherein the linker comprises a covalent bond selected from any one or more of: a semicarbazone bond, a hydrazone bond, an imine bond, an acetal bond including a 1,3-dioxolane bond, a ketal bond, an ester bond, an oxime bond, a disulfide bond, a thio-ether bond, an amide bond, a peptide bond, and an ester bond, preferably being a hydrazone bond or a semicarbazone bond; more preferably wherein the saponin either comprises an aldehyde group at position C-23 of the saponin's aglycone core structure, or a covalent bond at position C-23 of the saponin's aglycone core structure, the covalent bond covalently linking the saponin within the conjugate via the linker and comprised as part of the linker, preferably wherein the covalent bond at position C-23 is a cleavable bond that is subject to cleavage under conditions present in endosomes or lysosomes, more preferably, wherein the cleavable covalent bond at position C-23 is adapted to restore aldehyde group at position C-23 upon cleavage; or is a saponin that in at least an unconjugated state comprises an aldehyde group at position C-23 of the saponin's aglycone core structure and wherein said aldehyde group has been engaged in forming the covalent bond with the linker.

For example, the linker is a cleavable linker subject to cleavage under acidic, reductive, enzymatic and/or light-induced conditions;

preferably wherein the linker comprises a cleavable bond selected from:
a bond subject to cleavage under acidic conditions such as a semicarbazone bond, a hydrazone bond, an imine bond, an acetal bond including a 1,3-dioxolane bond, a ketal bond, an ester bond, and/or an oxime bond,
a bond susceptible to proteolysis, for example amide or peptide bond, preferably subject to proteolysis by Cathepsin B;
a red/ox-cleavable bond such as a disulfide bond, or a thiol-exchange reaction-susceptible bond such as a thio-ether bond,
   preferably being a bond subject to cleavage in vivo under acidic conditions present in endosomes and/or lysosomes of human cells, preferably at pH 4.0-6.5, and more preferably at pH≤5.5;
   more preferably being an acid-sensitive bond selected from any one or more of: a semicarbazone bond, a hydrazone bond, an imine bond, an acetal bond including a 1,3-dioxolane bond, a ketal bond, an ester bond, and/or an oxime bond, even more preferably selected from a semicarbazone bond and a hydrazone bond; most preferably being a hydrazone bond.

For example, when the saponin is conjugated by means of a covalent bond at position C-23 of the saponin's aglycone core structure (preferably being an acid-sensitive bond), it can be advantageous for said covalent bond at position C-23 to be selected such or adapted to restore the aldehyde group at position C-23 upon cleavage (e.g. under acidic conditions). Advantageously, such covalent bond can be selected from any one or more of: a semicarbazone bond, a hydrazone bond, an imine bond, an acetal bond including a 1,3-dioxolane bond, and/or an oxime bond, preferably being either a semicarbazone bond or a hydrazone bond.

For example, when the saponins of Group B and Group C described below and as claimed, are covalently bound in the conjugate via linker chemistry involving the aldehyde group (e.g. formation of a hydrazone bond or a semicarbazone bond), it is preferred that the aldehyde group is re-formed (restored) in the endosome or lysosome when the conjugate is endocytosed and the saponin is cleaved off from the remainder of the conjugate by cleavage of a cleavable bond. Examples of such saponins suitable for this purpose are listed in Table A1, and are for example the saponins of Groups A-C, in particular Group B and Group C, as outlined here below and as claimed. Examples of saponins from Table A1 that are particularly advantageous are SO1861 and SO1832 such as SO1861.

The inventors disclose here that covalently coupling saponins such as saponins present in the water-soluble fraction of Quillaja saponaria, saponins isolated from Saponaria officinalis, QS-21, SA1641, SO1861, SO1831, to the cell-surface molecule targeting molecule, e.g. an sdAb, 2-8 sdAbs such as a multivalent sdAb or a trivalent sdAb or a bivalent sdAb (comprising respectively multiple sdAbs (2-8), three or two sdAbs), preferably via linkers such as via a tri-functional linker, e.g. the tri-functional linker of Structure A (displayed here below), or via an oligomeric or polymeric structure (oligomeric molecule, polymeric molecule) of a scaffold comprising covalently bound saponins, results in improved targeting of a gene in the cell by the effector moiety in the conjugate, such as modulation of target gene expression (e.g. gene silencing) exerted by the effector moiety such as an oligonucleotide, comprised by the conjugate of the invention, under influence of the covalently coupled at least one saponin in the conjugate, preferably 1, 2, 3, 4, 5, 6, 7, 8, 9-16 copies of the saponin, more preferably 1-8, such as for example 1, 2, 4 or 8 copies of the saponin. Conjugates with a saponin comprising a gypsogenin aglycone or a quillaic acid aglycone are preferred. Typically, the conjugate comprises at least one saponin which has a quillaic acid aglycone core structure.

Thus, an aspect of the invention relates to a conjugate comprising an endosomal escape enhancing molecule, i.e. a saponin which is a triterpenoid saponin of the 12,13-dehydrooleanane type, preferably with an aglycone of the quillaic acid type or gypsogenin type, more preferably of the quillaic acid type, preferably comprising an aldehyde group at the C-23 position of the aglycone, an effector moiety and a binding molecule capable of binding to a cell-surface molecule, preferably capable of binding to an endocytic receptor of a target cell, e.g. at least one sdAb such as 1-8 sdAbs such as a multivalent, preferably bi-, tri- or tetravalent string of covalently linked sdAb domains (linearly linked together via peptide bonds, and with short linkers in between consecutive sdAb domains, known in the art (e.g. Gly₄-Ser linker)), thus a conjugate wherein the at least one glycoside molecule (saponin) and the at least one effector molecule are for example bound to one and the same binding molecule in the endosomal escape enhancing conjugate, here the at least one sdAb such as 2-4 sdAbs or a multivalent sdAb or bivalent sdAb, and wherein the endosomal escape enhancing conjugate is able to specifically bind to a target cell-specific surface molecule or structure, preferably an endocytic receptor, thereby inducing receptor-mediated endocytosis of the complex of the conjugate and the target cell-specific surface molecule (binding of conjugates to the receptor is followed by internalization of the conjugate/receptor complex). Without wishing to be bound by any theory, the inventors noticed that the combination of the saponin and the effector moiety in the endosomal escape enhancing conjugate enables augmentation of endosomal escape of said effector moiety by said saponin (i.e delivery of the effector moiety from the endosome into the cytosol of the cell). By doing so, the conjugate preferably improves the effect of the effector molecule compared to an ADC or AOC comprising the binding molecule (here the at least one sdAb) and the effector moiety without the saponin.

To explain the invention in more detail, the process of cellular uptake of substances and the used terminology in this invention is described first. The uptake of extracellular substances into a cell by vesicle budding is called endocytosis. Said vesicle budding can be characterized by (1) receptor-dependent ligand (here, the conjugate) uptake mediated by the cytosolic protein clathrin, (2) lipid-raft uptake mediated by the cholesterol-binding protein caveolin, (3) unspecific fluid uptake (pinocytosis), or (4) unspecific particle uptake (phagocytosis). All types of endocytosis run into the following cellular processes of vesicle transport and substance sorting called the endocytic pathways. The endocytic pathways are complex and not fully understood. Earlier, it was thought that organelles are formed de novo and mature into the next organelle along the endocytic pathway. Nowadays, it is hypothesized that the endocytic pathways involve stable compartments that are connected by vesicular traffic. A compartment is a complex, multifunctional membrane organelle that is specialized for a particular set of essential functions for the cell. Vesicles are considered to be transient organelles, simpler in composition, and are defined as membrane-enclosed containers that form de novo by budding from a preexisting compartment. In contrast to compartments, vesicles can undergo maturation, which is a physiologically irreversible series of biochemical changes. Early endosomes and late endosomes represent stable compartments in the endocytic pathway while primary endocytic vesicles, phagosomes, multivesicular bodies (also called endosome carrier vesicles), secretory granules, and even lysosomes represent vesicles. The endocytic vesicle, which arises at the plasma membrane, most prominently from clathrin-coated pits, first fuses with the early endosome, which is a major sorting compartment of approximately pH 6.5. A large part of the cargo and membranes internalized are recycled back to the plasma membrane through recycling vesicles (recycling pathway). Components that should be degraded are transported to the acidic late endosome (pH lower than 6) via multivesicular bodies. Lysosomes are vesicles that can store mature lysosomal enzymes and deliver them to a late endosomal compartment when needed. The resulting organelle is called the hybrid organelle or endolysosome. Lysosomes bud off the hybrid organelle in a process referred to as lysosome reformation. Late endosomes, lysosomes, and hybrid organelles are extremely dynamic organelles, and distinction between them is often difficult. Degradation of the endocytosed molecules occurs inside the endolysosomes. Endosomal escape is the active or passive release of a substance from the inner lumen of any kind of compartment or vesicle from the endocytic pathway, preferably from clathrin-mediated endocytosis, or recycling pathway into the cytosol. Endosomal escape thus includes but is not limited to release from endosomes, endolysosomes or lysosomes, including their intermediate and hybrid organelles. After entering the cytosol, said substance might move to other cell units such as the nucleus. Glycoside molecules (saponins; here the triterpenoid saponins of the 12,13-dehydrooleanane type)) in the context of the invention are compounds that are able to enhance the effect of an effector molecule comprised by the conjugate (and preferably released from or cleaved off from the conjugate molecule once present in the endosome/endolysosome/lysosome), in particular by facilitating the endosomal escape. Without wishing to be bound by any theory, the glycoside molecules interact with the membranes of compartments and vesicles of the endocytic and recycling pathway and make them leaky for said effector molecules resulting in augmented endosomal escape.

With the term “improving an effect of an effector molecule” is meant that a saponin according to the embodiments increases the functional efficacy of the effector molecule (e.g. the therapeutic index of a toxin or a drug or an oligonucleotide, preferably an oligonucleotide (nucleic acid); the metabolic efficacy of a modifier in biotechnological processes; the transfection efficacy of genes in cell culture research experiments), preferably by enabling or improving its target engagement. Acceleration, prolongation, or enhancement of antigen-specific immune responses are preferably not included. Therapeutic efficacy includes but is not limited to a stronger therapeutic effect with lower dosing and/or less side effects. “Improving an effect of an effector molecule” can also mean that an effector molecule, which could not be used because of lack of effect (and was e.g. not known as being an effector molecule), becomes effective when used in combination with the present invention. Any other effect, which is beneficial or desired and can be attributed to the combination of effector moiety and saponin in one conjugate, as provided by the invention, is considered to be “an improved effect”. In the context of the invention, a saponin of the invention is an “enhancer” of the functional efficacy of an effector molecule in the conjugate of the invention.

One major drawback of targeted toxin or nucleic acid (oligonucleotide) enhancement by glycosides, such as for instance saponins, up to the present invention is that the targeted toxins or targeted oligonucleotides are internalized by receptor-mediated endocytosis (binding of conjugates to the endocytic receptor is followed by internalization of the conjugate/receptor complex) while glycosides passively diffuse through the plasma membrane and reach the endosomal membranes presumably via interaction with cholesterol. In principal, glycosides can enter any cell, also non-target cells (off-target cells), resulting in inefficient enhancer availability in the target cells for effective release of the targeted toxin or oligonucleotide and possible side effects in non-target cells. One major problem is that entry of the targeted toxin or oligonucleotide (or peptide or protein) and the glycosides proceed with different kinetics and that these kinetics are different from cell (line) to cell (line) and from tissue to tissue, so that the correct time difference for the application of the two substances (ADC or AOC, free saponin) can widely vary from (tumor) (cell (line)) to (tumor) (cell (line)). Moreover, in living organisms, liberation, absorption, distribution, metabolism and excretion of these substances is also different. Furthermore, the a-specific uptake of glycosides by non-targeted cells may induce unwanted effects in these cells. This can, e.g., be cytosolic delivery of compounds that should have been delivered to the lysosomes, disturbed antigen presentation, etc. Non-targeted administration of the glycoside and the targeted drug may also be problematic in drug development and may hinder or at least postpone marketing authorization by the relevant authorities (e.g. FDA or EMA). With targeted toxin or targeted drug or targeted oligonucleotide or targeted peptide or targeted protein in the context of the present invention is meant that a toxin or drug or oligonucleotide or protein or peptide is specifically targeted to a membrane bound molecule on a target cell, e.g. a toxin or drug, oligonucleotide, peptide or protein bound to a ligand, here at least one sdAb, of a membrane receptor such as bound to a string of 2-8 sdAbs that specifically recognizes a structure on the cell membrane of a target cell, preferably an endocytic receptor of the target cell.

It is thus very useful to direct the glycoside (saponins as outlined herein) via the same route as the effector molecule, e.g., via a targeting sdAb or targeting sdAbs to the target cell in order for the enhancer to be available at effective concentration inside the acidic compartments of the endocytic pathway of the target cell and in order to exhibit a synergistic effect with the oligonucleotide, peptide, protein or toxin. The present invention, therefore, provides novel approaches to redirect both the effector molecule and the endosomal escape enhancer (i.e. a saponin of the invention) via a targeting ligand (binding molecule; i.e. at least one sdAb, preferably multiple sdAbs such as 2-8 sdAbs, such as a multivalent sdAb, for example a bi-, tri- or tetravalent sdAb, preferably a bivalent sdAb consisting of a first sdAb and a second sdAb (‘bivalent nanobody’)) to the acidic compartments of the endocytic pathway of the target cell.

The inventors established that an effector molecule which is part of the conjugate comprising the at least one sdAb is delivered inside a cell with high efficiency under influence of a saponin which is also comprised by the conjugate, when the effect of the effector molecule inside the cell is considered. Surprisingly, despite the relative small size of an sdAb such as a V_{HH}, binding of the conjugate comprising such sdAb to the cell surface receptor is still occurring when both an effector molecule and a saponin are comprised by the conjugate comprising the sdAb such as a V_{HH}. The binding of a saponin and the binding of an effector molecule together, to the sdAb such as a V_{HH}, does not result in e.g. steric hindrance when the capacity of the at least one V_{HH} to bind to the cell surface molecule is considered. That is to say, contacting e.g. tumor cells with a sub-optimal dose of e.g. an ADC or AOC does not result in intracellular effector molecule activity (for example, the target cell is not efficiently killed or the target gene is not efficiently silenced, upon biological activity of the effector molecule), in the absence of the at least one saponin covalently coupled to said ADC or AOC. However, when the target (tumor) cell is contacted with the conjugate of the invention comprising the effector molecule and comprising the saponin, and further comprising the target-cell binding one or multiple sdAb(s), such as 2, 3, 4, 5, 6, 7, or 8 sdAbs, preferably 2-4 sdAbs such as 2 or 3 sdAbs, preferably 2 sdAbs, for example in bivalent sdAb format, for example efficient tumor cell killing is achieved (if the effector moiety in the conjugate is a (proteinaceous) toxin, for example), or efficient gene silencing is achieved (if the effector moiety is a gene-silencing oligonucleotide).

By targeting a single cell-surface molecule such as a single kind of endocytic receptor, with the conjugate of the invention, the delivery of the saponin and the effector moiety bound to the cell-surface molecule targeting at least one sdAb, preferably multiple sdAbs (such as multivalent sdAb) in the conjugate of the invention, at and inside the cytosol of the targeted cell, which exposes the cell-surface molecule on the cell surface, is improved and more specific, compared to for example contacting the cell with only a regular ADC or AOC known in the art lacking the saponin of the invention, thus without the presence of the saponin as part of the conjugate of the invention. An aberrant cell selected for targeting by the cell-surface molecule targeting at least one sdAb of the conjugate, ideally bears the epitope on the cell-surface molecule to which the cell-surface molecule targeting molecule can bind, to a high extent (i.e. relatively higher expression of the targeted cell-surface molecule on the targeted cell such as for example a tumor cell or an auto-immune cell, than the expression of the same cell-surface molecule on a non-targeted cell such as for example a healthy cell) and/or expose the epitope in the targeted cell-surface molecule for binding of the cell-surface molecule targeting sdAb of the conjugate, specifically, when (neighboring) healthy cells in a patient are considered. Preferably, the cell-surface molecule targeted by the cell-surface molecule targeting at least one sdAb, preferably at least two sdAbs, of the conjugate of the invention is relatively highly and/or specifically expressed on the targeted (diseased, tumor) cell compared to healthy cells.

An embodiment is the conjugate of the invention, wherein the target cell-surface molecule for the cell-surface molecule targeting at least one sdAb, preferably at least two sdAbs, of the conjugate such as a tumor-cell receptor, is expressed specifically or to a relatively higher extent when compared to expression of the cell-surface molecule on the surface of a healthy (neighboring) cell. Thus, the epitope on the targeted cell-surface molecule is ideally unique to the targeted (diseased) cells, and is at least specifically present and exposed at the surface of the targeted cells. Binding of the conjugate of the invention to the epitope on the cell-surface molecule on a targeted cell is followed by endocytosis of the complex of the conjugate and the target cell-surface molecule (binding of conjugates to the receptor is followed by internalization of the conjugate/receptor complex: endocytosis). Since the conjugate only can enter the target cell through binding interaction with a cell-surface molecules specifically expressed to a sufficient extent or uniquely expressed on the targeted cell when compared to healthy cells that should not be targeted, accumulation of a therapeutically active amount of effector moiety and saponin comprised by the conjugate, inside the target cells is only possible and occurring if expression levels of the targeted cell-surface molecule is above a certain minimal expression threshold. At the same time, the fact that the effector moiety bound to the cell-surface molecule targeting one or more, preferably 2-4 sdAbs of the conjugate is only capable of exerting its intracellular (e.g. cytotoxic or gene silencing) activity in the presence of very same conjugate bearing the covalently bound saponin, also provides a safeguard against negative and undesired side effects of the effector moiety towards e.g. healthy cells and healthy tissue not meant to be targeted and affected by the effector moiety, when compared to exposure of cells to an ADC or AOC without the covalently bound saponin(s). That is to say, sufficiently low expression or even absence of exposed cell-surface molecule, to which a conjugate could bind via binding interaction between the 1-8 sdAbs of the conjugate and the endocytic receptor, does ideally not allow entrance into (non-targeted) healthy cells of the conjugate to amounts that would result in endosomal escape of the effector moiety under influence of the saponin comprised by the conjugate. Since the ADC with covalently coupled saponin or the AOC with covalently coupled saponin according to the invention can be used at lower dose compared to when the ADC or AOC without coupled saponin was applied in the therapeutic regimen, entrance of ADC with coupled saponin or entrance of AOC with coupled saponin in healthy cells to low extent already bears a lower risk for occurrence of unwanted side effects when for example the targeting and killing of target diseased cells such as tumor cells and auto-immune cells is considered, and/or when gene silencing is for example considered in the desired target cell.

Inclusion of at least one, preferably 2-8, sdAb(s) (e.g. multivalent, biparatopic, bivalent sdAb) in the conjugate has thus manifold advantages compared to inclusion of an antibody such as an IgG. Importantly, since sdAbs do not comprise the Fc tail present in IgGs, risk for off-target side effects due to binding of the conjugate to Fc receptors on cells such as endothelial cells of a host to whom the conjugate is administered, is absent. Thus, the risk profile of the conjugate of the invention is improved compared to IgG-based ADCs and AOCs, or compared to ADCs or AOCs comprising an Fc tail. In addition, since the conjugate of the invention cannot be bound by Fc receptors, the conjugate is already effective at a dose which is lower than the dose required for reaching the same effector molecule activity with full-length antibody-based ADCs and AOCs, due to less or no undesired capturing of the conjugate by cell-surface receptors, different from the aimed target cell-surface molecule, i.e. the endocytic receptor to which the at least one sdAb can bind. Furthermore, due to the relatively small size of sdAbs compared to e.g. Fab, scFv, IgG, tissue penetration is improved, which is beneficial for reaching the target cells once the conjugate is administered to a patient in need of therapy. All these advantages of the application of one or multiple, preferably 2-6 sdAb(s), such as a bivalent sdAb, e.g. a biparatopic sdAb, in the conjugate of the invention, when compared to the application of larger antibodies or fragments thereof, such as IgGs comprising an Fc tail, in similar ADCs or AOCs, result in an improved therapeutic window for the effector molecule, when comprised by the conjugate of the invention. For example, an ADC based on at least one sdAb may achieve improved target cell killing in case of a targeted tumor cell when the effector molecule is for example a toxin, at the same dose at which an ADC based on an IgG and comprising the same effector molecule, is not or only sub-optimally effective. Thanks to the aspects of the invention, it is now possible to treat patients with a lower dose of effector molecule as part of a conjugate comprising the sdAb, i.e. the conjugate of the invention, therewith reaching the same or improved effector molecule mediated effect in the target cells, compared to a higher dose required when an antibody-based ADC or AOC would be used, which comprises the same effector molecule. Administering such conjugate of the invention at lower dose lowers the risk for the patient for occurrence of side effects, e.g. by non-specific entrance of non-targeted, healthy cells. This is for example important when the cell-surface molecule that is targeted by the sdAb(s) comprised by the conjugate is expressed to a higher extent on target (tumor) cells, but is not uniquely expressed on such target cells. A lower dose of the conjugate lowers the risk for binding of the conjugate to such low expressors, such as non-tumor healthy cells.

The inventors also found that the therapeutic window of the conjugate of the invention is widened due to the incorporation of covalently bound saponin in the conjugate of the invention. That is to say, when the ADC or the AOC provided with a saponin (i.e. the conjugate of the invention) is contacted with target cells, upon binding of the at least one, preferably more than one sdAb to its binding partner at the surface of the target cell, the saponin that is comprised by the conjugate of the invention is also brought in close proximity, i.e. at the surface of the target cell, together with the effector molecule of the conjugate. When target cells that bear the cell-surface molecule, i.e. the target for the sdAb(s) comprised by the conjugate, are contacted with the conjugate of the invention, both the effective dose of the effector molecule and the effective dose of the saponin is lower than when the target cells are contacted with an ADC or AOC in the absence of saponin or in the presence of free (untargeted) saponin. The presence of the targeted saponin as part of the conjugate of the invention potentiates the activity of the effector molecule in the target cells, such that the therapeutic window of the conjugate, and therewith the therapeutic window of the effector molecule is widened. Sufficient effector molecule efficiency is achieved at lower dose when target cells are contacted with the conjugate of the invention. The similar effect is found by the inventors when an ADC or an AOC is contacted with the target cells in the presence of saponin or a functional derivative thereof, when effector-molecule potentiating activity of the saponin is considered, however, at a 100-fold to 1000-fold higher concentration of the free saponin (functional derivative) compared to the effective dose established when the conjugate of the invention is applied, which now comprises both the effector molecule and the effector molecule activity enhancing saponin, together with the sdAb(s), such as 2 or 3 sdAbs, for targeted binding of the conjugate to the target (tumor) cell. Thus, providing the saponin or the functional derivative thereof with a binding molecule (i.e. the sdAb(s) comprised by the conjugate of the invention) and also providing the very same conjugate with an effector molecule (i.e. the effector molecule comprised by the conjugate of the invention) results in an improved effector-molecule activity potentiating effect, when the conjugate of the invention is contacted with the target cell that expresses the cell-surface molecule on its surface, i.e. the binding target for the sdAb(s). Targeted saponin in the context of the conjugate comprising sdAb(s) is already effective at lower dose than free saponin, in delivery of the effector molecule inside the target cell, and in delivery from the endosome or lysosome of said cell into the cytosol, where the effector molecule should bind its target binding partner and should exerts its biological activity (e.g. cell killing in case of the target cell being a tumor cell and the effector molecule being e.g. a toxin), however the present inventors have found that the combination of saponin, targeting moiety (sdAb) and effector molecule (e.g. toxin, AON) in a single molecule is even more effective.

Moreover, providing a single conjugate is beneficial when compared to providing a combination of saponin linked to a cell-surface molecule binding ligand such as an sdAb and an effector molecule linked to a ligand for binding to the same or a different cell-surface molecule. Treating a patient with a single conjugate comprising the cell-targeting sdAb(s), the saponin and the effector moiety is less cumbersome than having to administer two separate compounds, perhaps as two different pharmaceutical compositions. Moreover, with the conjugate of the invention, selected target cells only have to bear a single type of cell-surface molecule such as an endocytic receptor, to which the sdAb(s) can bind, at an expression level sufficient for endocytosis of an effective amount of the effector moiety and the saponin combined together in the single conjugate. In contrast, when the saponin and the effector molecule are separately bound each to a different copy of a ligand (sdAb(s)) for binding to the same cell-surface molecule, the sdAb(s)-saponin molecule competes for binding to the cell-surface molecule with the binding of the sdAb(s)-effector moiety molecules on the same cell, therewith competing with regard to binding and endocytosis by the cell. This results in less efficient cytosolic delivery of the effector molecule, since building up an effective amount of saponin and effector molecule in the endosome of the target cell is hampered by the competitive binding of the two molecules. When the saponin is bound to a first ligand for binding to a first type of cell-surface molecule and the effector molecule is bound to a second ligand for binding to a second type of cell-surface molecule, efficiently building up an effective dose of the effector molecule in the cell bearing the first and second type of cell-surface molecule is hampered, when compared with the coinciding build up of the effective amount of effector molecule and saponin in the endosome of said cell when contacted with the conjugate of the invention, which requires only a single type of cell-surface molecule. Moreover, synchronizing the build up of sufficient amounts of saponin and effector molecule in the endosome is hampered when the two molecules comprising saponin or effector molecule and the first ligand and the second ligand, respectively, are applied and the first and second cell-surface molecule are expressed to different extents at the target cell and/or when the first and second cell-surface molecule have varying rates of endocytosis, for example. Providing the conjugate of the invention overcomes these drawbacks: the saponin and the effector molecule are accumulating in the endosome of the cells exposing the cell-surface molecule for binding of the one or more sdAb(s), together. Building up the effective amount of saponin and effector molecule in the endosome, for providing an effective amount of the effector molecule in the cytosol (and/or nucleus) of the target cell, is synchronized by applying the conjugate of the invention.

Hence, the inventors provide a pharmaceutical composition comprising the conjugate comprising the saponin (or a functional derivative), the effector molecule and the sdAb(s) (e.g. 2, 3 or 4, preferably 2, 3, more preferably 2 sdAbs) for targeted delivery of the conjugate at target cells and into the endosome of said cells, which pharmaceutical composition has an improved therapeutic window, less risk for inducing side effects when an effective dose of the effector molecule comprised by the conjugate is administered to a patient in need of effector molecule based therapy, and improved effector molecule activity due to improved delivery of the conjugate, and therewith the effector molecule, inside target cells under influence of the targeted saponin as part of the conjugate of the invention, more specifically inside the cytosol of such target cells, when compared to current ADCs and AOCs based on full-length antibodies or Fc comprising constructs thereof, which are not provided with a covalently linked saponin. It is part of the invention that such conjugates of the invention are administered to patients in need of effector molecule based therapy together with a dose of free saponin (or a functional derivative), although the application of the conjugate of sdAb(s), effector moiety and saponin alone is preferred.

An example of a saponin suitable for application in the conjugate of the invention is a mono-desmosidic or bi-desmosidic triterpene saponin belonging to the type of a 12,13-dehydrooleanane with optionally an aldehyde group in position C-23 and optionally comprising a glucuronic acid group in a carbohydrate substituent at the C-3beta-OH group of the saponin, preferably a bi-desmosidic triterpene saponin belonging to the type of a 12,13-dehydrooleanane with the aldehyde group in position C-23 and optionally comprising a glucuronic acid group in a carbohydrate substituent at the C-3beta-OH group of the saponin, more preferably, a bi-desmosidic triterpene saponin belonging to the type of a 12,13-dehydrooleanane with the aldehyde group in position C-23 and comprising a glucuronic acid group in a carbohydrate substituent at the C-3beta-OH group of the saponin. Typically, saponins comprising a quillaic acid aglycone or a gypsogenin aglycone are suitable for application in the conjugate. An exemplary saponin according to the invention comprises one, several, or all of the features of the saponin depicted as SAPONIN A and illustrated by the following structure:

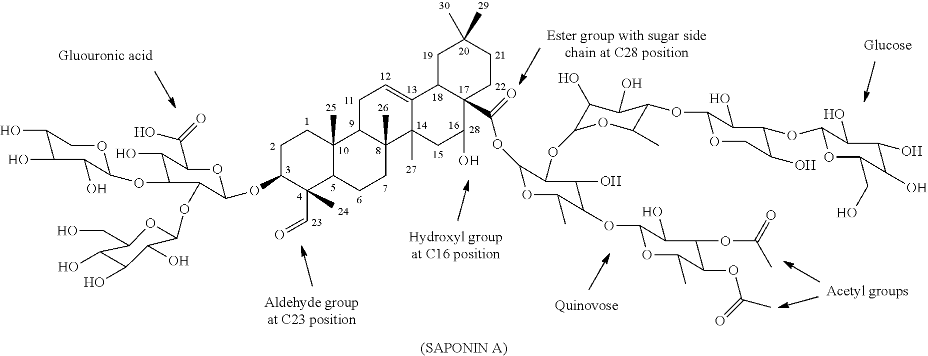

This group of saponins has demonstrated endosomal escape enhancing activity towards an effector moiety when the saponin and the effector moiety were present in the endosome of a cell. Typically, the saponins suitable for application in the conjugates are saponins with a triterpene backbone wherein the structure of the triterpene backbone is a pentacyclic C30 terpene skeleton (also referred to as sapogenin or aglycone). Table A1 lists saponins suitable for synthesizing a conjugate comprising at least one sdAb such as 1-8, preferably 2-6, more preferably 2-4 such as 2 or 3 V_{HH}('s) and comprising at least one saponin, such as 1-16 saponin moieties, 1-8, such as 2, 4 or 8 saponin moieties, wherein the saponin is for example SO1861, SO1832 or QS-21, preferably SO1861 or SO1832, more preferably SO1861.

An embodiment is the conjugate of the invention, comprising at least one sdAb which is any one or more of: a V_{H} domain derived from a heavy chain of an antibody, preferably of immunoglobulin G origin, preferably of human origin; a V_{L} domain derived from a light chain of an antibody, preferably of immunoglobulin G origin, preferably of human origin; a V_{HH} domain such as derived from a heavy-chain only antibody (HCAb) such as from Camelidae origin or Ig-NAR origin such as a variable heavy chain new antigen receptor (V_{NAR}) domain, preferably the HCAb is from Camelidae origin; and preferably the at least one sdAb is a V_{HH} domain derived from an HCAb from Camelidae origin (camelid V_{H}) such as derived from an HCAb from camel, lama, alpaca, dromedary, vicuna, guanaco and Bactrian camel.

An embodiment is the conjugate of the invention, wherein the sdAb(s) is/are selected from:
a V_{H} domain derived from a heavy chain of an antibody, preferably of immunoglobulin G origin, preferably of human origin;
a V_{L} domain derived from a light chain of an antibody, preferably of immunoglobulin G origin, preferably of human origin; and
a V_{HH} domain derived from a heavy-chain only antibody (HCAb), preferably from Camelidae origin or Ig-NAR origin such as a variable heavy chain new antigen receptor (V_{NAR}) domain, more preferably the HCAb is from Camelidae origin,

preferably the sdAb(s) of the bivalent nanobody is/are (a) V_{HH} domain(s) derived from an HCAb from Camelidae origin (camelid V_{H}) such as derived from an HCAb from camel, lama, alpaca, dromedary, vicuna, guanaco and Bactrian camel.

For example, the sdAb(s) is/are selected from:
a V_{H} domain derived from a heavy chain of an antibody, preferably of immunoglobulin G origin, and/or preferably of human origin;
a V_{L} domain derived from a light chain of an antibody, preferably of immunoglobulin G origin, and/or preferably of human origin; and
a V_{HH} domain derived from a heavy-chain only antibody (HCAb), preferably from Camelidae origin or Ig-NAR origin such as a variable heavy chain new antigen receptor (V_{NAR}) domain, more preferably the HCAb is from Camelidae origin,

preferably the sdAb(s) is/are (a) V_{HH} domain(s) derived from an HCAb from Camelidae origin (camelid V_{H}) such as derived from an HCAb from camel, lama, alpaca, dromedary, vicuna, guanaco and Bactrian camel.

In particular, V_{HH} domains are suitable for application in the conjugate of the invention. Such V_{HH} domains are commonly renowned for their high stability, i.e. resistance to unfolding, for their capability to bind to a binding partner without the requirement of the presence of a second V domain, such as present in e.g. IgG (Vₗ+Vₕ) and required for the IgG to bind to its binding partner via the two V domains, for their ease of production by techniques known in the art (camelid immunization, phage display techniques, etc.), for their capability of penetrating tissue to a higher extent than seen for full-length IgGs, which is beneficial when target (tumor) cells are located inside or as part of such (organ) tissue.

An embodiment is the conjugate of the invention, wherein the conjugate comprises 1-20 single domain antibodies, preferably at least one multivalent nanobody such as any of a divalent-hexavalent, preferably trivalent-pentavalent, nanobody, preferably at least one bivalent nanobody, preferably 1-8, more preferably 1-6, even more preferably 1-4 sdAb's or bivalent nanobodies, preferably 1, 2, 3 or 4 sdAb's or 1 or 2 tetravalent, trivalent and/or bivalent nanobodies, preferably 1 bivalent nanobody, or 1 bivalent nanobody and 1 further sdAb. The advantages of including more than one sdAb such as V_{HH} in the conjugate are manifold. For example, the conjugate comprises 1-20 sdAbs, preferably at least one multivalent nanobody such as any of a divalent-hexavalent, preferably trivalent-pentavalent, nanobody, preferably at least one bivalent nanobody, preferably 1-8, more preferably 1-6, even more preferably 1-4 sdAb's or 1-4 bivalent nanobodies, preferably 1, 2, 3 or 4 sdAb's or 1 or 2 tetravalent, trivalent and/or bivalent nanobodies, preferably 1 bivalent nanobody, or 1 bivalent nanobody and at least 1, preferably 1, further sdAb. For example, presence of more than one sdAb capable of binding to a cell-surface molecule present on the (same) cell, in the conjugate, increases binding avidity and affinity of the conjugate for the cell-surface molecule(s). For example, presence of more than one sdAb capable of binding to a cell-surface molecule present on the (same) cell, in the conjugate, can result in cell-surface molecule clustering on the cell surface, (further) facilitating the endocytosis of the conjugate. For example, the conjugate may comprise a multivalent sdAb, consisting of a string of covalently linked sdAbs via peptide bonds, such as 2-8 sdAbs linked together, preferably 2 or 3 sdAbs linked together. For example, such a multivalent sdAb is a bivalent or trivalent or tetravalent sdAb. For example, the sdAbs of such a multivalent sdAb bind to the same epitope of the cell-surface molecule (endocytic receptor), and/or bind to different epitopes of the cell-surface molecule, preferably non-overlapping epitopes. Preferably, for such multivalent sdAbs such as a bivalent sdAb, the first and second sdAb bind to non-overlapping epitopes and binding of the first sdAb to a first epitope of the cell-surface molecule preferably does not compete with binding of the second sdAb to a second epitope of the cell-surface molecule (referred to as multi-paratopic format, or bi-paratopic, when the conjugate comprises two sdAbs for binding to a first and second non-overlapping epitope wherein binding of the two sdAbs do not compete with each other).

An embodiment is the conjugate of the invention, comprising at least two sdAbs with a single first sdAb covalently bound to one of the at least one effector molecule and/or to one of the at least one saponin, or with two or more sdAbs of which at least one sdAb is bound to the at least one effector molecule and/or of which at least one sdAb is bound to the at least one saponin, or with all of the at least two sdAbs each bound separately to either an effector molecule of the at least one effector molecule or to a saponin of the at least one saponin, or both. A preferred embodiment is the conjugate of the invention wherein the at least one saponin and the at least one effector moiety are bound to the at least one, preferably 2-3 sdAbs, via a linker covalently bound to a peptide linker comprised by the sdAb(s), such as the tetra-Cys linker HRWCCPGCCKTF with SEQ ID NO: 77.

An embodiment is the conjugate of the invention, wherein the at least one sdAb comprises at least two sdAbs, which are the same sdAbs, preferably two-eight sdAbs, more preferably two-four sdAbs, or comprising at least two sdAbs which are different or the same, such as two different sdAbs (e.g. biparatopic sdAb), two or three sdAbs which are the same and one, two or three further sdAbs which are the same or different. For the conjugate comprising at least two different sdAb, a first sdAb may bind to a first cell surface molecule present on a first cell and a second sdAb different from the first sdAb may bind to a second molecule such as a second cell surface molecule present on the same first cell or present on a second cell different from the first cell.

An embodiment is the conjugate of the invention, comprising at least two sdAbs which are biparatopic, preferably comprising two sdAbs which are biparatopic.

An embodiment is the conjugate of the invention, wherein the bivalent nanobody is a hetero-bivalent nanobody, consisting of a first and second sdAb.

An embodiment is the conjugate of the invention, comprising one-eight sdAbs, capable of binding to a same binding site on a cell-surface molecule, wherein the at least one effector molecule and/or the at least one saponin is/are bound to a single first sdAb of the one-eight sdAbs or wherein the at least one effector molecule and/or the at least one saponin is/are bound to two or more of the sdAbs, if present, wherein the at least one effector molecule and the at least one saponin are bound to the same sdAb or are bound to different sdAbs, wherein preferably each of the at least one effector molecule is bound to a separate sdAb and/or each of the at least one saponin is bound to a separate sdAb, wherein an effector molecule and a saponin are bound to the same sdAb or are bound to separate sdAbs.

An embodiment is a conjugate of the invention, comprising at least one bivalent nanobody, preferably a single bivalent nanobody, comprising a first and second sdAb, wherein the first sdAb has an amino-acid sequence of SEQ ID NO: 75 or an amino-acid sequence with at least 95% sequence identity with SEQ ID NO: 75, preferably at least 96%, more preferably at least 97%, even more preferably at least 98%, even more preferably at least 99%, and the second sdAb has an amino-acid sequence of SEQ ID NO: 76 or an amino-acid sequence with at least 95% sequence identity with SEQ ID NO: 76, preferably at least 96%, more preferably at least 97%, even more preferably at least 98%, even more preferably at least 99%.

An embodiment is a conjugate of the invention, wherein the cell surface molecule is a cell surface receptor, preferably an endocytic cell-surface receptor, preferably a tumor-cell specific receptor, more preferably the cell surface molecule is selected from any one or more of: CD71, CA125, EpCAM(17-1A), CD52, CEA, CD44v6, FAP, EGF-IR, integrin, syndecan-1, vascular integrin alpha-V beta-3, HER2, EGFR, CD20, CD22, Folate receptor 1, CD146, CD56, CD19, CD138, CD27L receptor, prostate specific membrane antigen (PSMA), CanAg, integrin-alphaV, CA6, CD33, mesothelin, Cripto, CD3, CD30, CD239, CD70, CD123, CD352, DLL3, CD25, ephrinA4, MUC-1, Trop2, CEACAM5, CEACAM6, HER3, CD74, PTK7, Notch3, FGF2, C4.4A, FLT3, CD38, FGFR3, CD7, PD-L1, CTLA-4, CD52, PDGFRA, VEGFR1, VEGFR2, c-Met (HGFR), EGFR1, RANKL, ADAMTS5, CD16, CXCR7 (ACKR3), glucocorticoid-induced TNFR-related protein (GITR), even more preferably the cell surface molecule is selected from: HER2, c-Met, VEGFR2, CXCR7, CD71, EGFR and EGFR1, even more preferably the cell surface molecule is EGFR. In addition, the cell surface molecule is selected from CD63.

An embodiment is a conjugate of the invention, wherein the sdAb(s), preferably a multivalent nanobody, more preferably a single bivalent nanobody, are selected from: anti-CD71 sdAb(s), anti-HER2 sdAb(s), anti-CD20 sdAb(s), anti-CA125 sdAb(s), anti-EpCAM (17-1A) sdAb(s), anti-EGFR sdAb(s), anti-CD30 sdAb(s), anti-CD33 sdAb(s), anti-vascular integrin alpha-v beta-3 sdAb(s), anti-CD52 sdAb(s), anti-CD22 sdAb(s), anti-CEA sdAb(s), anti-CD44v6 sdAb(s), anti-FAP sdAb(s), anti-CD19 sdAb(s), anti-CanAg sdAb(s), anti-CD56 sdAb(s), anti-CD38 sdAb(s), anti-CA6 sdAb(s), anti-IGF-1R sdAb(s), anti-integrin sdAb(s), anti-syndecan-1 sdAb(s), anti-CD79b sdAb, anti-c-Met sdAb(s), anti-EGFR1 sdAb(s), anti-VEGFR2 sdAb(s), anti-CXCR7 sdAb(s) and anti-HIVgp41 sdAb(s), wherein the sdAbs are preferably V_{HH}(s), more preferably camelid V_{H}(s). For example the sdAb(s), preferably a multivalent nanobody, more preferably a single bivalent nanobody or one bivalent nanobody and one further sdAb, are at least selected from: anti-CD71 sdAb(s), anti-HER2 sdAb(s), anti-CD20 sdAb(s), anti-CA125 sdAb(s), anti-EpCAM (17-1A) sdAb(s), anti-EGFR sdAb(s), anti-CD30 sdAb(s), anti-CD33 sdAb(s), anti-vascular integrin alpha-v beta-3 sdAb(s), anti-CD52 sdAb(s), anti-CD22 sdAb(s), anti-CEA sdAb(s), anti-CD44v6 sdAb(s), anti-FAP sdAb(s), anti-CD19 sdAb(s), anti-CanAg sdAb(s), anti-CD56 sdAb(s), anti-CD38 sdAb(s), anti-CA6 sdAb(s), anti-IGF-1R sdAb(s), anti-integrin sdAb(s), anti-syndecan-1 sdAb(s), anti-CD79b sdAb, anti-c-Met sdAb(s), anti-EGFR1 sdAb(s), anti-VEGFR2 sdAb(s), anti-CXCR7 sdAb(s) and anti-HIVgp41 sdAb(s), and optionally also anti-albumin sdAb(s), wherein the sdAbs are preferably VHH(s), more preferably camelid VH(s). For example the sdAb(s), preferably a multivalent nanobody, more preferably a single bivalent nanobody or one bivalent nanobody and one further sdAb, are at least selected from: CD63. The albumin is preferably serum albumin.

An embodiment is a conjugate of the invention, wherein the sdAbs are derived from or based on any one or more of immunoglobulins: an anti-CD71 antibody such as IgG type OKT-9, an anti-HER2 antibody such as trastuzumab (Herceptin), pertuzumab, an anti-CD20 antibody such as rituximab, ofatumumab, tositumomab, obinutuzumab ibritumomab, an anti-CA125 antibody such as oregovomab, an anti-EpCAM (17-1A) antibody such as edrecolomab, an anti-EGFR antibody such as cetuximab, matuzumab, panitumumab, nimotuzumab, an anti-CD30 antibody such as brentuximab, an anti-CD33 antibody such as gemtuzumab, huMy9-6, an anti-vascular integrin alpha-v beta-3 antibody such as etaracizumab, an anti-CD52 antibody such as alemtuzumab, an anti-CD22 antibody such as epratuzumab, pinatuzumab, binding fragment (Fv) of anti-CD22 antibody moxetumomab, humanized monoclonal antibody inotuzumab, an anti-CEA antibody such as labetuzumab, an anti-CD44v6 antibody such as bivatuzumab, an anti-FAP antibody such as sibrotuzumab, an anti-CD19 antibody such as huB4, an anti-CanAg antibody such as huC242, an anti-CD56 antibody such as huN901, an anti-CD38 antibody such as daratumumab, OKT-10 anti-CD38 monoclonal antibody, an anti-CA6 antibody such as DS6, an anti-IGF-1R antibody such as cixutumumab, 3B7, an anti-integrin antibody such as CNTO 95, an anti-syndecan-1 antibody such as B-B4, an anti-CD79b such as polatuzumab, an anti-HIVgp41 antibody, preferably any one of an anti-HIVgp41 antibody, an anti-CD71 antibody, an anti-HER2 antibody and an anti-EGFR antibody, more preferably the sdAbs are derived from or based on any one or more of: trastuzumab, pertuzumab, cetuximab, matuzumab, an anti-CD71 antibody, OKT-9, even more preferably trastuzumab, cetuximab, the anti-CD71 antibody OKT-9. For example, the sdAbs are at least derived from or based on any one or more of immunoglobulins: an anti-CD71 antibody such as IgG type OKT-9, an anti-HER2 antibody such as trastuzumab (Herceptin), pertuzumab, an anti-CD20 antibody such as rituximab, ofatumumab, tositumomab, obinutuzumab ibritumomab, an anti-CA125 antibody such as oregovomab, an anti-EpCAM (17-1A) antibody such as edrecolomab, an anti-EGFR antibody such as cetuximab, matuzumab, panitumumab, nimotuzumab, an anti-CD30 antibody such as brentuximab, an anti-CD33 antibody such as gemtuzumab, huMy9-6, an anti-vascular integrin alpha-v beta-3 antibody such as etaracizumab, an anti-CD52 antibody such as alemtuzumab, an anti-CD22 antibody such as epratuzumab, pinatuzumab, binding fragment (Fv) of anti-CD22 antibody moxetumomab, humanized monoclonal antibody inotuzumab, an anti-CEA antibody such as labetuzumab, an anti-CD44v6 antibody such as bivatuzumab, an anti-FAP antibody such as sibrotuzumab, an anti-CD19 antibody such as huB4, an anti-CanAg antibody such as huC242, an anti-CD56 antibody such as huN901, an anti-CD38 antibody such as daratumumab, OKT-10 anti-CD38 monoclonal antibody, an anti-CA6 antibody such as DS6, an anti-IGF-1R antibody such as cixutumumab, 3B7, an anti-integrin antibody such as CNTO 95, an anti-syndecan-1 antibody such as B-B4, an anti-CD79b such as polatuzumab, an anti-HIVgp41 antibody, preferably any one of an anti-HIVgp41 antibody, an anti-CD71 antibody, an anti-HER2 antibody and an anti-EGFR antibody, more preferably the sdAbs are at least derived from or based on any one or more of: trastuzumab, pertuzumab, cetuximab, matuzumab, an anti-CD71 antibody, OKT-9, even more preferably trastuzumab, cetuximab, the anti-CD71 antibody OKT-9. For example, the sdAbs are at least derived from or based on anti-CD63 antibody.

An embodiment is a conjugate of the invention, wherein at least one of the sdAbs competes with binding of any one of the immunoglobulins listed in in the embodiment directly here above, to the cell surface molecule. For example, at least one of the sdAbs competes with binding of any one of the immunoglobulins listed here above to the cell surface molecule and/or wherein the binding site on the first cell-surface molecule for the at least one of the sdAbs is the same or overlaps with the binding site on the first cell-surface molecule for any one of the immunoglobulins listed here above.

An embodiment is a conjugate of the invention, wherein the sdAbs are capable of binding to HER2, CD71, HIVgp41 or EGFR, preferably EGFR, wherein the sdAbs preferably are a V_{HH}, more preferably a camelid V_{H}. For example, the sdAbs are capable of binding to at least HER2, CD71, HIVgp41 or EGFR, preferably EGFR, wherein the sdAbs preferably are a V_{HH}, more preferably a camelid V_{H}.

For example, the conjugate comprises at least one multivalent nanobody, preferably at least one bivalent nanobody comprising two sdAbs, of which nanobody at least one sdAb binds to the first cell surface molecule that is present on the first cell.

For example, the at least one multivalent nanobody comprised by the conjugate, preferably at least one bivalent nanobody comprising two sdAbs, comprised by the conjugate, comprises two sdAbs which are the same sdAbs or which are two different sdAbs.

For example, one of the sdAbs of the at least one multivalent nanobody comprised by the conjugate, preferably the at least one bivalent nanobody comprising two sdAbs, comprised by the conjugate, binds to the first cell surface molecule and at least one sdAb binds to albumin. Preferably, the albumin is serum albumin.

For example, the conjugate further comprises an albumin binding protein and/or albumin. Preferably, the albumin is serum albumin.

For example, at least one multivalent nanobody comprised by the conjugate, preferably at least one bivalent nanobody comprising two sdAbs, comprised by the conjugate, is multiparatopic such as biparatopic, and/or multi-specific such as bi-specific for the first cell-surface molecule and for a second cell-surface molecule also present at the first cell, or the second cell-surface molecule present at a second cell.

An embodiment is a conjugate of the invention,

wherein the sdAbs capable of binding to HER2 are selected from: sdAb produced by clone 11A4, clone 18C3, clone 22G12, clone Q17 or clone Q17-C-tag,
wherein the sdAbs capable of binding to EGFR is produced by clone anti-EGFR Q86-C-tag,
wherein the sdAbs capable of binding to CD71 is produced by clone anti-CD71 Q52-C-tag;
and wherein the sdAbs capable of binding to HIVgp41 is produced by clone anti-HIVgp41 Q8C-tag;
preferably wherein the sdAbs are encoded by a cDNA of any one of the SEQ ID NO: 1, 3, 5, 7, 9, 11, 13, 15, 17, 19, 21, 23, 25, 27, 29 and 31 or wherein the sdAbs have an amino-acid sequence according to any one or more of SEQ ID NO: 2, 4, 6, 8, 10, 12, 14, 16, 18, 20, 22, 24, 26, 28, 30, 32, 36-71 or 72, or an amino-acid sequence with at least 95% sequence identity with an amino-acid sequence according to any one or more of SEQ ID NO: 2, 4, 6, 8, 10, 12, 14, 16, 18, 20, 22, 24, 26, 28, 30, 32, 36-71 or 72, preferably at least 96%, more preferably at least 97%, even more preferably at least 98%, even more preferably at least 99%.

An embodiment is a conjugate of the invention, comprising at least two sdAbs which are biparatopic, preferably comprising two sdAbs which are biparatopic. An example of such a biparatopic tandem of sdAbs is the biparatopic tandem of sdAbs with amino-acid sequence as depicted as SEQ ID NO: 74. The bivalent tandem of sdAb having amino-acid sequence of SEQ ID NO: 74 binds to EGFR. A first sdAb, 7D12 with amino-acid sequence as depicted as SEQ ID NO: 75, binds to a first epitope on the EGFR and a second sdAb, 9G8, with amino-acid sequence as depicted as SEQ ID NO: 76, binds to a second epitope on the EGFR. Of course, the sdAb 7D12 or the sdAb 9G8 are also suitable for application in a conjugate of the invention which comprises a single sdAb, or which comprises at least one further sdAb different from 7D12 and 9G8, which at least one further sdAb binds to EGFR or to a further cell-surface molecule present on the same cell surface as at which the EGFR is exposed. For providing a conjugate of the invention, an effector molecule is covalently linked to the one or multiple sdAb's, such as a protein toxin such as dianthin (see example of 7D12-9G8-dianthin, in the Examples section, with amino-acid sequence as outlined in SEQ ID NO: 73), and at least one copy of a saponin such as QS-21 or SO1861, is covalently linked to the sdAb's, for example via linker and/or via an oligomeric molecule or polymeric molecule which oligomeric molecule or polymeric molecule is covalently linked to one or multiple saponin molecules and to the sdAb's.

An embodiment is a conjugate of the invention, wherein the hetero-bivalent nanobody is a biparatopic nanobody, preferably a biparatopic nanobody with amino-acid sequence of SEQ ID NO: 74 or an amino-acid sequence with at least 95% sequence identity with SEQ ID NO: 74, preferably at least 96%, more preferably at least 97%, even more preferably at least 98%, even more preferably at least 99%.

Providing a conjugate of the invention which comprises a (linear) string of multiple sdAbs covalently linked to each other (via peptide bonds), can provide the benefit of the capacity of the conjugate to bind with higher avidity to the target cell, which can result in improved uptake (endocytosis) of the conjugate by the target cell (binding of conjugates to the receptor is followed by internalization of the conjugate/receptor complex).

Synchronization is the missing link between a successful delivery strategy for application in humans, when the application of the endosomal escape enhancing effect of saponin towards effector molecules is considered. Indeed, the inventors established in a series of in vivo mouse tumor models that separately administering to the mice a dose of free saponin and a dose of e.g. ADC without coupled saponin, did not result in any desired anti-tumor activity such as delayed tumor growth, tumor regression, diminished and slower tumor growth, compared to control animals not treated with the ADC in the presence of free saponin. The free saponin was administered using various routes of administration and using various time points of administering the free saponin compared to the moment of administering the ADC (administering free saponin before, during and after administering the ADC). The ADC tested in in vivo tumor models was cetuximab-dianthin (with free SO1861), or trastuzumab-saporin (with free SO1861). Varying the dose of free saponin did not provide for an efficacious anti-tumor activity. The ADCs referred to were administered at a dose that in itself did not inflict any beneficial anti-tumor effect on the tumor-bearing animals. Surprisingly, the inventors now established that beneficial anti-tumor activity in various in vitro mammalian cell-based bioassays using human tumor cells can be achieved by treating the cells with conjugates according to the invention. The conjugates optionally comprising a scaffold according to the invention (see below, a covalent saponin conjugate comprising an oligomeric or polymeric structure with one or multiple saponin moieties covalently bound thereto). The scaffold for example being a tri-functional linker with at least one, preferably 2-16, such as 4-8, preferably 4 or 8, covalently bound saponin molecules (e.g. a saponin selected from Table A1, preferably a saponin comprising a quillaic acid based aglycone or gypsogenin based aglycone, preferably SO1832, SO1861, QS-21, more preferably SO1861 or SO1832, even more preferably SO1861) via a cleavable or non-cleavable linkage, preferably a cleavable bond between the saponin(s) and the scaffold, and/or with a covalently bound effector moiety (e.g. an oligonucleotide such as a gene-silencing oligonucleotide such as antisense BNA (e.g. BNA HSP27) via a non-cleavable bond or, preferably, a cleavable bond, the scaffold linked with a covalently bond to the cell-surface molecule targeting molecule of the conjugate, here at least one sdAb, preferably 2-4 sdAbs, or the scaffold being a dendron, for example G2-dendron or G3-dendron, to which for example four respectively 8 moieties can bind such as four and eight saponin molecules, or a G2 dendron for binding for example two saponins and two effector molecules, the dendron comprising a chemical group for (covalent) coupling to the cell-surface molecule targeting sdAb(s), of the conjugate. Reference is made to the further embodiments and the Examples section, exemplifying several of these scaffolds according to the invention, showing activity when gene silencing in the (tumor) cell is considered.

Without wishing to be bound by any theory, in view of the failures observed when treatment of tumor-bearing animals with an ADC together with free saponin is considered, it is preferred to synchronize the presence of both, the at least one saponin, and the effector moiety, preferably a toxin or an oligonucleotide, in compartments or vesicles of the endocytic pathway of the target cell, e.g. a tumor cell or an auto-immune cell. With ADC or AOC and free saponin, synchronizing the presence of the molecules in the late endosomes, in order to obtain the synergistic effects in vivo is more difficult to obtain.

An embodiment is the conjugate of the invention, wherein the at least one sdAb is a single sdAb or, preferably, are at least two, preferably two sdAbs, wherein the sdAb(s) is/are capable of binding to a cell-surface molecule of the cell such as HIVgp41 or wherein the sdAb(s) is/are capable of binding to a cell-surface receptor of the cell, such as a tumor-cell surface receptor of the cell, preferably a tumor-cell specific receptor, more preferably to a receptor selected from any one or more of: CD71, CA125, EpCAM(17-1A), CD52, CEA, CD44v6, FAP, EGF-IR, integrin, syndecan-1, vascular integrin alpha-V beta-3, HER2, EGFR, CD20, CD22, Folate receptor 1, CD146, CD56, CD19, CD138, CD27L receptor, prostate specific membrane antigen (PSMA), CanAg, integrin-alphaV, CA6, CD33, mesothelin, Cripto, CD3, CD30, CD239, CD70, CD123, CD352, DLL3, CD25, ephrinA4, MUC-1, Trop2, CEACAM5, CEACAM6, HER3, CD74, PTK7, Notch3, FGF2, C4.4A, FLT3, CD38, FGFR3, CD7, PD-L1, CTLA-4, CD52, PDGFRA, VEGFR1, VEGFR2, c-Met (HGFR), EGFR1, RANKL, ADAMTS5, CD16, CXCR7 (ACKR3), glucocorticoid-induced TNFR-related protein (GITR), most preferably selected from: HER2, c-Met, VEGFR2, CXCR7, CD71, EGFR and EGFR1. For example, the at least one sdAb is a single sdAb or, preferably, are at least two, preferably two sdAbs, wherein the sdAb(s) is/are capable of binding to CD63.

It is part of the invention that the sdAb(s) comprised by the conjugate of the invention has/have binding specificity for a cell-surface molecule that is specifically expressed on the target cell. ‘Specifically expressed’ should here be understood as the unique expression of the cell-surface molecule on the target cell only, wherein e.g. healthy cells that should not bind the conjugate, are not targeted due to the absence of cell-surface exposure of the targeted molecule, or should here be understood as the upregulated or relatively high expression of the target cell-surface molecule on the target cells, compared to lower expression of the cell-surface molecule on e.g. healthy cells that should not or at least to a much lower extent, bind the conjugate. These listed cell receptors are such cell-surface molecules that are sufficiently specific for the cells that are the target of the conjugate, and are therewith preferred candidates for binding by the conjugate. It will be appreciated that the higher the specificity of a certain cell-surface molecule, when expression of the cell-surface molecule on the target cell is compared to the expression on other cells not meant to be targeted by the conjugate of the invention, the better the therapeutic window is, when the activity of the effector molecule inside the cells is considered. For example, suitable targets for targeting by the conjugate are amongst other tumor cell specific receptors, HER2, EGFR, such as EGFR1, and CD71.

An embodiment is the conjugate of the invention, wherein the at least one sdAb comprises an sdAb that is capable of binding to HER2, CD71, HIVgp41 and/or EGFR, wherein said sdAb is preferably a V_{HH}, more preferably a camelid V_{H}.

An embodiment is the conjugate of the invention, wherein the at least one sdAb comprises an sdAb for binding to HER2 selected from: sdAb produced by clone 11A4, clone 18C3, clone 22G12, clone Q17 or clone Q17-C-tag; or comprises an sdAb for binding to EGFR and produced by clone anti-EGFR Q86-C-tag; or comprises an sdAb for binding to CD71 and produced by clone anti-CD71 Q52-C-tag; or comprises an sdAb for binding to HIVgp41 and produced by clone anti-HIVgp41 Q8C-tag; or comprises an sdAb encoded by a cDNA of any one of the SEQ ID NO: 1, 3, 5, 7, 9, 11, 13, 15, 17, 19, 21, 23, 25, 27, 29 and 31; or comprises any one of the sdAbs with an amino-acid sequence of SEQ ID NO: 2, 4, 6, 8, 10, 12, 14, 16, 18, 20, 22, 24, 26, 28, 30, 32, 36-72, or comprises the biparatopic tandem of V_{HH} domains binding to EGFR, consisting of V_{HH} 7D12 and V_{HH} 9G8 with the amino-acid sequence as depicted as SEQ ID NO: 74, or comprises V_{HH} 7D12 with the amino-acid sequence as depicted as SEQ ID NO: 75 and/or V_{HH} 9G8 with the amino-acid sequence as depicted as SEQ ID NO: 76, wherein optionally the conjugate further comprises a further sdAb, different from the at least one sdAb, the further sdAb for binding to albumin, preferably serum albumin, such as any one or more of the further sdAbs with an amino-acid sequence of SEQ ID NO: 33, 34 and 35, preferably the further sdAb is a V_{HH}, more preferably a camelid V_{H}.

An embodiment is the conjugate of the invention, wherein the conjugate comprises an sdAb for binding to HER2 selected from: sdAb produced by clone 11A4, clone 18C3, clone 22G12, clone Q17, clone Q17-C-tag; or an sdAb for binding to EGFR and produced by clone anti-EGFR Q86-C-tag; or an sdAb for binding to CD71 and produced by clone anti-CD71 Q52-C-tag; or an sdAb for binding to HIVgp41 and produced by clone anti-HIVgp41 Q8-C-tag; or an sdAb encoded by a cDNA of any one of the SEQ ID NO: 1, 3, 5, 7, 9, 11, 13, 15, 17, 19, 21, 23, 25, 27, 29 and 31; or any one of the sdAbs with an amino-acid sequence of SEQ ID NO: 2, 4, 6, 8, 10, 12, 14, 16, 18, 20, 22, 24, 26, 28, 30, 32, 36-72, or the biparatopic tandem of sdAbs 7D12 and 9G8 with an amino-acid sequence of SEQ ID NO: 74 or sdAb 7D12 with an amino-acid sequence of SEQ ID NO: 75 or sdAb 9G8 with an amino-acid sequence of SEQ ID NO: 76, wherein optionally the conjugate further comprises an sdAb for binding to albumin, preferably serum albumin, such as any one or more of sdAbs with an amino-acid sequence of SEQ ID NO: 33, 34 and 35. For example, the conjugate of a saponin, an effector moiety and at least one V_{HH} comprises the tandem of biparatopic sdAbs with the amino-acid sequence of SEQ ID NO: 74, or comprises one or more copies of 7D12 and/or one or more copies of 9G8.

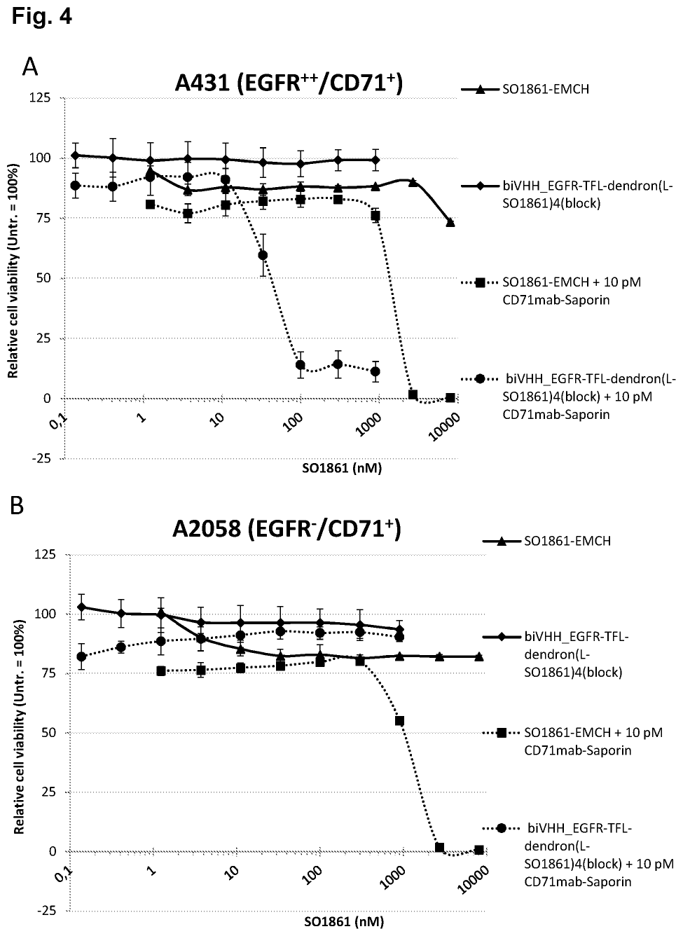

V_{HH}S suitable for incorporation in the conjugate of the invention are for example found in the single domain antibody database (Wilton, E. E. et al. (2018)), in patent applications US20160251440 (anti-CD123, anti-CEACAM), U.S. Pat. No. 9,683,045 (anti-c-Met), US20090252681 (anti-EGFR, anti-IGF-1R), U.S. Pat. No. 9,969,805 (anti-HER2), US20190023796A1 (anti-HER3), and in Kijanka et al. (2013), for anti-HER2 and in Mercier et al. (2019) for anti-HER2. The amino-acid sequences and/or the cDNA sequences of a series of suitable V_{HH}S is also provided here below for anti-HER2, anti-HER3, anti-CD123, anti-CEACAM, anti-c-Met, anti-EGFR, anti-IGF-1R, anti-PD-L1, anti-CTLA-4, anti-CD19, anti-HER1 and anti-VGFR2, as SEQ ID NOs 1-32 and 36-72 and 74 (tandem of V_{HH}'s 7D12 and 9G8) and V_{HH} 7D12 with the amino-acid sequence as depicted as SEQ ID NO: 75 and/or V_{HH} 9G8 with the amino-acid sequence as depicted as SEQ ID NO: 76, in view of their ability to bind to tumor-cell specific receptors. In particular, a V_{HH} capable of binding to a binding site on any of the tumor-cell specific receptors HER2, VEGFR, EGFR and CD71 is suitable for incorporation in the conjugate of the invention. In particular, a V_{HH} capable of binding to a binding site on tumor-cell specific receptor EGFR is suitable for incorporation in the conjugate of the invention, such as V_{HH} 7D12 with the amino-acid sequence as depicted as SEQ ID NO: 75 and V_{HH} 9G8 with the amino-acid sequence as depicted as SEQ ID NO: 76. The inventors revealed that an ADC comprising a V_{HH} that targets any one of such receptors is effective in delivery of the effector molecule bound to the sdAb. See for example the Examples section, and FIGS. 4-7.

An embodiment is the conjugate of the invention, wherein the effector molecule comprises or consists of at least one of a small molecule such as a drug molecule, a toxin such as a protein toxin, an oligonucleotide such as a BNA, a xeno nucleic acid or an siRNA, an enzyme, a peptide, a protein, or any combination thereof.

An embodiment is the conjugate of the invention, wherein the cell is an aberrant cell such as a tumor cell, an auto-immune cell, an infected cell such as a virally infected cell, or a cell comprising a gene defect or an enzyme defect, preferably wherein the cell is a tumor cell, and/or wherein the cell is a liver cell or an aberrant liver cell such as a tumor cell.

An embodiment is the conjugate of the invention, wherein the effector molecule has a molecular weight of less than 200 kDa, preferably less than 150 kDa, more preferably less than 100 kDa, more preferably less than 50 kDa and/or, when the at least one effector molecule is an oligonucleotide, wherein the oligonucleotide has a size of 150 nt or less, preferably 5-150 nt, more preferably 8-100 nt, even more preferably 10-50 nt. Typically, the oligonucleotides comprised by the conjugate have a size of 8-40 nt, such as 12-25 nt.

An embodiment is the conjugate according to the invention, wherein the effector molecule is a pharmaceutically active substance, such as a toxin such as a proteinaceous toxin, a drug, a polypeptide or a polynucleotide. A pharmaceutically active substance in this invention is an effector molecule that is used to achieve a beneficial outcome in an organism, preferably a vertebrate, more preferably a human being. Benefits include diagnosis, prognosis, treatment, cure and prevention of diseases and/or symptoms. The pharmaceutically active substance may also lead to undesired harmful side effects. In this case, pros and cons must be weighed to decide whether the pharmaceutically active substance is suitable in the particular case. If the effect of the pharmaceutically active substance inside a cell is predominantly beneficial for the organism as a whole, e.g. a human patient, the cell is called a target cell. If the effect inside a cell is predominantly harmful for the organism as a whole, the cell is called an off-target cell. In artificial systems such as cell cultures and bioreactors, target cells and off-target cells depend on the purpose and are defined by the user. Examples of effector molecules are a drug, a toxin, a polypeptide (such as an enzyme), and a polynucleotide, including polypeptides and polynucleotides that comprise non-natural amino acids or nucleic acids. Effector molecules include, amongst others: DNA: single stranded DNA (e.g. DNA for adenine phosphoribosyltransferase); linear doubled stranded DNA; circular double stranded DNA (e.g. plasmids); RNA: -mRNA (e.g. TAL effector molecule nucleases), tRNA, rRNA, siRNA, miRNA, asRNA, LNA and BNA; Protein and peptides; toxins (e.g. saporin, dianthin, gelonin, (de)bouganin, agrostin, ricin (toxin A chain); pokeweed antiviral protein, apoptin, diphtheria toxin, pseudomonas exotoxin) metabolic enzymes (argininosuccinate lyase, argininosuccinate synthetase), enzymes of the coagulation cascade, repairing enzymes; enzymes for cell signalling; cell cycle regulation factors; gene regulating factors (transcription factors such as NF-κB or gene repressors such as methionine repressor). A toxin, as used in this invention, is defined as a pharmaceutically active substance that is able to kill or inactivate a cell. Preferably, a targeted toxin is a toxin that is only, or at least predominantly, toxic for target cells but not for off-target cells. The net effect of the targeted toxin is preferably beneficial for the organism as a whole.

An embodiment is the conjugate of the invention, wherein the at least one effector molecule is an oligonucleotide selected from: deoxyribonucleic acid (DNA) oligomer, ribonucleic acid (RNA) oligomer, anti-sense oligonucleotide (ASO, AON), short interfering RNA (siRNA), anti-microRNA (anti-miRNA), DNA aptamer, RNA aptamer, mRNA, mini-circle DNA, peptide nucleic acid (PNA), phosphoramidate morpholino oligomer (PMO), locked nucleic acid (LNA), bridged nucleic acid (BNA), 2′-deoxy-2′-fluoroarabino nucleic acid (FANA), 2′-O-methoxyethyl-RNA (MOE), 3′-fluoro hexitol nucleic acid (FHNA), glycol nucleic acid (GNA), xeno nucleic acid oligonucleotide and threose nucleic acid (TNA), preferably the oligonucleotide is a BNA, more preferably the oligonucleotide is a BNA for silencing HSP27 protein expression or a BNA for silencing apolipoprotein B expression.

For example, the at least one effector molecule comprised by the conjugate is an oligonucleotide selected from deoxyribonucleic acid (DNA) oligomer, ribonucleic acid (RNA) oligomer, anti-sense oligonucleotide (ASO, AON), short interfering RNA (siRNA), anti-microRNA (anti-miRNA), DNA aptamer, RNA aptamer, mRNA, mini-circle DNA, peptide nucleic acid (PNA), phosphoramidate morpholino oligomer (PMO), phosphorothioate-modified antisense oligonucleotide (PS-ASO), 2′-O-methyl (2′-OMe) phosphorothioate RNA, 2′-O-methoxyethyl (2′-O-MOE) RNA {2′-O-methoxyethyl-RNA (MOE)}, locked nucleic acid (LNA), bridged nucleic acid (BNA), 2′-deoxy-2′-fluoroarabino nucleic acid (FANA), 2′-O-methoxyethyl-RNA (MOE), 3′-fluoro hexitol nucleic acid (FHNA), glycol nucleic acid (GNA), xeno nucleic acid oligonucleotide and threose nucleic acid (TNA), preferably the oligonucleotide is a BNA, more preferably the oligonucleotide is a BNA for silencing HSP27 protein expression or a BNA for silencing apolipoprotein B expression.

For completeness, with regard to the nucleic acids or oligonucleotides advantageously being part of the disclosed herein conjugates, as used herein the term oligonucleotide shall be understood as encompassing both the oligomers that are made of naturally occurring nucleotides and hence, chemically are oligonucleotides, as well as oligomers comprising modified oligonucleotides or analogues thereof. For example, a synthetic oligomer may comprise e.g. 2′ modified nucleosides which can be selected from: 2′-fluoro (2′-F), 2′-O-methyl (2′-O-Me), 2′-O-methoxyethyl (2′-MOE). 2′-O-aminopropyl (2′O-AP), 2′-O-dimethylaminoethyl (2′-O-DMAOE), 2′-O-dimethylaminopropyl (2′-O-DMAP),2′-O-dimethylaminoethyloxyethyl (2′-O-DMAEOE), 2′-O-N-methylacetamido (2′-O-NMA), locked nucleic acid (LNA), ethylene-bridged nucleic acid (ENA), and (S)-constrained ethylbridged nucleic acid (cEt), etc. In line with this, in a possible embodiment, the oligonucleotide can structurally or functionally be defined as any of: a deoxyribonucleic acid (DNA) oligomer, ribonucleic acid (RNA) oligomer, anti-sense oligonucleotide (ASO, AON), short interfering RNA (siRNA), anti-microRNA (anti-miRNA), DNA aptamer, RNA aptamer, mRNA, mini-circle DNA, peptide nucleic acid (PNA), phosphoramidate morpholino oligomer (PMO), locked nucleic acid (LNA), bridged nucleic acid (BNA), 2′-deoxy-2′-fluoroarabino nucleic acid (FANA), 2′-O-methoxyethyl-RNA (MOE), 3′-fluoro hexitol nucleic acid (FHNA), glycol nucleic acid (GNA), threose nucleic acid (TNA), BNA-based siRNA, and BNA-based antisense oligonucleotide (BNA-AON), an siRNA, such as BNA-based siRNA, selected from chemically modified siRNA, metabolically stable siRNA and chemically modified, metabolically stable siRNA, or any other category known in the art.

In a related embodiment, a conjugate is provided, wherein the oligonucleotide comprises or consists of any one of the following: morpholino phosphorodiamidate oligomer (PMO), 2′-O-methyl (2′-OMe) phosphorothioate RNA, 2′-O-methoxyethyl (2′-O-MOE) RNA {2′-O-methoxyethyl-RNA (MOE)}, locked or bridged nucleic acid (LNA or BNA), 2′-O,4′-aminoethylene bridged nucleic acid (BNANC), peptide nucleic acid (PNA), 2′-deoxy-2′-fluoroarabino nucleic acid (FANA), 3′-fluoro hexitol nucleic acid (FHNA), glycol nucleic acid (GNA), threose nucleic acid (TNA), silencing RNA (siRNA), short hairpin RNA (shRNA), microRNA (miRNA), antagomir (miRNA antagonists), aptamer RNA or aptamer DNA, single-stranded RNA or single-stranded DNA, double-stranded RNA (dsRNA) or double-stranded DNA.

In particularly preferred embodiments, the conjugate is provided, wherein the oligonucleotide comprises or consists of a morpholino phosphorodiamidate oligomer (PMO) or a 2′-O-methyl (2′-OMe) phosphorothioate RNA.

An embodiment is the conjugate of the invention, wherein the at least one effector molecule is an oligonucleotide selected from any one or more of a(n): short interfering RNA (siRNA), short hairpin RNA (shRNA), anti-hairpin-shaped microRNA (miRNA), single-stranded RNA, aptamer RNA, double-stranded RNA (dsRNA), anti-microRNA (anti-miRNA, anti-miR), antisense oligonucleotide (ASO), mRNA, DNA, antisense DNA, locked nucleic acid (LNA), bridged nucleic acid (BNA), 2′-O,4′-aminoethylene bridged nucleic Acid (BNA^{NC}), BNA-based siRNA, and BNA-based antisense oligonucleotide (BNA-AON).

An embodiment is the conjugate of the invention, wherein the at least one effector molecule is an oligonucleotide selected from any one of an anti-miRNA, a BNA-AON or an siRNA, such as BNA-based siRNA, preferably selected from chemically modified siRNA, metabolically stable siRNA and chemically modified, metabolically stable siRNA.

An embodiment is the conjugate of the invention, wherein the at least one effector molecule is an oligonucleotide that is capable of silencing a gene, when present in a cell comprising such gene, wherein the gene is any one of genes: apolipoprotein B (apoB), HSP27, transthyretin (TTR), proprotein convertase subtilisin/kexin type 9 (PCSK9), delta-aminolevulinate synthase 1 (ALAS1), antithrombin 3 (AT3), glycolate oxidase (GO), complement component C5 (CC5), X gene of hepatitis B virus (HBV), S gene of HBV, alpha-1 antitrypsin (AAT) and lactate dehydrogenase (LDH), and/or is capable of targeting an aberrant miRNA when present in a cell comprising such aberrant miRNA.

For example, the at least one effector molecule comprised by the conjugate is an oligonucleotide that is capable of silencing a gene, when present in a cell comprising such gene, wherein the gene is for example any one of genes: apolipoprotein B (apoB), HSP27, transthyretin (TTR), proprotein convertase subtilisin/kexin type 9 (PCSK9), delta-aminolevulinate synthase 1 (ALAS1), antithrombin 3 (AT3), glycolate oxidase (GO), complement component C5 (CC5), X gene of hepatitis B virus (HBV), S gene of HBV, alpha-1 antitrypsin (AAT) and lactate dehydrogenase (LDH), and/or is capable of targeting an aberrant miRNA when present in a cell comprising such aberrant miRNA.

An embodiment is the conjugate of the invention, wherein the effector molecule is an oligonucleotide that is capable of targeting an mRNA, when present in a cell comprising such mRNA, wherein the mRNA is involved in expression of any one of proteins: apoB, HSP27, TTR, PCSK9, ALAS1, AT3, GO, CC5, expression product of X gene of HBV, expression product of S gene of HBV, AAT and LDH, or is capable of antagonizing or restore an miRNA function such as inhibiting an oncogenic miRNA (onco-miR) or suppression of expression of an onco-miR, when present in a cell comprising such an miRNA.

For example, the at least one effector molecule comprised by the conjugate is an oligonucleotide that is capable of targeting an mRNA, when present in a cell comprising such mRNA, wherein for example the mRNA is involved in expression of any one of proteins: apoB, HSP27, TTR, PCSK9, ALAS1, AT3, GO, CC5, expression product of X gene of HBV, expression product of S gene of HBV, AAT and LDH, or is for example capable of antagonizing or restoring an miRNA function such as inhibiting an oncogenic miRNA (onco-miR) or suppressing of expression of an onco-miR, when present in a cell comprising such an miRNA.

The inventors show that a tumor-cell targeting monoclonal antibody provided with covalently coupled antisense BNA such as BNA(HSP27) and provided with covalently coupled saponin of the invention, that is contacted with tumor cells, both the BNA and the saponin coupled to the antibody (e.g. cetuximab) via a cleavable bond, is capable of silencing HSP27 in vivo in tumors, compared to control and compared to the AOC bearing the BNA only and not the saponin (SO1861, Quil-A). Administering an ADC-saponin conjugate of the invention based on sdAb(s), or an sdAb(s)-oligonucleotide-saponin conjugate of the invention (AOC-saponin), such as an sdAb(s)-BNA-saponin conjugate, thus endows ADC-saponin based on sdAb(s) or AOC-saponin based on sdAb(s) with anti-tumor cell activity not seen with only the sdAb(s)-based ADC or only the sdAb(s)-based AOC, which do not have the covalently saponins bound to the sdAb(s), at the same dose. Noteworthy, the AOC and the separate monoclonal antibody with covalently coupled saponin as a combination of two separate conjugates, increase HSP27 expression in tumor cells, when administered to tumor-bearing mice separately in separate groups of mice, compared to a control group (vehicle administered, only). Only administration of the AOC-saponin conjugate comprising the effector moiety, displays reduced HSP27 expression when compared to controls. The antisense BNA (HSP27) was a BNA with oligonucleic acid sequence according to Zhang et al. (2011) [Y Zhang, Z Qu, S Kim, V Shi, B Liao1, P Kraft, R Bandaru, Y Wu, L M Greenberger and I D Horak, Down-modulation of cancer targets using locked nucleic acid (LNA)-based antisense oligonucleotides without transfection, Gene Therapy (2011) 18, 326-333]. Noteworthy, to the best of the knowledge of the inventors, BNA is designed for application as a free nucleic acid. The inventors are the first to demonstrate that the antisense BNA can be covalently coupled through a (non-)cleavable linker with sdAb(s), in a way that gene-silencing activity is retained in the tumor cells. This approach of providing BNA based AOCs comprising sdAb(s) as the cell-targeting ligand for binding to a cell-surface molecule on the target cell (e.g. an endocytic receptor) opens new ways to administer targeted BNA to human (cancer) patients in need thereof.

An embodiment is the conjugate of the invention, wherein the at least one effector molecule comprises or consists of at least one proteinaceous molecule, preferably selected from any one or more of a peptide, a protein, an enzyme and a protein toxin. The inventors found that very effective tumor cell killing is achieved when sdAbs are selected that bind any of HER2, VEGFR, CD71, which sdAb is combined in the conjugate with a toxin such as a protein toxin, such as dianthin or saporin, and which sdAb is combined with the saponin. Examples demonstrating the high efficacy of certain conjugates comprising an sdAb and an effector molecule are provided in the Examples section.

An embodiment is the conjugate of the invention, wherein the at least one effector molecule comprises or consists of a toxin. An embodiment is the conjugate of the invention, wherein the toxin is selected from the list consisting of: a viral toxin, a bacterial toxin, a plant toxin including ribosome-inactivating proteins and the A chain of type 2 ribosome-inactivating proteins, an animal toxin, a human toxin and a fungal toxin, more preferably the toxin is a plant toxin including ribosome-inactivating proteins and the A chain of type 2 ribosome-inactivating proteins. An embodiment is the conjugate of the invention, wherein the toxin is selected from the list consisting of: apoptin, Shiga toxin, Shiga-like toxin, Pseudomonas aeruginosa exotoxin (PE), full-length or truncated diphtheria toxin (DT), cholera toxin, alpha-sarcin, dianthin, saporin, bouganin, de-immunized derivative debouganin of bouganin, shiga-like toxin A, pokeweed antiviral protein, ricin, ricin A chain, modeccin, modeccin A chain, abrin, abrin A chain, volkensin, volkensin A chain, viscumin, viscumin A chain, frog RNase, granzyme B, human angiogenin; preferably the toxin is dianthin and/or saporin.

An embodiment is the conjugate of the invention, wherein the toxin is selected from the list consisting of: a proteinaceous toxin, a ribosome-inactivating protein, a protein toxin, a bacterial toxin, a plant toxin, more preferably selected from any one or more of a viral toxin such as apoptin; a bacterial toxin such as Shiga toxin, Shiga-like toxin, Pseudomonas aeruginosa exotoxin (PE) or exotoxin A of PE, full-length or truncated diphtheria toxin (DT), cholera toxin; a fungal toxin such as alpha-sarcin; a plant toxin including ribosome-inactivating proteins and the A chain of type 2 ribosome-inactivating proteins such as dianthin e.g. dianthin-30 or dianthin-32, saporin e.g. saporin-S3 or saporin-S6, bouganin or de-immunized derivative debouganin of bouganin, shiga-like toxin A, pokeweed antiviral protein, ricin, ricin A chain, modeccin, modeccin A chain, abrin, abrin A chain, volkensin, volkensin A chain, viscumin, viscumin A chain; or an animal or human toxin such as frog RNase, or granzyme B or human angiogenin, or any toxic fragment or toxic derivative thereof; preferably the protein toxin is dianthin and/or saporin.

An embodiment is the conjugate of the invention, wherein the at least one effector molecule comprises or consists of: a toxin targeting ribosomes, a toxin targeting elongation factors, a toxin targeting tubulin, a toxin targeting DNA and a toxin targeting RNA, more preferably the toxin is selected from the list consisting of: emtansine, pasudotox, maytansinoid derivative DM1, maytansinoid derivative DM4, monomethyl auristatin E (MMAE, vedotin), monomethyl auristatin F (MMAF, mafodotin), a Calicheamicin, N-Acetyl-γ-calicheamicin, a pyrrolobenzodiazepine (PBD) dimer, a benzodiazepine, a CC-1065 analogue, a duocarmycin, Doxorubicin, paclitaxel, docetaxel, cisplatin, cyclophosphamide, etoposide, docetaxel, 5-fluorouracyl (5-FU), mitoxantrone, a tubulysin, an indolinobenzodiazepine, AZ13599185, a cryptophycin, rhizoxin, methotrexate, an anthracycline, a camptothecin analogue, SN-38, DX-8951f, exatecan mesylate, truncated form of Pseudomonas aeruginosa exotoxin (PE38), a Duocarmycin derivative, an amanitin, a-amanitin, a spliceostatin, a thailanstatin, ozogamicin, tesirine, Amberstatin269 and soravtansine.

An embodiment is the conjugate of the invention, wherein the effector moiety is a protein, such as an enzyme, preferably selected from: urease, Cre-recombinase.

An embodiment is the conjugate of the invention, wherein the effector moiety is an oligonucleotide. Conjugates of the invention comprising an oligonucleotide are preferred. Conjugates wherein the effector moiety is an oligonucleotide are preferred.

An embodiment is the conjugate of the invention, wherein the at least one effector molecule is a toxin.

An embodiment is the conjugate of the invention, wherein the at least one effector molecule is a drug molecule. For example, the at least one effector molecule comprised by the conjugate is a pharmaceutically active substance.

An effector moiety useful in the present invention preferably relies on late endosomal escape for exerting its effect. Some effector molecules, such as, e.g., a pseudomonas exotoxin, are rerouted to other organelles prior to the “late endosomal stage” and, thus, would normally not benefit from incorporation in the conjugate according to the present invention. However, such toxin may be adapted for use with the present invention, e.g., by deleting the signal peptide responsible for rerouting. In particular toxins that are highly toxic and would require only one molecule to escape the endosomes to kill a cell maybe modified to be less potent. It is preferred to use a toxin that kills a cell if at least 2, more preferably at least 5, more preferably at least 10, more preferably at least 20, more preferably at least 50, most preferably at least 100 toxin molecules escape the endosome (and enter the cytosol). It is further preferred that a conjugate of the invention comprises a covalently conjugated functionalized scaffold, i.e. a scaffold such as an oligomeric or polymeric scaffold or a tri-functional linker, comprising covalently bound effector moiety or moieties for targeting the scaffold comprising the bound effector moiety/moieties at a target cell such as a tumor cell or an auto-immune cell. Further, in order to reduce off-target toxicity, cell membrane non-permeable small molecule toxins are preferred effector molecules over cell membrane permeable toxins.

Preferably, the effector moiety comprised by the conjugate of the invention, which effect is enhanced by the saponins comprised by the conjugate, detaches from the conjugate, e.g. detaches from the single or, preferably, multiple sdAb(s), present in the conjugate as the cell-surface molecule targeting moiety of the conjugate, when endocytosed. This can be achieved by a cleavable bond that breaks, e.g. under acidic, reductive, enzymatic or light-induced conditions.

An embodiment is the conjugate of the invention, wherein the conjugate comprises 1-16 effector molecules, preferably oligonucleotide(s), preferably 1-4 effector molecules, most preferably 1 effector molecule, wherein the effector moiety is preferably covalently bound in the conjugate via a cleavable bond, preferably an acid-labile cleavable bond that is cleaved under acidic conditions such as for example present in endosomes and/or lysosomes of mammalian cells, preferably human cells such as tumor cells, preferably at pH 4.0-6.5, and more preferably at pH≤5.5, wherein preferably the cleavable bond is a hydrazone bond or a semicarbazone bond, more preferably a hydrazone bond.

For example, the conjugate comprises 1-16 effector molecules, preferably oligonucleotide(s), preferably 1-4 effector molecules, most preferably 1 effector molecule, wherein the effector moiety is preferably covalently bound in the conjugate via a cleavable bond, preferably selected from:
a bond subject to cleavage under acidic conditions such as a semi-carbazone bond, a hydrazone bond, an imine bond, an acetal bond including a 1,3-dioxolane bond, a ketal bond, an ester bond, and/or an oxime bond,
a bond susceptible to proteolysis, for example amide or peptide bond, preferably subject to proteolysis by Cathepsin B;
a red/ox-cleavable bond such as a disulfide bond, or a thiol-exchange reaction-susceptible bond such as a thio-ether bond.

For example, the conjugate comprises 1-16 effector molecules, preferably oligonucleotide(s), preferably 1-4 effector molecules, most preferably 1 effector molecule, wherein the effector moiety is preferably covalently bound in the conjugate via a cleavable bond, preferably selected from any one or more of: a semi-carbazone bond, a hydrazone bond, an imine bond, an acetal bond including a 1,3-dioxolane bond, a ketal bond, an ester bond, and/or an oxime bond, preferably wherein the acid-sensitive linker comprises a semi-carbazone bond or a hydrazone bond.

For example, the conjugate comprises 1-16 effector molecules, preferably oligonucleotide(s), preferably 1-4 effector molecules, most preferably 1 or 2 effector molecule(s), wherein the effector molecule(s) is/are preferably covalently bound in the conjugate via a cleavable bond, selected from:
a bond subject to cleavage under acidic conditions such as a semi-carbazone bond, a hydrazone bond, an imine bond, an acetal bond including a 1,3-dioxolane bond, a ketal bond, an ester bond, and/or an oxime bond,
a bond susceptible to proteolysis, for example an amide bond or a peptide bond, preferably subject to proteolysis by Cathepsin B,
a red/ox-cleavable bond such as a disulfide bond, or a thiol-exchange reaction-susceptible bond such as a thio-ether bond,
preferably a hydrazone bond or a semicarbazone bond, more preferably a hydrazone bond.

Preferably, such a cleavable bond is stable (does not cleave) under the conditions (pH) as apparent in the circulation of a human subject, and is susceptible to cleavage under the conditions (pH) as apparent in the endosome and/or lysosome of the target cell of said human subject in which the conjugate is delivered via binding of the sdAbs in the conjugate to the cell-surface molecule (endocytic receptor) on the target cell of the human subject. Examples of such cleavable bonds suitable for the purpose are a semicarbazone bond and a hydrazone bond.

An embodiment is the conjugate of the invention, wherein the conjugate comprises an antibody-drug conjugate (ADC) comprising at least one sdAb derived from or based on the Vₕ domain and/or the drug moiety selected from ADCs: gemtuzumab ozogamicin, brentuximab vedotin, trastuzumab emtansine, inotuzumab ozogamicin, moxetumomab pasudotox and polatuzumab vedotin, and/or comprising at least one effector molecule which is a toxin present in any one or more of: gemtuzumab ozogamicin, brentuximab vedotin, trastuzumab emtansine, inotuzumab ozogamicin, moxetumomab pasudotox and polatuzumab vedotin, and/or selected from dianthin and saporin. It will be appreciated that when an sdAb is derived from such a human antibody, the Vₕ domain of such a human antibody may require some improvements with regard to domain stability (‘camelization’ of the human Vₕ domain), known in the art.

An embodiment is the conjugate of the invention, wherein the at least one saponin comprises an aglycone core structure selected from:
2alpha-hydroxy oleanolic acid;
16alpha-hydroxy oleanolic acid;
hederagenin (23-hydroxy oleanolic acid);
16alpha,23-dihydroxy oleanolic acid;
gypsogenin;
quillaic acid;
protoaescigenin-21(2-methylbut-2-enoate)-22-acetate;
23-oxo-barringtogenol C-21,22-bis(2-methylbut-2-enoate);
23-oxo-barringtogenol C-21(2-methylbut-2-enoate)-16,22-diacetate;
digitogenin;
3,16,28-trihydroxy oleanan-12-en;
gypsogenic acid; or
a derivative thereof,
   preferably, the at least one saponin comprises an aglycone core structure selected from quillaic acid and gypsogenin, more preferably the at least one saponin comprises aglycone core structure quillaic acid.

An embodiment is the conjugate of the invention, wherein the at least one saponin is a triterpenoid saponin of the 12,13-dehydrooleanane type comprising an aldehyde group at position C-23, and preferably comprises an aglycone core structure selected from quillaic acid and gypsogenin, more preferably the at least one saponin comprises the aglycone core structure quillaic acid.

Without wishing to be bound by any theory, presence of an aldehyde group (or derivative thereof which derivative is formed into an aldehyde group once the conjugate comprising the covalently bound saponin is present in the endosome or lysosome of the cell bearing the cell-surface molecule) in the aglycone core structure of the saponin (here, also referred to as ‘aglycone’) is beneficial for the capacity of the saponin to stimulate and/or potentiate the endosomal escape of the effector molecule comprised by the conjugate of the invention, when such a saponin co-localizes in a cell, in the endosome of said cell, with these effector molecules, as part of the conjugate of the invention or when in free form inside the endosome (e.g. split off from the conjugate once the conjugate is delivered inside the target cell endosome or lysosome). Therefore, the conjugates of the invention comprising saponin which has an aglycone with an aldehyde group is preferred. In quillaic acid and in gypsogenin the aldehyde group is at the C₂₃ atom of the aglycone (see as an example the structure of SAPONIN A, here above, and saponins listed in Table A1).

An embodiment is the conjugate of the invention, wherein the at least one saponin comprises one or both of: a first saccharide chain bound to the C₃ atom or to the C₂₈ atom of the aglycone core structure of the at least one saponin, preferably bound to the C₃ atom, and a second saccharide chain bound to the C₂₈ atom of the aglycone core structure of the at least one saponin, and preferably the at least one saponin comprises the first and the second saccharide chain. Thus, when the saponin comprised by the conjugate of the invention bears two glycans (saccharide chains), the first saccharide chain is bound at position C₃ of the aglycone core structure and the second saccharide chain is typically bound at position C₂₈ of the aglycone core structure of the saponin, although for some saponins lacking the aldehyde group at position C-23 position, the second glycan can be bound at said C-23 position (see Table A1). Preferred is a saponin with a first glycan bound at position C₃ of the aglycone core structure and a second glycan bound at position C₂₈ of the aglycone core structure of the saponin.

An embodiment is the conjugate of the invention, wherein the at least one saponin comprises the first saccharide chain that is selected from (Group AA):
GlcA-,
Glc-,
Gal-,
Rha-(1→2)-Ara-,
Gal-(1→2)-[Xyl-(1→3)]-GlcA-,
Glc-(1→2)-[Glc-(1→4)]-GlcA-,
Glc-(1→2)-Ara-(1→3)-[Gal-(1→2)]-GlcA-,
Xyl-(1→2)-Ara-(1→3)-[Gal-(1→2)]-GlcA-,
Glc-(1→3)-Gal-(1→2)-[Xyl-(1→3)]-Glc-(1→4)-Gal-,
Rha-(1→2)-Gal-(1→3)-[Glc-(1→2)]-GlcA-,
Ara-(1→4)-Rha-(1→2)-Glc-(1→2)-Rha-(1→2)-GlcA-,
Ara-(1→4)-Fuc-(1→2)-Glc-(1→2)-Rha-(1→2)-GlcA-,
Ara-(1→4)-Rha-(1→2)-Gal-(1→2)-Rha-(1→2)-GlcA-,
Ara-(1→4)-Fuc-(1→2)-Gal-(1→2)-Rha-(1→2)-GlcA-,
Ara-(1→4)-Rha-(1→2)-Glc-(1→2)-Fuc-(1→2)-GlcA-,
Ara-(1→4)-Fuc-(1→2)-Glc-(1→2)-Fuc-(1→2)-GlcA-,
Ara-(1→4)-Rha-(1→2)-Gal-(1→2)-Fuc-(1→2)-GlcA-,
Ara-(1→4)-Fuc-(1→2)-Gal-(1→2)-Fuc-(1→2)-GlcA-,
Xyl-(1→4)-Rha-(1→2)-Glc-(1→2)-Rha-(1→2)-GlcA-,
Xyl-(1→4)-Fuc-(1→2)-Glc-(1→2)-Rha-(1→2)-GlcA-,
Xyl-(1→4)-Rha-(1→2)-Gal-(1→2)-Rha-(1→2)-GlcA-,
Xyl-(1→4)-Fuc-(1→2)-Gal-(1→2)-Rha-(1→2)-GlcA-,
Xyl-(1→4)-Rha-(1→2)-Glc-(1→2)-Fuc-(1→2)-GlcA-,
Xyl-(1→4)-Fuc-(1→2)-Glc-(1→2)-Fuc-(1→2)-GlcA-,
Xyl-(1→4)-Rha-(1→2)-Gal-(1→2)-Fuc-(1→2)-GlcA-, and
Xyl-(1→4)-Fuc-(1→2)-Gal-(1→2)-Fuc-(1→2)-GlcA-,
and/or
the at least one saponin comprises the second saccharide chain selected from (Group BB):
Glc-,
Gal-,
Rha-(1→2)-[Xyl-(1→4)]-Rha-,
Rha-(1→2)-[Ara-(1→3)-Xyl-(1→4)]-Rha-,
Ara-,
Xyl-,
Xyl-(1→4)-Rha-(1→2)-[R1-(→4)]-Fuc- wherein R1 is 4E-Methoxycinnamic acid,
Xyl-(1→4)-Rha-(1→2)-[R2-(→4)]-Fuc- wherein R2 is 4Z-Methoxycinnamic acid,
Xyl-(1→4)-[Gal-(1→3)]-Rha-(1→2)-4-OAc-Fuc-,
Xyl-(1→4)-[Glc-(1→3)]-Rha-(1→2)-3,4-di-OAc-Fuc-,
Xyl-(1→4)-[Glc-(1→3)]-Rha-(1→2)-[R3-(→4)]-3-OAc-Fuc- wherein R3 is 4E-Methoxycinnamic acid,
Glc-(1→3)-Xyl-(1→4)-[Glc-(1→3)]-Rha-(1→2)-4-OAc-Fuc-,
Glc-(1→3)-Xyl-(1→4)-Rha-(1→2)-4-OAc-Fuc-,
(Ara- or Xyl-)(1→3)-(Ara- or Xyl-)(1→4)-(Rha- or Fuc-)(1→2)-[4-OAc-(Rha- or Fuc-)(1→4)]-(Rha- or Fuc-),
Xyl-(1→3)-Xyl-(1→4)-Rha-(1→2)-[Qui-(1→4)]-Fuc-,
Api-(1→3)-Xyl-(1→4)-[Glc-(1→3)]-Rha-(1→2)-Fuc-,
Xyl-(1→4)-[Gal-(1→3)]-Rha-(1→2)-Fuc-,
Xyl-(1→4)-[Glc-(1→3)]-Rha-(1→2)-Fuc-,
Ara/Xyl-(1→4)-Rha/Fuc-(1→4)-[Glc/Gal-(1→2)]-Fuc-,
Api-(1→3)-Xyl-(1→4)-[Glc-(1→3)]-Rha-(1→2)-[R4-(→4)]-Fuc- wherein R4 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R5-(→4)]-Fuc- wherein R5 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[Rha-(1→3)]-4-OAc-Fuc-,
Api-(1→3)-Xyl-(1→4)-[Glc-(1→3)]-Rha-(1→2)-[Rha-(1→3)]-4-OAc-Fuc-,
6-OAc-Glc-(1→3)-Xyl-(1→4)-Rha-(1→2)-[3-OAc-Rha-(1→3)]-Fuc-,
Glc-(1→3)-Xyl-(1→4)-Rha-(1→2)-[3-OAc--Rha-(1→3)]-Fuc-,
Xyl-(1→3)-Xyl-(1→4)-Rha-(1→2)-[Qui-(1→4)]-Fuc-,
Glc-(1→3)-[Xyl-(1→4)]-Rha-(1→2)-[Qui-(1→4)]-Fuc-,
Glc-(1→3)-Xyl-(1→4)-Rha-(1→2)-[Xyl-(1→3)-4-OAc-Qui-(1→4)]-Fuc-,
Xyl-(1→3)-Xyl-(1→4)-Rha-(1→2)-[3,4-di-OAc-Qui-(1→4)]-Fuc-,
Glc-(1→3)-[Xyl-(1→4)]-Rha-(1→2)-Fuc-,
6-OAc-Glc-(1→3)-[Xyl-(1→4)]-Rha-(1→2)-Fuc-,
Glc-(1→3)-[Xyl-(1→3)-Xyl-(1→4)]-Rha-(1→2)-Fuc-,
Xyl-(1→3)-Xyl-(1→4)-Rha-(1→2)-[Xyl-(1→3)-4-OAc-Qui-(1→4)]-Fuc-,
Api/Xyl-(1→3)-Xyl-(1→4)-[Glc-(1→3)]-Rha-(1→2)-[Rha-(1→3)]-4OAc-Fuc-,
Api-(1→3)-Xyl-(1→4)-[Glc-(1→3)]-Rha-(1→2)-[Rha-(1→3)]-4OAc-Fuc-,
Api/Xyl-(1→3)-Xyl-(1→4)-[Glc-(1→3)]-Rha-(1→2)-[R6-(→4)]-Fuc- wherein R6 is 5-O-[5-O-Rha-(1→2)-Ara/Api-3,5-dihydroxy-6-methyl-octanoyl]-3,5-dihydroxy-6-methyl-octanoic acid),
Api/Xyl-(1→3)-Xyl-(1→4)-[Glc-(1→3)]-Rha-(1→2)-[R7-(→4)]-Fuc- wherein R7 is 5-O-[5-O-Ara/Api-3,5-dihydroxy-6-methyl-octanoyl]-3,5-dihydroxy-6-methyl-octanoic acid),
Api/Xyl-(1→3)-Xyl-(1→4)-[Glc-(1→3)]-Rha-(1→2)-[R8-(→4)]-Fuc- wherein R8 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R9-(→4)]-Fuc- wherein R9 is 5-O-[5-O-Ara/Api-3,5-dihydroxy-6-methyl-octanoyl]-3,5-dihydroxy-6-methyl-octanoic acid),
Xyl-(1→3)-Xyl-(1→4)-Rha-(1→2)-[R10-(→4)]-Fuc- wherein R10 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R11-(→3)]-Fuc- wherein R11 is 5-O-[5-O-Ara/Api-3,5-dihydroxy-6-methyl-octanoyl]-3,5-dihydroxy-6-methyl-octanoic acid),
Xyl-(1→3)-Xyl-(1→4)-Rha-(1→2)-[R12-(→3)]-Fuc- wherein R12 is 5-O-[5-O-Ara/Api-3,5-dihydroxy-6-methyl-octanoyl]-3,5-dihydroxy-6-methyl-octanoic acid), and
Glc-(1→3)-[Glc-(1→6)]-Gal-. For example, the at least one saponin comprises the second saccharide chain [4,6-di-OAc-Glc-(1→3)]-[Xyl-(1→4)]-Rha-(1→2)-[3,4-di-OAc-Qui-(1→4)]-Fuc-.

Thus, when the saponin comprised by the conjugate of the invention bears two glycans (saccharide chains), the first saccharide chain is bound at position C₃ of the aglycone core structure of the saponin and the second saccharide chain is preferably bound at position C₂₈ of the aglycone core structure of the saponin. Preferably, the saponin has an aldehyde group in the aglycone. Preferably, the aglycone is gypsogenin or quillaic acid, more preferably quillaic acid.

An embodiment is the conjugate of the invention, wherein the at least one saponin comprises a first saccharide chain, such as selected from Group AA and comprises a second saccharide chain, such as selected from Group BB or [4,6-di-OAc-Glc-(1→3)]-[Xyl-(1→4)]-Rha-(1→2)-[3,4-di-OAc-Qui-(1→4)]-Fuc-, wherein preferably the first saccharide chain comprises more than one saccharide moiety and the second saccharide chain comprises more than one saccharide moiety, and wherein the aglycone core structure preferably is quillaic acid or gypsogenin, more preferably is quillaic acid.

An embodiment is the conjugate of the invention, wherein the at least one saponin comprises a first saccharide chain bound to the C₃ atom of the aglycone core structure of the at least one saponin, wherein the first saccharide chain is Gal-(1→2)-[Xyl-(1→3)]-GlcA, and wherein preferably the aglycone core structure is quillaic acid or gypsogenin, more preferably quillaic acid.

An embodiment is the conjugate of the invention, wherein the at least one saponin comprises the first saccharide chain and comprises the second saccharide chain according to Group AA and Group BB or [4,6-di-OAc-Glc-(1→3)]-[Xyl-(1→4)]-Rha-(1→2)-[3,4-di-OAc-Qui-(1→4)]-Fuc-, respectively, wherein the first saccharide chain comprises more than one saccharide moiety and the second saccharide chain comprises more than one saccharide moiety, and wherein the aglycone core structure preferably is quillaic acid or gypsogenin, more preferably quillaic acid, wherein one, two or three, preferably one or two, of:
i. an aldehyde group in the aglycone core structure has been derivatised,
ii. a carboxyl group of a glucuronic acid moiety in the first saccharide chain has been derivatised, and
iii. at least one acetoxy (Me(CO)O—) group in the second saccharide chain has been derivatised.

For example, the conjugate comprises one, two or three, preferably one or two, more preferably one, of:
i. the at least one saponin has a quillaic acid aglycone core that is derivatised on the C23 position of the aglycone core or a gypsogenin aglycone core that is derivatised on the C23 position of the aglycone core,
ii. the at least one saponin is derivatised in the first saccharide chain linked to the C3 position of the aglycone core of the at least one saponin, if present, and
iii. the at least one saponin is derivatised in the second saccharide chain linked to the C28 position of the at least one saponin, if present;
the at least one saponin comprises an aldehyde function at position C4.

An embodiment is the conjugate of the invention, wherein one, two or three, preferably one or two, more preferably one, of:
i. an aldehyde group in the aglycone core structure of the at least one saponin has been derivatised when present,
ii. a carboxyl group of a glucuronic acid moiety in a first saccharide chain of the at least one saponin has been derivatised when present in the at least one saponin, and
   at least one acetoxy (Me(CO)O—) group in a second saccharide chain of the at least one saponin has been derivatised if present.

An embodiment is the conjugate of the invention, wherein the at least one saponin comprises:
i. an aglycone core structure comprising an aldehyde group which has been derivatised by:
   reduction to an alcohol;
   transformation into a hydrazone bond through reaction with N-s-maleimidocaproic acid hydrazide (EMCH) wherein the maleimide group of the EMCH is optionally derivatised by formation of a thioether bond with mercaptoethanol;
   transformation into a hydrazone bond through reaction with N-[B-maleimidopropionic acid]hydrazide (BMPH) wherein the maleimide group of the BMPH is optionally derivatised by formation of a thioether bond with mercaptoethanol; or
   transformation into a hydrazone bond through reaction with N-[K-maleimidoundecanoic acid]hydrazide (KMUH) wherein the maleimide group of the KMUH is optionally derivatised by formation of a thioether bond with mercaptoethanol; or
ii. a first saccharide chain comprising a carboxyl group, preferably a carboxyl group of a glucuronic acid moiety, which has been derivatised by transformation into an amide bond through reaction with 2-amino-2-methyl-1,3-propanediol (AMPD) or N-(2-aminoethyl)maleimide (AEM); or
iii. a second saccharide chain comprising an acetoxy group (Me(CO)O—) which has been derivatised by transformation into a hydroxyl group (HO—) by deacetylation; or
any combination of two or three derivatisations i., ii. and/or iii., preferably any combination of two derivatisations of i., ii. and iii.

For example, the at least one saponin comprised by the conjugate comprises:
i. an aglycone core structure comprising:
   a hydroxyl group at position C23;
   a hydrazone bond at position C23 for example through reaction of the aldehyde function at position C4 (also referred to as the aldehyde group at the C-23 position of the aglycone; see SAPONIN A, for example) of the at least one saponin with N-s-maleimidocaproic acid hydrazide (EMCH) wherein the maleimide group of the EMCH is optionally derivatised by formation of a thioether bond with mercaptoethanol;
   hydrazone bond at position C23 for example through reaction of the aldehyde function at position C4 of the at least one saponin with N-[B-maleimidopropionic acid]hydrazide (BMPH) wherein the maleimide group of the BMPH is optionally derivatised by formation of a thioether bond with mercaptoethanol; or
   a hydrazone bond at position C23 for example through reaction of the aldehyde function at position C4 of the at least one saponin with N-[K-maleimidoundecanoic acid]hydrazide (KMUH) wherein the maleimide group of the KMUH is optionally derivatised by formation of a thioether bond with mercaptoethanol; or
   a semicarbazone bond at position C23 of the at least one saponin; or
ii. a first saccharide chain comprising an amide bond such as obtainable through reaction of a carboxyl group of a glucuronic acid moiety comprised by the first saccharide chain, with 2-amino-2-methyl-1,3-propanediol (AMPD) or N-(2-aminoethyl)maleimide (AEM); or
iii. a second saccharide chain comprising a hydroxyl group (HO—) such as obtainable by deacetylation of an acetoxy group (Me(CO)O—) comprised by the second saccharide chain; or
iv. any combination of i., ii. and/or iii., preferably any combination of two of i., ii. and iii.

An embodiment is the conjugate of the invention, wherein the at least one saponin is any one or more of:
a) saponin selected from any one or more of list A:
   Quillaja saponaria saponin mixture, or a saponin isolated from Quillaja saponaria, for example Quil-A, QS-17-api, QS-17-xyl, QS-21, QS-21A, QS-21B, QS-7-xyl;
   Saponinum album saponin mixture, or a saponin isolated from Saponinum album;
   Saponaria officinalis saponin mixture, or a saponin isolated from Saponaria officinalis; and
   Quillaja bark saponin mixture, or a saponin isolated from Quillaja bark, for example Quil-A, QS-17-api, QS-17-xyl, QS-21, QS-21A, QS-21B, QS-7-xyl; or
b) a saponin comprising a gypsogenin aglycone core structure, selected from list B:
   SA1641, gypsoside A, NP-017772, NP-017774, NP-017777, NP-017778, NP-018109, NP-017888, NP-017889, NP-018108, SO1658 and Phytolaccagenin; or
c) a saponin comprising a quillaic acid aglycone core structure, selected from list C:
   AG1856, AG1, AG2, Agrostemmoside E, GE1741, Gypsophila saponin 1 (Gyp1), NP-017674, NP-017810, NP-003881, NP-017676, NP-017677, NP-017705, NP-017706, NP-017773, NP-017775, SA1657, Saponarioside B, SO1542, SO1584, SO1674, SO1700, SO1730, SO1772, SO1832, SO1861, SO1862, SO1904, QS-7, QS-7 api, QS-17, QS-18, QS-21A-apio, QS-21A-xylo, QS-21 B-apio and QS-21 B-xylo; or
d) a saponin comprising a 12, 13-dehydrooleanane type aglycone core structure without an aldehyde group at the C-23 position of the aglycone, selected from list D:
   Aescin Ia, aescinate, alpha-Hederin, AMA-1, AMR, AS6.2, AS64R, Assamsaponin F, dipsacoside B, esculentoside A, macranthoidin A, NP-005236, NP-012672, Primula acid 1, saikosaponin A, saikosaponin D, Teaseed saponin I and Teaseedsaponin J,
   preferably, the at least one saponin is any one or more of a saponin selected from list A, B or C, more preferably from list B or C, most preferably a saponin selected from list C.

An embodiment is the conjugate of the invention, wherein the at least one saponin is a saponin derivative based on any one of the saponins of list A, B, C, D, preferably of list B or C, more preferably of list C.

For example, the at least one saponin comprised by the conjugate is any one or more of:
a) saponin selected from any one or more of list A:
   Quillaja saponaria saponin mixture, or a saponin isolated from Quillaja saponaria, for example Quil-A, QS-17-api, QS-17-xyl, QS-21, QS-21A, QS-21B, QS-7-xyl;
   Saponinum album saponin mixture, or a saponin isolated from Saponinum album;
   Saponaria officinalis saponin mixture, or a saponin isolated from Saponaria officinalis; and Quillaja bark saponin mixture, or a saponin isolated from Quillaja bark, for example Quil-A, QS-17-api, QS-17-xyl, QS-21, QS-21A, QS-21B, QS-7-xyl; or
b) a saponin comprising a gypsogenin aglycone core structure, selected from list B: SA1641, gypsoside A, NP-017772, NP-017774, NP-017777, NP-017778, NP-018109, NP-017888, NP-017889, NP-018108, SO1658 and Phytolaccagenin; or
c) a saponin comprising a quillaic acid aglycone core structure, selected from list C: AG1856, AG1, AG2, Agrostemmoside E, GE1741, Gypsophila saponin 1 (Gyp1), NP-017674, NP-017810, NP-003881, NP-017676, NP-017677, NP-017705, NP-017706, NP-017773, NP-017775, SA1657, Saponarioside B, SO1542, SO1584, SO1674, SO1700, SO1730, SO1772, SO1832, SO1861, SO1862, SO1904, TQL-1055, GPI-0100, QS-7, QS-7 api, QS-17, QS-18, QS-21A-apio, QS-21A-xylo, QS-21 B-apio and QS-21 B-xylo; or
d) a saponin comprising a 12, 13-dehydrooleanane type aglycone core structure without an aldehyde group at the C-23 position of the aglycone, selected from list D: Aescin Ia, aescinate, alpha-Hederin, AMA-1, AMR, AS6.2, AS64R, Assamsaponin F, dipsacoside B, esculentoside A, macranthoidin A, NP-005236, NP-012672, Primula acid 1, saikosaponin A, saikosaponin D, Teaseed saponin I and Teaseedsaponin J, preferably, the at least one saponin is any one or more of a saponin selected from list A, B or C, more preferably from list B or C, most preferably a saponin selected from list C.

An embodiment is the conjugate of the invention, wherein the at least one saponin is any one or more of AG1856, GE1741, a saponin isolated from Quillaja saponaria, Quil-A, QS-17, QS-21, QS-7, SA1641, a saponin isolated from Saponaria officinalis, Saponarioside B, SO1542, SO1584, SO1658, SO1674, SO1700, SO1730, SO1772, SO1832, SO1861, SO1862 and SO1904, or a derivative thereof, or a stereoisomer thereof, and/or any combinations thereof, preferably the at least one saponin is any one or more of QS-21, SO1832, SO1861, SA1641 and GE1741, or a derivative thereof, or a stereoisomer thereof, and/or any combinations thereof, more preferably the at least one saponin is QS-21, SO1832 or SO1861, even more preferably the at least one saponin is SO1861, or a derivative thereof, or a stereoisomer thereof, and/or any combinations thereof.

For example, the at least one saponin comprised by the conjugate is any one or more of AG1856, GE1741, a saponin isolated from Quillaja saponaria, Quil-A, QS-17, QS-21, QS-7, SA1641, a saponin isolated from Saponaria officinalis, Saponarioside B, SO1542, SO1584, SO1658, SO1674, SO1700, SO1730, SO1772, SO1832, SO1861, SO1862 and SO1904, preferably the at least one saponin is any one or more of QS-21, SO1832, SO1861, SA1641 and GE1741, more preferably the at least one saponin is QS-21, SO1832, SO1861 or AG1856, even more preferably the at least one saponin is SO1832, SO1861 or AG1856, most preferably, the at least one saponin is SO1832 or SO1861, or is SO1861.

Preferred is the saponin-comprising conjugate of the invention, wherein the at least one saponin is a saponin isolated from Saponaria officinalis, preferably the at least one saponin is any one or more of Saponarioside B, SO1542, SO1584, SO1658, SO1674, SO1700, SO1730, SO1772, SO1832, SO1861, SO1862 and SO1904, more preferably the at least one saponin is any one or more of SO1542, SO1584, SO1658, SO1674, SO1700, SO1730, SO1772, SO1832, SO1861, SO1862 and SO1904, even more preferably the at least one saponin is any one or more of SO1832, SO1861 and SO1862, even more preferably SO1832 and SO1861, even more preferably the at least one saponin is SO1861. It is part of the invention that the at least one saponin comprised by the conjugate is a functional derivative of any one of the afore listed saponins (see e.g. Table A1), the functional derivative lacking an aldehyde group in the aglycone core structure of the at least one saponin in its native, non-conjugated form, and/or without a glucuronic acid moiety comprising a carboxyl group in a first saccharide chain of the at least one saponin when in its native, non-conjugated form. Typically, the saponin is a saponin selected from Table A1, known for the endosomal escape enhancing activity towards an effector molecule when contacted with a selected cell together with the effector molecule (such as part as an effector moiety of an ADC or an AOC). Preferably, the at least one saponin is a saponin selected from Group B or Group C, more preferably selected from Group C.

Such saponins of the triterpene glycoside type are capable of enhancing the endosomal escape of the effector molecules comprised by the conjugate, and that are present in the endosome (or lysosome) of a cell, when the saponin as part of the conjugate or in free form co-localizes with such effector molecule inside the cell. The inventors established that the endosomal escape enhancing activity of these saponins is about 100 to 1000 times more potent when the saponin is contacted with a cell as part of the conjugate of the invention. The free saponin is capable of stimulating the delivery of effector molecules in the cytosol of cells, when such cells are contacted with the effector molecules as part of a certain cell-targeting conjugate such as an ADC or an AOC, and the saponin, at 100-1000 times higher saponin concentration, compared to the concentration of the same saponin which is comprised by the conjugate of the invention, required to achieve the same extent of delivery of the effector molecule from outside the target cell to inside the endosome and finally in the cytosol of said cell. Saponins which display such endosomal escape enhancing activity are listed in Table A1. When the saponin is part of the conjugate of the invention, the targeted delivery of the saponin upon binding of the sdAb(s) of the conjugate, to the targeted cell-surface binding site on the target cell (cell-surface molecule; endocytic receptor), on said cell, and after endocytosis, into the endosome of said cell, is thus about 100 to 1000 times more effective compared to contacting the same cell with free, untargeted saponin (derivative) which is not provided with a binding molecule such as at least one sdAb for binding to cell-surface molecule of the target cell.

The small size of an sdAb of the conjugates of the invention, compared to e.g. IgG type of antibodies, or fragments thereof such as Fab, scFv, contributes to efficient uptake by the target cell that exposes the binding site for binding of the sdAb comprised by the conjugate, e.g. uptake by endocytosis. Typically, the sdAbs in the conjugates of the invention are capable of binding to a cell-surface receptor of a target cell, such as a tumor cell specific cell-surface receptor. This way, the conjugate of the invention is particularly suitable for endocytosis into e.g. tumor cells expressing the cell-surface receptor.

An embodiment is the conjugate of the invention, wherein the at least one sdAb comprises an sdAb for binding to a cell-surface molecule of the cell wherein the cell is an aberrant cell such as a tumor cell, an auto-immune cell, an infected cell such as a virally infected cell, or a cell comprising a gene defect or an enzyme defect. The tumor cell is for example related to a cancer such as bladder cancer such as metastatic bladder cancer, urothelial carcinoma, cancer of the urinary tract, urologic neoplasms, prostate cancer such as castration resistant prostate cancer, breast cancer, malignant neoplasm of pancreas, ovary cancer, lung cancer such as non-squamous non-small cell lung cancer and squamous cell carcinoma of lung.

An embodiment is the conjugate of the invention, wherein the at least one sdAb comprises an sdAb for binding to a cell-surface molecule of the cell, the sdAb derived from or based on any one or more of immunoglobulins: an anti-CD71 antibody such as IgG type OKT-9, an anti-HER2 antibody such as trastuzumab (Herceptin), pertuzumab, an anti-CD20 antibody such as rituximab, ofatumumab, tositumomab, obinutuzumab ibritumomab, an anti-CA125 antibody such as oregovomab, an anti-EpCAM (17-1A) antibody such as edrecolomab, an anti-EGFR antibody such as cetuximab, matuzumab, panitumumab, nimotuzumab, an anti-CD30 antibody such as brentuximab, an anti-CD33 antibody such as gemtuzumab, huMy9-6, an anti-vascular integrin alpha-v beta-3 antibody such as etaracizumab, an anti-CD52 antibody such as alemtuzumab, an anti-CD22 antibody such as epratuzumab, pinatuzumab, binding fragment (Fv) of anti-CD22 antibody moxetumomab, humanized monoclonal antibody inotuzumab, an anti-CEA antibody such as labetuzumab, an anti-CD44v6 antibody such as bivatuzumab, an anti-FAP antibody such as sibrotuzumab, an anti-CD19 antibody such as huB4, an anti-CanAg antibody such as huC242, an anti-CD56 antibody such as huN901, an anti-CD38 antibody such as daratumumab, OKT-10 anti-CD38 monoclonal antibody, an anti-CA6 antibody such as DS6, an anti-IGF-1R antibody such as cixutumumab, 3B7, an anti-integrin antibody such as CNTO 95, an anti-syndecan-1 antibody such as B-B4, an anti-CD79b such as polatuzumab, an anti-HIVgp41 antibody, preferably any one of an anti-HIVgp41 antibody, an anti-CD71 antibody, an anti-HER2 antibody and an anti-EGFR antibody, more preferably any one of: trastuzumab, pertuzumab, cetuximab, matuzumab, an anti-CD71 antibody, OKT-9, most preferably trastuzumab, cetuximab, the anti-CD71 antibody OKT-9. Preferably, the cell surface molecule is an endocytic receptor, and more preferably, binding of the at least one sdAb to such an endocytic receptor induces endocytosis of the conjugate comprising said at least one sdAb. For example, the sdAb is derived from or based on an anti-CD63 antibody.

These cell-surface molecules are typically present on tumor cells with tumor cell specificity, at least to a certain extent. Tumor cell specificity makes these receptors suitable targets for the conjugates of the invention, and therefore the sdAb(s) in the conjugate is capable of binding to such a cell-surface receptor. Since the saponins comprised by the conjugate of the invention are capable of stimulating the release and delivery of the effector molecules comprised by the conjugate of the invention, in the cytosol of cells, such as the (tumor) cells targeted by the sdAb(s) comprised by the conjugate of the invention, it is particularly suitable to select as the target (tumor) cell surface molecule for the sdAb(s), a cell-surface receptor known for its suitability to serve as the target for e.g. ADCs and AOCs. The conjugate of the invention is therewith suitable for co-delivery of the effector molecule that is part of the conjugate, together with the saponin comprised by the very same conjugate of the invention, which conjugate is an improved ADC or an improved AOC comprising sdAb(s), preferably 2 or 3 sdAbs such as a bivalent nanobody or a biparatopic sdAb or a trivalent nanobody, and comprising a saponin. Targeting a tumor cell specific receptor with the conjugate of the invention promotes endocytosis and delivery of the saponin as part of the conjugate into the target cell endosome and/or lysosome. When the tumor cell is contacted with the conjugate of the invention, the effector molecule comprised by the conjugate of the invention is co-delivered into the endosome or lysosome, and under influence of the co-localized saponin, the effector molecule is subsequently transferred into the cytosol of the target cell. As explained herein earlier, the application of targeted saponin as part of the conjugate of the invention results in an about 100-fold to 1000-fold improvement of the potentiating effect of the saponin, when biological activity of the effector molecule comprised by the conjugate of the invention is considered, compared to the application of free saponin lacking a cell-targeting binding molecule such as one, two, three or more sdAb(s).

Application of the small sdAb such as a camelid V_{H} in the conjugate of the invention prevents or slows down clearance of the conjugate of the invention from the circulation and from the body of a human subject to whom the conjugate was administered, when compared to clearance rates commonly observed for whole IgG based ADCs. In addition, due to the relatively small size of the sdAbs, the risk for limiting or hampering the saponin activity inside a target cell due to the presence of the linked protein domain is limited, compared to the larger size of e.g. an antibody when such an antibody would be bound to the saponin. In general, the smaller the size of the molecule linked to the saponin, the smaller the risk for interference with the saponin activity inside cells due to the presence of the bound molecule, e.g. an sdAb such as a V_{HH}. Moreover, the relative small size of the sdAbs results in their rapid distribution in tissue, such as tumor tissue, allowing for improved reaching of target cells by the conjugate of the invention, and therewith to improved (extent of) binding to the target cells, compared to the relatively large-sized IgGs commonly applied in e.g. ADCs, OACs. One of the many benefits of applying sdAbs in the conjugates of the invention, is the absence of an Fc tail common to regular antibodies of e.g. the IgG type. Absence of an Fc tail in the sdAb in the conjugate of the invention prevents occurrence of Fcγ-Receptor mediated off-target effects such as undesired side effects relating to Fcγ-Receptor activation, when the conjugate is administered to a patient in need thereof. Absence of an Fc tail eliminates the risk of side effects generated by the binding of an Fc to cells of a patient to whom e.g. an antibody-based ADC is administered. The sdAb comprising conjugates of the invention do not bear this risk for Fc-mediated undesired side effects. As said, to the surprise of the inventors, despite the relatively small size of the sdAbs in the conjugate, activity of all three moieties in the conjugate of the invention was apparent: cell-surface molecule dependent endocytosis is established, effector molecule specific activity inside the cell is apparent (e.g. gene silencing, and enhancement of the biological activity of the effector molecule under influence of the saponin in the conjugate is apparent. Covalently linking 1, 4 or 8 saponins to the sdAb(s) does not hamper endocytic receptor-mediated uptake (endocytosis) of the conjugate by the target cell. Apparent cleaving off of the saponins from the conjugate once the conjugate arrived in the endosome/lysosome is not hampered by the presence of bound sdAbs and effector moiety in the conjugate. The acid-labile cleavable bonds between the saponins and the remainder of the conjugate are still susceptible for cleavage at the endosomal pH, apparently. Furthermore, arriving at an effective cytosolic amount of the effector molecule of the conjugate is not hampered by linking the effector molecule, the saponins and the sdAbs together in a single conjugate molecule.

An embodiment is the conjugate of the invention, wherein the at least one effector molecule is covalently bound to at least one sdAb, preferably to one, of the at least one sdAb and/or to at least one, preferably one, of the at least one saponin, either via a linker or bound directly to the sdAb and/or to the saponin, and/or wherein the at least one saponin is covalently bound to at least one sdAb, preferably to one, of the at least one sdAb and/or to at least one effector molecule, preferably one, of the at least one effector molecule, either via a linker or bound directly to the sdAb and/or to the effector molecule. Equally preferred is the binding of the effector moiety/moieties and/or the at least one saponin to a peptide linker comprised by the at least one sdAb, such as two or three sdAbs, the binding of the effector moiety/moieties and/or saponins being through a linker bound to the peptide linker and bound to the effector moiety/moieties and/or saponins, for example a single linker linking both the effector moiety and the saponin(s) to the peptide linker of the at least one, preferably multiple sdAbs, such as 1-8. 2-7, 3-6, 4-5 sdAbs, preferably 2 or 3 sdAbs such as a biparatopic sdAb comprising a peptide linker at the N-terminus or C-terminus, preferably at the C-terminus of the string of sdAbs.

It is advantageous if the at least one saponin is covalently bound in the conjugate via a cleavable bond, wherein the cleavable bond is subject to cleavage under for example acidic, reductive, enzymatic and/or light-induced conditions. The cleavable bond being subject to cleavage under acidic conditions present in endosomes and/or lysosomes of human cells is preferred. Examples of suitable cleavable bonds are cleavable bonds, preferably comprised by a linker, selected from:
a bond subject to cleavage under acidic conditions such as a semicarbazone bond or a hydrazone bond, and/or
a bond susceptible to proteolysis, for example proteolysis by Cathepsin B, and/or a bond susceptible for cleavage under reductive conditions such as a disulfide bond;
   preferably being a bond subject to cleavage in vivo under acidic or enzymatic conditions present in endosomes and/or lysosomes of human cells, preferably of pH≤6.5, preferably pH≤6, more preferably pH≤5.5, more preferably being a bond selected from a semicarbazone bond and a hydrazone bond; most preferably being a hydrazone bond. It is preferred that such a cleavable bond is not susceptible, or only to a minor extent, to cleavage when the conjugate is present outside the endosome and lysosome of the cell, such as outside the cell or in the endocytosed vesicle after the conjugate engaged with an endocytic receptor by binding of the at least one, preferably at least two, sdAbs to the target cell-surface molecule. For example, the cleavable bond is preferably less susceptible to cleavage when the conjugate is present in the circulation of a human subject and/or is present extracellularly in an organ of the human subject, compared to the susceptibility for cleavage of the bond when the conjugate is in the endosome or in the lysosome of a target cell that endocytosed the conjugate. Therefore, it is preferred that the covalent bond, preferably comprised by a linker, preferably comprised by an acid-sensitive linker, is selected from any one or more of: a semicarbazone bond, a hydrazone bond, an imine bond, an acetal bond including a 1,3-dioxolane bond, a ketal bond, an ester bond, and/or an oxime bond, preferably wherein the acid-sensitive linker comprises a semicarbazone bond or a hydrazone bond. Preferred is a cleavable bond that is subject to cleavage in vivo under acidic conditions present in endosomes and/or lysosomes of human cells, preferably of pH≤6.5, preferably pH≤6, more preferably pH≤5.5, more preferably being a bond selected from a semicarbazone bond and a hydrazone bond; most preferably being a hydrazone bond. When the saponin is cleaved off from the remainder of the conjugate, the saponin improvingly exerts its endosomal escape enhancing activity to further molecules present in the endosome together with the saponin, such as the effector moiety of the conjugate, preferably a toxin or an oligonucleotide, more preferably an oligonucleotide. Saponins comprising an aldehyde group at the C-23 position of the aglycone are particularly preferred since these saponins have potent endosomal escape enhancing activity towards e.g. toxins and oligonucleotides such as antisense oligonucleotides, e.g. antisense BNA. Therefore, in the conjugate saponins are preferred that comprise or form an aldehyde group at position C-23 of the saponin's aglycone core structure under acidic conditions present in endosomes and/or lysosomes of human cells. For example, when the saponins of Group B and Group C are covalently bound in the conjugate via linker chemistry involving the aldehyde group (e.g. formation of a hydrazone bond or a semicarbazone bond), it is preferred that the aldehyde group is formed in the endosome or lysosome when the conjugate is endocytosed and the saponin is cleaved off from the remainder of the conjugate by cleavage of a cleavable bond. Therefore, it is preferred that the covalent bond, preferably comprised by a linker, preferably comprised by an acid-sensitive linker, is adapted to restore aldehyde function upon cleavage (e.g. under acidic conditions), preferably being the aldehyde function at position C-23 of the saponin, advantageously the covalent bond being selected from any one or more of: a semicarbazone bond, a hydrazone bond, an imine bond, an acetal bond including a 1,3-dioxolane bond, and/or an oxime bond, preferably wherein the bond is either a semicarbazone bond or a hydrazone bond. Examples of such saponins suitable for this purpose are listed in Table A1, and are for example the saponins of Groups A-C, in particular Group B and Group C, as outlined here above. For example, saponins comprising the aldehyde group at position C-23 of the gypsogenin or quillaic acid aglycone that were tested for their endosomal escape enhancing activity were amongst others (activity was confirmed in for example cell-based assays and in in vivo animal tumor models, LDL lowering therapy models, with either free unconjugated native saponin, or when the saponin was initially conjugated with a linker or via such linker with e.g. an sdAb, involving a cleavable bond from which the aldehyde group is re-formed after cleavage once the saponin arrives in the endosome): QS-21, SO1700, SO1730, SO1772, SO1832, SO1861, SO1862, SO1904, GE1741, SA1641, Quil-A.

Similar to saponin, it is (also) advantageous if the at least one effector moiety, preferably 1-4 effector moieties, more preferably a single copy of the effector moiety such as an oligonucleotide, is covalently bound in the conjugate via a cleavable bond, wherein the cleavable bond is subject to cleavage under for example acidic, reductive, enzymatic and/or light-induced conditions. The cleavable bond being subject to cleavage under acidic conditions present in endosomes and/or lysosomes of human cells is preferred. Examples of suitable cleavable bonds are cleavable bonds, preferably comprised by a linker, selected from:
a bond subject to cleavage under acidic conditions such as a semicarbazone bond or a hydrazone bond, and/or
a bond susceptible to proteolysis, for example proteolysis by Cathepsin B, and/or
a bond susceptible for cleavage under reductive conditions such as a disulfide bond;
   preferably being a bond subject to cleavage in vivo under acidic or enzymatic conditions present in endosomes and/or lysosomes of human cells, preferably of pH≤6.5, preferably pH≤6, more preferably pH≤5.5, more preferably being a bond selected from a semicarbazone bond and a hydrazone bond; most preferably being a hydrazone bond. It is preferred that such a cleavable bond is not susceptible, or only to a minor extent, to cleavage when the conjugate is present outside the endosome and lysosome of the cell, such as outside the cell or in the endocytosed vesicle after the conjugate engaged with an endocytic receptor by binding of the at least one, preferably at least two, sdAbs to the target cell-surface molecule. For example, the cleavable bond is preferably less susceptible to cleavage when the conjugate is present in the circulation of a human subject and/or is present extracellularly in an organ of the human subject, compared to the susceptibility for cleavage of the bond when the conjugate is in the endosome or in the lysosome of a target cell that endocytosed the conjugate. Therefore, it is preferred that the covalent bond, preferably comprised by a linker, preferably comprised by an acid-sensitive linker, is selected from any one or more of: a semicarbazone bond, a hydrazone bond, an imine bond, an acetal bond including a 1,3-dioxolane bond, a ketal bond, an ester bond, and/or an oxime bond, preferably wherein the acid-sensitive linker comprises a semicarbazone bond or a hydrazone bond.

Preferred is a cleavable bond that is subject to cleavage in vivo under acidic conditions present in endosomes and/or lysosomes of human cells, preferably of pH≤6.5, preferably pH≤6, more preferably pH≤5.5, more preferably being a bond selected from a semicarbazone bond and a hydrazone bond; most preferably being a hydrazone bond. When the effector molecule is cleaved off from the remainder of the conjugate, the effector molecule improvingly escapes the endosome or lysosome, e.g. where the saponin of the conjugate exerts its endosomal escape enhancing activity to further molecules present in the endosome together with the saponin, such as the effector moiety of the conjugate, preferably a toxin or an oligonucleotide, more preferably an oligonucleotide. For example, effector moieties in the conjugate were oligonucleotides (e.g. antisense oligonucleotide targeting ApoB gene or HSP27 gene, for silencing such gene) and proteinaceous toxins (e.g. dianthin, saporin), for which the activity inside cells bearing the cell-surface molecule for binding of the at least one, preferably at least two sdAb(s) of the conjugate, is improved when covalently bound in the conjugate with a cleavable bond, that is cleaved when the conjugate is in the endosome or lysosome of a mammalian cell, preferably a human cell. Such a cleavable bond is preferably a cleavable bond that is subject to cleavage in vivo under acidic conditions present in endosomes and/or lysosomes of human cells, preferably of pH≤6.5, preferably pH≤6, more preferably pH≤5.5, more preferably being a bond selected from a semicarbazone bond and a hydrazone bond; most preferably being a hydrazone bond.

It is preferred when both the at least one saponin and the at least one effector moiety are covalently coupled in the conjugate via a cleavable bond, preferably selected from:
a bond subject to cleavage under acidic conditions such as a semicarbazone bond, a hydrazone bond, an imine bond, an acetal bond including a 1,3-dioxolane bond, a ketal bond, an ester bond, and/or an oxime bond,
a bond susceptible to proteolysis, for example amide or peptide bond, preferably subject to proteolysis by Cathepsin B,
a red/ox-cleavable bond such as a disulfide bond, or a thiol-exchange reaction-susceptible bond such as a thio-ether bond,
   preferably selected from any one or more of: a semicarbazone bond, a hydrazone bond, an imine bond, an acetal bond including a 1,3-dioxolane bond, a ketal bond, an ester bond, and/or an oxime bond, preferably wherein the acid-sensitive linker comprises a semicarbazone bond or a hydrazone bond, preferably a cleavable bond that is subject to cleavage in vivo under acidic conditions present in endosomes and/or lysosomes of human cells, preferably of pH≤6.5, preferably pH≤6, more preferably pH≤5.5, more preferably being a bond selected from a semicarbazone bond and a hydrazone bond; most preferably being a hydrazone bond. The cleavable bond with which the saponin is bound in the conjugate is the same as or different from the cleavable bond with which the effector moiety is bound in the conjugate. Hydrazone bond and semicarbazone bond are preferred. The saponin is preferably a saponin with an aldehyde group at position C-23 of the aglycone, when in its non-conjugated native form, such as a saponin selected from Group B and C or a saponin selected from Table A1. The effector molecule is preferably a proteinaceous toxin, a protein or an oligonucleotide, more preferably an oligonucleotide such as an antisense nucleic acid. The conjugate comprises preferably at least two sdAbs, such as 2, 3, 4, 5, 6, 7 or 8 V_{HH} domains. Preferred conjugates are conjugates with 2, 3 or 4 V_{HH} domains, preferably linearly linked together via peptide bonds, preferably with short linker sequences in between consecutive domains known in the art. Examples of preferred conjugates are conjugates with a bivalent sdAb or a trivalent sdAb, such as a biparatopic sdAb. In the context of the invention, it is to be understood that phrases such as “bivalent sdAb” and “trivalent V_{HH}” and “biparatopic sdAb” mean two, three, and two sdAb domains or V_{HH} domains linked together, respectively. The bivalent sdAb thus refers for example to sdAb-sdAb, i.e. two sdAbs that bind to the same type of e.g. receptor. It is preferred that the at least one, preferably at least two sdAb(s) of the conjugate comprise a C-terminal amino-acid linker sequence for covalently binding the saponin(s) and effector moiety/moieties. A suitable linker is the tetra-Cys linker of SEQ ID NO: 77. Alternatively, such a linker comprises 1-6 Cys residues, such as 1, 2 or 3 Cys residues, for binding at least one, preferably one or two, more preferably one, linker to which both the at least one saponin and the at least one effector moiety are bound, typically each via a further linker. That is to say, preferred is a conjugate comprising one copy of the at least one sdAb, preferably at least two sdAbs, such as a bivalent or trivalent sdAb, such as a biparatopic sdAb, one copy of the at least saponin, such as 1-16 saponins, preferably 1-8 saponins, such as 1, 2, 4, 6 or 8 saponins (on average), preferably 1, 4 or 8 saponins, and one copy of the at least one effector moiety, such as 1-8 effector moieties, preferably 1-4 effector moieties, more preferably 1 or 2 effector moieties, most preferably a single effector moiety. An example of a preferred conjugate comprises 1 bivalent or trivalent sdAb, 1, 4 or 8 saponin molecules and 1-4, preferably 1 effector molecule, preferably an oligonucleotide.

Such a conjugate is for example schematically depicted as: sdAb-sdAb(-saponin(s))(-effector moiety), or as depicted as “CONJUGATE C”:

NH₂-sdAb1-linker1-sdAb2-coupling linker-COOH(-linker2(-linker3-(1-8 saponins))(-linker4-(1-4 effector moieties))) (CONJUGATE C)

wherein the coupling linker is bound to the linker 2, and wherein the linker 3 bearing the bound saponin(s) is bound to linker 2 and the linker 4 bearing the effector moiety/moieties is bound to linker 2. Typically, linker 2 is a trifunctional linker. Bonds between the saponin(s) and linker 3, and/or bonds between the effector moiety/moieties and linker 4 are preferably cleavable bonds as herein described. Equally preferred is the conjugate comprising a further sdAb3 and for example a further sdAb4 (conjugates comprising 3 or 4 sdAbs, such as tri- or tetravalent sdAbs, or two bivalent sdAbs, or a bivalent sdAb and a further sdAb).

An embodiment is the conjugate of the invention, wherein the conjugate comprises at least one first linker with each of the at least one sdAb bound thereto, preferably at least one bivalent nanobody, more preferably a single bivalent nanobody, the at least one saponin bound thereto and the at least one effector molecule covalently bound thereto, preferably separately, either directly, or via a first, second and third additional linker for the at least one sdAb, the at least one saponin and the at least one effector molecule, respectively. Preferably, the conjugate comprises at least one of a first linker with one bivalent nanobody, at least one saponin and at least one, preferably one, effector molecule covalently bound to that first linker, separately, either directly, or via a first, second and third additional linker for conjugating the at least one bivalent nanobody, the at least one saponin and the at least one effector molecule, respectively. For example, the conjugate comprises at least one first linker with:
each of the at least one sdAb bound thereto, preferably at least one bivalent nanobody, more preferably a single bivalent nanobody; and
the at least one saponin bound thereto; and
the at least one effector molecule covalently bound thereto,
   preferably bound to said at least one first linker separately, either directly, or via a first, second and third additional linker for the at least one sdAb, the at least one saponin and the at least one effector molecule, respectively;

preferably, the conjugate comprises at least one of a first linker with one bivalent nanobody, at least one saponin and at least one, preferably one, effector molecule covalently bound to that first linker, separately, either directly, or via a first, second and third additional linker for conjugating the at least one bivalent nanobody, the at least one saponin and the at least one effector molecule, respectively. For example, the first linker is linker2 of CONJUGATE C. An embodiment is the conjugate of the invention, wherein the conjugate comprises a trifunctional linker with each of the at least one sdAb, the at least one saponin and the at least one effector molecule covalently bound to the trifunctional linker, preferably separately, either directly, or via a linker, and preferably, the conjugate comprises a trifunctional linker with one sdAb, the at least one saponin and at least one, preferably one, effector molecule covalently bound to the trifunctional linker, separately, either directly, or via a linker.

Coupling of the saponin to the (at least two) sdAb and/or to the effector molecule via a linker provides flexibility when the binding site for coupling of the saponin to the sdAb and/or to the effector molecule is considered. Furthermore, such a linker may act as a spacer between the sdAb and the saponin and the effector molecule, such that the sdAb maintains its capability to bind to a binding site on a cell surface molecule and the saponin maintains its capability to enhance endosomal escape of the effector molecule comprised by the conjugate, and the effector molecule maintains its biological activity towards its intracellular binding partner.

An embodiment is the conjugate according to the invention wherein the first linker is a trifunctional linker, preferably wherein the conjugate comprises 1-4 of said trifunctional linkers for every at least one sdAb or every multivalent nanobody, preferably bivalent nanobody, comprised by the conjugate, more preferably 1-2, even more preferably 1 trifunctional linker,

or
wherein the first linker is a trifunctional linker, preferably wherein the conjugate comprises on average 1-4, preferably on average 1.2-1.8 of said trifunctional linkers.

An embodiment is the conjugate of the invention, wherein the conjugate comprises (on average) 1-4 of the trifunctional linkers for every at least one sdAb, preferably at least two sdAbs, comprised by the conjugate, more preferably being 1-2 trifunctional linkers, most preferably being 1.2-1.8. trifunctional linkers on average.

An embodiment is the conjugate of the invention, wherein the at least one sdAb or the multivalent nanobody such as the bivalent nanobody comprises a first additional linker comprising at least one cysteine residue, preferably 1-4 cysteine residues, such as 1, 2, 3 or 4, preferably a tetracysteine repeat such as sequence HRWCCPGCCKTF (SEQ ID NO: 77), and wherein each of the trifunctional linkers, preferably one trifunctional linker, is bound to this first additional linker, more preferably wherein the conjugate comprises a single multivalent nanobody, preferably a trivalent or bivalent nanobody, comprising said first additional linker comprising at least one cysteine residue, preferably 1-4 cysteine residues, preferably a tetracysteine repeat such as sequence HRWCCPGCCKTF (SEQ ID NO: 77), and all of the one or more trifunctional linkers, preferably one trifunctional linker, are each/is separately bound to a cysteine residue of the tetracysteine repeat of the first additional linker. Such first additional linker preferably is an amino-acid sequence at the C-terminal side of the at least one, preferably at least two sdAb(s) of the conjugate.

An embodiment is the conjugate of the invention, wherein the conjugate comprises any one of one multivalent nanobody such as a trivalent nanobody or a bivalent nanobody, 1-4 sdAb's, 1-2 sdAb's and 1 bivalent nanobody, preferably one bivalent nanobody and/or 3 sdAbs preferably comprising a bivalent nanobody.

An embodiment is the conjugate of the invention, wherein the at least one saponin is originating from a mono-desmosidic or bi-desmosidic triterpene saponin, or derivative thereof, belonging to the type of a 12,13-dehydrooleanane saponin with an aldehyde function in position C₂₃ and optionally comprising a glucuronic acid unit in a first saccharide chain bound at the C₃beta-OH group of the aglycone core structure of the saponin, preferably at least one saponin originating from a bi-desmosidic triterpene saponin, belonging to the type of a 12,13-dehydrooleanane saponin with an aldehyde function in position C₂₃ and comprising a glucuronic acid unit in a first saccharide chain bound at the C₃beta-OH group of the aglycone core structure of the saponin, wherein the aglycone core structure is gypsogenin or quillaic acid, preferably quillaic acid.

An embodiment is the conjugate of the invention, wherein the conjugate comprises more than one copy of the saponin, preferably 1-64 copies of the saponin, more preferably 2-32 copies of the saponin, even more preferably 4-16 copies of the saponin, most preferably 4-8 copies of the saponin. Typically, the conjugate comprises 1, 4 or 8 saponin molecules, for example 1, 4 or 8 saponin molecules per each of the at least one, preferably at least two, such as two or three, sdAb(s) comprised by the conjugate.

An embodiment is the conjugate of the invention, wherein the conjugate comprises 1-16 molecules of the saponin per 1 molecule of the at least one, preferably two or three, sdAb(s);

preferably 2-8 molecules of the saponin per 1 molecule of the at least one sdAb;
more preferably 3-4 molecules of the saponin per 1 molecule of the at least one, preferably two or three, sdAb(s);
most preferably wherein the conjugate comprises on average 1, 4 or 8 molecules of the saponin per 1 molecule of the at least one, preferably two or three, sdAb(s). Preferred are conjugates with two or three sdAbs.

An embodiment is the conjugate of the invention, wherein the conjugate comprises 1-8 molecules of the nucleic acid per 1 molecule of the at least one, preferably two or three, sdAb(s); preferably 1-4 molecules of the nucleic acid per 1 molecule of the at least one, preferably two or three, sdAb(s);

more preferably wherein the conjugate comprises on average 1 or 2 molecules of the nucleic acid per 1 molecule of the at least one, preferably two or three, sdAb(s). Typically, the conjugate comprises on average 1-2, preferably 1 or 2, copies of the effector moiety, and on average 1-16, preferably 1, 4 or 8, copies of the saponin. Preferably, the conjugate comprises two or three sdAbs, such as a bivalent sdAb.

An embodiment is the conjugate of the invention, wherein the at least one saponin is covalently bound directly to an amino-acid residue of the first linker, preferably to a cysteine and/or to a lysine, and/or is covalently bound via the first additional linker, wherein preferably said first additional linker is a cleavable linker.

An embodiment is the conjugate of the invention, wherein the first additional linker to which the one or more saponins are covalently bound comprises a polymeric molecule or an oligomeric molecule to which the one or more saponins are covalently bound, the polymeric molecule or the oligomeric molecule selected from: a dendron, a poly-ethylene glycol such as any one of PEG₃-PEG₃₀, preferably any one of PEG₄-PEG₁₂, preferably the polymeric molecule or the oligomeric molecule of the conjugate is a dendron such as a poly-amidoamine (PAMAM) dendrimer.

An embodiment is the conjugate of the invention, wherein the first additional linker that covalently binds the one or more saponins to the first linker is a dendron to which the one or more saponins are covalently bound, preferably a G2 dendron, a G3 dendron, a G4 dendron or a G5 dendron, more preferably a G2 dendron or a G3 dendron. For example, said first additional linker that covalently binds the one or more saponins to the first linker is a dendron to which the one or more saponins are covalently bound, preferably a G2 dendron, a G3 dendron, a G4 dendron or a G5 dendron or a poly-amidoamine (PAMAM) dendrimer, more preferably a G2 dendron or a G3 dendron or a poly-amidoamine (PAMAM) dendrimer, more preferably a G2 dendron or a G3 dendron.

An embodiment is the conjugate of the invention, wherein the at least one saponin is covalently bound via a cleavable first additional linker to the first linker.

An embodiment is the conjugate of the invention, wherein the cleavable first additional linker is subject to cleavage under acidic conditions, reductive conditions, enzymatic conditions and/or light-induced conditions, and preferably the first additional linker is an acid-sensitive linker and preferably the cleavable first additional linker comprises a cleavable bond selected from

preferably, a semicarbazone bond or a hydrazone bond, more preferably a hydrazone bond subject to cleavage under acidic conditions, and/or a bond susceptible to proteolysis, for example proteolysis by Cathepsin B, and/or a bond susceptible for cleavage under reductive conditions such as a disulfide bond.

For example, such a cleavable first additional linker comprised by the conjugate is subject to cleavage under acidic conditions, reductive conditions, enzymatic conditions and/or light-induced conditions, and preferably the cleavable first additional linker comprises a cleavable bond selected from
a bond subject to cleavage under acidic conditions such as a semi-carbazone bond, a hydrazone bond, an imine bond, an acetal bond including a 1,3-dioxolane bond, a ketal bond, an ester bond, and/or an oxime bond,
a bond susceptible to proteolysis, for example an amide or a peptide bond, preferably subject to proteolysis by Cathepsin B,
a red/ox-cleavable bond such as a disulfide bond, or a thiol-exchange reaction-susceptible bond such as a thio-ether bond,

An embodiment is the conjugate of the invention, wherein the cleavable first additional linker is subject to cleavage in vivo under acidic conditions such as for example present in endosomes and/or lysosomes of mammalian cells, preferably human cells such as tumor cells, preferably at pH 4.0-6.5, and more preferably at pH 5.5.

An embodiment is the conjugate of the invention, wherein the at least one saponin is covalently bound to the first additional linker or cleavable first additional linker, preferably an acid-sensitive linker, via any one or more of a semicarbazone bond, an imine bond, a hydrazone bond, an oxime bond, a 1,3-dioxolane bond, a disulfide bond, a thio-ether bond, an amide bond, a peptide bond or an ester bond, preferably a semicarbazone bond or a hydrazone bond, more preferably a hydrazone bond. For example, the at least one saponin comprised by the conjugate is covalently bound to the first additional linker or cleavable first additional linker via any one or more of: a semi-carbazone bond, a hydrazone bond, an imine bond, an acetal bond including a 1,3-dioxolane bond, a ketal bond, an ester bond, and/or an oxime bond, preferably a semicarbazone bond or a hydrazone bond, more preferably a hydrazone bond.

An embodiment is the conjugate of the invention, wherein the at least one saponin, when in its free non-conjugated form, comprises an aglycone core structure comprising an aldehyde function in position C₂₃, which aldehyde function is involved in the covalent bonding to the first linker, the first additional linker or the cleavable first additional linker, preferably the cleavable first additional linker.

An embodiment is the conjugate of the invention, wherein the conjugate is obtained by conjugating at least one multivalent nanobody, preferably at least one bivalent nanobody comprising two single domain antibodies (sdAbs), with at least one saponin, comprising an aglycone core structure comprising an aldehyde function in position C₂₃, which aldehyde function is involved in the covalent bonding to the first linker, the first additional linker or the cleavable first additional linker, preferably the cleavable first additional linker. For example, the conjugate is obtained by conjugating the at least one effector molecule, the at least one sdAb, or the at least one multivalent nanobody, preferably the at least one bivalent nanobody comprising two sdAbs, with at least one saponin wherein said at least one saponin comprises an aglycone core structure comprising an aldehyde function in position C₂₃, which aldehyde function is involved in the covalent bonding to the first linker, the first additional linker or the cleavable first additional linker, preferably the cleavable first additional linker.

An embodiment is the conjugate of the invention, wherein the first linker is a trifunctional linker that is in its non-conjugated form represented by Structure A:

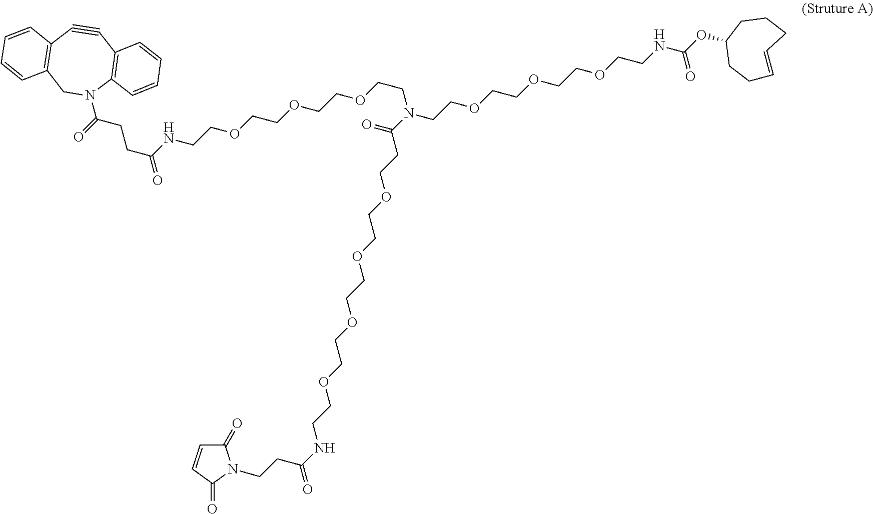

An embodiment is the conjugate of the invention, wherein the first linker of the conjugate is the trifunctional linker of Structure A as represented here above and wherein the conjugate has a molecular structure represented by Structure B:

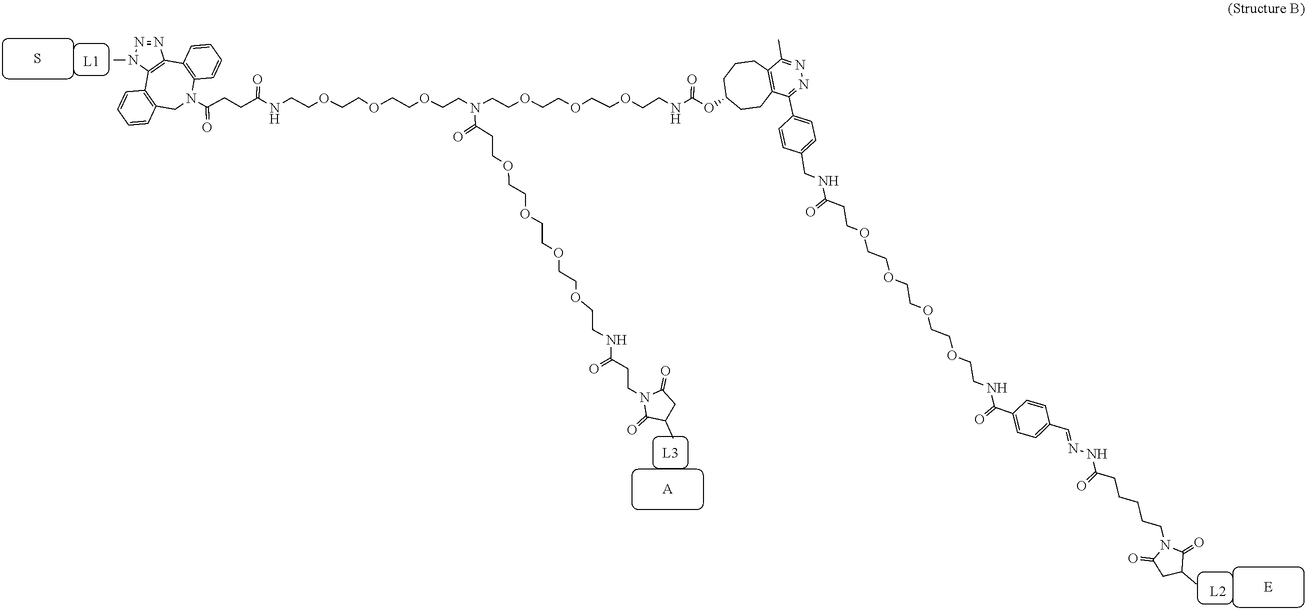

wherein S represents the at least one saponin of any one of the previous embodiment, preferably a saponin selected from Group A, B or C, more preferably Group B or C, most preferably Group C, E is the at least one, preferably one, effector molecule, A is the at least one sdAb such as a two, three or four sdAbs, preferably a bivalent nanobody comprising a first sdAb and a second sdAb according to the invention, L1 is the first additional linker to which the at least one saponin is covalently bound, L1 optionally comprising the oligomeric or polymeric molecule as here above described for certain embodiments (preferably a PEG linker selected from PEG3-PEG30, or a G2, G3 or G4 dendron) to which the at least one saponin is covalently bound, L2 is the second additional linker to which the at least one, preferably one or two effector molecule(s) is/are covalently bound and L3 is the third additional linker to which the at least one nanobody (sdAb), preferably one bivalent nanobody, is covalently bound, wherein L1, L2 and L3 are the same or different, preferably different. For example, the first linker of the conjugate is the trifunctional linker of Structure A as represented here above and wherein the conjugate has a molecular structure represented by Structure B as represented here above, wherein S represents the at least one saponin, E is the at least one, preferably one, effector molecule, A is the at least one sdAb such as a single sdAb, or the at least one multivalent nanobody, preferably the at least one bivalent nanobody comprising a first sdAb and a second sdAb, L1 is the first additional linker to which the at least one saponin is covalently bound, L1 optionally comprising the oligomeric or polymeric molecule to which the at least one saponin is covalently bound, L2 is the second additional linker to which the at least one, preferably one effector molecule is covalently bound and L3 is the third additional linker to which the at least one sdAb, the at least one multivalent nanobody, preferably the at least one bivalent nanobody, more preferably one bivalent nanobody, is covalently bound, wherein L1, L2 and L3 are the same or different, preferably different.

An embodiment is the conjugate of the invention, wherein the at least one saponin is covalently bound via a thio-ether bond to a sulfhydryl group in one of the at least one sdAb and/or in one of the at least one effector molecule, the covalent bonding preferably via linker N-s-maleimidocaproic acid hydrazide (EMCH) that is covalently bound to an aldehyde group in position C23 of the aglycone core structure of the saponin and that is covalently bound to the sulfhydryl group in the sdAb and/or in the effector molecule, such as a sulfhydryl group of a cysteine.

An embodiment is the conjugate of the invention, wherein the at least one saponin is a bi-desmosidic triterpene saponin or derivative thereof belonging to the type of a 12,13-dehydrooleanane with optionally an aldehyde function in position C23 and comprising a glucuronic acid unit in a first saccharide chain bound at the C3beta-OH group of the aglycone core structure of the saponin, wherein the saponin is covalently bound to an amino-acid residue of the at least one sdAb and/or of the at least one effector molecule via the carboxyl group of the glucuronic acid unit in the first saccharide chain, preferably via a linker, wherein the amino-acid residue preferably is selected from cysteine and lysine.

An embodiment is the conjugate of the invention, wherein the at least one saponin comprises a glucuronic acid unit in the first saccharide chain at the C3beta-OH group of the aglycone core structure of the saponin, which glucuronic acid unit is covalently bound to a linker, which linker is preferably covalently bound via an amide bond to an amine group in the at least one sdAb and/or in the at least one effector molecule, such as an amine group of a lysine or an N-terminus of the sdAb and/or of the effector molecule, preferably said linker is 1-[Bis(dimethylamino)methylene]-1H-1,2,3-triazolo[4,5-b]pyridinium 3-oxid hexafluorophosphate (HATU).

An embodiment is the conjugate of the invention, comprising more than one covalently bound saponin moieties of the at least one saponin, preferably 2, 3, 4, 5, 6, 8, 10, 16, 32, 64, 128 or 1-100 of such moieties, or any number of such moieties therein between, such as 7, 9, 12 saponin moieties.

An embodiment is the conjugate of the invention, wherein the conjugate comprises 1-100 saponin moieties of the at least one saponin, preferably 2-64 saponin moieties, more preferably 4-32 saponin moieties, most preferably 8-16 saponin moieties, or any number therein between. Typically, the conjugate of the invention comprises 1, 2, 4, 8 or 16 saponin moieties (copies of the saponin molecule). For example, 4 saponin molecules are covalently linked to a G2 dendron and at least one, such as one or two, of the dendron-(saponin)₄ conjugate (saponin conjugate) is covalently linked to an sdAb. For example, 8 saponin molecules are covalently linked to a G3 dendron and at least one, such as one or two, of the dendron-(saponin)₈ conjugate (saponin conjugate) is covalently linked to an sdAb.

An embodiment is the conjugate of the invention wherein the conjugate comprises more than one saponin moieties wherein the saponin moieties are the same or different. That is to say, if more than one saponins are covalently linked to the sdAb(s) in the conjugate of the invention, these saponins can all be copies of the same saponin, or the saponin are different saponins. Preferred is the conjugate comprising multiple saponin moieties, wherein the saponins that are bound to the sdAb(s) are the same. For example, 2, 4, 8, 16 saponin molecules covalently linked to the sdAb's in the conjugate, for example 2-16 SO1861 copies or QS-21 copies, preferably SO1861, preferably 1, 4 or 8 copies of SO1861.

An embodiment is the conjugate of the invention, wherein the more than one covalently bound saponin moieties are covalently bound directly to an amino-acid residue of the at least one, preferably at least two, sdAb(s) and/or of the at least one effector molecule, preferably to a cysteine and/or to a lysine, and/or are covalently bound via a linker and/or via a cleavable linker.

An embodiment is the endosomal and/or lysosomal escape enhancing conjugate according to the invention, essentially having the molecular format of molecular structure (II):

(saponin-linker-)ₐ(sdAb)ₙ-effector moiety (STRUCTURE (II)),
wherein a=1-8, preferably 1, 2, 4, 8, and wherein n is 1-8, preferably at least 2, such as 2, 3 or 4, wherein the sdAbs are the same or different, and wherein the sdAbs bind the same epitope on the same receptor, or bind different (non-overlapping) epitopes on the same receptor,
or essentially having the molecular format of molecular structure (Ill):

(saponin-dendron(-saponin)ₓ)_{b}-(sdAb)ₘ-effector moiety (STRUCTURE (III)),
wherein x=between 1 and 100, preferably 1-63, 1-31, 1-15, 1-7, or 3; b=1-4, preferably 1, 2, 4, and wherein m is 1-8, preferably at least 2, such as 2, 3 or 4, wherein the sdAbs are the same or different, and wherein the sdAbs bind the same epitope on the same receptor, or bind different (non-overlapping) epitopes on the same receptor,
or essentially having the molecular format of molecular structure (IV):

(saponin-trifunctional linker(-effector moiety))_{c}-(sdAb)ₒ (STRUCTURE (IV)),
wherein c=1-4, preferably 1, 2, 4, and wherein o is 1-8, preferably at least 2, such as 2, 3 or 4, wherein the sdAbs are the same or different, and wherein the sdAbs bind the same epitope on the same receptor, or bind different (non-overlapping) epitopes on the same receptor,
or essentially having the molecular format of molecular structure (V):

((saponin-dendron(-saponin)_{y})-trifunctional linker(-effector moiety))_{d}-(sdAb)ₚ (STRUCTURE (V)),
wherein y=between 1 and 100, preferably 1-63, 1-31, 1-15, 1-7, or 3; d=1-4, preferably 1, 2, 4, and wherein p is 1-8, preferably at least 2, such as 2, 3 or 4, wherein the sdAbs are the same or different, and wherein the sdAbs bind the same epitope on the same receptor, or bind different (non-overlapping) epitopes on the same receptor.

Preferably, x or y is 3, 7 or 15. Preferably, b or d is 1, 2 or 4, although in some embodiments, b or d is 3, and for the single sdAb such as a single V_{HH} domain, a is preferably 1, b is preferably 1, c is preferably 1 and d is preferably 1. The Dendron is for example a G2, G3, G4 dendron or a G5 dendron. Preferably, the saponin is bound to the linker via a cleavable bond, such as a hydrazone bond that is cleaved intracellularly under pH conditions of <6.5 (i.e. the pH in the endosome, endolysosome, lysosome). Preferably the linker is EMCH. Preferably, the trifunctional linker is the linker with Structure A as displayed hereunder. For Structure II and Ill, preferably, the effector moiety is bound to the one or more, preferably 2 or 3, sdAb(s) via a linker such as a cleavable linker. The saponin is selected according to any of the previous embodiments, and preferably a saponin selected from Table A1 or Group B or Group C, more preferably from Group C, such as SO1832 and SO1861. The effector molecule is preferably an oligonucleotide such as an antisense oligonucleotide.

An embodiment is the conjugate of the invention, wherein the more than one covalently bound saponin moieties are part of a covalent saponin conjugate comprising at least one oligomeric molecule or polymeric molecule and the more than one saponin covalently bound thereto, wherein the covalent saponin conjugate is covalently bound to at least one of the at least one sdAb and/or to at least one of the at least one effector molecule.

An embodiment is the conjugate of the invention, wherein the at least one saponin is part of a covalent saponin conjugate comprising an oligomeric molecule or a polymeric molecule to which the saponin is covalently bound, and wherein the sdAb is also covalently bound to the same oligomeric molecule or polymeric molecule as to which the saponin is bound. Thus, the oligomeric molecule or the polymeric molecule comprises the one or more covalently bound saponin moieties and is covalently bound to the at least one, preferably two or three, sdAb(s) of the conjugate. The saponin and the sdAbs are covalently bound to each other via the polymeric molecule or the oligomeric molecule, preferably via (a) linker(s). The oligomeric molecule or the polymeric molecule links the sdAb(s) and the saponin(s) together, providing a saponin-comprising conjugate of the invention. In addition, the effector molecule is either bound to the oligomeric molecule or the polymeric molecule, or is bound to the sdAb(s), although the latter is preferred, the binding of the effector molecule(s) preferably involving (a) linker(s) between the at least one sdAb and the effector molecule(s).

Such a covalent saponin conjugate of saponins bound to the oligomeric or polymeric molecule serves as a carrier (support, scaffold) for multiple saponin moieties, which can be bound to the sdAb(s) comprised by the conjugate, via a single bond, preferably via a (cleavable) linker. Since the covalent saponin conjugate can bear any selected number of covalently bound saponin moieties, such as 1-200 saponin moieties, relating to the type of selected oligomeric or polymeric structure comprising binding sites for covalent linking these saponins, application of such covalent saponin conjugate provides freedom when the number of saponin moieties in the conjugate of the invention is considered. For example, for cytosolic delivery of the effector molecule comprised by the conjugate of the invention, the number of saponins present in the conjugate of the invention can be adapted by providing the covalent saponin conjugate with a number of saponin moieties sufficient and enough for stimulating the cytosolic delivery of the effector molecule, when the covalent saponin conjugate is part of the conjugate of the invention, and when the effector molecule co-localizes with the saponins as integral part of the very same conjugate in the endosome or lysosome of a target cell in which the effector molecule should exert its biological activity. Typically, 1-8 saponin molecules for each effector molecule is/are present in the conjugate for each copy of the effector molecule of the conjugate, such as 1, 4 or 8, preferably 1 or 4, copies of the saponin molecule.

Preferably, 1-8 of the covalent saponin conjugates are bound to the sdAb(s), preferably to the two or three sdAbs, and/or to the effector molecule, more preferably 1-4 of such covalent saponin conjugates, wherein the at least one covalent saponin conjugate is optionally based on a dendron (for example a G2 dendron, G3 dendron, G4 dendron or a G5 dendron), such as a PAMAM, wherein optionally 1-32 saponin moieties, preferably 2, 3, 4, 5, 6, 8, 10, 16, 32 of such moieties, or any number of such moieties therein between, such as 7, 9, 12 saponin moieties, are covalently bound to the oligomeric molecule or to the polymeric molecule of the at least one covalent saponin conjugate, either directly or via a linker. For example, 4 saponin moieties are bound to a G2 dendron, 8 saponin moieties are bound to a G3 dendron, 16 saponin moieties are bound to a G4 dendron, wherein the saponin for example is SO1861, SO1832 or QS21, preferably SO1861.

An embodiment is the conjugate of the invention, wherein the more than one covalently bound saponin moieties are part of a covalent saponin conjugate comprising an oligomeric molecule or a polymeric molecule to which the saponin is covalently bound, and wherein the sdAb is also covalently bound to the same oligomeric molecule or polymeric molecule as to which the saponin is bound and wherein the effector moiety is covalently bound to the sdAb or to the oligomeric molecule or the polymeric molecule, preferably 1-8 of such oligomeric molecules or polymeric molecules comprising the saponin(s) is/are covalently bound to the sdAb, or 2-4 of the oligomeric molecules or polymeric molecules comprising the saponin(s) are covalently bound to the sdAb, wherein the oligomeric molecule or the polymeric molecule of the covalent saponin conjugate is optionally a dendron such as a G2 dendron, G3 dendron, G4 dendron or G5 dendron with 4, 8, 16 and 32 binding sites for covalently binding 4, 8, 16 or 32 saponin moieties, respectively, wherein optionally 1-32 saponin moieties, preferably 2, 3, 4, 5, 6, 8, 10, 16, 32 of such moieties, or any number of such moieties therein between, such as 7, 9, 12 saponin moieties, are covalently bound to the oligomeric molecule or to the polymeric molecule of the at least one covalent saponin conjugate, either directly or via a linker.

Preferably, one or two of the covalent saponin conjugates is/are bound to a single sdAb in the conjugate of the invention. For many purposes, coupling of a single saponin or coupling of a single covalent saponin conjugate to a single sdAb comprised by the conjugate, suffices for efficient stimulation of the effector molecule delivery into a target cell and into the cytosol of said cell, wherein the effector molecule is comprised by the conjugate of the invention. Typically, 4, 8 or 16 saponins are comprised by the conjugate of the invention, such as 4 or 8 saponins comprised by a single covalent saponin conjugate coupled to the sdAb in the conjugate of the invention. For example, the 4, 8 or 16 saponin moieties are covalently coupled to a G2 dendron, G3 dendron or G4 dendron, respectively. Typically, such conjugates of the invention comprise a single sdAb, to which the saponin or saponins or the covalent saponin conjugate(s) is/are bound, preferably a single saponin or a single covalent saponin conjugate is part of the conjugate.

An embodiment is the conjugate of the invention, wherein the oligomeric molecule or the polymeric molecule of the covalent saponin conjugate is a dendron, such as a G2 dendron, G3 dendron, G4 dendron or G5 dendron, wherein 1-32 saponin moieties, preferably 2, 3, 4 (for example for a G2 dendron), 5, 6, 8 (for example for a G3 dendron), 10, 16 (for example for a G4 dendron), 32 (for example for a G5 dendron) saponin moieties, or any number of saponin moieties therein between, such as 7, 9, 12 saponin moieties, are covalently bound to the oligomeric molecule or to the polymeric molecule of the covalent saponin conjugate.

An embodiment is the saponin-comprising conjugate of the invention, wherein 1-8 of the oligomeric molecules or polymeric molecules comprising the saponin(s) is/are covalently bound to the sdAb.

An embodiment is the saponin-comprising conjugate of the invention, wherein 2-4 of the oligomeric molecules or polymeric molecules comprising the saponin(s) are covalently bound to the sdAb.

An embodiment is the saponin-comprising conjugate of the invention, wherein the oligomeric molecule or the polymeric molecule of the covalent saponin conjugate is a dendron. Preferred is an oligomeric molecule or a polymeric molecule such as a dendron or a polyethylene glycol that is biologically sufficiently inactive or inert, preferably biologically inactive or inert.

An embodiment is the saponin-comprising conjugate of the invention, wherein the oligomeric molecule or the polymeric molecule of the covalent saponin conjugate is a G2 dendron, a G3 dendron, a G4 dendron or a G5 dendron with 4, 8, 16 and 32 binding sites for covalently binding 4, 8, 16 or 32 saponin moieties, respectively.

An embodiment is the saponin-comprising conjugate of the invention, wherein 1-32 saponin moieties, preferably 2, 3, 4, 5, 6, 8, 10, 16, 32 saponin moieties, or any number of saponin moieties therein between, such as 7, 9, 12 saponin moieties, are covalently bound to the oligomeric molecule or to the polymeric molecule of the covalent saponin conjugate. It is to be understood that “saponin-comprising conjugate” in the context of the invention is referring to the conjugate of at least one saponin molecule and at least one sdAb and at least one effector molecule and is not limited to a conjugate of the invention comprising a single sdAb and a single saponin molecule. For example, the conjugate comprises or consists of one or two sdAb's such as V_{HH}('s), and 1-32 saponin molecules, such as 2, 4, 8, 16 saponin molecules, and at least one effector moiety.

An embodiment is the conjugate of the invention, wherein the at least one saponin is covalently bound to at least one of the at least one sdAb and/or to at least one of the at least one effector molecule via a cleavable linker.

An embodiment is the conjugate of the invention, wherein the cleavable linker is subject to cleavage under acidic conditions, reductive conditions, enzymatic conditions and/or light-induced conditions, and preferably the cleavable linker comprises a cleavable bond which is a hydrazone bond subject to cleavage under acidic conditions, and/or a bond susceptible to proteolysis, for example proteolysis by Cathepsin B, and/or a bond susceptible for cleavage under reductive conditions such as a disulfide bond.

An embodiment is the conjugate of the invention, wherein the cleavable linker is subject to cleavage under conditions present in endosomes or lysosomes, for example acidic or enzymatic conditions present in endosomes or lysosomes, preferably wherein the linker comprises a cleavable bond selected from:
a bond subject to cleavage under acidic conditions such as a semicarbazone bond, a hydrazone bond, an imine bond, an acetal bond including a 1,3-dioxolane bond, a ketal bond, an ester bond, and/or an oxime bond,
a bond susceptible to proteolysis, for example amide or peptide bond, preferably subject to proteolysis by Cathepsin B;
a red/ox-cleavable bond such as a disulfide bond, or a thiol-exchange reaction-susceptible bond such as a thio-ether bond.

An embodiment is the conjugate of the invention, wherein the cleavable linker is an acid-sensitive linker that comprises a covalent bond selected from any one or more of: a semicarbazone bond, a hydrazone bond, an imine bond, an acetal bond including a 1,3-dioxolane bond, a ketal bond, an ester bond, and/or an oxime bond, preferably wherein the acid-sensitive linker comprises a semicarbazone bond or a hydrazone bond.

An embodiment is the conjugate of the invention, wherein the cleavable linker is an acid-sensitive linker that comprises a covalent bond adapted to restore aldehyde function upon cleavage (e.g. under acidic conditions), preferably being the aldehyde function at position C-23 of the saponin, advantageously the covalent bond being selected from any one or more of: a semicarbazone bond, a hydrazone bond, an imine bond, an acetal bond including a 1,3-dioxolane bond, and/or an oxime bond, preferably wherein the bond is either a semicarbazone bond or a hydrazone bond.

An embodiment is the conjugate of the invention, wherein the cleavable linker is subject to cleavage in vivo under acidic conditions as for example present in endosomes and/or lysosomes of mammalian cells, preferably human cells, preferably at pH 4.0-6.5, and more preferably at pH≤5.5.

Such cleavable linkers that are cleavable under the conditions as apparent in endosomes and lysosomes facilitates the delivery of free saponin inside the endosome or lysosome, upon cleavage (splitting off) of the saponin from the remainder of the conjugate of the invention. This way, the conjugate of the invention combines the benefits of cell-targeted delivery of the saponin upon specific binding of the sdAb, to the cell-surface molecule on the target cell, and of the presence of the free saponin inside the cell, i.e. inside the endosome (or lysosome), which contributes to the ability of the free saponin to stimulate and/or facilitate the delivery of the effector molecule comprised by the conjugate of the invention, out of the endosome (or lysosome) and into the cytosol of the target cell.

An embodiment is the conjugate of the invention, wherein the oligomeric molecule or the polymeric molecule of the covalent saponin conjugate is covalently bound to at least one of the at least one sdAb and/or to at least one of the at least one effector molecule, preferably to an amino-acid residue of the sdAb and/or of the effector molecule.

An embodiment is the conjugate of the invention, wherein the at least one saponin is covalently bound to the oligomeric molecule or to the polymeric molecule of the covalent saponin conjugate via a cleavable linker according to the invention.

An embodiment is the conjugate of the invention, wherein the at least one saponin is covalently bound to the oligomeric molecule or to the polymeric molecule of the covalent saponin conjugate via any one or more of an imine bond, a hydrazone bond, an oxime bond, a 1,3-dioxolane bond, a disulfide bond, a thio-ether bond, an amide bond, a peptide bond or an ester bond, preferably via a linker.

An embodiment is the conjugate of the invention, wherein the at least one saponin comprises an aglycone core structure comprising an aldehyde function in position C₂₃ and the at least one saponin comprises optionally a glucuronic acid function in a first saccharide chain at the C₃beta-OH group of the aglycone core structure of the saponin, which aldehyde function is involved in the covalent bonding to the oligomeric molecule or to polymeric molecule of the covalent saponin conjugate, and/or, if present, the glucuronic acid function is involved in the covalent bonding to the oligomeric molecule or to the polymeric molecule of the covalent saponin conjugate, the bonding of the saponin either via a direct covalent bond, or via a linker, wherein the linker is a cleavable linker or a stable linker. Here, stable refers to a bond between the saponin and the sdAb or the effector molecule, or to a bond between the saponin and the oligomeric or polymeric structure, which bond remains intact (is not cleaved) under the acidic conditions inside a cell, in particular the acidic conditions in the endosome or lysosome of such a cell. In addition, such a stable bond remains intact (i.e. is not cleaved) in e.g. the circulation and in the organs of a human subject to whom the conjugate of the invention comprising the covalent saponin conjugate, is administered. In contrast, a cleavable linker in the context of the binding of a saponin to the sdAb or to the effector molecule comprised by the conjugate, or to an oligomeric structure or a polymeric structure refers to a bond that is cleaved under the acidic conditions as apparent inside endosomes and lysosomes of mammalian cells such as a human cell, e.g. a tumor cell, whereas such cleavable linker remains intact (is not cleaved) when a conjugate comprising such cleavable bonds is present in the circulation or in organs, i.e. outside cells, of e.g. a human subject to whom the conjugate of the invention is administered.

An embodiment is the conjugate of the invention, wherein the aldehyde function in position C₂₃ of the aglycone core structure of the at least one saponin is covalently bound to linker EMCH, which EMCH is covalently bound via a thio-ether bond to a sulfhydryl group in the oligomeric molecule or in the polymeric molecule of the covalent saponin conjugate, such as a sulfhydryl group of a cysteine.

Binding of the EMCH linker to the aldehyde group of the aglycone of the saponin results in formation of a hydrazone bond. Such a hydrazone bond is a typical example of a cleavable bond under the acidic conditions inside endosomes and lysosomes. A saponin that is coupled to the sdAb comprised by the conjugate of the invention or to the effector molecule comprised by the conjugate of the invention, or to an oligomeric structure or polymeric structure of a covalent saponin conjugate, wherein such a covalent saponin conjugate is coupled to the sdAb or to the effector molecule of the conjugate, is releasable from the conjugate of the invention once delivered in the endosome or lysosome of a target cell that exposes the cell-surface molecule to which the sdAb of the conjugate can bind. This way, saponin coupled to the sdAb or to the effector molecule in the conjugate of the invention is transferred from outside the cell into the endosome (or lysosome), and in the endosome (or the lysosome), the saponin is released from the remainder of the conjugate upon pH driven cleavage of the hydrazone bond. In the endosome (or the lysosome) the free saponin can exert its stimulatory activity when the delivery of the effector molecule comprised by the conjugate of the invention, into the cytosol is considered. Surprisingly, the inventors established that for the saponin it is not a prerequisite for endosomal escape enhancing activity of the saponin, that the saponin is present in endosomes or lysosomes in free form. Also saponins comprised by e.g. certain conjugates, are potentiating the delivery of an effector molecule, out of the endosome/lysosome into the cytosol of targeted cells, once the effector molecule and the saponin as part of a certain conjugate are both contacted with the same target cell.

An embodiment is the conjugate of the invention, wherein the glucuronic acid function in the first saccharide chain at the C₃beta-OH group of the aglycone core structure of the saponin is covalently bound to linker HATU, which HATU is covalently bound via an amide bond to an amine group in the oligomeric molecule or in the polymeric molecule of the covalent saponin conjugate, such as an amine group of a lysine or an N-terminus of a protein. When the HATU linker is coupled to the saponin and to the sdAb or the effector molecule of the conjugate of the invention, the saponin is for example bound to the N-terminus of the sdAb or the effector molecule (if such effector molecule is a proteinaceous effector molecule such as a protein toxin) or to the amine group of a lysine present in the sdAb or present in the effector molecule.

An embodiment is the conjugate of the invention, wherein the polymeric molecule or the oligomeric molecule of the covalent saponin conjugate is bound to at least one, preferably one, of the at least one sdAb and/or to at least one, preferably one, of the at least one effector molecule, preferably to an amino-acid residue of the sdAb and/or to an amino-acid residue of the effector molecule, involving a click chemistry group on the polymeric molecule or the oligomeric molecule of the covalent saponin conjugate, the click chemistry group preferably selected from a tetrazine, an azide, an alkene or an alkyne, or a cyclic derivative of these groups, more preferably the click chemistry group is an azide.

An embodiment is the conjugate of the invention, wherein the polymeric molecule or the oligomeric molecule of the covalent saponin conjugate comprises a polymeric structure and/or an oligomeric structure selected from: a linear polymer, a branched polymer and/or a cyclic polymer, an oligomer, a dendrimer, a dendron such as a G2 dendron or a G3 dendron or a G4 dendron or a G5 dendron, a dendronized polymer, a dendronized oligomer, a DNA, a polypeptide, a poly-lysine, a poly-ethylene glycol, an oligo-ethylene glycol (OEG), such as OEG₃, OEG₄ and OEG₅, or an assembly of these polymeric structures and/or oligomeric structures which assembly is preferably built up by covalent cross-linking, preferably the polymeric molecule or the oligomeric molecule of the covalent saponin conjugate is a dendron such as a poly-amidoamine (PAMAM) dendrimer.

An embodiment is the conjugate of the invention, wherein the polymeric molecule or the oligomeric molecule of the covalent saponin/sdAb/effector moiety conjugate comprises a polymeric structure and/or an oligomeric structure selected from: a dendrimer, a dendron, a dendronized polymer, a dendronized oligomer, a DNA, for example 2-200 nucleic acids, a poly-ethylene glycol, an oligo-ethylene glycol (OEG), such as OEG₃, OEG₄ and OEG₅. The oligomeric molecule or the polymeric molecule is selected for the absence of intrinsic biological activity. Typically, the selected oligomeric molecule or the polymeric molecule is an inert molecule when biological activity is considered that would or could pose a health risk or that would or could result in adverse events in a human subject when the saponin-comprising conjugate of the invention which comprises such an oligomeric molecule or polymeric molecule is administered to said human subject. Driven by the number of selected saponins to be incorporated in the conjugate of the invention, the type and size or length of the oligomeric structure or polymeric structure is selected. That is to say, the number of saponins to be coupled to the sdAb or to the effector molecule comprised by the conjugate, for formation of the conjugate of the invention, can determine the selection of a suitable oligomeric or polymeric structure, bearing the sufficient amount of binding sites for coupling the desired number of saponins, therewith providing a covalent saponin conjugate bearing the selected number of saponin moieties to be coupled to the sdAb or to the effector molecule, for provision of the conjugate of the invention. For example, length of an OEG or size of a Dendron or poly-lysine molecule determines the maximum number of saponins which can be covalently linked to such oligomeric or polymeric structure.

A conjugate according to the invention thus comprises at least one saponin. With “at least one” in this context is meant that the conjugate comprises one saponin molecule but may also comprise a couple (e.g. two, three or four) of saponins or a multitude (e.g. 10, 20 or 100) of saponins. Depending on the application, the conjugate may comprise a covalently bound scaffold (covalent saponin conjugate) with covalently bound saponins, wherein the scaffold may be designed such that it comprises a defined number of saponins. Preferably, a conjugate according to the invention comprises a defined number or range of saponins, rather than a random number. This is especially advantageous for drug development in relation to marketing authorization. A defined number in this respect means that a conjugate preferably comprises a previously defined number of saponins. This is, e.g., achieved by designing a scaffold comprising a polymeric structure with a certain number of possible moieties for the saponin(s) to attach. Under ideal circumstances, all of these moieties are coupled to a saponin and the scaffold than comprises the prior defined number of saponins. It is envisaged to offer a standard set of scaffolds, comprising, e.g., two, four, eight, sixteen, thirty-two, sixty-four, etc., saponins so that the optimal number can be easily tested by the user according to his needs.

An embodiment is the conjugate of the invention comprising the scaffold of the invention (covalent saponin conjugate of the invention), wherein the saponin is present in a defined range as, e.g., under non-ideal circumstances, not all moieties present in a polymeric structure bind a saponin. Such ranges may for instance be 2-4 saponin molecules per scaffold, 3-6 saponin molecules per scaffold, 4-8 saponin molecules per scaffold, 6-8 saponin molecules per scaffold, 6-12 saponin molecules per scaffold and so on. In such case, a conjugate comprising a scaffold according to the invention thus comprises 2, 3 or 4 saponins if the range is defined as 2-4.

The scaffold is fundamentally independent of the type of saponin covalently bound to the scaffold, the scaffold subsequently (in sequential order) covalently coupled to the conjugate. Thus, the conjugate of the invention comprising the scaffold (covalent saponin conjugate of the invention) is the basis product for a platform technology. Since the at least one covalently bound saponin mediates intracellular delivery of the effector moiety bound to the cell-surface molecule targeting sdAb comprised by the conjugate of the invention, the scaffold technology according to the invention is a system that mediates controlled intracellular effector moiety delivery by saponins. The scaffold provides an optimized and functionally active unit that can be linked to the saponin(s) and to the cell-surface molecule targeting sdAb comprised by the conjugate, at a single and defined position in the sdAb.

An embodiment is the conjugate of the invention comprising a scaffold according to the invention (covalent saponin conjugate of the invention), wherein the number of monomers of the polymeric or oligomeric structure is an exactly defined number or range. Preferably, the polymeric or oligomeric structure comprises structures such as poly(amines), e.g., polyethylenimine and poly(amidoamine), or structures such as polyethylene glycol, poly(esters), such as poly(lactides), poly(lactams), polylactide-co-glycolide copolymers, poly(dextrin), or a peptide or a protein, or structures such as natural and/or artificial polyamino acids, e.g. poly-lysine, DNA polymers, such as a DNA comprising 2-100 nucleotides, stabilized RNA polymers or PNA (peptide nucleic acid) polymers, for example comprising 2-200 nucleotides, either appearing as linear, branched or cyclic polymer, oligomer, dendrimer, dendron (for example any of a G2, G3, G4 or G5 dendron, for maximally covalently binding of 4, 8, 16 or 32 saponin moieties, respectively), dendronized polymer, dendronized oligomer or assemblies of these structures, either sheer or mixed. Preferably, the polymeric or oligomeric structures are biocompatible, wherein biocompatible means that the polymeric or oligomeric structure does not show substantial acute or chronic toxicity in organisms and can be either excreted as it is or fully degraded to excretable and/or physiological compounds by the body's metabolism. Assemblies can be built up by covalent cross-linking or non-covalent bonds and/or attraction. They can therefore also form nanogels, microgels, or hydrogels, or they can be attached to carriers such as inorganic nanoparticles, colloids, liposomes, micelles or particle-like structures comprising cholesterol and/or phospholipids. Said polymeric or oligomeric structures preferably bear an exactly defined number or range of coupling moieties (chemical groups) for the coupling of glycoside molecules (and/or effector molecules and/or carrier molecules such as a ligand, monoclonal antibody or a fragment thereof such as an sdAb). Preferably at least 50%, more preferably at least 75%, more preferably at least 85%, more preferably at least 90%, more preferably at least 95%, more preferably at least 98%, more preferably at least 99%, most preferably (about) 100% of the exactly defined number or range of coupling moieties (chemical groups) in the polymeric or oligomeric structure is occupied by a glycoside molecule (saponin of the invention) in a scaffold according to the invention (covalent saponin conjugate of the invention).

Preferably, a dendron is a branched, clearly defined tree-like polymer with a single chemically addressable group at the origin of the tree, called the focal point. A dendrimer is a connection of two or more dendrons at their focal point. A dendronized polymer is a connection of the focal point of one or more dendrons to a polymer.

In a preferred embodiment, a scaffold according to the invention is provided, wherein the polymeric or oligomeric structure comprises a linear, branched or cyclic polymer, oligomer, dendrimer, dendron, dendronized polymer, dendronized oligomer or assemblies of these structures, either sheer or mixed, wherein assemblies can be built up by covalent cross-linking or non-covalent attraction and can form nanogels, microgels, or hydrogels, and wherein, preferably, the polymer is a derivative of a poly(amine), e.g., polyethylenimine and poly(amidoamine), and structures such as polyethylene glycol, poly(esters), such as poly(lactids), poly(lactams), polylactide-co-glycolide copolymers, and poly(dextrin), and structures such as natural and/or artificial polyamino acids such as poly-lysine, or a peptide or a protein or DNA polymers, such as a DNA comprising 2-100 nucleotides, stabilized RNA polymers or PNA (peptide nucleic acid) polymers, for example comprising 2-200 nucleotides. Preferably, the polymeric or oligomeric structures are biocompatible.

An embodiment is the conjugate of the invention, wherein the at least one saponin is covalently bound to at least one, preferably one, of the at least one sdAb and is covalently bound to at least one, preferably one, of the at least one effector molecule via a tri-functional linker, preferably the trifunctional linker represented by Structure A:

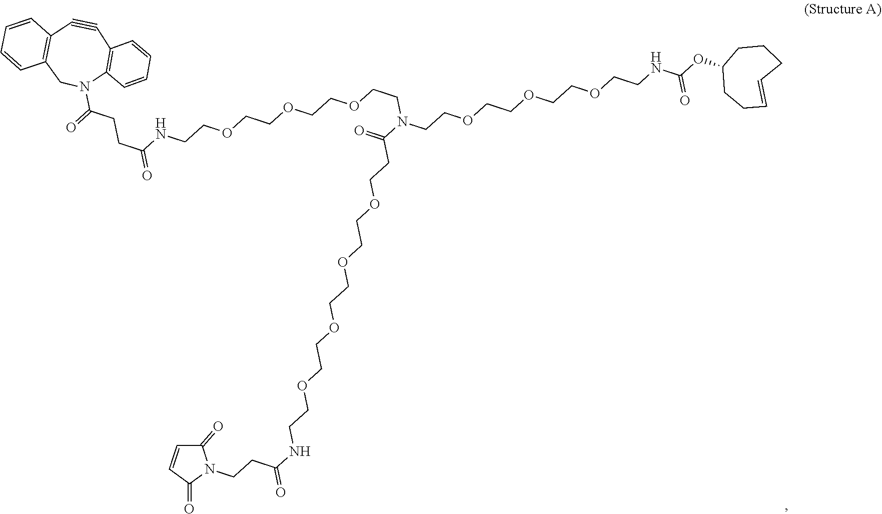

the conjugate preferably comprising the trifunctional linker of Structure A and having a molecular structure represented by Structure B:

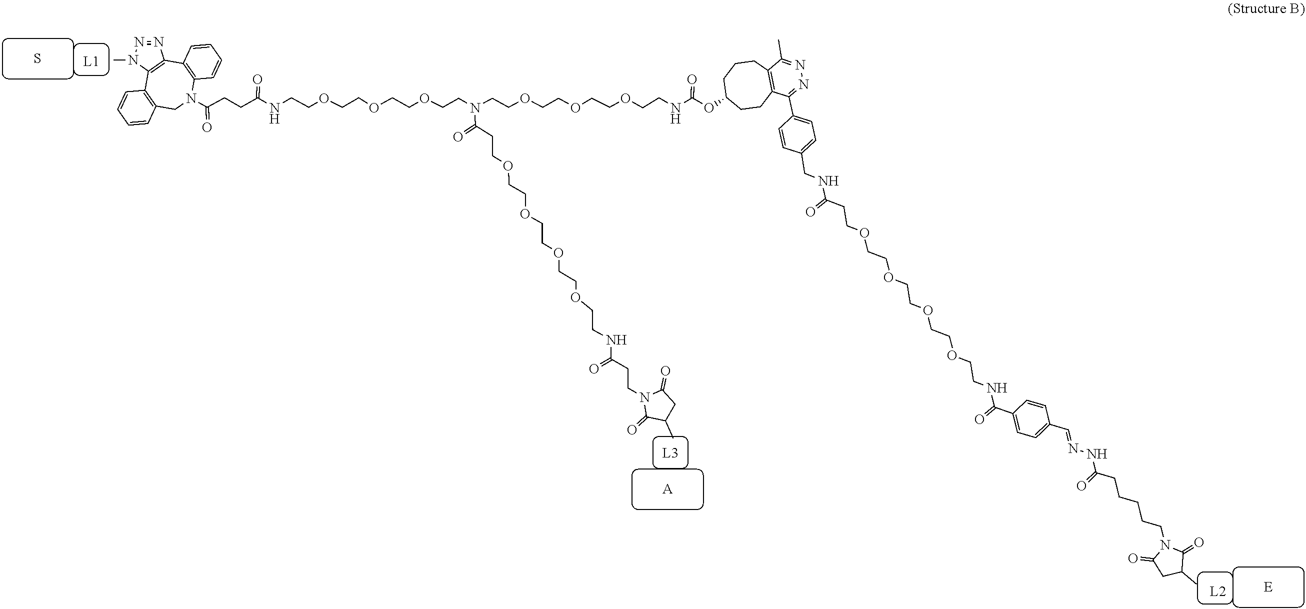

wherein S is the at least one saponin or the covalent saponin conjugate according to the invention, E is the at least one, preferably one, effector molecule, A is the at least one sdAb such as a single sdAb, L1, L2 and L3 are each individually a bond between the trifunctional linker and the saponin or the covalent saponin conjugate, the effector molecule, and the sdAb, respectively, or L1, L2 and L3 are a linker, wherein L1, L2 and L3 are the same or different.

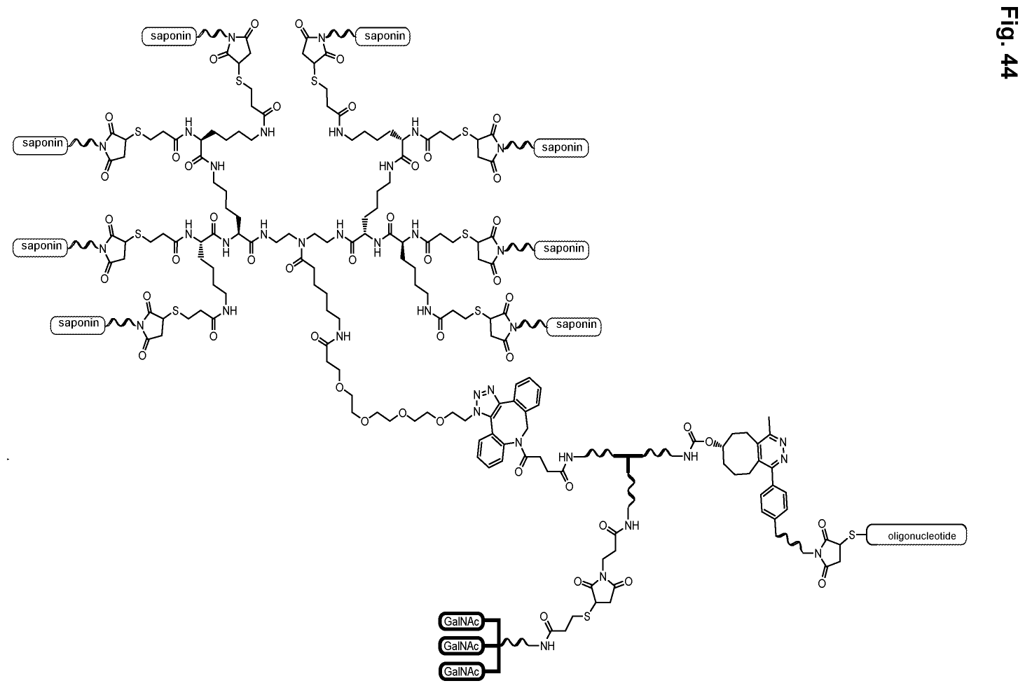

An example of a conjugate based on the trifunctional linker with Structure A and having a structure according to the structure generically depicted as Structure B, is depicted in FIG. 44 as molecule 16; chemical synthesis of molecule 16 is depicted in FIGS. 40-44 (see also the Examples section, here below). The effector moiety E is BNA(ApoB), the saponin are four SO1861 molecules covalently linked to a dendron molecule, the sdAb is here a V_{HH} produced by cell clone anti-HIVgp41 Q8C-tag and is thus an sdAb for binding to HIVgp41. In another example, the saponin is V_{HH} 7D12 with the amino-acid sequence as depicted as SEQ ID NO: 75 and/or V_{HH} 9G8 with the amino-acid sequence as depicted as SEQ ID NO: 76, or a linear multimer of one or more 7D12 domains and one or more 9G8 domains such as the tandem 7D12-9G8 (amino-acid sequence is according to SEQ ID NO: 73, for example).

Unless specifically indicated otherwise and in particular when relating to the endosomal escape mechanism of the saponin of the invention, whenever the word “endosome” or “endosomal escape” is used herein, it also includes the endolysosome and lysosome, and escape from the endolysosome and lysosome, respectively. After entering the cytosol, said substance might move to other cell units such as the nucleus.

In formal terms, a glycoside is any molecule in which a sugar group is bound through its anomeric carbon to another group via a glycosidic bond. Glycoside molecules, such as saponins, in the context of the invention are such molecules that are further able to enhance the effect of an effector moiety, without wishing to be bound by any theory, in particular by facilitating the endosomal escape of the effector moiety. Without wishing to be bound by any theory, the glycoside molecules (saponins of the invention, such as those exemplified herein and in the claims) interact with the membranes of compartments and vesicles of the endocytic and recycling pathway and make them leaky for said effector moieties resulting in augmented endosomal escape. With the term “the scaffold is able to augment endosomal escape of the effector moiety” is meant that the at least one saponin (glycoside molecule), which is coupled via a linker or directly to the cell-surface molecule targeting antibody such as an sdAb or via the polymeric or oligomeric structure of the scaffold (covalent saponin conjugate of the invention), is able to enhance endosomal escape of an effector moiety when both molecules are within an endosome, e.g. a late endosome, optionally and preferably after the at least one saponin is released from the conjugate such as from a linker or polymeric or oligomeric structure comprised by said conjugate, e.g., by cleavage of a cleavable bond between the at least one glycoside (saponin) and the conjugate (for example via a polymeric or oligomeric structure of a scaffold and/or via a linker). Even though a bond between the at least one saponin according to the invention and the cell-surface molecule targeting sdAb of the conjugate of the invention, optionally via a linker or a scaffold, may be a “stable bond”, that does not mean that such bond cannot be cleaved in the endosomes by, e.g., enzymes. For instance, the saponin, optionally together with a linker or a part of the oligomeric or polymeric structure of a scaffold, may be cleaved off from the remaining linker fragment or oligomeric or polymeric structure. It could, for instance be that a protease cuts a (proteinaceous) linker or proteinaceous polymeric structure, e.g., albumin, thereby releasing the at least one saponin. It is, however, preferred that the glycoside molecule (preferably saponin) is released in an active form, preferably in the original form that it had before it was (prepared to be) coupled to the cell-surface molecule targeting sdAb of the conjugate of the invention optionally via a linker and/or an oligomeric or polymeric scaffold (covalent saponin conjugate of the invention); thus the glycoside (saponin) has its natural structure after such cleavage or the glycoside (saponin) has (part of) a chemical group or linker bound thereto, after such cleavage, while glycoside biological activity (saponin biological activity), e.g. endosomal/lysosomal escape enhancing activity towards an effector moiety present in the same endosome or lysosome, is maintained or restored upon said cleavage of the bond between the glycoside (saponin) and the cell-surface molecule targeting antibody such as an sdAb, optionally comprising a linker and/or a scaffold of the invention. With regard to the present invention the term “stable” with respect to bonds between e.g. saponins and amino-acid residues of the cell-surface molecule targeting sdAb in the conjugate, a linker, a polymeric or oligomeric structures (of the scaffold, a.k.a. the covalent saponin conjugate of the invention), ligands, (monoclonal) immunoglobulins or binding domains or -fragments thereof, and/or effectors (effector moieties, effector molecules), is meant that the bond is not readily broken or at least not designed to be readily broken by, e.g., pH differences, salt concentrations, or UV-light, reductive conditions. With regard to the present invention the term “cleavable” with respect to bonds between e.g. saponins and the cell-surface molecule targeting sdAb, linkers, amino-acid residues, polymeric or oligomeric structures of the covalent saponin conjugate, ligands, antibodies and/or effectors, is meant that the bond is designed to be readily broken by, e.g., pH differences, salt concentrations, under reductive conditions, and the like. The skilled person is well aware of such cleavable bonds and how to prepare them.

Before the present invention one of the major hurdles of introducing ADCs and AOCs on the market was the small therapeutic window: a therapeutically effective dose of an ADC or an AOC is accompanied with (unacceptable) side effects, hampering development and implication in treatment of patients with the ADCs. By the application of the conjugate of the invention, such as ADC-saponin conjugate and AOC-saponin conjugate, it has now become possible to guide one or multiple glycoside molecules (saponin(s)) to a (target) cell, together with the ADC carrying a payload or together with a (monoclonal) antibody (sdAb) conjugated with an oligonucleotide such as a BNA according to the invention. In particular, it was previously not possible to specifically guide an effector moiety of an ADC or AOC or any other conjugate of a payload and a (proteinaceous) cell-surface molecule targeting molecule, and a (predefined, controllable) particular number or range of glycoside molecules (saponins) per effector moiety at the same time to the cytosol of cells, such as via the endocytic pathway of a cell.

A solution provided for by the invention comprises the covalent binding of at least one saponin to the cell-surface molecule targeting molecule of the conjugate of the invention, i.e. an sdAb. A further solution provided for by the invention comprises (first) polymerizing the glycoside molecules (saponins) using an oligomeric or polymeric scaffold, and providing the cell-surface molecule targeting molecule comprised by the conjugate of the invention with a cluster of covalently bound saponins, enabling re-monomerization of the one or more saponins at the intracellular site where the mode of action of the saponin is desired, e.g. after endocytosis. “Polymerizes” in this context means the reversible and/or irreversible multiple conjugation of saponin molecules to the sdAb, either via linker, or directly or via a polymeric or oligomeric structure to form a scaffold (covalent saponin conjugate of the invention) or the reversible and/or irreversible multiple conjugation of (modified) saponins thereby forming a polymeric or oligomeric structure to form a scaffold (covalent saponin conjugate of the invention). “Re-monomerization” in this context means the cleavage of the saponins from the conjugate, from the linker linking the saponin(s) to the cell-surface molecule targeting sdAb of the conjugate or from the scaffold, for example after endocytosis, and regaining the (native) chemical state of the unbound saponins, which unbound saponins may or may not comprise additional chemical groups such as a chemical group for linking the saponin to a linker, an amino-acid residue of the conjugate or to the scaffold, and/or a (chemical) linker bound to a chemical group of the saponin such as an aldehyde group or carboxylic acid group. Due to the complex chemistry of the saponins for example the ‘polymerization’ of saponins at a scaffold or other linking linker and their ‘re-monomerization’ at a desired location such as intracellularly e.g. after endocytosis, was a challenging task. In particular, the chemical reactions used for providing the linkers and the scaffold comprising covalently linked glycosides for covalent binding to the conjugate, e.g. triterpenoid saponins (polymerization of the glycosides), normally occur in water-free organic solvents, but saponins and for example biocompatible polymers applied as a scaffold for bearing bound saponins, are water-soluble molecules. The chemical properties of the unmodified saponin further prohibited polymerization by itself and, one other possible solution, to bind multiple saponins (directly) to the effector molecule was estimated not to be very promising, as an effector molecule (drug, toxin, polypeptide or polynucleotide) does typically not provide sufficient binding sites and because the coupling product would become quite heterogeneous and/or coupling biologically active molecules such as a saponin and e.g. a peptide, a toxin, a nucleic acid together bears the risk for influencing and hampering the activity of one or even both molecules bound together in such saponin-comprising conjugate. Further, there was a considerable risk that the effector moiety comprised by the conjugate of the invention loses its function when a saponin is coupled to the e.g. ADC or antibody-oligonucleotide conjugate (AOC). Embodiments of the present invention solves at least one of these drawbacks.

An aspect of the invention relates to a pharmaceutical composition comprising the conjugate of the invention, and optionally a pharmaceutically acceptable excipient and/or pharmaceutically acceptable diluent.

Whether or not a conjugate of the invention comprising saponins, either or not further comprising one or more (cleavable) linkers and/or optionally a scaffold (covalent saponin conjugate of the invention), is able to disturb the acidic environment and inhibit the endosomal escape function of the at least one glycoside (saponin) can be easily determined with an assay as described in the examples section, and as known in the art. The inhibition is described as “fold amount increases of glycoside (saponin of the invention) necessary to induced 50% cell killing”. It is preferred that the scaffold does not lead to an increase that is at least the increase in glycoside molecules (saponins) necessary to obtain 50% cell killing observed when using Chloroquine as a positive control. Alternatively, and preferably, the conjugate comprising saponins, either or not further comprising one or more (cleavable) linkers and/or optionally a scaffold does not lead to an at least 4-fold increase of glycoside molecules to induce 50% cell killing, more preferably does not lead to an at least 2-fold increase. The fold increase is to be measured in assay, wherein Chloroquine, as a positive control, induces a 2-fold increase in glycoside amount, preferably saponin amount wherein the saponin is any one or more of the saponins of the invention (previous embodiments) to observe 50% cell killing.

As said before, the at least one saponin that is comprised by the conjugate according to the invention increases the efficacy of at least current and new effector moieties as defined in this invention. Potential side-effects will be decreased due to lowering of dosing of the effector moiety comprised by the conjugate, without lowering the efficacy. Therefore, the invention provides a conjugate according to the invention for use in medicine or for use as a medicament.

An aspect of the invention relates to a pharmaceutical composition comprising the conjugate of any one of the invention, and optionally a pharmaceutically acceptable excipient and/or pharmaceutically acceptable diluent.

An aspect of the invention relates to a pharmaceutical composition of the invention, for use as a medicament.

An aspect of the invention relates to the pharmaceutical composition of the invention or to the conjugate of the invention, for use as a medicament.

An aspect of the invention relates to the pharmaceutical composition of the invention or the conjugate of the invention, for use in the treatment or the prophylaxis of any one or more of: a cancer, an auto-immune disease such as rheumatoid arthritis, an enzyme deficiency, a disease related to an enzyme deficiency, a gene defect, a disease relating to a gene defect, an infection such as a viral infection, hypercholesterolemia, primary hyperoxaluria, haemophilia A, haemophilia B, alpha-1 antitrypsin related liver disease, acute hepatic porphyria, an amyloidosis and transthyretin-mediated amyloidosis, preferably a cancer such as bladder cancer such as metastatic bladder cancer, urothelial carcinoma, cancer of the urinary tract, urologic neoplasms, prostate cancer such as castration resistant prostate cancer, breast cancer, malignant neoplasm of pancreas, ovary cancer, lung cancer such as non-squamous non-small cell lung cancer and squamous cell carcinoma of lung.

An embodiment is the pharmaceutical composition for use of the invention or the conjugate for use of the invention, wherein:
said use is in the treatment or prevention of cancer in a human subject, preferably a cancer selected from bladder cancer such as metastatic bladder cancer, urothelial carcinoma, cancer of the urinary tract, urologic neoplasms, prostate cancer such as castration resistant prostate cancer, breast cancer, malignant neoplasm of pancreas, ovary cancer, lung cancer such as non-squamous non-small cell lung cancer and squamous cell carcinoma of lung, and/or
said use is in the treatment or prophylaxis of cancer in a patient in need thereof, wherein the at least one nanobody, preferably a bivalent nanobody, binds to a cell-surface molecule of the cell, preferably to a tumor-cell surface molecule of the cell, more preferably to a tumor cell-specific surface molecule of the cell, wherein preferably the cancer is a cancer selected from bladder cancer such as metastatic bladder cancer, urothelial carcinoma, cancer of the urinary tract, urologic neoplasms, prostate cancer such as castration resistant prostate cancer, breast cancer, malignant neoplasm of pancreas, ovary cancer, lung cancer such as non-squamous non-small cell lung cancer and squamous cell carcinoma of lung; and/or
the pharmaceutical composition, preferably a therapeutically effective amount of the pharmaceutical composition, is administered to a patient in need thereof, preferably a human patient.

An example is the pharmaceutical composition for use or the conjugate for use, wherein:
said use is in the treatment or prevention of cancer in a human subject, preferably a cancer selected from bladder cancer such as metastatic bladder cancer, urothelial carcinoma, cancer of the urinary tract, urologic neoplasms, prostate cancer such as castration resistant prostate cancer, breast cancer, malignant neoplasm of pancreas, ovary cancer, lung cancer such as non-squamous non-small cell lung cancer and squamous cell carcinoma of lung; and/or
said use is in the treatment or prophylaxis of cancer in a patient in need thereof, wherein the at least one sdAb, the at least one multivalent nanobody, preferably the at least one bivalent nanobody comprising two sdAbs, binds to the first cell-surface molecule of the first cell, preferably to a tumor-cell surface molecule of the cell, more preferably to a tumor cell-specific surface molecule of the cell, wherein preferably the cancer is a cancer selected from bladder cancer such as metastatic bladder cancer, urothelial carcinoma, cancer of the urinary tract, urologic neoplasms, prostate cancer such as castration resistant prostate cancer, breast cancer, malignant neoplasm of pancreas, ovary cancer, lung cancer such as non-squamous non-small cell lung cancer and squamous cell carcinoma of lung; and/or
the pharmaceutical composition, preferably a therapeutically effective amount of the pharmaceutical composition is administered to a patient in need thereof, preferably a human patient.

A number of preferred features can be formulated for endosomal escape enhancers comprised by the conjugate of the invention, i.e. a saponin of the invention: (1) they are preferably not toxic and do not invoke an immune response, (2) they preferably do not mediate the cytosolic uptake of the effector moiety into off-target cells, (3) their presence at the site of action is preferably synchronized with the presence of the effector moiety, (4) they are preferably biodegradable or excretable, and (5) they preferably do not substantially interfere with biological processes of the organism unrelated to the biological activity of the effector molecule with which the endosomal escape enhancer is combined with, e.g. interact with hormones. Examples of saponins of the invention that fulfill the before mentioned criteria, at least to some extent, are bidesmosidic triterpenes, preferably bidesmosidic triterpene saponins, such as those of Group B and Group C as listed here above, and for example SO1832, SO1861, SA1641, QS-21, GE1741, and the further saponins listed throughout the specification and more specifically in Table A1. Preferred is SO1861. SO1832 is also preferred, since the inventors established that SO1861 and SO1832 display endosomal escape enhancing activity to a similar extent in cell-based bio-assays.

Also provided is the use of a conjugate according to the invention for manufacturing a medicament. Especially cancer medicines, and in particular the classical chemotherapy medicaments, are notorious for their side effects. Because of targeting and synchronization in time and place of both the pharmaceutically active substance comprised by the conjugate and the saponin comprised by the very same conjugate molecule, a therapeutic conjugate according to the invention is especially valuable for use as a medicament, in particular for use in a method of treating cancer. The invention thus provides a therapeutic conjugate according to the invention for use in a method of treating cancer. The invention also provides a therapeutic conjugate according to the invention for use in a method of treating acquired or hereditary disorders, in particular monogenic deficiency disorders. The therapeutic conjugate thus comprises the at least one saponin and the at least one effector moiety, and an sdAb for targeting the conjugate at an aberrant target cell such as a tumor cell or an auto-immune cell.

Thus, an aspect of the invention relates to a therapeutic conjugate according to the invention, wherein the conjugate comprises a covalently bound effector moiety and comprises a covalently bound saponin, and a cell-surface molecule binding antibody such as an sdAb, preferably two or three sdAbs, for use in a method for the treatment of a cancer or an auto-immune disease. The cancer is for example a cancer such as bladder cancer such as metastatic bladder cancer, urothelial carcinoma, cancer of the urinary tract, urologic neoplasms, prostate cancer such as castration resistant prostate cancer, breast cancer, malignant neoplasm of pancreas, ovary cancer, lung cancer such as non-squamous non-small cell lung cancer and squamous cell carcinoma of lung.

A further application of the conjugate of the invention in medicine is the substitution of intracellular enzymes in target cells that produce these enzymes in insufficient amount or insufficient functionality. The resulting disease might be hereditary or acquired. In most cases, only symptomatic treatment is possible and for a number of rare diseases, insufficient treatment options lead to a shortened life span of concerned patients. An example for such a disease is phenylketonuria, which is an inborn error of metabolism that results in decreased metabolism of the amino acid phenylalanine. The disease is characterized by mutations in the gene for the hepatic enzyme phenylalanine hydroxylase. Phenylketonuria is not curable to date. The incidence is approximately 1:10,000 with the highest known incidence in Turkey with 1:2,600. A cell-surface molecule targeting antibody comprised by the conjugate of the invention, preferably an sdAb such as a V_{HH}, with bound phenylalanine hydroxylase or with a bound polynucleotide that encodes phenylalanine hydroxylase can be used to target liver cells by use of a suitable specific antibody or sdAb, and to substitute the defect enzyme in hepatocytes. This is one example of use of the therapeutic conjugate of the invention comprising a saponin bound thereto and the enzyme or the oligonucleotide bound thereto according to the invention, for substitution or gene therapy.

In a preferred embodiment, a therapeutic conjugate according to the invention for use in a method of gene therapy or substitution therapy is provided.

With the conjugate of the invention it has now become possible to design and manufacture a one-component, non-viral clinically applicable gene delivery technology. For example, the conjugate of the invention allows for development of non-viral based gene delivery technology, which enhances therapeutic efficacy with lower therapeutic dose thereby improving the health of patients. The conjugate of the invention, in particular when comprising a covalently bound cell-surface molecule targeting antibody such as a monoclonal antibody or sdAb for binding to a (tumor, auto-immune) cell-surface specific molecule, and when bound to an effector moiety such as an oligonucleotide for example a BNA, allows for overcoming a longstanding and major bottleneck in the field of gene delivery, namely efficient, safe and cost-effective transfer of gene therapeutic products across the endosomal membrane into the cytosol/nucleosol. Indeed, gene therapy is one of the most promising treatment options for future advanced therapies in a broad range of diseases. Successful gene delivery requires the recognition of target cells as well as cytosolic and nucleosolic uptake of the gene. One of the major problems in the field of non-viral gene therapy is the inefficient and insufficiently safe delivery of genetic material for therapeutic use in patients.

Thus, when applying the conjugate of the invention, comprising a cell-targeting cell-surface molecule targeting molecule such as a ligand or preferably an antibody (fragment, domain thereof, preferably sdAb) and comprising an oligonucleotide such as an antisense BNA, the inventors now made it possible to overcome a longstanding and major bottleneck in the field of gene delivery: safe transfer of gene therapeutic products across the endosomal membrane into the cytosol/nucleosol. The conjugate of the invention represents technology designed for allowing targeting of any addressable cell type with all known genetic agents, thereby ensuring better patient therapy not limited to inherited disorders, but also for cancer therapy and therefore of importance for large patient groups. The technology based on the conjugate of the invention may comprise a polymeric or oligomeric scaffold (covalent saponin conjugate of the invention) that serves as a carrier for endosomal escape enhancers (EEEs), such as the saponins as exemplified herein, and the saponins of the embodiments according to the invention, for the cell-surface molecule targeting molecule such as a targeting ligand or (monoclonal) (tumor-cell specific) antibody, or a fragment thereof, or preferably an sdAb such as a V_{HH}, and for the effector moiety, here an effector gene such as an LNA or BNA. Use of the conjugate of the invention, e.g. comprising a cell-targeting antibody (fragment) or sdAb and an oligonucleotide such as a BNA, has potential to bring any kind of biological macromolecules into the cytosol and the nucleus. Development of new targeting ligands, sdAbs and monoclonal (human, humanized) antibodies is under continuous investigation by numerous research groups and companies worldwide. The same for the oligonucleotides that are aimed for delivery in the cytosol of diseases cells such as cancer cells. The conjugate of the invention thus also presents as a molecular interface in which present and future targeting sdAbs and antibodies and present and future therapeutic oligonucleotides (as well as payloads such as protein toxins) are linked or can be linked to for example an oligomeric or polymeric scaffold module of the invention (covalent saponin conjugate of the invention) by click chemistry, allowing for customized drug applications and for future developments in the field of tissue and cell targeting techniques. The conjugate of the invention can comprise antibodies and ligands as the cell-surface molecule targeting molecule, but an sdAb is preferred. The worldwide market of gene therapeutics is rapidly growing and is covering potential treatments for a wide range of disease areas such as, cancer, cardiovascular diseases, Parkinson's, Alzheimer, HIV and many rare (monogenetic) diseases. The current viral vector-based gene therapeutic technologies have significant challenges, such as safety, manufacturing logistics, and associated high costs. The conjugate of the invention allows for use in a technology platform which represents an alternative for a current viral gene delivery technology. Therefore, the conjugate of the invention is suitable for implementing in approaches for developing non-viral gene treatments for diseases such as cancers, cardiovascular diseases, Parkinson's disease, Alzheimer's disease, HIV infection and many rare (monogenetic) diseases. The conjugate of the invention is suitable for developing novel treatments for transforming the field of antibody-drug conjugates (ADCs) and oligonucleotide-based therapeutics by making non-viral vector based gene therapeutics such as based on targeted antisense BNA. The application of the conjugate of the invention, in particular in a covalent conjugate with an antibody such as an sdAb and an oligonucleotide such as a BNA and at least one saponin, is one of the many beneficial approaches made possible due to the present invention. For example, use of the conjugate of the invention now allows for exploitation of the endocytic pathway of mammalian cells. Endocytosis is exploited for the delivery of therapeutics, wherein the conjugate of the invention contributes to improved uptake and endosomal escape of e.g. siRNAs which are comprised by the conjugate. The conjugate of the invention is suitably used together with small molecules that act as delivery enhancers for e.g. payloads, oligonucleotides. Herewith, the conjugate of the invention bearing the covalently coupled oligonucleotide such as a BNA and bearing the covalently coupled cell targeting moiety such as a ligand and preferably an antibody (domain or fragment, preferably a V_{HH}) and bearing the saponins of the invention, provides a solution for the current problem seen with current endosomal escape enhancers and gene therapeutic product, relating to their application as two components, thus complicating therapeutic approval and clinical applicability, since such a conjugate of the invention is a single-conjugate therapeutic molecule encompassing the saponin, gene product such as a BNA and the (tumor) cell targeting moiety such as a (monoclonal) antibody or sdAb.

Thus the invention provides a non-viral gene delivery technology where endosomal escape enhancers (e.g. the glycosides of the embodiments of the invention and of the examples provided), gene therapeutic product (oligonucleotides according to the invention such as a BNA) and targeting ligand or antibody (according to e.g. the embodiments of the invention and the sdAbs exemplified here below in the Examples section) are all comprised by the conjugate of the invention. Such a conjugate of the invention thus provides therapeutic opportunities for current and future macromolecule drugs for a broad range of diseases and large patient groups. With the application of such a conjugate of the invention comprising at least one saponin, at least one oligonucleotide and at least one specific cell-targeting moiety such as an immunoglobulin or sdAb, the problem is addressed which is apparent for current methods of applying endosomal escape enhancers and gene therapeutic product separately, which current methods do not ensure that both compounds are at the same time at the site of interaction. This problem is now overcome by using the conjugate of the invention. That is to say, such a conjugate of the invention provides a non-viral gene delivery technology with increased synchronization (in time and place) of both compounds, i.e. the saponin and the gene product such as a BNA.

Gene therapies could help with hereditary, previously incurable diseases such as cystic fibrosis, chorea, Huntington's disease or hemophilia. However, currently some problems have not been overcome: for example, the therapeutic genes must precisely reach specific target cells in the body. On the other hand, the therapeutic genes should be absorbed by the targeted cells, but the therapeutic genes should not be destroyed. The current gene therapy approaches use viruses as a ferry for genes. However, these procedures involve considerable risks and cannot be transferred to the introduction of other biomolecules.

An embodiment is the conjugate of the invention comprising (plant-derived) glycosides (e.g. any one of the saponins of the invention) for use a platform technology that allows not only delivery of genes when comprised by the conjugate as the carrier molecule, but also allows for the delivery of different therapeutic biomolecules to be introduced into target cells. Therefore, the conjugate of the invention is used for developing treatments based on nucleic acids for cystic fibrosis, chorea, Huntington's disease or hemophilia. Herewith, with the conjugate of the invention, a new gene therapy strategy is available for improving the health of patients with genetic diseases, including those patients with cystic fibrosis, Huntington's disease, and hemophilia. As part of the invention, a non-viral gene delivery technology is developed that combines plant-derived endosomal escape enhancers (glycosides; i.e. the saponins of the invention), gene therapeutic products, and a targeting ligand (i.e. an sdAb) that are all comprised in a single conjugate. The resulting non-viral gene therapy based on the conjugate of the invention displays about 40 times increased delivery efficiency at a lower dosage over currently available strategies. Herewith, the conjugate of the invention is for use in clinical applications such as for the repair or replacement of defective genes, like in cystic fibrosis patients, and for the targeted delivery of specific genes, for instance, to destroy cancer cells. In fact, the conjugate of the invention is suitable for application in treatment regimens for any disease caused by a genetic defect—such as cystic fibrosis, Huntington's disease and hemophilia and which are currently incurable. Gene therapy which makes use of the conjugate of the invention helps in overcoming two current problems: Firstly, it is possible with the conjugate of the invention to deliver therapeutic genes to specific target cells in the body; secondly, the therapeutic genes enter the interior of these cells, but are not destroyed, due to the presence of saponin(s), the oligonucleotide product and a targeting moiety such as an antibody or an sdAb for binding a target cell, all covalently linked together in the conjugate of the invention, for example by using an oligomeric or polymeric scaffold of the invention (covalent saponin conjugate of the invention).

The present invention also provides a method of treating cancer, the method comprising administering a medicament comprising a therapeutic conjugate according to the invention to a patient in need thereof, preferably administering an effective dose of said medicament to a patient in need thereof, preferably a human cancer patient.

Considerations concerning forms suitable for administration are known in the art and include toxic effects, solubility, route of administration, and maintaining activity. For example, pharmacological compositions injected into the bloodstream should be soluble.

Suitable dosage forms, in part depend upon the use or the route of entry, for example transdermal or by injection. Such dosage forms should allow the compound to reach a target cell whether the target cell is present in a multicellular host. Other factors are known in the art, and include considerations such as toxicity and dosage form which retard the compound or composition from exerting its effect.

An aspect of the invention relates to a pharmaceutical composition of the invention, for use in the treatment or the prophylaxis of any one or more of: a cancer, an auto-immune disease such as rheumatoid arthritis, an enzyme deficiency, a disease related to an enzyme deficiency, a gene defect, a disease relating to a gene defect, an infection such as a viral infection, hypercholesterolemia, primary hyperoxaluria, haemophilia A, haemophilia B, alpha-1 antitrypsin related liver disease, acute hepatic porphyria, an amyloidosis and transthyretin-mediated amyloidosis.

An embodiment is the pharmaceutical composition for use of the invention, wherein the saponin of the conjugate is SO1861, a SO1861 derivative, QS-21, or a QS-21 derivative, preferably a SO1861 derivative or a QS-21 derivative, more preferably a SO1861 derivative according to the invention.

An embodiment is the pharmaceutical composition for use of the invention, wherein:
said use is in the treatment or prevention of cancer in a human subject; and/or
said use is in the treatment or prophylaxis of cancer in a patient in need thereof, wherein the at least one sdAb binds to a cell-surface molecule of the cell, preferably to a tumor-cell surface molecule of the cell, more preferably to a tumor cell-specific surface molecule of the cell; and/or
the pharmaceutical composition, preferably a therapeutically effective amount of the pharmaceutical composition, is administered to a patient in need thereof, preferably a human patient.

An aspect of the invention relates to an in vitro or ex vivo method for transferring the effector molecule of the invention from outside a cell to inside said cell, preferably to the cytosol of said cell, comprising the steps of:
a) providing a cell which expresses on its cell surface the binding site for the at least one sdAb comprised by the conjugate of the invention, said binding site preferably being present on a cell-surface molecule of the cell, said cell preferably being selected from a liver cell, an aberrant cell such as a virally infected cell, an auto-immune cell, a cell comprising a gene defect, a cell comprising an enzyme deficiency and a tumor cell;
b) providing the conjugate of the invention, said conjugate comprising the effector molecule to be transferred into the cell provided in step a); and
c) contacting the cell of step a) in vitro or ex vivo with the conjugate of step b), therewith effecting the transfer of said conjugate comprising the effector molecule from outside the cell to inside said cell, and by effecting the transfer of said conjugate effecting the transfer of the effector molecule from outside the cell inside said cell, preferably into the cytosol of said cell.

An aspect of the invention relates to an in vitro or ex vivo method for transferring the conjugate of the invention from outside a cell to inside said cell, comprising the steps of:
a) providing a cell which expresses on its cell surface the binding site for the at least one sdAb comprised by the conjugate of the invention, said binding site preferably being present on a cell-surface molecule of the cell, said cell preferably being selected from a liver cell, an aberrant cell such as a virally infected cell, an auto-immune cell, a cell comprising a gene defect, a cell comprising an enzyme deficiency and a tumor cell;
b) providing the conjugate of the invention; and
c) contacting the cell of step a) in vitro or ex vivo with the conjugate of step b), therewith effecting the transfer of the conjugate from outside the cell to inside said cell.

The tumor cell is for example related to a cancer such as bladder cancer such as metastatic bladder cancer, urothelial carcinoma, cancer of the urinary tract, urologic neoplasms, prostate cancer such as castration resistant prostate cancer, breast cancer, malignant neoplasm of pancreas, ovary cancer, lung cancer such as non-squamous non-small cell lung cancer and squamous cell carcinoma of lung.

An aspect of the invention relates to a kit comprising a container containing an endosomal escape enhancing conjugate according to the invention the kit further comprising instructions for using the conjugate.

An embodiment is the conjugate according to the invention, wherein the cell-surface molecule targeting molecule is selected from an sdAb derived from V_{H} or V_{L} of cetuximab, trastuzumab, OKT-9 (i.e. the sdAb is based on the V_{H} or V_{L}, preferably the V_{H}, of such monoclonal antibodies and is capable of specifically binding to the target endocytic receptor on the cell-surface of a target cell), and or wherein the effector moiety is selected from dianthin, saporin and antisense BNA(HSP27) or antisense BNA(ApoB), and/or wherein the saponin is selected from Table A1, such as SO1861, SO1832, GE1741, SA1641, Quil-A, QS-21, preferably SO1861 or SO1832 or QS-21, more preferably the saponin is SO01861 or SO01832.

TABLE A1
Saponins displaying (late) endosomal/lysosomal escape enhancing activity,
and with a aglycone core of the 12,13-dehydrooleanane type⁴⁾

| | Aglycone core with | Carbohydrate | Carbohydrate |
|---|---|---|---|
| | an aldehyde group | substituent at | substituent at |
| Saponin Name | at the C-23 position | the C-3beta-OH group | the C-28-OH group |
| NP-017777 | Gypsogenin | Gal-(1→2)-[Xyl- | Xyl-(1→4)-Rha-(1→2)-[R-(→4)]-Fuc-(R = |
| | | (1→3)]-GlcA- | 4E-Methoxycinnamic acid) |
| NP-017778 | Gypsogenin | Gal-(1→2)-[Xyl- | Xyl-(1→4)-Rha-(1→2)-[R-(→4)]-Fuc-(R = |
| | | (1→3)]-GlcA- | 4Z-Methoxycinnamic acid) |
| NP-017774 | Gypsogenin | Gal-(1→2)-[Xyl- | Xyl-(1→4)-[Gal-(1→3)]-Rha-(1→2)-4- |
| | | (1→3)]-GlcA- | OAc-Fuc- |
| NP-018110^{c}, | Gypsogenin | Gal-(1→2)-[Xyl- | Xyl-(1→4)-[Glc-(1→3)]-Rha-(1→2)-3,4- |
| NP-017772^{d} | | (1→3)]-GlcA- | di-OAc-Fuc- |
| NP-018109 | Gypsogenin | Gal-(1→2)-[Xyl- | Xyl-(1→4)-[Glc-(1→3)]-Rha-(1→2)-[R- |
| | | (1→3)]-GlcA- | (→4)]-3-OAc-Fuc-(R = 4E- |
| | | | Methoxycinnamic acid) |
| NP-017888 | Gypsogenin | Gal-(1→2)-[Xyl- | Glc-(1→3)-Xyl-(1→4)-[Glc-(1→3)]-Rha- |
| | | (1→3)]-GlcA- | (1→2)-4-OAc-Fuc- |
| NP-017889 | Gypsogenin | Gal-(1→2)-[Xyl- | Glc-(1→3)-Xyl-(1→4)-Rha-(1→2)-4-OAc- |
| | | (1→3)]-GlcA- | Fuc- |
| NP-018108 | Gypsogenin | Gal-(1→2)-[Xyl- | Ara/Xyl-(1→3)-Ara/Xyl-(1→4)-Rha/Fuc- |
| | | (1→3)]-GlcA- | (1→2)-[4-OAc-Rha/Fuc-(1→4)]-Rha/Fuc- |
| SA1641^{a}, AE | Gypsogenin | Gal-(1→2)-[Xyl- | Xyl-(1→3)-Xyl-(1→4)-Rha-(1→2)-[Qui- |
| X55^{b} | | (1→3)]-GlcA- | (1→4)]-Fuc- |
| SO1658 | Gypsogenin | Gal-(1→2)-[Xyl- | Glc-(1→3)-[Xyl-(1→3)-Xyl-(1→4)]-Rha- |
| | | (1→3)]-GlcA- | (1→2)-Fuc- |
| gypsoside A⁶⁾ | Gypsogenin | Gal-(1→4)-Glc | Xyl-(1→3)-Fuc-(1→4)-[Xyl-(1→3)-Xyl- |
| | | (1→4)-[Ara- | (1→3)]-Rha- |
| | | (1→3)]-GlcA- | |
| phytolaccagenin | Gypsogenin | absent | absent |
| Gypsophila | Quillaic acid | Gal-(1→2)-[Xyl- | Glc-(1→3)-[Xyl-(1→4)]-Rha-(1→2)-Fuc- |
| saponin 1 | | (1→3)]-GlcA- | |
| (Gyp1) | | | |
| NP-017674 | Quillaic acid | Gal-(1→2)-[Xyl- | Api-(1→3)-Xyl-(1→4)-[Glc-(1→3)]-Rha- |
| | | (1→3)]-GlcA- | (1→2)-Fuc- |
| NP-017810 | Quillaic acid | Gal-(1→2)-[Xyl- | Xyl-(1→4)-[Gal-(1→3)]-Rha-(1→2)-Fuc- |
| | | (1→3)]-GlcA- | |
| AG1 | Quillaic acid | Gal-(1→2)-[Xyl- | Xyl-(1→4)-[Glc-(1→3)]-Rha-(1→2)-Fuc- |
| | | (1→3)]-GlcA- | |
| NP-003881 | Quillaic acid | Gal-(1→2)-[Xyl- | Ara/Xyl-(1→4)-Rha/Fuc-(1→4)-[Glc/Gal- |
| | | (1→3)]-GlcA- | (1→2)]-Fuc- |
| NP-017676 | Quillaic acid | Gal-(1→2)-[Xyl- | Api-(1→3)-Xyl-(1→4)-[Glc-(1→3)]-Rha- |
| | | (1→3)]-GlcA- | (1→2)-[R-(→4)]-Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| NP-017677 | Quillaic acid | Gal-(1→2)-[Xyl- | Api-(1→3)-Xyl-(1→4)-Rha-(1→2)-[R- |
| | | (1→3)]-GlcA- | (→4)]-Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| NP-017706 | Quillaic acid | Gal-(1→2)-[Xyl- | Api-(1→3)-Xyl-(1→4)-Rha-(1→2)-[Rha- |
| | | (1→3)]-GlcA- | (1→3)]-4-OAc-Fuc- |
| NP-017705 | Quillaic acid | Gal-(1→2)-[Xyl- | Api-(1→3)-Xyl-(1→4)-[Glc-(1→3)]-Rha- |
| | | (1→3)]-GlcA- | (1→2)-[Rha-(1→3)]-4-OAc-Fuc- |
| NP-017773 | Quillaic acid | Gal-(1→2)-[Xyl- | 6-OAc-Glc-(1→3)-Xyl-(1→4)-Rha-(1→2)- |
| | | (1→3)]-GlcA- | [3-OAc-Rha-(1→3)]-Fuc- |
| NP-017775 | Quillaic acid | Gal-(1→2)-[Xyl- | Glc-(1→3)-Xyl-(1→4)-Rha-(1→2)-[3- |
| | | (1→3)]-GlcA- | OAc -- Rha-(1→3)]-Fuc- |
| SA1657 | Quillaic acid | Gal-(1→2)-[Xyl- | Xyl-(1→3)-Xyl-(1→4)-Rha-(1→2)-[Qui- |
| | | (1→3)]-GlcA- | (1→4)]-Fuc- |
| AG2 | Quillaic acid | Gal-(1→2)-[Xyl- | Glc-(1→3)-[Xyl-(1→4)]-Rha-(1→2)-[Qui- |
| | | (1→3)]-GlcA- | (1→4)]-Fuc- |
| GE1741 | Quillaic acid | Gal-(1→2)-[Xyl- | Xyl-(1→3)-Xyl-(1→4)-Rha-(1→2)-[3,4-di- |
| | | (1→3)]-GlcA- | OAc-Qui-(1→4)]-Fuc- |
| SO1542 | Quillaic acid | Gal-(1→2)-[Xyl- | Glc-(1→3)-[Xyl-(1→4)]-Rha-(1→2)-Fuc- |
| | | (1→3)]-GlcA- | |
| SO1584 | Quillaic acid | Gal-(1→2)-[Xyl- | 6-OAc-Glc-(1→3)-[Xyl-(1→4)]-Rha- |
| | | (1→3)]-GlcA- | (1→2)-Fuc- |
| SO1674 | Quillaic acid | Gal-(1→2)-[Xyl- | Glc-(1→3)-[Xyl-(1→3)-Xyl-(1→4)]-Rha- |
| | | (1→3)]-GlcA- | (1→2)-Fuc- |
| SO17003) | Quillaic acid | Gal-(1→2)-[Xyl- | Xyl-(1→4)-Rha-(1→2)-[Xyl-(1→3)-4- |
| | | (1→3)]-GlcA- | OAc-Qui-(1→4)]-Fuc- |
| Saponarioside | Quillaic acid | Gal-(1→2)-[Xyl- | Xyl-(1→3)-Xyl-(1→4)-Rha-(1→2)-[4- |
| B¹⁾ | | (1→3)]-GlcA- | OAc-Qui-(1→4)]-Fuc- |
| SO1730³⁾ | Quillaic acid | Gal-(1→2)-[Xyl- | Glc-(1→3)-Xyl-(1→4)-Rha-(1→2)-[-4- |
| | | (1→3)]-GlcA- | OAc-Qui-(1→4)]-Fuc- |
| SO1772³⁾ | Quillaic acid | Gal-(1→2)-[Xyl- | 6-OAc-Glc-(1→3)-[Xyl-(1→4)]-Rha- |
| | | (1→3)]-GlcA- | (1→2)-[4-OAc-Qui-(1→4)]-Fuc-- |
| SO1832¹⁾ | Quillaic acid | Gal-(1→2)-[Xyl- | Xyl-(1→3)-Xyl-(1→4)-Rha-(1→2)-[Xyl- |
| (protonated | | (1→3)]-GlcA- | (1→3)-4-OAc-Qui-(1→4)]-Fuc- |
| SO1831) = | | | |
| Saponarioside | | | |
| A | | | |
| SO1861 | Quillaic acid | Gal-(1→2)-[Xyl- | Glc-(1→3)-Xyl-(1→4)-Rha-(1→2)-[Xyl- |
| (deprotonated | | (1→3)]-GlcA- | (1→3)-4-OAc-Qui-(1→4)]-Fuc- |
| SO1862) | | | |
| SO1862 | Quillaic acid | Gal-(1→2)-[Xyl- | Glc-(1→3)-Xyl-(1→4)-Rha-(1→2)-[Xyl- |
| (protonated | | (1→3)]-GlcA- | (1→3)-4-OAc-Qui-(1→4)]-Fuc- |
| SO1861), also | | | |
| referred to as | | | |
| Sapofectosid⁵⁾ | | | |
| SO1904³⁾ | Quillaic acid | Gal-(1→2)-[Xyl- | 6-OAc-Glc-(1→3)-[Xyl-(1→4)]-Rha- |
| | | (1→3)]-GlcA- | (1→2)-[Xyl-(1→3)-4-OAc-Qui-(1→4)]- |
| | | | Fuc- |
| QS-7 (also | Quillaic acid | Gal-(1→2)-[Xyl- | Api/Xyl-(1→3)-Xyl-(1→4)-[Glc-(1→3)]- |
| referred to as | | (1→3)]-GlcA- | Rha-(1→2)-[Rha-(1→3)]-40Ac-Fuc- |
| QS1861) | | | |
| QS-7 api (also | Quillaic acid | Gal-(1→2)-[Xyl- | Api-(1→3)-Xyl-(1→4)-[Glc-(1→3)]-Rha- |
| referred to as | | (1→3)]-GlcA- | (1→2)-[Rha-(1→3)]-40Ac-Fuc- |
| QS1862) | | | |
| QS-17 | Quillaic acid | Gal-(1→2)-[Xyl- | Api/Xyl-(1→3)-Xyl-(1→4)-[Glc-(1→3)]- |
| | | (1→3)]-GlcA- | Rha-(1→2)-[R-(→4)]-Fuc- |
| | | | (R = 5-O-[5-O-Rha-(1→2)-Ara/Api-3,5- |
| | | | dihydroxy-6-methyl-octanoyl]-3,5- |
| | | | dihydroxy-6-methyl-octanoic acid) |
| QS-18 | Quillaic acid | Gal-(1→2)-[Xyl- | Api/Xyl-(1→3)-Xyl-(1→4)-[Glc-(1→3)]- |
| | | (1→3)]-GlcA- | Rha-(1→2)-[R-(→4)]-Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 A-apio | Quillaic acid | Gal-(1→2)-[Xyl- | Api-(1→3)-Xyl-(1→4)-Rha-(1→2)-[R- |
| | | (1→3)]-GlcA- | (→4)]-Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 A-xylo | Quillaic acid | Gal-(1→2)-[Xyl- | Xyl-(1→3)-Xyl-(1→4)-Rha-(1→2)-[R- |
| | | (1→3)]-GlcA- | (→4)]-Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 B-apio | Quillaic acid | Gal-(1→2)-[Xyl- | Api-(1→3)-Xyl-(1→4)-Rha-(1→2)-[R- |
| | | (1→3)]-GlcA- | (→3)]-Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 B-xylo | Quillaic acid | Gal-(1→2)-[Xyl- | Xyl-(1→3)-Xyl-(1→4)-Rha-(1→2)-[R- |
| | | (1→3)]-GlcA- | (→3)]-Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 | Quillaic acid | Gal-(1→2)-[Xyl- | Combination of the carbohydrate chains |
| | | (1→3)]-GlcA- | depicted for QS-21 A-apio, A-xylo, B-apio, |
| | | | B-xylo, for this position at the aglycone |
| | | | (see also the structure depicted as |
| | | | (Scheme Q)) |
| Agrostemmoside | Quillaic acid | Gal-(1→2)-[Xyl- | [4,6-di-OAc-Glc-(1→3)]-[Xyl-(1→4)]-Rha- |
| E (AG1856, | | (1→3)]-GlcA- | (1→2)-[3,4-di-OAc-Qui-(1→4)]-Fuc- |
| AG2.8)²⁾ | | | |
| | Aglycone core without | Carbohydrate | Carbohydrate |
| | an aldehyde group at | substituent at the | substituent at the |
| Saponin Name | the C-23 position | C-3beta-OH group | C-28-OH group |
| NP-005236 | 2alpha- | GlcA- | Glc/Gal- |
| | Hydroxyoleanolic | | |
| | acid | | |
| AMA-1 | 16alpha- | Glc- | Rha-(1→2)-[Xyl-(1→4)]-Rha- |
| | Hydroxyoleanolic | | |
| | acid | | |
| AMR | 16alpha- | Glc- | Rha-(1→2)-[Ara-(1→3)-Xyl-(1→4)]-Rha- |
| | Hydroxyoleanolic | | |
| | acid | | |
| alpha-Hederin | Hederagenin (23- | Rha-(1→2)-Ara- | Not present |
| | Hydroxyoleanolic | | |
| | acid) | | |
| NP-012672 | 16alpha,23- | Ara/Xyl-(1→4)- | Ara/Xyl- |
| | Dihydroxyoleanolic | Rha/Fuc-(1→2)- | |
| | acid | Glc/Gal-(1→2)- | |
| | | Rha/Fuc-(1→2)- | |
| | | GICA- | |
| beta-Aescin | Protoaescigenin- | Glc-(1→2)-[Glc- | Not present |
| (described: | 21(2-methylbut-2- | (1→4)]-GlcA- | |
| Aescin Ia) | enoate)-22-acetat | | |
| aescinate | Aglycone core without | present | Not present |
| | an aldehyde group at | | |
| | the C-23 position | | |
| dipsacoside B | Aglycone core without | present | present |
| | an aldehyde group at | | |
| | the C-23 position | | |
| esculentoside A | Aglycone core without | present | Not present |
| | an aldehyde group at | | |
| | the C-23 position | | |
| Teaseed | 23-Oxo- | Glc-(1→2)-Ara- | Not present |
| saponin I | barringtogenol C - | (1→3)-[Gal- | |
| | 21,22-bis(2- methylbut-2- | (1→2)]-GlcA- | |
| | enoate) | | |
| Teaseedsaponin | 23-Oxo-barringtogenol | Xyl-(1→2)-Ara- | Not present |
| J | C-21,22-bis(2- | (1→3)-[Gal- | |
| | methylbut-2-enoate) | (1→2)]-GlcA- | |
| Assamsaponin F | 23-Ox0- | Glc-(1→2)-Ara- | Not present |
| | barringtogenol C - | (1→3)-[Gal- | |
| | 21(2-methylbut-2- | (1→2)]-GlcA- | |
| | enoate)-16,22-diacetat | | |
| Primula acid 1 | 3,16,28- | Rha-(1→2)-Gal- | Not present |
| | Trihydroxyoleanan- | (1→3)-[Glc- | |
| | 12-en | (1→2)]-GlcA- | |
| AS64R | Gypsogenic acid | absent | Glc-(1→3)-[Glc-(1→6)]-Gal- |
| Macranthoidin A | Aglycone core without | present | present |
| | an aldehyde group at | | |
| | the C-23 position | | |
| saikosaponin A | Aglycone core without | present | absent |
| | an aldehyde group at | | |
| | the C-23 position | | |
| saikosaponin D | Aglycone core without | present | absent |
| | an aldehyde group at | | |
| | the C-23 position | | |
| | | Carbohydrate | |
| | | substituent at | |
| | | the C-23-OH group | |
| AS6.2 | Gypsogenic acid | Gal- | Glc-(1→3)-[Glc-(1→6)]-Gal- |

^{a, b}Different names refer to different isolates of the same structure
^{c, d}Different names refer to different isolates of the same structure
¹⁾Jia et al., Major Triterpenoid Saponins from Saponaria officinalis, J. Nat. Prod. 1998, 61, 11, 1368-1373, Publication Date: Sept. 19, 1998, https://doi.org/10.1021/np980167u
²⁾The structure of Agrostemmoside E (also referred to as AG1856 or AG2.8) is given in FIG. 4 of J. Clochard et al, A new acetylated triterpene saponin from Agrostemma githago L. modulates gene delivery efficiently and shows a high cellular tolerance, International Journal of Pharmaceutics, Volume 589, 15 Nov. 2020, 119822.
³⁾Structures of SO1700, SO1730, SO1772, SO1904 are given in Moniuszko-Szajwaj et al., Highly Polar Triterpenoid Saponins from the Roots of Saponaria officinalis L., Helv. Chim. Acta, V99, pp. 347-354, 2016 (doi.org/10.1002/hlca.201500224).
⁴⁾See for example: thesis by Dr Stefan Bottger (2013): Untersuchungen zur synergistischen Zytotoxizitat zwischen Saponinen und Ribosomen inaktivierenden Proteinen Typ I, and Sama et al., Structure-Activity Relationship of Transfection-Modulating Saponins - A Pursuit for the Optimal Gene Trafficker, Planta Med. Volume 85, pp. 513-518, 2019 (doi: 10.1055/a-0863-4795) and Fuchs et al., Glycosylated Triterpenoids as Endosomal Escape Enhancers in Targeted Tumor Therapies, Biomedicine, Volume 5, issue 14, 2017 (doi: 10.3390/biomedicines5020014).
⁵⁾Sama et al., Sapofectosid - Ensuring non-toxic and effective DNA and RNA delivery, International Journal of Pharmaceutics, Volume 534, Issues 1-2, 20 December 2017, Pages 195-205 (dx.doi.org/10.1016/j.ijpharm.2017.10.016) & Moniuszko-Szajwaj et al., Highly Polar Triterpenoid Saponins from the Roots of Saponaria officinalis L., Helv. Chim. Acta, V99, pp. 347-354, 2016 (doi.org/10.1002/hlca.201500224).
⁶⁾See for example: doi:10.1016/s0040-4039(01)90658-6, Tetrahedron Letters No. 8, pp. 477-482, 1963 and pubchem.ncbi.nlm.nih.gov/compound/Gipsoside

Suitable sources for isolating saponins according to the invention, i.e. those that display endosomal escape enhancing activity, are Quillaja saponaria, Saponinum album, Saponaria officinalis, and Quillaja bark. Saponin suitable for the conjugate of the invention are thus for example:
Quillaja saponaria saponin, saponin isolated from Quillaja saponaria, for example Quil-A, QS-17-api, QS-17-xyl, QS-21, QS-21A, QS-21B, QS-7-xyl,
Saponinum album, saponin isolated from Saponinum album
Saponaria officinalis saponin, saponin isolated from Saponaria officinalis (preferred),
Quillaja bark saponin, saponin isolated from Quillaja bark saponin, for example Quil-A, QS-17-api, QS-17-xyl, QS-21, QS-21A, QS-21B, QS-7-xyl.

In addition, apart from QS-21, also the individual saponins present in QS-21 are suitable saponins for the conjugate of the invention, i.e. the saponins depicted as the saponins of SCHEME Q:

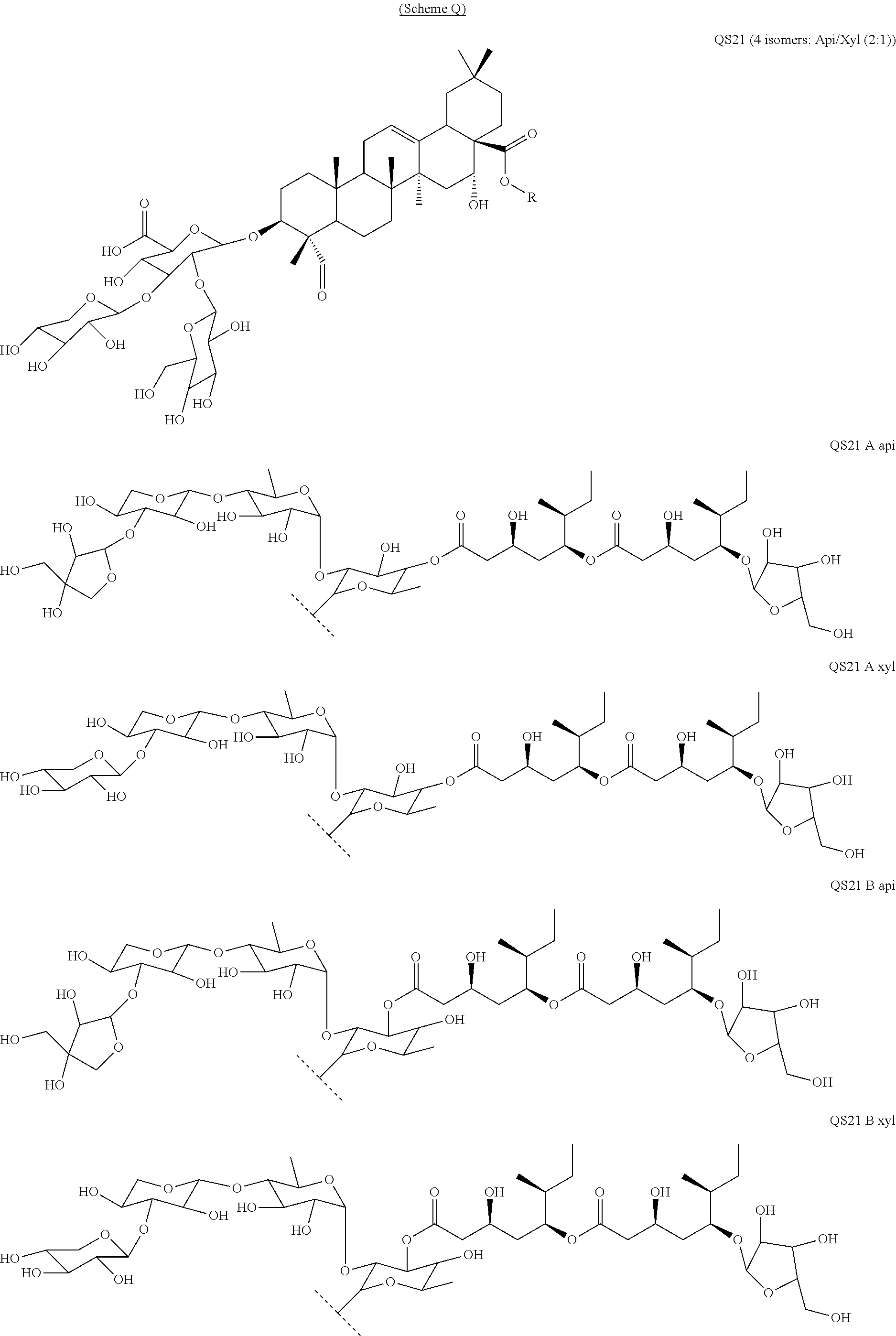

An aspect of the invention relates to a conjugate such as an ADC or an AOC, or to a semi-finished ADC conjugate or a semi-finished AOC conjugate, comprising a cell-surface molecule targeting molecule comprising at least an sdAb and preferably at least a bivalent sdAb, and comprising at least one effector moiety of the invention and/or comprising at least one saponin of the invention, of Structure C:

A(-S)_{b}(-E)_{c} (Structure C)

wherein A is the cell-surface molecule targeting molecule i.e. the one or more sdAb, preferably at least one bivalent sdAb (sdAb-sdAb tandem);
S is the saponin;
E is the effector moiety;
b=0-64, preferably 0, 1, 2, 3, 4, 8, 16, 32, 64 or any whole number (or fraction) therein between, preferably 1-8, more preferably 1, 2, 4 or 8, most preferably 1, 4 or 8 saponin moieties;
c=0-8, preferably 0, 1, 2, 3, 4, 6, 8 or any whole number (or fraction) therein between, preferably 1 or 2 copies of the same effector moiety or different effector moieties, more preferably a single copy of the effector moiety,
wherein S is coupled to A and/or to E, E is coupled to A and/or to S, preferably S is coupled to A and E is coupled to A, more preferably, S and E are coupled covalently to a trifunctional linker, wherein preferably the trifunctional linker is coupled to A. Optionally, more than one trifunctional linker each with the covalently bound one or more S and with the covalently bound E, are covalently bound to A, for example 1-4 of such trifunctional linkers which are functionalized with coupled A and E moieties, preferable 1-2, for example (on average) 1, 1.1, 1.2, 1.3, 1.4, 1.5, 1.6, 1.7, 1.8, 1.9 or 2 of such trifunctional linkers. Preferably, the A is at least a tandem of sdAbs, e.g. a bivalent sdAb such as a biparatopic sdAb. Typically, the conjugate comprises 1, 4 or 8 saponin moieties, or a multiple thereof when more than one (trifunctional) linker to which the saponin(s) are bound, are linked to the sdAb(s).

For example, when on average 1.6 of such (trifunctional) linkers are bound to for example a bivalent sdAb, the number of saponin moieties in the conjugate would be 1.6, 6.4 and 12.8 when the (trifunctional) linker contains 1, 4 or 8 bound saponin moieties, respectively. Typically, the conjugate comprises a single copy of the effector moiety, or a multiple thereof when more than one (trifunctional) linker to which the effector moiety is bound, are linked to the sdAb(s). For example, when on average 1.6 of such (trifunctional) linkers are bound to for example a bivalent sdAb, the average number of effector moieties in the conjugate would be 1.6. It is to be understood that in the conjugate such bivalent sdAbs have for example a single linker or two linkers bound, these linkers each comprising the bound at least one saponin and the bound at least one effector moiety. The linker is typically a trifunctional linker. The at least one saponin is a saponin as claimed, preferably SO1861. The at least one effector moiety is an effector moiety as claimed, preferably an oligonucleotide. The at least one sdAb is preferably a bivalent sdAb or a string of 3-6 sdAb's preferably comprising at least one bivalent antibody. The binding partner for the at least one sdAb in the conjugate is for example an endocytic receptor present on the target cell, such as a tumor-cell specific receptor such as for example CD71 and EGFR, or is another receptor as claimed.

An embodiment is the Structure C of the invention, wherein A is at least one, preferably at least two, such as two or three, sdAb(s) derived from an anti-EGFR antibody such as cetuximab, an anti-HER2 antibody such as trastuzumab, an anti-HIVgp41 antibody such as sdAb Q8c, an anti-CD71 antibody such as OKT-9, and/or wherein S is a triterpenoid saponin and/or a bidesmosidic triterpene saponin belonging to the type of a 12,13-dehydrooleanane preferably with an aldehyde function in position C-23 of the aglycone core structure of the saponin, said aglycone preferably being quillaic acid or gypsogenin, more preferably quillaic acid, and the saponin optionally comprising a glucuronic acid function in a carbohydrate substituent at the C-3beta-OH group of the saponin, preferably the saponin is SO1832, SO1861, GE1741, SA1641, Quil-A or QS-21, or any of the saponins depicted in Table A1, and/or wherein E is any one or more of an oligonucleotide, an antisense oligonucleotide, an siRNA, an antisense BNA, and an antisense BNA(HSP27) or an antisense BNA(ApoB), and/or any one or more of a proteinaceous toxin, a ribosome inactivating protein, dianthin and saporin, preferably an oligonucleotide.

An embodiment is the Structure C (conjugate) of the invention, the conjugate of the invention, wherein the saponin, and/or the effector moiety, is covalently coupled via at least one linker, such as a cleavable linker, and/or via a covalent saponin conjugate (i.e. at least one oligomeric or polymeric scaffold such as a PEG selected from PEG3-PEG30, such as PEG4-PEG20, any of PEG5-PEG12, or such as a dendron such as a PAMAM, preferably a G2, G3 or G4 dendron), such as a linker based on N-s-maleimidocaproic acid hydrazide (EMCH), succinimidyl 3-(2-pyridyldithio)propionate or 3-(2-Pyridyldithio)propionic acid N-hydroxysuccinimide ester (SPDP), and 1-[Bis(dimethylamino)methylene]-1H-1,2,3-triazolo[4,5-b]pyridinium 3-oxid hexafluorophosphate (HATU), and preferably such as a covalent saponin conjugate (a scaffold) based on a Dendron such as a G2- or G3-dendron or G4-Dendron (See Examples section for embodiments of the invention, with dendrons with 4 or 8 saponin moieties covalently bound to the dendron) or a tri-functional linker such as the tri-functional linker of Structure A (see for example the Examples section), and/or wherein at least a lysine side chain and/or a cysteine side chain of the cell-surface molecule targeting at least one sdAb, preferably 2-6 sdAbs such as at least a bivalent V_{HH} tandem according to the invention, preferably a biparatopic nanobody, is involved in the covalent bond with the saponin and/or the effector moiety and/or the linker and/or the cleavable linker and/or the covalent saponin conjugate, wherein preferably the saponin and/or the effector moiety is covalently linked to the cell-surface molecule targeting molecule, preferably 2-6 sdAbs such as at least one bivalent sdAb such as a biparatopic nanobody, wherein the covalent link comprises or consists of an amide bond, a hydrazone bond, a semicarbazone bond, a disulphide bond, preferably an acid labile cleavable bond such as a hydrazone bond or a semicarbazone bond, more preferably a hydrazone bond.

An aspect of the invention relates to the use of any of the aforementioned conjugates, ADCs comprising a covalently linked saponin, AOCs comprising a covalently linked saponin, as a medicament.

An aspect of the invention relates to the use of any of the aforementioned conjugates, ADCs comprising a covalently linked saponin, AOCs comprising a covalently linked saponin, for use in the treatment or prophylaxis of a cancer or an auto-immune disease. The cancer is for example any one of: bladder cancer such as metastatic bladder cancer, urothelial carcinoma, cancer of the urinary tract, urologic neoplasms, prostate cancer such as castration resistant prostate cancer, breast cancer, malignant neoplasm of pancreas, ovary cancer, lung cancer such as non-squamous non-small cell lung cancer and squamous cell carcinoma of lung.

The invention is further illustrated by the following examples, which should not be interpreted as limiting the present invention in any way. Modifications and alternative implementations of some parts or elements or compounds or application or use are possible, and are included in the scope of protection as defined in the appended claims.

### EXAMPLES AND EXEMPLARY EMBODIMENTS

### Example A. Bivalent V_{HH} (saponin)4(BNA) Materials

Trastuzumab (Tras, Herceptin®, Roche), Cetuximab (Cet, Erbitux®, Merck KGaA), Tris(2-carboxyethyl)phosphine hydrochloride (TCEP, 98%, Sigma-Aldrich), 5,5-Dithiobis(2-nitrobenzoic acid) (DTNB, Ellman's reagent, 99%, Sigma-Aldrich), Zeba™ Spin Desalting Columns (2 mL, Thermo-Fisher), NuPAGE™ 4-12% Bis-Tris Protein Gels (Thermo-Fisher), NuPAGE™ MES SDS Running Buffer (Thermo-Fisher), Novex™ Sharp Pre-stained Protein Standard (Thermo-Fisher), PageBlue™ Protein Staining Solution (Thermo-Fischer), Pierce™ BCA Protein Assay Kit (Thermo-Fisher), N-Ethylmaleimide (NEM, 98%, Sigma-Aldrich), 1,4-Dithiothreitol (DTT, 98%, Sigma-Aldrich), Sephadex G25 (GE Healthcare), Sephadex G50 M (GE Healthcare), Superdex 200P (GE Healthcare), Isopropyl alcohol (IPA, 99.6%, VWR), Tris(hydroxymethyl)aminomethane (Tris, 99%, Sigma-Aldrich), Tris(hydroxymethyl)aminomethane hydrochloride (Tris.HCL, Sigma-Aldrich), L-Histidine (99%, Sigma-Aldrich), D-(+)-Trehalose dehydrate (99%, Sigma-Aldrich), Polyethylene glycol sorbitan monolaurate (TWEEN 20, Sigma-Aldrich), Dulbecco's Phosphate-Buffered Saline (DPBS, Thermo-Fisher), Guanidine hydrochloride (99%, Sigma-Aldrich), Ethylenediaminetetraacetic acid disodium salt dihydrate (EDTA-Na₂, 99%, Sigma-Aldrich), sterile filters 0.2 μm and 0.45 μm (Sartorius), Succinimidyl 4-(N-maleimidomethyl)cyclohexane-1-carboxylate (SMCC, Thermo-Fisher), Dianthin-Cys (Dia-Cys, Dianthin mutant with a single C-terminal cysteine function, Proteogenix), Vivaspin T4 and T15 concentrator (Sartorius), Superdex 200PG (GE Healthcare), Tetra(ethylene glycol) succinimidyl 3-(2-pyridyldithio)propionate (PEG₄-SPDP, Thermo-Fisher), HSP27 BNA disulfide oligonucleotide (Biosynthesis), [O-(7-Azabenzotriazol-1-yl)-N,N,N,N-tetramethyluronium-hexafluorphosphat](HATU, 97%, Sigma-Aldrich), Dimethyl sulfoxide (DMSO, 99%, Sigma-Aldrich), N-(2-Aminoethyl)maleimide trifluoroacetate salt (AEM, 98%, Sigma-Aldrich), L-Cysteine (98.5%, Sigma-Aldrich), deionized water (DI) was freshly taken from Ultrapure Lab Water Systems (MilliQ, Merck), Nickel-nitrilotriacetic acid agarose (Ni-NTA agarose, Protino), Glycine (99.5%, VWR), 5,5-Dithiobis(2-nitrobenzoic acid (Ellman's reagent, DTNB, 98%, Sigma-Aldrich), S-Acetylmercaptosuccinic anhydride Fluorescein (SAMSA reagent, Invitrogen) Sodium bicarbonate (99.7%, Sigma-Aldrich), Sodium carbonate (99.9%, Sigma-Aldrich), PD MiniTrap desalting columns with Sephadex G-25 resin (GE Healthcare), PD10 G25 desalting column (GE Healthcare), Zeba Spin Desalting Columns in 0.5, 2, 5, and 10 mL (Thermo-Fisher), Vivaspin Centrifugal Filters T4 10 kDa MWCO, T4 100 kDa MWCO, and T15 (Sartorius), Biosep s3000 aSEC column (Phenomenex), Vivacell Ultrafiltration Units 10 and 30 kDa MWCO (Sartorius), Nalgene Rapid-Flow filter (Thermo-Fisher),

### Monoclonal Antibodies and SO1861:

CD71 monoclonal antibody was purchased from BioCell (Okt9, #BE0023). cetuximab (Erbitux®) and matuzumab were purchased from the pharmacy (Charite, Berlin). SO1861 was isolated and purified by Analyticon Discovery GmbH from raw plant extract obtained from Saponaria officinalis L.

### Methods

### MALDI-TOF-MS

Matrix-assisted laser desorption/ionization time of flight (MALDI-TOF) spectra were recorded on a MALDI-Mass Spectrometer (Bruker Ultrafex III). Typically, the sample dissolved in MilliQ water in nanomole to micromole range was spotted on the target (MTP 384 target plate polished steel T F, Bruker Daltons) using either super-DHB (99%, Fluka) or sinapinic acid (SA, 99%, Sigma-Aldrich) as the matrix that was dissolved in acetonitrile (MADLI-TOF-MS tested, Sigma)/0.1% TFA (7:3 v/v) via the dried-droplet-method. PepMix (Peptide Calibration Standard, Bruker Daltons) or ProteoMass (Protein Calibration Standard, Sigma-Aldrich) served as calibration standards. RP mode refers to reflector positive mode. RN mode refers to reflector negative mode. LP mode refers to linear positive mode.

### Dialysis

Regenerated cellulose membranes: MWCO=1 and 2 kDa (Spectra/Por), and MWCO=12-14 kDa (Carl Roth) were used to perform dialysis. Typically, dialysis was carried out for 24 h with 1 L of solvent that was exchanged after first 6 h of the process.

### Lyophilization

Freeze-drying was performed on an Alpha 1-2 LD plus (Martin Christ Gefriertrocknungsanlagen GmbH). Typically, samples were frozen with liquid nitrogen and placed into the freeze-dryer at high vacuum.

### UV-Vis

Absorbance measurements were performed on a Perkin Elmer Lambda 25 UV-Vis or on a Thermo NanoDrop ND-2000 spectrophotometer in the spectral range of 200-750 nm.

Concentrations were determined using a Thermo Nanodrop 2000 or Lambda 25 spectrometer using the following parameters:
mass ε280 value for bivalent V_{HH}; ε 280=2.50 (mg/ml)⁻¹ cm⁻¹
Trastuzumab OD₂₈₀=1.5 (mg/ml)⁻¹ cm⁻¹
Cetuximab OD₂₈₀=1.4 (mg/ml)⁻¹ cm⁻¹
HSP27 Oligonucleotide OD₂₆₀=153,000 M⁻¹ cm⁻¹; Rz_{260:280}=1.819
Dia-Cys OD₂₈₀=0.57 (mg/ml)⁻¹ cm⁻¹
PEG₄-SPDP (PDT) OD₃₄₃=8,080 M⁻¹ cm⁻¹
SAMSA-Fluorescein OD₄₉₅=64,500 M⁻¹ cm⁻¹; Rz_{280:495}=0.232
Ellmans (TNB) OD₄₁₂=14,150 M⁻¹ cm⁻¹

Ellman's assay was carried out using a Perkin Elmer Lambda 365 Spectrophotometer and a literature molar ε412 value of 14150 M-1 cm-1 for TNB.

### Immobilized Metal Ion Affinity Chromatography (IMCA)

Nickel-nitrilotriacetic acid (Ni-NTA) chromatography was performed to purify histidine-tagged protein and protein-conjugates. Briefly, Ni-NTA agarose (10 mL) was pipetted into a gravity flow column for 5 mL bed volume. The resin was washed with 20 mL deionized water and recharged with 5 mL of 100 mM NiSO4 solution. The resin was washed again five times with 5 mL deionized water and equilibrated five times with DPBS. The protein solution was incubated with the washed Ni-NTA agarose rotating at 4° C. for 30 min. Afterwards, the Ni-NTA protein solution was pipetted back into the gravity flow column. The flow through was collected and the resin was washed three times with 5 mL DPBS. The immobilized sample was then eluted by increasing the imidazole concentration from 50 mL of 125 mM, pH 8 to 50 mL of 250 mM, pH 8. Elution fractions were dialyzed against PBS pH 7.4 to obtain the purified sample.

### Size Exclusion Chromatography

Size exclusion chromatography (SEC) was carried out on an AKTA purifier 10 system. Samples were analyzed by SEC using either a Biosep SEC-S3000 column or an Sephadex G50M column (10×40 cm) eluting with TBS/isopropyl alcohol solution (85:15 v/v) or eluting with DPBS:IPA (85:15). Sample purities were determined by integration of the antibody sample peak with respect to the trace aggregate peaks. Bivalent conjugate purity was determined by integration of the Conjugate peak with respect to impurities/aggregate forms.

### SDS-PAGE

Select VHH samples were analysed under heat denaturing non-reducing conditions by SDS-PAGE against a protein ladder using a 4-12% bis-tris gel and MES as running buffer (200V, ˜40 minutes). Samples were prepared to highest possible concentration, comprising LDS sample buffer and MOPS running buffer as diluent. DTT was added to a final concentration of 50 mM. Samples were heat treated for 2 minutes at 90-95° C. and 15 μl (˜3 μg) added to each well. Protein ladder (10 μl) was loaded without pre-treatment. Empty lines were filled with 1×LDS sample buffer (10 μl). After the gel was run, it was washed thrice with DI water (100 ml) with shaking (15 minutes, 200 rpm). Coomassie staining was performed by shaker-incubating the gel with PAGEBlue protein stain (30 ml) (60 minutes, 200 rpm). Excess staining solution was removed, rinsed twice with DI water (100 ml) and destained with DI water (100 ml) (60 minutes, 200 rpm). The resulting gel was imaged and processed using imageJ and MyCurveFit (point-to-point correlation of protein ladder).

### Ellman's Assay

Ellman's test (or Ellman's assay) was carried out to quantitatively determine thiol concentration in a sample via spectrophotometry. Presence of thiols was indicated via the stoichiometric release of the 2-nitro-5-thiobenzoate (TNB) from Ellman's reagent in the presence of thiols. TNB obtains an absorption maximum at 412 nm and an extinction coefficient of OD₄₁₂=14,150 M⁻¹ cm⁻¹ in buffered systems. Briefly, 2 μL of a 0.5 mg/mL solution of the Ellman's reagent (5,5-Dithiobis(2-nitrobenzoic acid), DTNB) in phosphate buffer (0.1 M, 1 mM EDTA, pH 7.4) was mixed with 20 μL of a thiol containing sample in buffer. The mixture was vortexed for 5 sec. Then, UV-Vis absorbance at 412 nm was measured on a Thermo Nanodrop 2000 to determine TNB concentration and thus thiol content of the sample.

### Dianthin Production

Dianthin was expressed in a bacterium culture and the protein was purified following conventional cell culturing and protein purification steps known in the art. Dianthin-C(dianthin with a terminal cysteine) was conjugated to the terminal cysteine residues of the V_{HH} targeting HER2, V_{HH} targeting CD71 or V_{HH} targeting EGFR producing HER2V_{HH}-dianthin (DAR1), CD71V_{HH}-dianthin (DAR1) and EGFRV_{HH}-dianthin (DAR1). Dianthin-Cys (Dia-Cys or Dianthin-C, Dianthin mutant with a single C-terminal cysteine, was produced by Proteogenix, France. In more detail:

### Procedure for the Conjugation of V_{HH}-Dianthin

Dianthin-Cys was concentrated by ultrafiltration using a vivaspin T15 10 KDa MWCO centrifugal filter and buffer exchanged into TBS pH 7.5. To the concentrated Dianthin-Cys was added an aliquot of freshly prepared TCEP solution (10.0 mg/ml), the mixture vortexed briefly then incubated for 60 minutes at 20° C. with roller-mixing. After incubation, the resulting Dianthin-SH was purified by gel filtration using a zeba spin desalting column then repeated centrifugal-wash cycles using a vivaspin T15 10 KDa MWCO centrifugal filter into TBS pH 7.5. The resulting Dianthin-SH was reacted with freshly prepared DTME solution (10 mg/ml) in DMSO, the mixture vortexed briefly then incubated for 60 minutes at 20° C. After, the Dianthin-DTME was obtained following purification by gel filtration using a zeba spin desalting column into TBS pH 7.5. The Dianthin-DTME was stored at 20° C. until conjugated. At the same time, an aliquot of V_{HH} was concentrated by ultrafiltration using a vivaspin T15 10 KDa MWCO centrifugal filter and buffer exchanged into TBS pH 7.5. To the concentrated V_{HH} was added an aliquot of freshly prepared TCEP solution (10.0 mg/ml), the mixture vortexed briefly then incubated for 60 minutes at 37° C. with roller-mixing. After incubation, the resulting V_{HH} was purified by gel filtration using a zeba spin desalting column then repeated centrifugal-wash cycles using a vivaspin T4 5 KDa MWCO centrifugal filter into TBS pH 7.5. An aliquot of the resulting V_{HH}-SH was reacted with Dianthin-DTME, the mixture vortexed briefly then incubated overnight at 20° C. After, the reaction mixture was concentrated using a vivaspin T4 10 KDa MWCO centrifuge tube and purified by gel filtration using a 1.6×35 cm Superdex 200PG column eluting into DPBS pH 7.5.

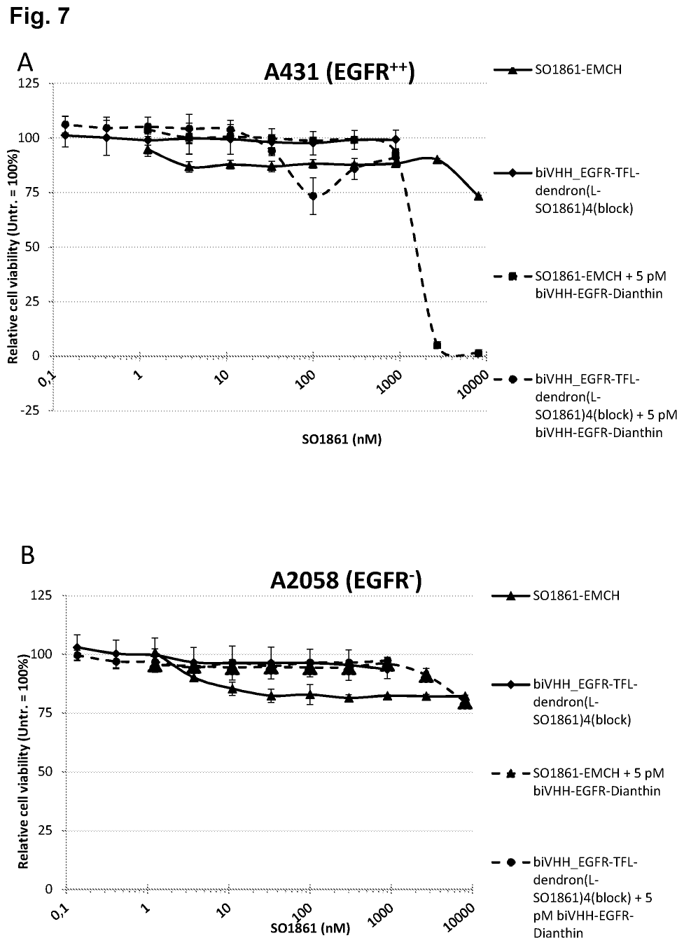

Expression of bivalent-V_{HH} EGFR-dianthin-tetraCys (referred to as bivalent V_{HH}-EGFR-dianthin; see FIGS. 7, 14-16)

The protein bivalent V_{HH}-EGFR-dianthin was expressed in E. coli and purified according to standard procedures known in the art (GenScript). The amino-acid sequence is as provided as [SEQ ID NO: 78]. The protein comprises the tetra-Cys repeat according to the SEQ-ID NO: 77, i.e. HRWCCPGCCKTF. The presence of this tetra-Cys repeat makes the bivalent V_{HH}-EGFR-dianthin suitable for providing a 1-component conjugate, by coupling a linker with covalently bound saponin(s) thereto to the bivalent V_{HH}-EGFR-dianthin. The amino acid sequence of the bivalent V_{HH}-EGFR-dianthin is as follows:

SEQ ID NO: 78:
QVKLEESGGGSVQTGGSLRLTCAASGRTSRSYGMGWFRQAPGKEREFVSG
ISWRGDSTGYADSVKGRFTISRDNAKNTVDLQMNSLKPEDTAIYYCAAAA
GSAWYGTLYEYDYWGQGTQVTVSSGGGGSGGGGS
EVQLVESGGGLVQAGGSLRLSCAASGRTFSSYAMGWFRQAPGKEREFVVA
INWSSGSTYYADSVKGRFTISRDNAKNTMYLQMNSLKPEDTAVYYCAAGY
QINSGNYNFKDYEYDYWGQGTQVTVSSGGGGSGGGGS
AAATAYTLNLANPSASQYSSFLDQIRNNVRDTSLIYGGTDVAVIGAPSTT
DKFLRLNFQGPRGTVSLGLRRENLYVVAYLAMDNANVNRAYYFKNQITSA
ELTALFPEVVVANQKQLEYGEDYQAIEKNAKITTGDQSRKELGLGINLLI
TMIDGVNKKVRVVKDEARFLLIAIQMTAEAARFRYIQNLVTKNFPNKFDS
ENKVIQFQVSWSKISTAIFGDCKNGVFNKDYDFGFGKVRQAKDLQMGLLK
YLGRPKGGGGSGGGGSHRWCCPGCCKTF
GGGGS
HHHHHHHHHH
HRWCCPGCCKTF.

The number of amino acid residues is 576; the molecular weight is 62493.78 Da, the theoretical pl is 9.04. The bivalent V_{HH}-EGFR-dianthin with the sequence of SEQ ID NO: 78 is also referred to as 7D12-(g4s1)2-9G8-(g4s1)2-Dianthin-(g4s1)-10His-tetraCys, referring to the presence of, from N-terminus to C-terminus, the sequence of sdAb 7D12 (SEQ ID NO: 75), to linker repeats, sdAb sequence of 9G8 (SEQ ID NO: 76), two linker repeats, the dianthin sequence, two linker repeats, 10 His residues, followed by the tetra-Cys sequence of SEQ ID NO: 77.

### Production of Saporin Conjugates

Custom trastuzumab-saporin cetuximab-saporin, CD71 mab-saporin conjugates were produced and purchased from Advanced Targeting Systems (San Diego, CA). IgG-saporin and saporin was purchased from Advanced Targeting Systems

BivalentVHH (Icosagen) and bivalent VHH dianthin conjugate (Fleet) were purchased.

### FACS Analyses

Cells were seeded in DMEM (PAN-Biotech GmbH) supplemented with 10% fetal calf serum (PAN-Biotech GmbH) and 1% penicillin/streptomycin (PAN-Biotech GmbH), at 500,000 c/plate in 10 cm dishes and incubated for 48 hrs (5% CO₂, 37° C.), until a confluency of 90% was reached. Next, the cells were trypsinized (TryplE Express, Gibco Thermo Scientific) to single cells. 0.75×10⁶ Cells were transferred to a 15 mL falcon tube and centrifuged (1,400 rpm, 3 min). The supernatant was discarded while leaving the cell pellet submerged. The pellet was dissociated by gentle tapping the falcon tube on a vortex shaker and the cells were washed with 4 mL cold PBS (Mg²⁺ and Ca²⁺ free, 2% FBS). After washing the cells were resuspended in 3 mL cold PBS (Mg²⁺ and Ca²⁺ free, 2% FBS) and divided equally over 3 round bottom FACS tubes (1 mL/tube). The cells were centrifuged again and resuspended in 200 pL cold PBS (Mg²⁺ and Ca²⁺ free, 2% FBS) or 200 μL antibody solution; containing 5 μL antibody in 195 μL cold PBS (Mg²⁺ and Ca²⁺ free, 2% FBS). APC Mouse IgG1, K Isotype Ctrl FC (#400122, Biolegend) was used as isotype control, and APC anti-human EGFR (#352906, Biolegend) was used to stain the EGFR receptor. CD71: APC anti-human CD71 #334108, Biolegend. Samples were incubated for 30 min at 4° C. on a tube roller mixer. Afterwards, the cells were washed 3× with cold PBS (Mg²⁺ and Ca²⁺ free, 2% FBS) and fixated for 20 min at room temperature using a 2% PFA solution in PBS. Cells were washed 2× with cold PBS, and resuspended in 250-350 μL cold PBS for FACS analysis. Samples were analyzed with a BD FACSCanto II flow cytometry system (BD Biosciences) and FlowJo software. See Table A for the expression levels of EGFR, HER2 and CD71 of various cells.

TABLE A
Expression levels of EGFR, HER2 and CD71 of various cells

| | EGFR expression | CD71 expression |
|---|---|---|
| Cell line | level (MFI) | level (MFI) |
| A431 | 1593 | 322 |
| A2058 | 1 | 59 |

### Cell Culture

Cells were cultured in DMEM (PAN-Biotech GmbH) supplemented with 10% fetal bovine serum (FBS) (PAN-Biotech GmbH) at 37° C. and 5% CO₂.

### Cell Viability Assay

Cells were seeded in a 96 well plate at 5.000-10.000 c/w in 100 μL/well and incubated overnight at 37° C. The next day 10× concentrated treatment-mix samples were prepared in PBS, which contain antibody-conjugated SO1861 (i.e. a ‘binding molecule’ or an ‘endosomal escape enhancing conjugate’ of the invention) and targeted-toxin (i.e. a ‘binding molecule’) both at 10× final concentration. The media was removed from the cell culture plate and replaced by 180 μL culture media, followed by the addition of 20 μL treatment-mix/well. For control, 10× treatment-mix samples were prepared that contained the corresponding concentrations of only antibody-conjugated SO1861, only antibody, only SO1861, only targeted-toxin, or PBS without compound as vehicle control. After treatment the cells were incubated for 72 hr at 37° C. before the cell viability was determined by a MTS-assay, performed according to the manufacturer's instruction (CellTiter 96® AQueous One Solution Cell Proliferation Assay, Promega). Briefly, the MTS solution was diluted 20× in DMEM without phenol red (PAN-Biotech GmbH) supplemented with 10% FBS (PAN-Biotech GmbH). The cells were washed once with 200 μL PBS per well, after which 100 μL diluted MTS solution was added per well. The plate was incubated for approximately 20-30 minutes at 37° C. Subsequently, the optical density at 492 nm was measured on a Thermo Scientific Multiskan FC plate reader (Thermo Scientific). For quantification the background signal of ‘medium only’ wells was subtracted from all other wells, before the ratio of untreated/treated cells was calculated, by dividing the background corrected signal of untreated wells over the background corrected signal of the treated wells.

### Trifunctional Linker-(Dendron(L-SO1861)4)-(L-BNA Oligo)-(Bivalent-VHH) Synthesis

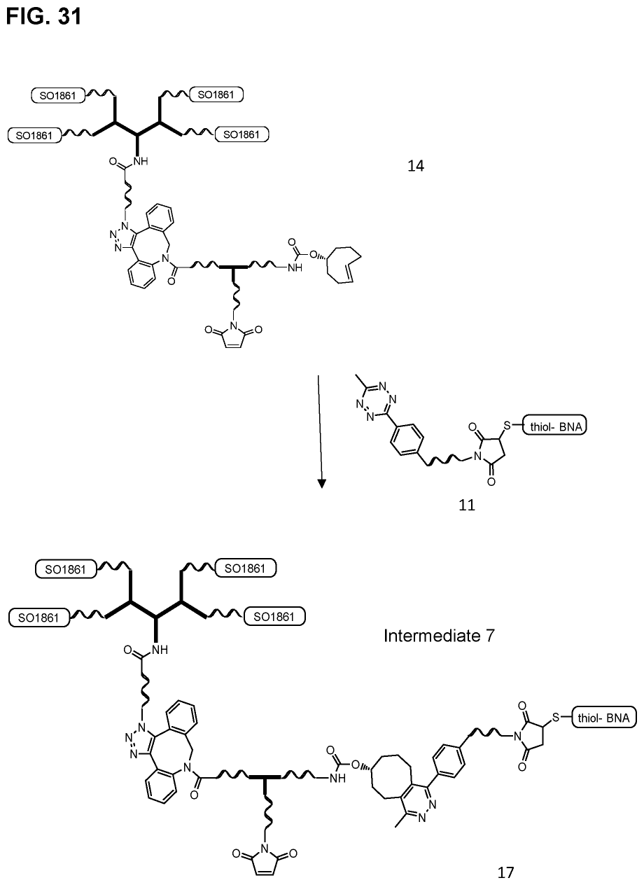

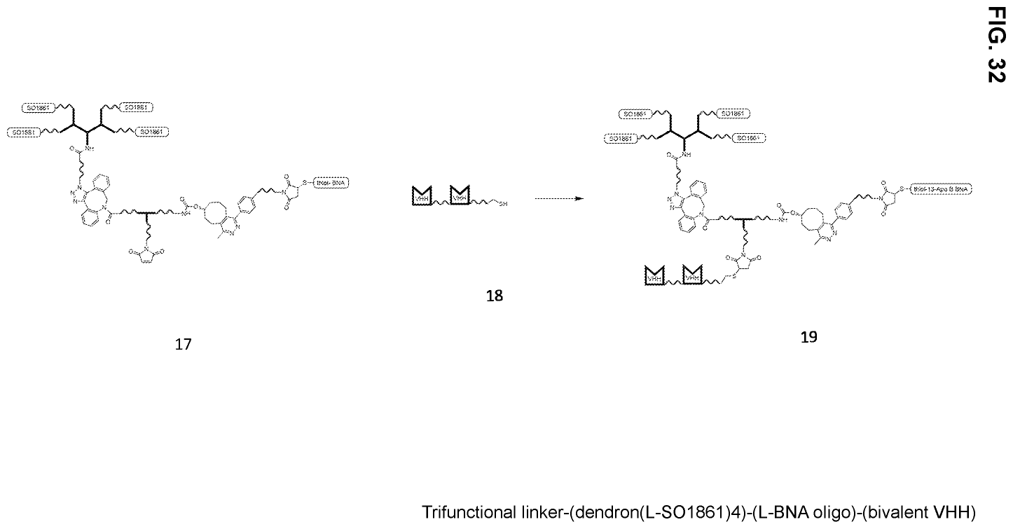

Referring to FIG. 31-32

### Intermediate 7:

### Trifunctional Linker-(Dendron(L-SO1861)4)-(L-BNA Oligo)-(Maleimide) (Molecule 17)

Methyltetrazine-BNA oligo (molecule 11) (4.4 mg, 0.8 μmol) was dissolved in 1 mL DPBS/acetonitrile mixture (4:1 v/v) and the resulting solution was directly added to trifunctional linker-(dendron(-L-SO1861)4)-(TCO)-(Maleimide) (molecule 14) (7.8 mg, 0.7 μmol). The reaction mixture was shaken for 1 min and left standing at room temperature. After 30 min the reaction mixture was frozen and lyophilized overnight to yield the crude title product (molecule 17) as a fluffy solid. To the crude product was added a solution of 20 mM ammonium bicarbonate (1.50 mL) and the resulting suspension was filtered over a 0.45 μm syringe filter. The filtrate was lyophilized overnight to yield the title product (10.8 mg, 79%) as a fluffy solid. Purity based on LC-MS 92%. See also FIG. 31.

### Trifunctional Linker-(Dendron(L-SO1861)4)-(L-BNA Oligo)-(Bivalent VHH) (Molecule 19)

To bivalent VHH (5.00 mg, 0.162 μmol, 1.0 mg/ml) was added a freshly prepared solution of TCEP (5 mole equivalents, 0.81 μmol, 46.4 μl) in TBS pH 7.5 (5 mg/ml), the mixture vortexed briefly and roller mixed at 20° C. for 120 minutes. The resulting bivalent VHH-SH (molecule 18) was purified by gel filtration using Zeba desalting columns eluting with TBS pH 7.5. Bivalent VHH-SH was analysed by UV-vis and Ellman's assay (0.842 mg/ml, VHH:SH=3.70).

To an aliquot of bivalent VHH-SH (1.35 mg, 43.7 nmol, 1.60 ml) was added an aliquot of freshly prepared TFL-(dSPT4)-(L-BNA)-(Maleimide) (molecule 17) (4 mole equivalents, 0.175 μmol, 100 μl) dissolved in DMSO (20 mg/ml). The reaction mixture was roller mixed at 20° C. overnight. After incubation, the reaction mixture was analysed by Ellman's assay to ascertain incorporation by depletion, then quenched by the addition of a freshly prepared solution of NEM (10 mole equivalents, 10.9 μl) in TBS pH 7.5 (5 mg/ml). The bivalent VHH-TFL conjugate (molecule 19) was purified by gel filtration using a dedicated 2.6×40 cm Superdex 200 column eluting with DPBS pH 7.5 and filtered to 0.2 μm. From the purification run, a sample from each fraction of interest was taken and analysed by SDS-PAGE. Fractions corresponding to components labelled ‘DAR1’ and ‘DAR2’ were analysed by BCA colorimetric assay to ascertain new EC260/EC280 values, concentrated by centrifugal filtration (2000 g, 5° C.) and pooled. The conjugate was terminally filtered to 0.2 μm to obtain VHH-TFL conjugate (0.3 mg, 0.124 mg/ml, 19%).

### Trifunctional Linker-(Dendron(L-SO1861)4)-(Blocked TCO)-(Bivalent VHH) Synthesis

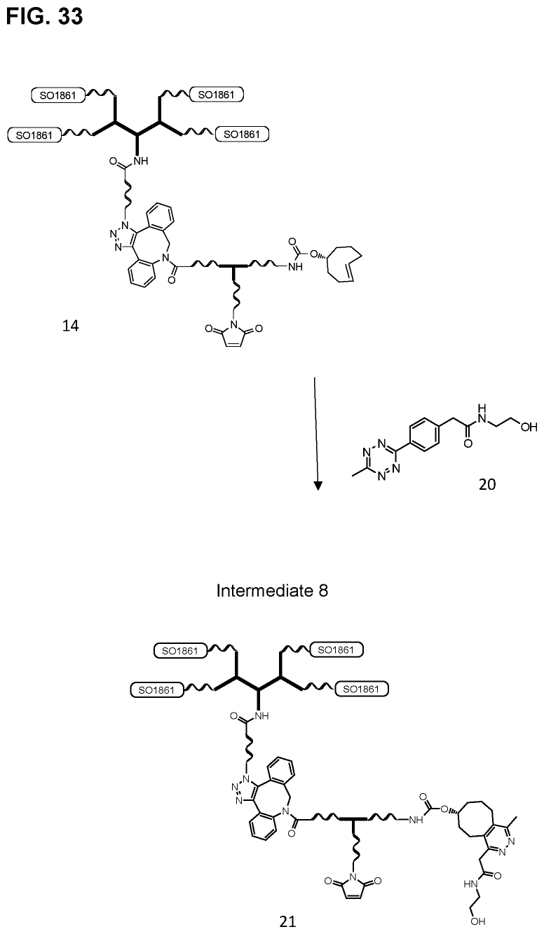

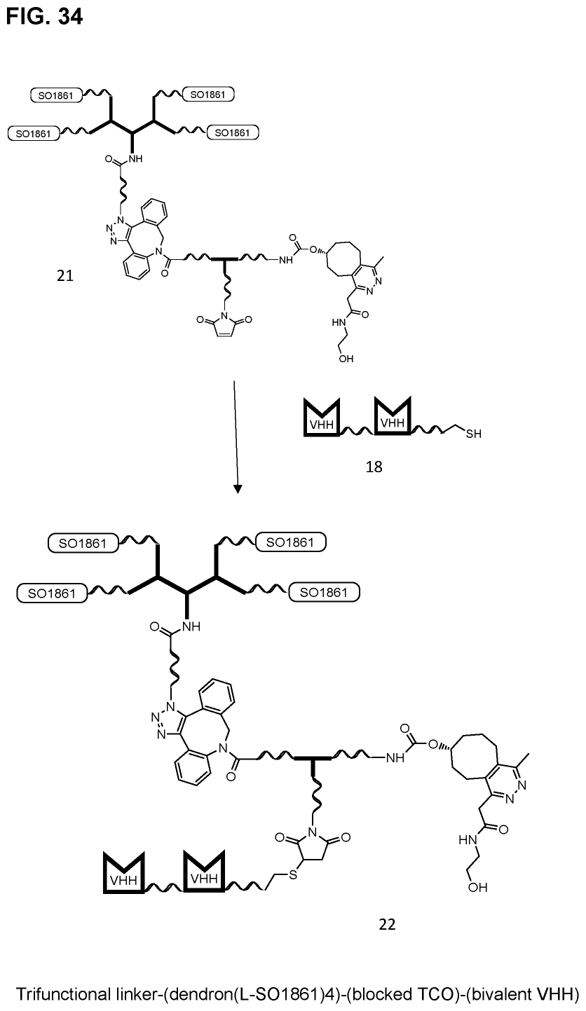

Referring to FIG. 33-34

### Intermediate 8:

Trifunctional linker-(dendron(L-SO1861)4)-(blocked TCO)-(Maleimide) (molecule 21) A solution of trifunctional linker-(dendron(-L-SO1861)4)-(TCO)-(Maleimide) (molecule 14) (5.4 mg, 0.49 μmol) in DMF (2.0 mL) was added to N-(2-hydroxyethyl)-2-(4-(6-methyl-1,2,4,5-tetrazin-3-yl)phenyl)acetamide (molecule 20) (0.16 mg, 0.58 μmol). The reaction mixture was shaken for 1 min and left standing at room temperature. After 30 min the reaction mixture was submitted to preparative MP-LC.^{1C} Fractions corresponding to the product (molecule 21) were immediately pooled together, frozen and lyophilized overnight to give the title compound (3.9 mg, 71%) as a white fluffy solid. Purity based on LC-MS 96%. See also FIG. 33.

### Trifunctional Linker-(Dendron(L-SO1861)4)-(Blocked TCO)-(Bivalent VHH) (Molecule 22)

To bivalent VHH (5.00 mg, 0.162 μmol, 1.0 mg/ml) was added a freshly prepared solution of TCEP (5 mole equivalents, 0.81 μmol, 46.4 μl) in TBS pH 7.5 (5 mg/ml), the mixture vortexed briefly and roller mixed at 20° C. for 120 minutes. The resulting bivalent VHH-SH (molecule 18) was purified by gel filtration using Zeba desalting columns eluting with TBS pH 7.5. Bivalent VHH-SH was analysed by UV-vis and Ellman's assay (0.842 mg/ml, VHH:SH=3.70).

To an aliquot of freshly reduced bivalent VHH-SH (1.35 mg, 43.7 nmol, 1.60 ml) was added an aliquot of freshly prepared TFL-(dSPT4)-(blocked TCO)-(Maleimide) (molecule 21) (4 mole equivalents, 0.175 μmol, 100 μl) in DMSO (20 mg/ml). The reaction mixture was roller mixed at 20° C. overnight. After incubation, the reaction mixture was analysed by Ellman's assay to ascertain incorporation by depletion, then quenched by the addition of a freshly prepared solution of NEM (10 mole equivalents, 10.9 μl) in TBS pH 7.5 (5 mg/ml). The bivalent VHH-TFL conjugate (molecule 22) was purified by gel filtration using a dedicated 2.6×40 cm Superdex 200 column eluting with DPBS pH 7.5 and filtered to 0.2 μm. From the purification run, a sample from each fraction of interest was taken and analysed by SDS. Fractions corresponding to components labelled ‘DAR1’ and ‘DAR2’ were analysed by BCA colorimetric assay to ascertain new EC260/EC280 values, concentrated by centrifugal filtration (2000 g, 5° C.) and pooled. The conjugate was terminally filtered to 0.2 μm to obtain VHH-TFL conjugate (0.2 mg, 0.124 mg/ml, 15%). See also FIG. 34.

### Trifunctional Linker-(Blocked DBCO)-(L-BNA Oligo)-(Bivalent VHH) Synthesis

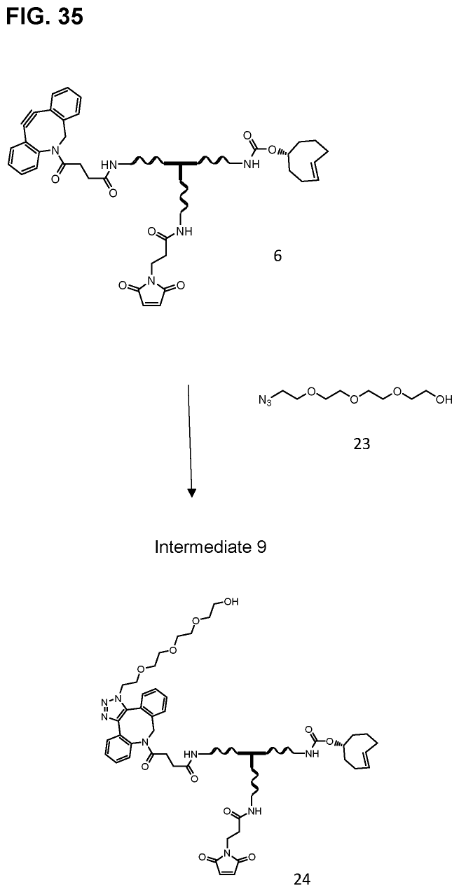

Referring to FIG. 35-37

### Intermediate 9:

### Trifunctional Linker-(Blocked DBCO)-(TCO)-(Maleimide) (Molecule 24)

A solution of 1-azido-3,6,9-trioxaundecane-11-ol (molecule 23) (2.17 mg, 9.88 μmol) in DMF (0.50 mL) was added to TFL-(DBCO)-(TCO)-(Maleimide) (molecule 6) (5.9 mg, 4.94 μmol). The reaction mixture was shaken for 1 min and left standing at room temperature. After 2 hours the reaction mixture was submitted to preparative MP-LC.^{1C} Fractions corresponding to the product (molecule 24) were immediately pooled together, frozen and lyophilized overnight to give the title compound (6 mg, 86%) as a white solid. Purity based on LC-MS 98%. See FIG. 35.

### Intermediate 10:

### Trifunctional Linker-(Blocked DBCO)-(L-BNA Oligo)-(Maleimide) (Molecule 25)

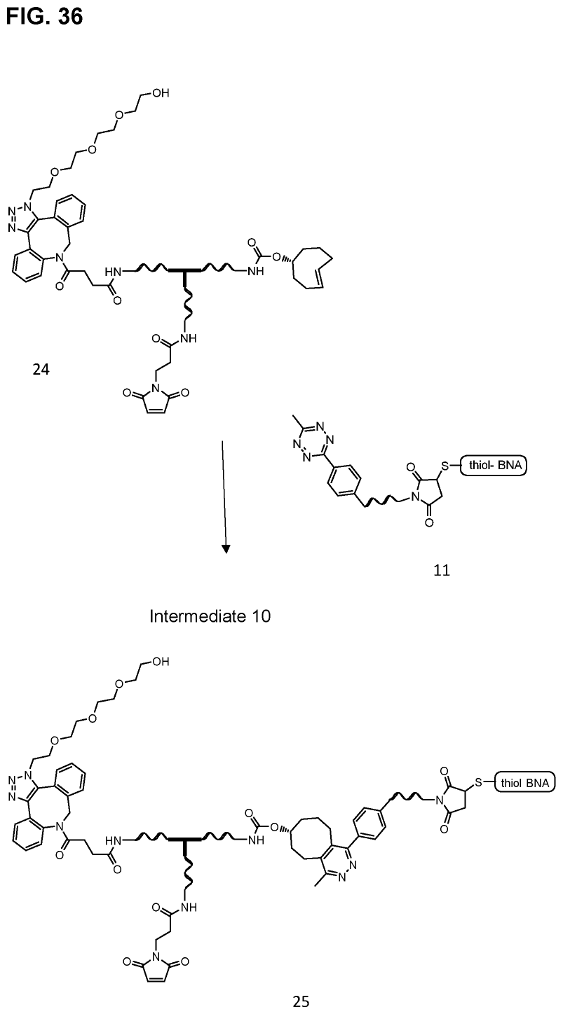

Methyltetrazine-BNA oligo (molecule 11) (3.8 mg, 0.68 μmol) was dissolved in 1 mL DPBS/acetonitrile mixture (4:1 v/v) and the resulting solution was directly added to trifunctional linker-(blocked DBCO)-(TCO)-(Maleimide) (molecule 24) (0.85 mg, 0.6 μmol). The reaction mixture was shaken for 1 min and left standing at room temperature. After 30 min the reaction mixture was frozen and lyophilized overnight to yield the crude title product as a fluffy solid. To the crude product was added a solution of 20 mM ammonium bicarbonate (1.50 mL) and the resulting suspension was filtered over a 0.45 μm syringe filter. The filtrate was lyophilized overnight to yield the title product (molecule 25) (3.2 mg, 76%) as a fluffy solid. Purity based on LC-MS 95%. See FIG. 36.

### Trifunctional Linker-(Blocked DBCO)-(L-BNA Oligo)-(Bivalent VHH) (Molecule 26)

To bivalent VHH (5.00 mg, 0.162 μmol, 1.0 mg/ml) was added a freshly prepared solution of TCEP (5 mole equivalents, 0.81 μmol, 46.4 μl) in TBS pH 7.5 (5 mg/ml), the mixture vortexed briefly and roller mixed at 20° C. for 120 minutes. The resulting bivalent VHH-SH (molecule 18) was purified by gel filtration using Zeba desalting columns eluting with TBS pH 7.5. Bivalent VHH-SH was analysed by UV-vis and Ellman's assay (0.842 mg/ml, VHH:SH=3.70).

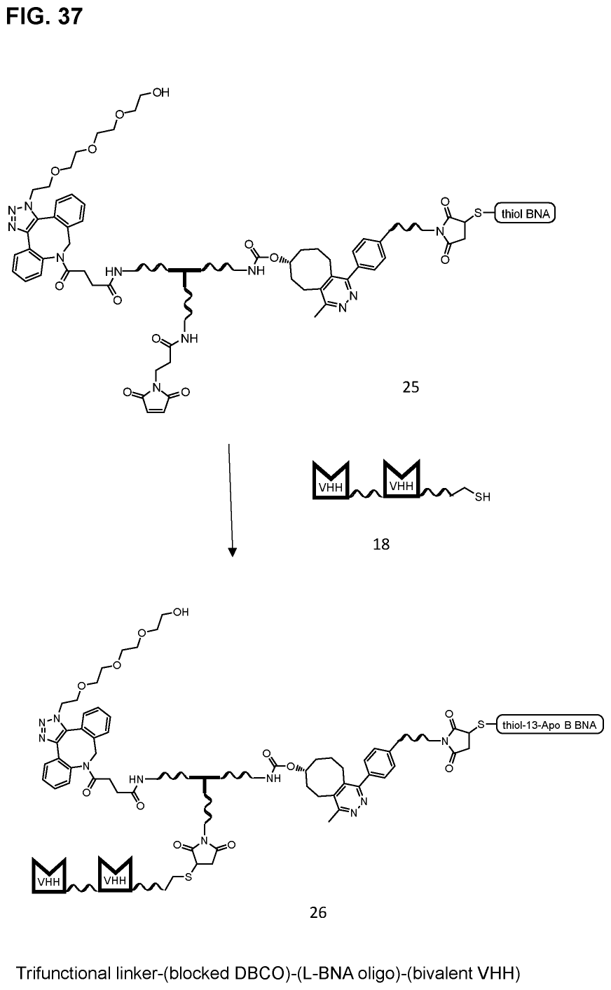

To an aliquot of freshly reduced bivalent VHH-SH (1.35 mg, 43.7 nmol, 1.60 ml) was added an aliquot of freshly prepared TFL-(blocked DBCO)-(L-BNA oligo)-(Maleimide) (molecule 25) (4 mole equivalents, 0.175 μmol, 100 μl) in DMSO (20 mg/ml). The reaction mixture was roller mixed at 20° C. overnight. After incubation, the reaction mixture was analysed by Ellman's assay to ascertain incorporation by depletion, then quenched by the addition of a freshly prepared solution of NEM (10 mole equivalents, 10.9 μl) in TBS pH 7.5 (5 mg/ml). The bivalent VHH-TFL conjugate (molecule 26) was purified by gel filtration using a dedicated 2.6×40 cm Superdex 200 column eluting with DPBS pH 7.5 and filtered to 0.2 μm. From the purification run, a sample from each fraction of interest was taken and analysed by SDS-PAGE. Fractions corresponding to components labelled ‘DAR1’ and ‘DAR2’ were analysed by BCA colorimetric assay to ascertain new EC260/EC280 values, concentrated by centrifugal filtration (2000 g, 5° C.) and pooled. The conjugate was terminally filtered to 0.2 μm to obtain VHH-TFL conjugate (0.2 mg, 0.124 mg/ml, 16%). See FIG. 37.

### Results

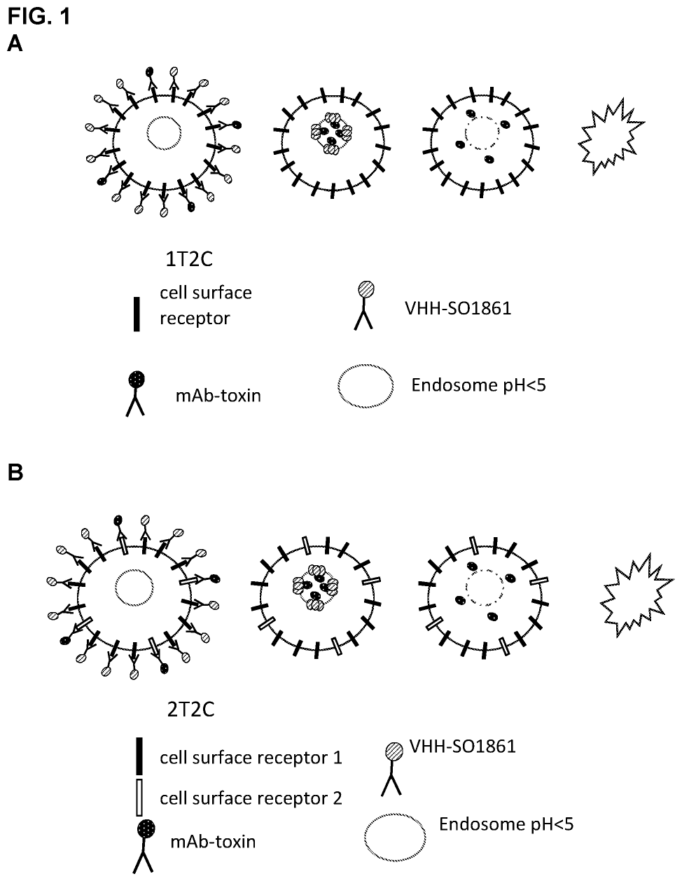

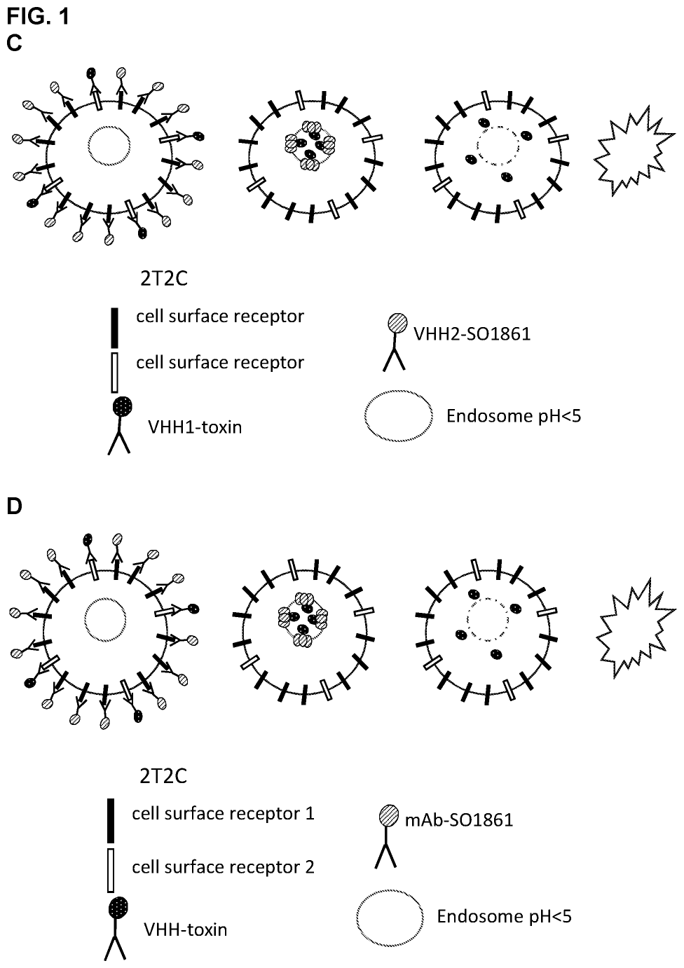

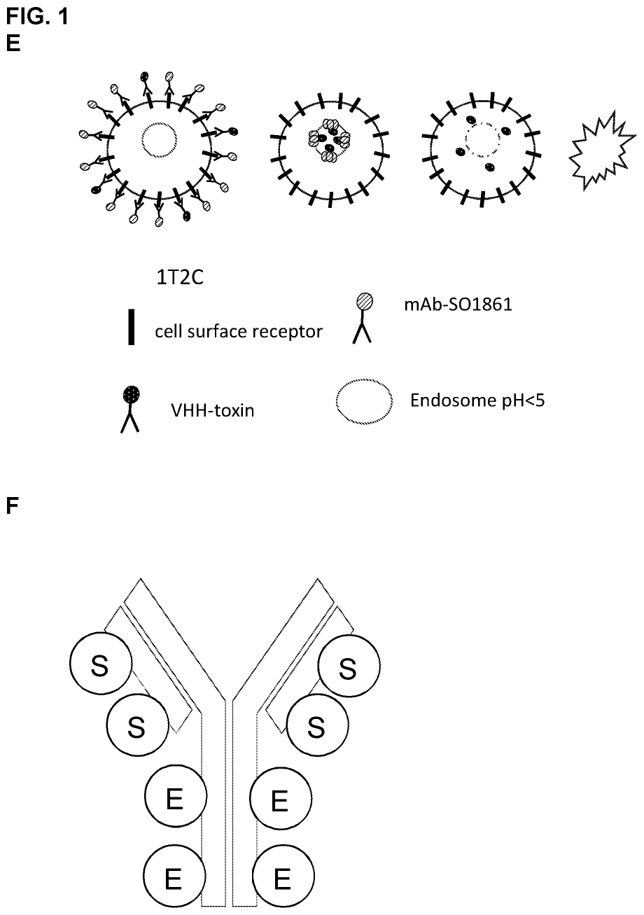

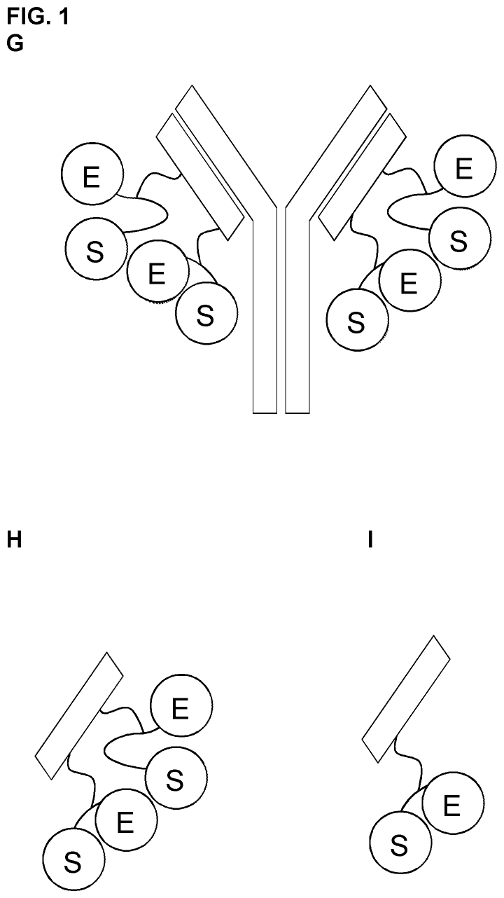

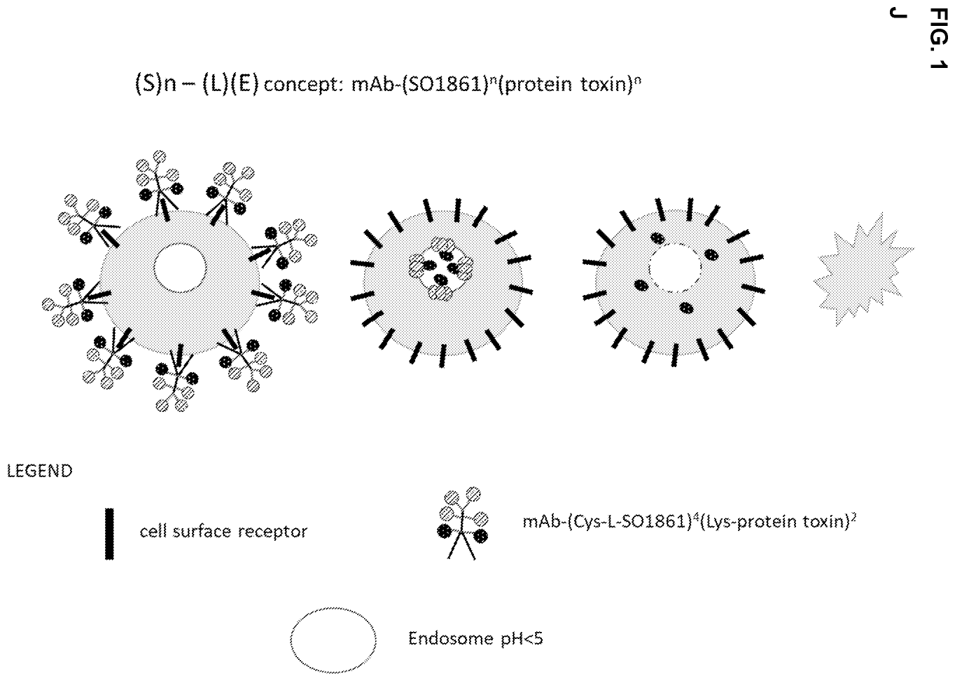

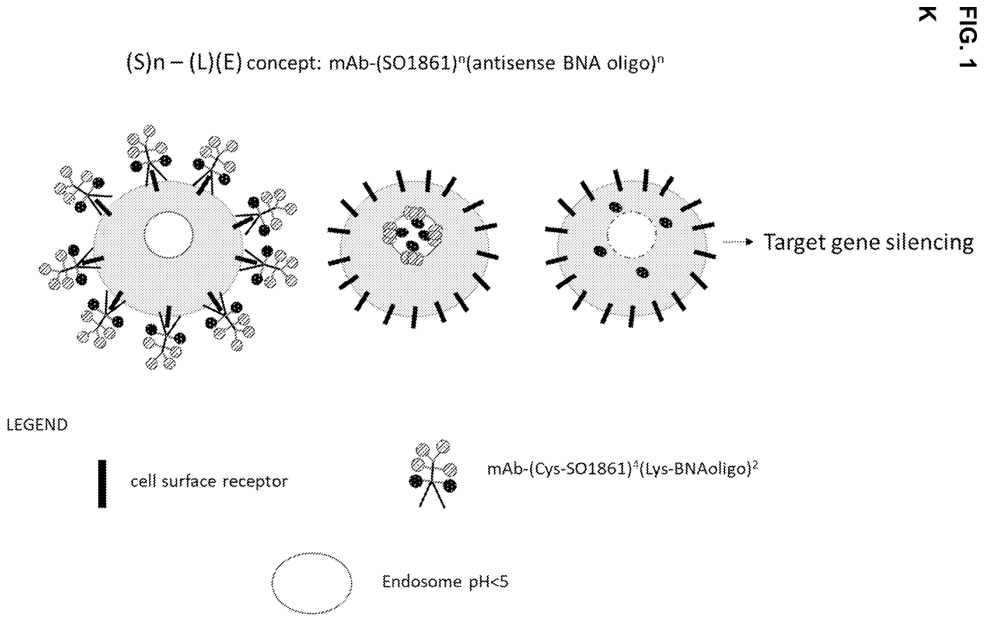

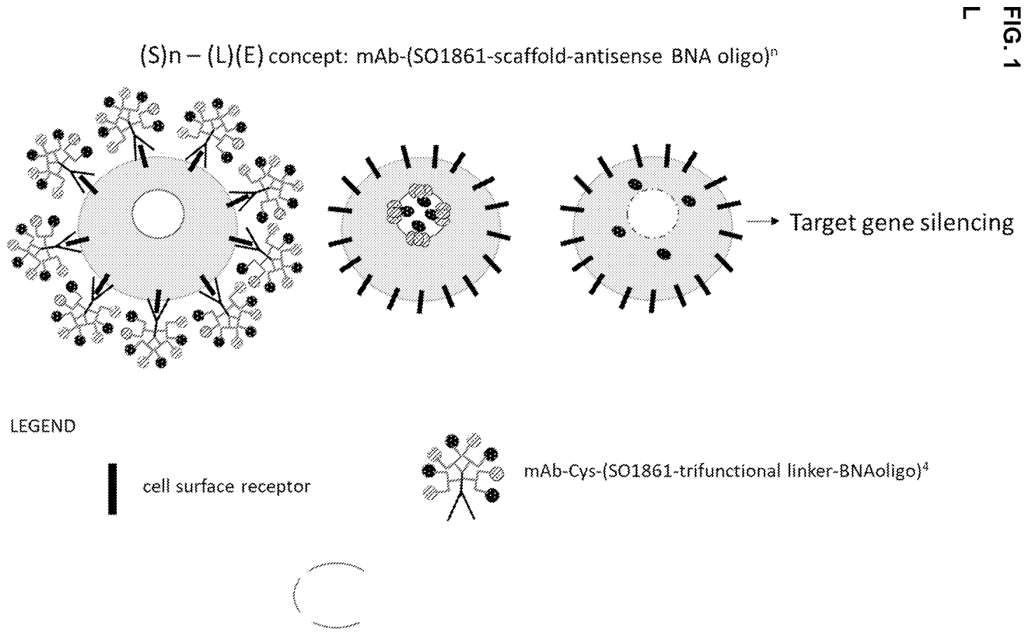

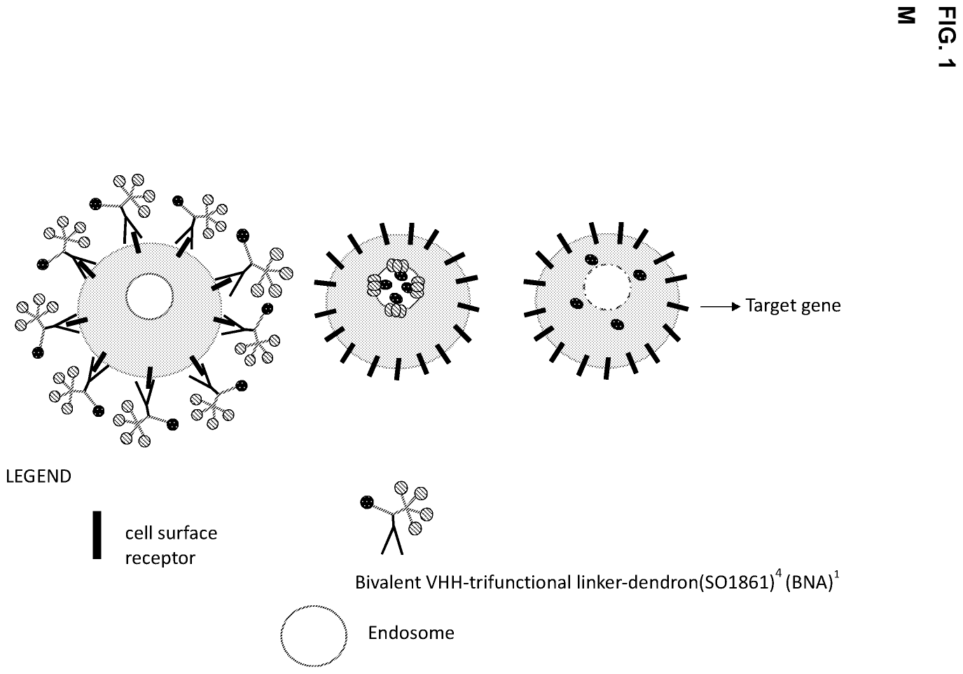

The targeted 1-components system (T1C) is the bivalent V_{HH}-SO1861-BNA according to the invention as illustrated in FIG. 1M.

For this, SO1861-EMCH (labile, L) and the HSP27BNA oligonucleotide (labile, L) were conjugated to the bivalent V_{HH}_EGFR (synthesis is outlined here-above and displayed in FIG. 31-32). Bivalent V_{HH}_EGFR-TFL-dendron(L-SO1861)4(L-HSP27BNA) (DAR1), combination therapies and controls were tested for cell viability and enhanced HSP27 gene silencing in A431 cells (EGFR⁺⁺) and A2058 (EGFR-) cells.

This revealed in A431 cells (EGFR⁺⁺) that the bivalent V_{HH}_EGFR-TFL-dendron(L-SO1861)4(L-HSP27BNA) and combi therapies showed no effect on cell viability up to 100 nM (FIG. 2A), whereas V_{HH}_EGFR-TFL-(block)(HSP27BNA) showed toxicity at higher concentrations (IC50=1000 nM; FIG. 2A). biVHH_EGFR-TFL(block)(HSP27BNA)+4000 nM SO1861-EMCH showed strong cell viability reduction at low concentrations conjugate (IC50=5 nM; FIG. 2A). In A2058 (EGFR⁻) cells no significant decrease in cell viability was observed up to 100 nM for all the conjugates or combinations FIG. 2B).

Next, BNA-mediated gene silencing of HSP27 was determined and this revealed in A431 cells (EGFR⁺⁺) that according to the invention the bivalent V_{HH}_EGFR-TFL-dendron-(L-SO1861)⁴(L-HSP27BNA) efficiently induces HSP27 gene silencing in A431 cells (IC50=8 nM; FIG. 3A) compared to bivalent V_{HH}_EGFR-TFL-(block)(HSP27BNA) alone (IC50=1000 nM; FIG. 3A) that also showed cell viability reduction at the same concentration range (see FIG. 2A). The combination treatment V_{HH}_EGFR-TFL-(block)(HSP27BNA)+25 nM V_{HH}_EGFR-TFL-dendron(L-SO1861)4(block) or V_{HH}_EGFR-TFL-dendron-(L-SO1861)4(block)+20 nM V_{HH}_EGFR-TFL-(block)(HSP27BNA) showed also activity around IC50=5 nM. However titration of higher concentrations of the variable conjugate revealed reduced activity due receptor binding competition, this is not observed in the bivalent V_{HH}_EGFR-TFL-dendron-(L-SO1861)⁴(L-HSP27BNA) conjugate according to the invention. biVHH_EGFR-TFL-(block)(HSP27BNA)+4000 nM SO1861-EMCH showed already HSP27 gene silencing at very low concentrations (IC50=0.01 nM; FIG. 3A), but also showed cell viability reduction at low nM concentrations (see FIG. 2A).

In A2058 cells (EGFR⁻) no gene silencing activity can be observed at low concentrations V_{HH}_EGFR-TFL-dendron(L-SO1861)4(L-HSP27BNA). At concentrations with more than 100 nM V_{HH}_EGFR-TFL-dendron(L-SO1861)4(L-HSP27BNA) HSP27 gene silencing is observed (FIG. 3B), however with similar concentrations also cell viability reduction is observed (see FIG. 2B). biVHH_EGFR-TFL(block)(HSP27BNA)+4000 nM SO1861-EMCH shows HSP27 gene silencing at IC50=1 nM, indicating non-receptor mediated uptake of biVHH_EGFR-TFL(block)(HSP27BNA) in A2058 cells.

This all shows and enables that conjugation of SO1861 and HSP27BNA on a bivalent V_{HH}, according to the invention, efficiently induces receptor targeted, SO1861-mediated enhanced cytoplasmic delivery of a therapeutic oligo nucleotide in the target cells, inducing very efficient targeted gene silencing with low concentrations oligo nucleotide.

### Bivalent VHH_EGFR-SO1861+CD71 Targeted Protein Toxin (2T2C)

The 2 target 2-components system (2T2C) is the combination treatment of bivalent V_{HH}-SO1861 and mAb-protein toxin, (FIG. 1B).

Bivalent V_{HH}_EGFR-TFL-dendron(L-SO1861)4(block) was produced as here-above described and displayed in FIG. 33-34. the Bivalent V_{HH}_EGFR binds with one arm the binding site of cetuximab and with the other arm the binding site of matuzumab on the EGFR receptor.

Bivalent V_{HH}_EGFR-TFL-dendron(L-SO1861)4(block) was titrated on a fixed concentration of 10 pM CD71mab-saporin and targeted protein toxin-mediated cell killing on A431 (EGFR⁺⁺/CD71⁺) and A2058 (EGFR⁻/CD71⁺) cells was determined. This revealed that bivalent V_{HH}_EGFR-TFL-dendron(L-SO1861)4(block)+10 pM CD71mab-saporin showed activity at IC50=50 nM in A431 (EGFR⁺⁺/CD71⁺), whereas SO1861-EMCH+10 pM CD71mab-saporin showed only activity at IC50=2000 nM (FIG. 4A). In A2058 (EGFR⁻/CD71⁺) cells, bivalent V_{HH}_EGFR-TFL-dendron(L-SO1861)4(block)+10 pM CD71mab-saporin was not effective, whereas SO1861-EMCH+10 pM CD71mab-saporin showed activity at IC50=1500 nM (FIG. 4B).

### Bivalent V_{HH}_EGFR-SO1861+EGFR Targeted Protein Toxins (1T2C)

The 1 target 2-components system (1T2C) (competing and non-competing) is the combination treatment of bivalent V_{HH}-SO1861 and mAb-SO1861, as illustrated in FIG. 1A, E.

Bivalent V_{HH}_EGFR-TFL-dendron(L-SO1861)4(block) was produced as described here-above and displayed in FIG. 33-34. the Bivalent V_{HH}_EGFR binds with one arm the binding site of cetuximab and with the other arm the binding site of matuzumab on the EGFR receptor.

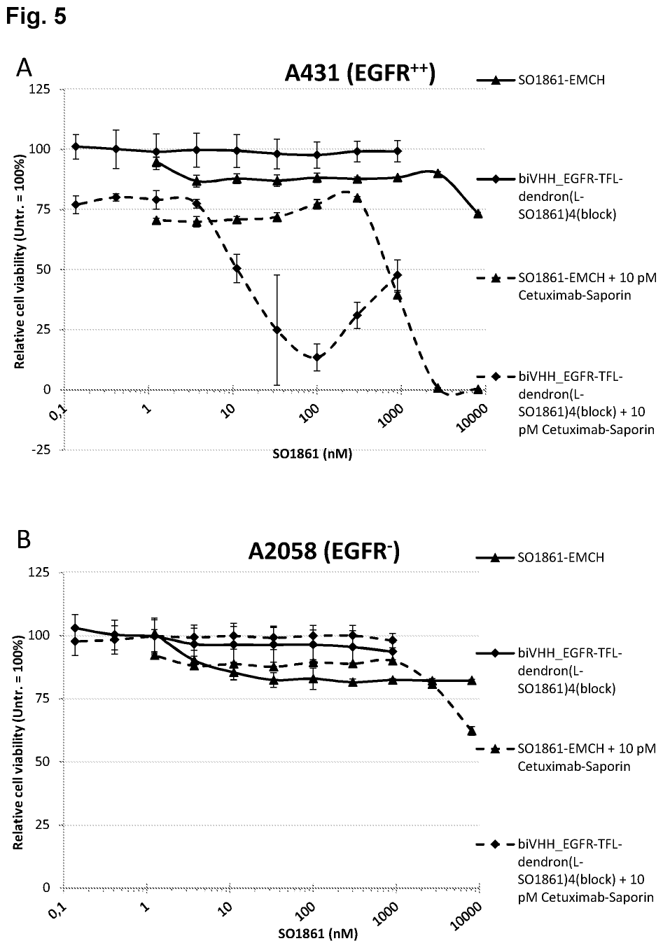

bivalent V_{HH}_EGFR-TFL-dendron(L-SO1861)4(block) was titrated on a fixed concentration of 10 pM 10 pM cetuximab-saporin, and targeted protein toxin-mediated cell killing on A431 (EGFR⁺⁺) and A2058 (EGFR⁻) was determined. This revealed that bivalent V_{HH}_EGFR-TFL-dendron(L-SO1861)4(block)+10 pM cetuximab-saporin showed activity at IC50=10 nM in A431 (EGFR⁺⁺), whereas higher concentrations V_{HH}_EGFR-TFL-dendron(L-SO1861)4(block) inhibit the activity due to receptor binding competition with cetuximab-saporin (FIG. 5A). SO1861-EMCH+10 pM cetuximab-saporin showed activity at IC50=1000 nM (FIG. 5A). In A2058 (EGFR⁻) cells, bivalent V_{HH}_EGFR-TFL-dendron(L-SO1861)4(block)+10 pM cetuximab-saporin or SO1861-EMCH+10 pM cetuximab-saporin was not effective up to 1000 nM (FIG. 5B).

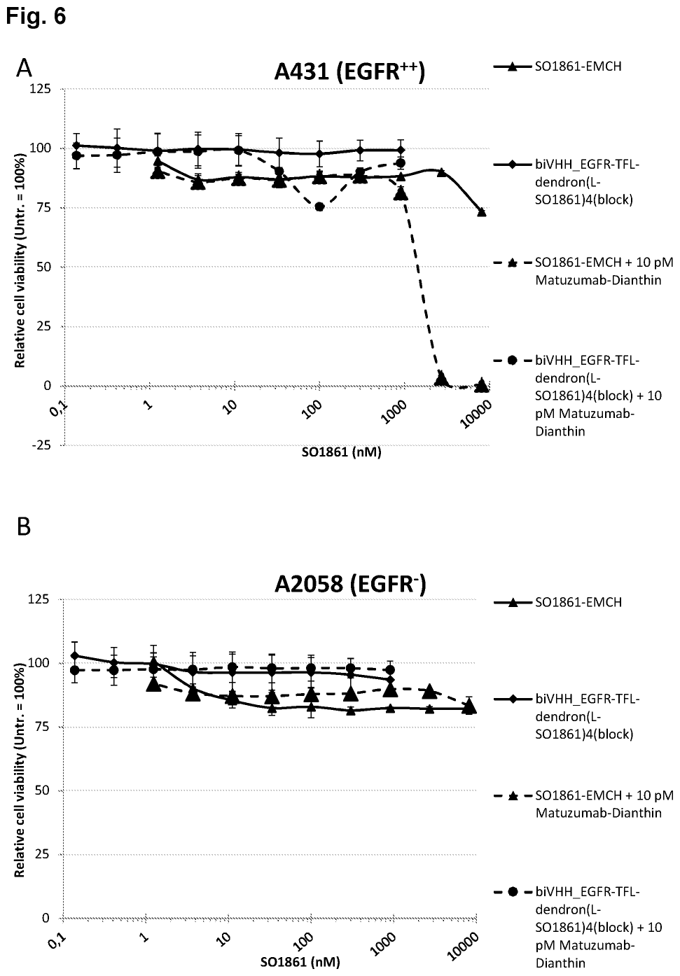

Next, bivalent V_{HH}_EGFR-TFL-dendron(L-SO1861)4(block) was titrated on a fixed concentration of 10 pM matuzumab-dianthin and targeted protein toxin-mediated cell killing on A431 (EGFR⁺⁺) and A2058 (EGFR⁻) cells was determined. This revealed that in A431 (EGFR⁺⁺) cells, bivalent V_{HH}_EGFR-TFL-dendron(L-SO1861)4(block)+10 pM matuzumab-dianthin showed only 25% cell killing activity around 100 nM, whereas higher concentrations V_{HH}_EGFR-TFL-dendron(L-SO1861)4(block) completely blocked the activity due to receptor binding competition with matuzumab-dianthin (FIG. 6A). SO1861-EMCH+10 pM matuzumab-dianthin showed activity at IC50=1500 nM (FIG. 6A). In A2058 (EGFR⁻) cells, bivalent V_{HH}_EGFR-TFL-dendron(L-SO1861)4(block)+10 pM matuzumab-dianthin or SO1861-EMCH+10 pM matuzumab-dianthin was not effective (FIG. 6B).

Next, bivalent V_{HH}_EGFR-TFL-dendron(L-SO1861)4(block) was titrated on a fixed concentration of 5 pM bivalent V_{HH}-EGFR-dianthin and targeted protein toxin-mediated cell killing on A431 (EGFR⁺⁺) and A2058 (EGFR⁻) cells was determined. This revealed that in A431 (EGFR⁺⁺) cells, bivalent V_{HH}_EGFR-TFL-dendron(L-SO1861)4(block)+5 pM bivalent V_{HH}-EGFR-dianthin showed only 25% cell killing activity around 100 nM, whereas higher concentrations V_{HH}_EGFR-TFL-dendron(L-SO1861)4(block) completely blocked the activity due to receptor binding competition with bivalent V_{HH}-EGFR-dianthin (FIG. 7A). SO1861-EMCH+5 pM bivalent V_{HH}-EGFR-dianthin showed activity at IC50=1500 nM (FIG. 7A). In A2058 (EGFR⁻) cells, bivalent V_{HH}_EGFR-TFL-dendron(L-SO1861)4(block)+5 pM bivalent V_{HH}-EGFR-dianthin or SO1861-EMCH+5 pM bivalent V_{HH}-EGFR-dianthin was not effective (FIG. 7B).

### Example 2. V_{HH}-SO1861+V_{HH}-dianthin (2T2C)

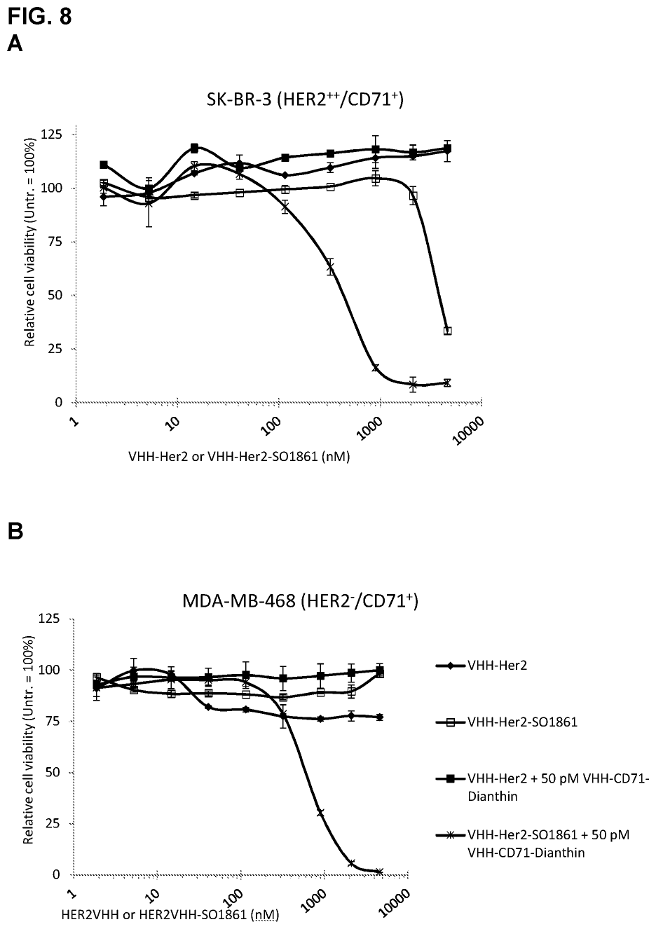

The 2 target 2-components system (2T2C) is the combination treatment of V_{HH}1-SO1861 and V_{HH}2-protein toxin, where each V_{HH} recognizes another cell surface receptor (FIG. 1C). SO1861-EMCH was conjugated to the terminal cysteine residues of the V_{HH} targeting HER2, producing HER2V_{HH}-SO1861 (DAR1). HER2V_{HH}-SO1861 was titrated on a fixed concentration of 50 pM CD71V_{HH}-dianthin and targeted protein toxin mediated cell killing on SK-BR-3 (HER2⁺⁺/CD71⁺) and MDA-MB-468 (HER2-/CD71⁺) was determined. This revealed enhanced cell killing at relatively low concentrations of V_{HH}HER2-L-SO1861 (SK-BR-3: IC50=300 nM; FIG. 8A). Equivalent concentrations of HER2V_{HH}-SO1861 alone induced cell killing at high concentrations (IC50=4.000 nM), whereas equivalent concentrations of HER2V_{HH}, HER2V_{HH}+50 pM CD71V_{HH}-dianthin could not induce cell killing (IC50>5.000 nM; FIG. 8A). In MDA-MB-468 (HER2⁻/CD71⁺) the combination of HER2V_{HH}-SO1861+50 pM CD71V_{HH}-dianthin revealed cell killing activity at higher concentrations (IC50=600 nM; FIG. 8B), whereas equivalent concentrations of HER2V_{HH}, HER2V_{HH}-SO1861 or HER2V_{HH}+50 pM CD71V_{HH}-dianthin could not induce cell killing activity (IC50>5.000 nM; FIG. 8B).

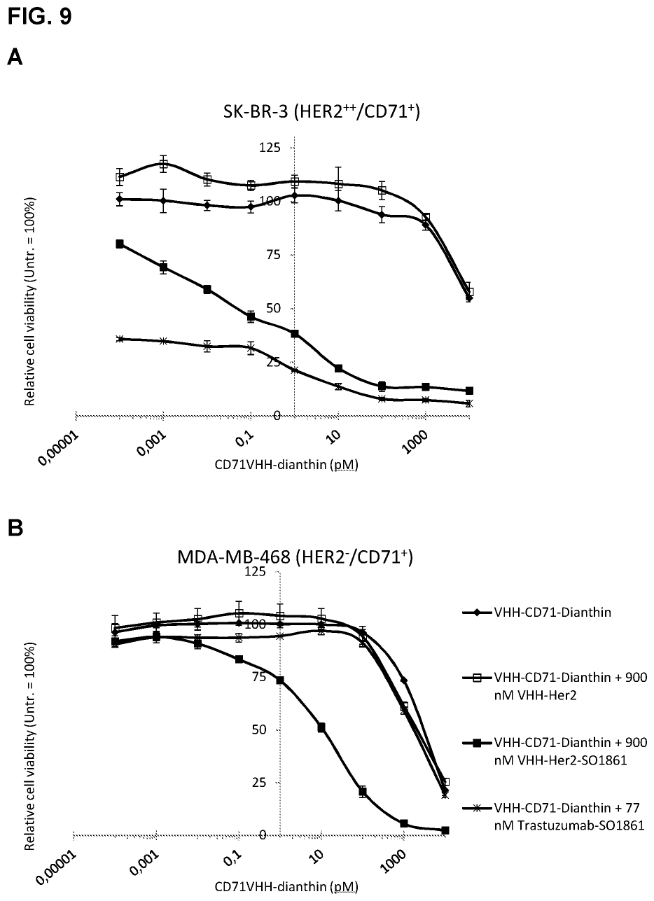

Next, CD71V_{HH}-dianthin was titrated on a fixed concentration of 900 nM HER2V_{HH}-SO1861 and targeted protein toxin mediated cell killing on SK-BR-3 (HER2⁺⁺/CD71⁺) and MDA-MB-468 (HER2-/CD71⁺) was determined. This revealed that 900 nM HER2V_{HH}-SO1861 in combination with low concentrations CD71V_{HH}-dianthin induced efficient cell killing of SK-BR-3 cells (IC50=0.05 pM; FIG. 9A), whereas CD71V_{HH}dianthin or CD71V_{HH}-dianthin+900 nM HER2V_{HH} could only induce cell killing at high concentrations (IC50>10.000 pM); FIG. 9A). Besides, CD71V_{HH}-dianthin was also titrated on a fixed concentration of 77 nM trastuzumab-SO1861 (DAR4) and this revealed also a strong enhancement in cell killing activity in SK-BR-3 (HER2+/CD71⁺) cells ((IC50<0,0001 pM). In MDA-MB-468 cells (HER2⁻/CD71⁺) CD71V_{HH}-dianthin+900 nM HER2V_{HH}-SO1861 showed cell killing only at much higher concentrations (IC50=10 pM, FIG. 9B), whereas CD71V_{HH}-dianthin, CD71V_{HH}-dianthin+900 nM HER2V_{HH} or CD71V_{HH}-dianthin+trastuzumab-SO1861 (DAR4) showed cell killing only at IC50=2.000 pM; FIG. 9B).

All this shows that relatively low concentrations of V_{HH}CD71-dianthin can be effective and induce cell killing in combination with low V_{HH}HER2-SO1861 conjugate concentrations in high HER2/CD71 expressing cells.

The combination according to the invention in MDA-MB-468 cells (HER2⁻/CD71⁺) did not reveal any cell killing activity. This shows that in the absence of sufficient receptor expression, effective intracellular delivered SO1861 concentrations are not reached (threshold) to induce endosomal escape and cytoplasmic delivery of the protein toxin.

### Example 2. V_{HH}-dianthin+mAb-SO1861 (1T2C and 2T2C)

The 1 target 2-components system (1T2C) is the combination treatment of mAb-SO1861 and V_{HH}-protein toxin, where mAb and V_{HH} recognize and bind the same cell surface receptor (FIG. 1E). The 2 target 2-components system (2T2C) is also the combination treatment of mAb-SO1861 and V_{HH}-protein toxin, where the mAb and V_{HH} recognize another cell surface receptor (FIG. 1D).

Dianthin-C(dianthin with a terminal cysteine) was conjugated to the terminal cysteine residues of the V_{HH} targeting HER2, V_{HH} targeting CD71 or V_{HH} targeting EGFR producing HER2V_{HH}-dianthin (DAR1), CD71V_{HH}-dianthin (DAR1) and EGFRV_{HH}-dianthin (DAR1).

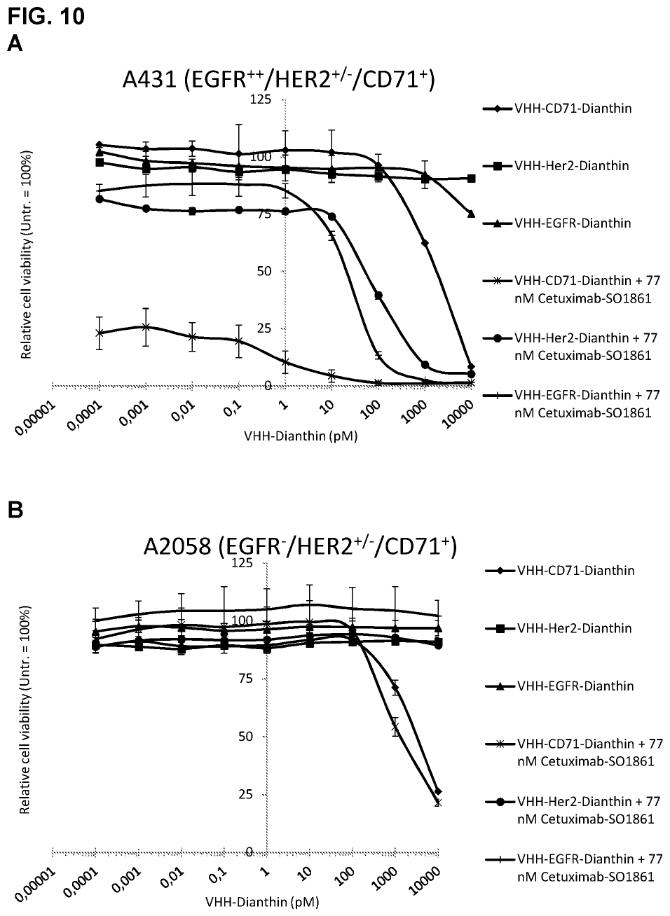

CD71V_{HH}-dianthin, HER2V_{HH}-dianthin or EGFRV_{HH}-dianthin was titrated on a fixed concentration of cetuximab-SO1861 (DAR4) and targeted protein toxin mediated cell killing on A431 (EGFR⁺⁺/HER2^{+/-}/CD71⁺) and A2058 (EGFR⁻/HER2^{+/-}/CD71⁺) was determined. This revealed that very low concentrations CD71V_{HH}-dianthin in combination with 77 nM cetuximab-SO1861 induced efficient cell killing of A431 cells (IC50<0,0001 pM; FIG. 10A), whereas CD71V_{HH}-dianthin alone showed activity at IC50=2000 pM. The other two combinations EGFRV_{HH}-dianthin+77 nM cetuximab-SO1861 and HER2V_{HH}-dianthin+77 nM cetuximab-SO1861 showed efficient cell killing at respectively IC50=20 pM and IC50=50 pM, whereas EGFRV_{HH}-dianthin or HER2V_{HH}-dianthin alone could not induce efficient cell killing in A431 cells (IC50>10.000 pM; FIG. 10A). In A2058 cells (EGFR⁻/HER2^{+/-}/CD71⁺), CD71V_{HH}-dianthin and CD71V_{HH}-dianthin+77 nM cetuximab-SO1861 showed cell killing activity at respectively, IC50=3.000 pM and IC50=1.000 pM, whereas all other treatments or combinations showed no cell killing up to IC50=10.000 pM V_{HH}-toxin in A2058 cells (FIG. 10B).

This shows that cetuximab-SO1861 (DAR4) can efficiently induce endosomal escape of three different V_{HH}-dianthin conjugates, thereby inducing enhanced cell killing in A431 cells.

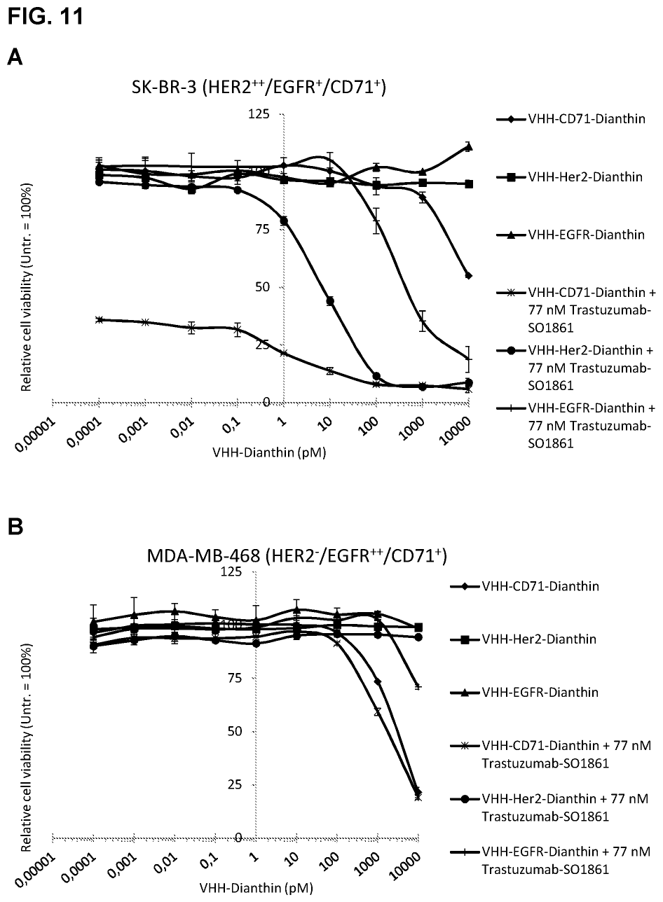

Next, CD71V_{HH}-dianthin, HER2V_{HH}-dianthin or EGFRV_{HH}-dianthin was titrated on a fixed concentration of trastuzumab-SO1861 (DAR4) and targeted protein toxin mediated cell killing on SK-BR-3 (HER2⁺⁺/EGFR⁻/CD71⁺) and MDA-MB-468 cells (HER2⁻/EGFR⁺⁺/CD71⁺) was determined. This revealed that very low concentrations CD71V_{HH}-dianthin in combination with 77 nM trastuzumab-SO1861 induced efficient cell killing of SK-BR-3 cells (IC50<0,0001 pM; FIG. 11A), whereas CD71V_{HH}-dianthin alone showed activity at IC50=10.000 pM. The other two combinations EGFRV_{HH}-dianthin+77 nM trastuzumab-SO1861 and HER2V_{HH}-dianthin+77 nM trastuzumab-SO1861 showed efficient cell killing at respectively IC50=400 pM and IC50=6 pM, whereas EGFRV_{HH}-dianthin or HER2V_{HH}-dianthin alone could not induce efficient cell killing in SK-BR-3 cells (IC50>10.000 pM; FIG. 11A). In MDA-MB-468 cells (HER2⁻/EGFR⁺⁺/CD71⁺), CD71V_{HH}-dianthin and CD71V_{HH}-dianthin+77 nM cetuximab-SO1861 showed cell killing activity at respectively, IC50=3000 and IC50=2000 pM., whereas all other treatments or combinations showed no cell killing up to IC50=10.000 pM V_{HH}-dianthin in MDA-MB-468 cells (FIG. 11B). This shows that trastuzumab-SO1861 (DAR4) can efficiently induce endosomal escape of three different V_{HH}-dianthin conjugates, thereby inducing enhanced cell killing in SK-BR-3 cells.

### Materials and Methods

### Materials

SO1861 was isolated and purified by Analyticon Discovery GmbH from raw plant extract obtained from Saponaria officinalis. V_{HH} were purchased from QVQ, Utrecht, The Netherlands (HER2V_{HH}: clone name: Q17c; CD71V_{HH}: clone name: Q52c EGFRV_{HH}: clone name: Q86c). Trastuzumab (Tras, Herceptin®, Roche), Cetuximab (Cet, Erbitux®, Merck KGaA) were purchased from the pharmacy (Charite, Berlin). CD71 monoclonal antibody was purchased from BioCell (Okt9, #BE0023). Custom trastuzumab-saporin and antiCD71mab-saporin conjugate was produced and purchased from Advanced Targeting Systems (San Diego, CA). Dianthin-Cys (Dia-Cys, Dianthin mutant with a single C-terminal cysteine was produced by Proteogenix, France.

Tris(2-carboxyethyl)phosphine hydrochloride (TCEP, 98%, Sigma-Aldrich), 5,5-Dithiobis(2-nitrobenzoic acid) (DTNB, Ellman's reagent, 99%, Sigma-Aldrich), Zeba™ Spin Desalting Columns (2 mL, Thermo-Fisher), NuPAGE™ 4-12% Bis-Tris Protein Gels (Thermo-Fisher), NuPAGE™ MES SDS Running Buffer (Thermo-Fisher), Novex™ Sharp Pre-stained Protein Standard (Thermo-Fisher), PageBlue™ Protein Staining Solution (Thermo-Fischer), Pierce™ BCA Protein Assay Kit (Thermo-Fisher), N-Ethylmaleimide (NEM, 98%, Sigma-Aldrich), 1,4-Dithiothreitol (DTT, 98%, Sigma-Aldrich), Sephadex G25 (GE Healthcare), Sephadex G50 M (GE Healthcare), Superdex 200P (GE Healthcare), Isopropyl alcohol (IPA, 99.6%, VWR), Tris(hydroxymethyl)aminomethane (Tris, 99%, Sigma-Aldrich), Tris(hydroxymethyl)aminomethane hydrochloride (Tris.HCL, Sigma-Aldrich), L-Histidine (99%, Sigma-Aldrich), D-(+)-Trehalose dehydrate (99%, Sigma-Aldrich), Polyethylene glycol sorbitan monolaurate (TWEEN 20, Sigma-Aldrich), Dulbecco's Phosphate-Buffered Saline (DPBS, Thermo-Fisher), Guanidine hydrochloride (99%, Sigma-Aldrich), Ethylenediaminetetraacetic acid disodium salt dihydrate (EDTA-Na₂, 99%, Sigma-Aldrich), sterile filters 0.2 μm and 0.45 μm (Sartorius), Succinimidyl 4-(N-maleimidomethyl)cyclohexane-1-carboxylate (SMCC, Thermo-Fisher), Vivaspin T4 and T15 concentrator (Sartorius), Superdex 200PG (GE Healthcare), Tetra(ethylene glycol) succinimidyl 3-(2-pyridyldithio)propionate (PEG₄-SPDP, Thermo-Fisher), HSP27 BNA disulfide oligonucleotide (Biosynthesis), [O-(7-Azabenzotriazol-1-yl)-N,N,N,N-tetramethyluronium-hexafluorphosphat](HATU, 97%, Sigma-Aldrich), Dimethyl sulfoxide (DMSO, 99%, Sigma-Aldrich), N-(2-Aminoethyl)maleimide trifluoroacetate salt (AEM, 98%, Sigma-Aldrich), L-Cysteine (98.5%, Sigma-Aldrich), deionized water (DI) was freshly taken from Ultrapure Lab Water Systems (MilliQ, Merck), Nickel-nitrilotriacetic acid agarose (Ni-NTA agarose, Protino), Glycine (99.5%, VWR), 5,5-Dithiobis(2-nitrobenzoic acid (Ellman's reagent, DTNB, 98%, Sigma-Aldrich), S-Acetylmercaptosuccinic anhydride Fluorescein (SAMSA reagent, Invitrogen) Sodium bicarbonate (99.7%, Sigma-Aldrich), Sodium carbonate (99.9%, Sigma-Aldrich), PD MiniTrap desalting columns with Sephadex G-25 resin (GE Healthcare), PD10 G25 desalting column (GE Healthcare), Zeba Spin Desalting Columns in 0.5, 2, 5, and 10 mL (Thermo-Fisher), Vivaspin Centrifugal Filters T4 10 kDa MWCO, T4 100 kDa MWCO, and T15 (Sartorius), Biosep s3000 aSEC column (Phenomenex), Vivacell Ultrafiltration Units 10 and 30 kDa MWCO (Sartorius), Nalgene Rapid-Flow filter (Thermo-Fisher),

### Methods

### SO1861-EMCH Synthesis

To SO1861 (121 mg, 0.065 mmol) and EMCH.TFA (110 mg, 0.325 mmol) was added methanol (extra dry, 3.00 mL) and TFA (0.020 mL, 0.260 mmol). The reaction mixture stirred at room temperature. After 1.5 hours the reaction mixture was subjected to preparative MP-LC.¹ Fractions corresponding to the product were immediately pooled together, frozen and lyophilized overnight to give the title compound (120 mg, 90%) as a white fluffy solid. Purity based on LC-MS 96%.

LRMS (m/z): 2069 [M−1]¹⁻

LC-MS r.t. (min): 1.08⁴

### Cell Viability Assay

After treatment the cells were incubated for 72 hr at 37° C. before the cell viability was determined by a MTS-assay, performed according to the manufacturer's instruction (CellTiter 96@AQueous One Solution Cell Proliferation Assay, Promega). Briefly, the MTS solution was diluted 20× in DMEM without phenol red (PAN-Biotech GmbH) supplemented with 10% FBS. The cells were washed once with 200 μL/PBS well, after which 100 μL diluted MTS solution was added/well. The plate was incubated for approximately 20-30 minutes at 37° C. Subsequently, the OD at 492 nm was measured on a Thermo Scientific Multiskan FC plate reader (Thermo Scientific). For quantification the background signal of ‘medium only’ wells was subtracted from all other wells, before the cell viability percentage of treated/untreated cells was calculated, by dividing the background corrected signal of treated wells over the background corrected signal of the untreated wells (×100).

### FACS Analysis

Cells were seeded in DMEM (PAN-Biotech GmbH) supplemented with 10% fetal calf serum (PAN-Biotech GmbH) and 1% penicillin/streptomycin (PAN-Biotech GmbH), at 500,000 c/plate in 10 cm dishes and incubated for 48 hrs (5% CO₂, 37° C.), until a confluency of 90% was reached. Next, the cells were trypsinized (TryplE Express, Gibco Thermo Scientific) to single cells. 0.75×10⁶ Cells were transferred to a 15 mL falcon tube and centrifuged (1,400 rpm, 3 min). The supernatant was discarded while leaving the cell pellet submerged. The pellet was dissociated by gentle tapping the falcon tube on a vortex shaker and the cells were washed with 4 mL cold PBS (Mg²⁺ and Ca²⁺ free, 2% FBS). After washing the cells were resuspended in 3 mL cold PBS (Mg²⁺ and Ca²⁺ free, 2% FBS) and divided equally over 3 round bottom FACS tubes (1 mL/tube). The cells were centrifuged again and resuspended in 200 μL cold PBS (Mg²⁺ and Ca²⁺ free, 2% FBS) or 200 μL antibody solution; containing 5 μL antibody in 195 μL cold PBS (Mg²⁺ and Ca²⁺ free, 2% FBS). APC Mouse IgG1, K APC anti-human EGFR (#352906, Biolegend) was used to stain the EGFR receptor. PE anti-human HER2 APC anti-human CD340 (erbB2/HER-2) (#324408 Biolegend) was used to stain the HER2 receptor, PE Mouse IgG2a, K Isotype Ctrl FC (#400212, Biolegend) was used as its matched isotype control. PE anti-human CD71 (#334106, Biolegend) was used to stain the CD71 receptor, PE Mouse IgG2a, K Isotype Ctrl FC (#400212, Biolegend) was used as its matched isotype control. Samples were incubated for 30 min at 4° C. on a tube roller mixer. Afterwards, the cells were washed 3× with cold PBS (Mg²⁺ and Ca²⁺ free, 2% FBS) and fixated for 20 min at room temperature using a 2% PFA solution in PBS. Cells were washed 2× with cold PBS and resuspended in 250-350 μL cold PBS for FACS analysis. Samples were analyzed with a BD FACSCanto II flow cytometry system (BD Biosciences) and FlowJo software. Results of the analyses of the cell-surface expression of EGFR, HER2 and CD71 on the various cells is summarized in Table A2.

TABLE A2
Cell surface expression levels of EGFR,
HER2 and CD71 of various cells

| | EGFR | HER2 | CD71 |
|---|---|---|---|
| | expression | expression | expression |
| Cell line | level (MFI) | level (MFI) | level (MFI) |
| MDA-MB-468 | 1656 | 1 | 186 |
| A431 | 1593 | 10 | 322 |
| SK-BR-3 | 28 | 1162 | 331 |
| A2058 | 1 | 5 | 59 |

### Procedure for the Conjugation of V_{HH}-SO1861

To an aliquot of V_{HH} was added an aliquot of freshly prepared TCEP solution (10.0 mg/ml), the mixture vortexed briefly then incubated for 30 minutes at 20° C. with roller-mixing. After incubation, the resulting V_{HH}-SH was purified by gel filtration using zeba spin desalting column into TBS pH 7.5. To the resulting V_{HH}-SH was added freshly prepared SPT-EMCH solution the mixture vortexed briefly then incubated overnight at 20° C.

After incubation, an aliquot of V_{HH}-SO1861 mixture was removed and characterised by Ellman's assay to ascertain SO1861 incorporation. The conjugate was purified by 1.6×35 cm Superdex 200PG column eluting with DPBS pH 7.5 to give purified V_{HH}-SO1861. The aliquot was filtered to 0.2 μm, concentrated and normalised to 1.0 mg/ml to afford V_{HH}-SO1861.

### Procedure for the Conjugation of V_{HH}-Dianthin

Dianthin-Cys was concentrated by ultrafiltration using a vivaspin T15 10 KDa MWCO centrifugal filter and buffer exchanged into TBS pH 7.5. To the concentrated Dianthin-Cys was added an aliquot of freshly prepared TCEP solution (10.0 mg/ml), the mixture vortexed briefly then incubated for 60 minutes at 20° C. with roller-mixing. After incubation, the resulting Dianthin-SH was purified by gel filtration using a zeba spin desalting column then repeated centrifugal-wash cycles using a vivaspin T15 10 KDa MWCO centrifugal filter into TBS pH 7.5. The resulting Dianthin-SH was reacted with freshly prepared DTME solution (10 mg/ml) in DMSO, the mixture vortexed briefly then incubated for 60 minutes at 20° C. After, the Dianthin-DTME was obtained following purification by gel filtration using a zeba spin desalting column into TBS pH 7.5. The Dianthin-DTME was stored at 20° C. until conjugated. At the same time, an aliquot of V_{HH} was concentrated by ultrafiltration using a vivaspin T15 10 KDa MWCO centrifugal filter and buffer exchanged into TBS pH 7.5. To the concentrated V_{HH} was added an aliquot of freshly prepared TCEP solution (10.0 mg/ml), the mixture vortexed briefly then incubated for 60 minutes at 37° C. with roller-mixing. After incubation, the resulting V_{HH} was purified by gel filtration using a zeba spin desalting column then repeated centrifugal-wash cycles using a vivaspin T4 5 KDa MWCO centrifugal filter into TBS pH 7.5. An aliquot of the resulting V_{HH}-SH was reacted with Dianthin-DTME, the mixture vortexed briefly then incubated overnight at 20° C. After, the reaction mixture was concentrated using a vivaspin T4 10 KDa MWCO centrifuge tube and purified by gel filtration using a 1.6×35 cm Superdex 200PG column eluting into DPBS pH 7.5.

### Antibody-(L-SO1861)4

Trastuzumab, Cetuximab, are referred hereafter as “Ab”. Ab was conjugated to the saponin SO18161-EMCH via Michael-type thiol-ene conjugation reaction at DARs of 1, 2, 3, 4, 5, and 6. The SO1861-EMCH molecule obtains a labile (L) hydrazone bond between its structure and its maleimide function generating a labile bond between the saponin and Ab. The procedure is exemplary described for Trastuzumab-(L-SO1861)4: To a solution of Cetuximab (40 mg, 8.0 ml) was added 10 μl/ml each of Tris concentrate (127 mg/ml, 1.05 M), Tris.HCl concentrate (623 mg/ml, 3.95 M) and EDTA-Na₂ concentrate (95 mg/ml, 0.26 M) to give a 50 mM TBS, 2.5 mM EDTA buffer pH 7.5.

To Cetuximab divided into four portions (each of 9.73 mg, 4.864 mg/ml, 65 nmol) was added an aliquot of freshly prepared TCEP solution (0.5-2.0 mg/ml, 1.15-7.02 mole equivalents, 75-455 nmol), the mixtures vortexed briefly then incubated for 300 minutes at 20° C. with roller-mixing. After incubation (prior to addition of SO1861-EMCH), a ca. 1 mg (0.210 ml) aliquot of Ab-SH was removed from each mixture and purified by gel filtration using a zeba spin desalting column into TBS pH 7.5. These aliquots were characterized by UV-vis analysis and Ellman's assay (thiol to Ab ratio=2.0, 4.2, 5.9 and 6.8 respectively). To each of the bulk Ab-SH was added an aliquot of freshly prepared SO1861-EMCH solution (2 mg/ml, 1.3 mole equivalents per ‘thiol’, 0.15-0.61 μmol, 0.16-0.63 ml), the mixtures vortexed briefly then incubated for 120 minutes at 20° C. Besides each conjugation reaction, two aliquots of desalted Ab-SH (0.25 mg, 1.67 nmol) were reacted with NEM (1.3 mole equivalents per ‘thiol’, 4.3-17.4 nmol, 2.2-8.7 μl of a 0.25 mg/ml solution) or TBS pH 7.5 buffer (2.2-8.7 μl) for 120 minutes at 20° C., as positive and negative controls, respectively. After incubation (prior to addition of NEM), a 0.200 ml aliquot of Ab-SO1861-EMCH mixture was removed and purified by gel filtration using zeba spin desalting column into TBS pH 7.5. This aliquot was characterized by UV-vis and alongside positive and negative controls were characterized by Ellman's assay to obtain SO1861-EMCH incorporations. To the bulk Ab-SO1861-EMCH mixture was added an aliquot of freshly prepared NEM solution (2.5 mg/ml, 2.5-10 mole equivalents, 0.15-0.58 μmol) and the mixtures purified by zeba spin desalting columns eluting with DPBS pH 7.5 to give purified Cetuximab-(L-SO1861) conjugates. The products were normalized to 2.5 mg/ml and filtered to 0.2 μm prior to dispensing for biological evaluation. The reaction conditions and results for Trastuzumab-L-SO1861 conjugates and the reaction conditions and results for Cetuximab-L-SO1861 conjugates are summarized in Table A3 and Table A4.

TABLE A3
Summarized reaction conditions and results
for Trastuzumab-L-SO1861 conjugates

| | | | | | Purity by | |
|---|---|---|---|---|---|---|
| | | TCEP feed | | | analytical | |
| | | mole | SO1861- | Obtained | SEC | |
| Batch | Ab feed | equivalents | EMCH feed | DAR | (%) | Yield (%) |
| Tras-(L- | 9.91 mg | 3.83 | 0.46 μmol | 4.0 | 98.4 | 81 |
| SO1861)₄ | 66 nmol | | | | | |

TABLE A4
Summarized reaction conditions and results for Cetuximab-L-SO1861 conjugates

| | | | | | Purity by | |
|---|---|---|---|---|---|---|
| | | TCEP feed | | | analytical | |
| | | mole | SO1861- | Obtained | SEC | |
| Batch | Ab feed | equivalents | EMCH feed | DAR | (%) | Yield (%) |
| Cet-(L- | 9.73 mg | 4.19 | 0.46 μmol | 4.1 | 99.0 | 77 |
| SO1861)₄ | 65 nmol | | | | | |

### Materials

Throughout the description, claims and drawings, ‘VHH’, ‘Vhh’, ‘Vₕₕ’ and ‘V_{HH}’ should be understood as referring to the same type of single domain antibody. Similar for single domain antibodies of the type referred to as any of ‘V_{H}’, ‘Vh’, ‘Vₕ’ and ‘VH’.

HER2-V_{HH}, EGFR-V_{HH}, CD71-V_{HH} (purchased), Tris(2-carboxyethyl)phosphine hydrochloride (TCEP, 98%, Sigma-Aldrich), 5,5-Dithiobis(2-nitrobenzoic acid) (DTNB, Ellman's reagent, 99%, Sigma-Aldrich), Zeba™ Spin Desalting Columns (2 mL, Thermo-Fisher), NuPAGE™ 4-12% Bis-Tris Protein Gels (Thermo-Fisher), NuPAGE™ MES SDS Running Buffer (Thermo-Fisher), Novex™ Sharp Pre-stained Protein Standard (Thermo-Fisher), PageBlue™ Protein Staining Solution (Thermo-Fischer), Pierce™ BCA Protein Assay Kit (Thermo-Fisher), N-Ethylmaleimide (NEM, 98%, Sigma-Aldrich), 1,4-Dithiothreitol (DTT, 98%, Sigma-Aldrich), Sephadex G25 (GE Healthcare), Sephadex G50 M (GE Healthcare), Superdex 200P (GE Healthcare), Isopropyl alcohol (IPA, 99.6%, VWR), Tris(hydroxymethyl)aminomethane (Tris, 99%, Sigma-Aldrich), Tris(hydroxymethyl)aminomethane hydrochloride (Tris.HCL, Sigma-Aldrich), L-Histidine (99%, Sigma-Aldrich), D-(+)-Trehalose dehydrate (99%, Sigma-Aldrich), Polyethylene glycol sorbitan monolaurate (TWEEN 20, Sigma-Aldrich), Dulbecco's Phosphate-Buffered Saline (DPBS, Thermo-Fisher), Guanidine hydrochloride (99%, Sigma-Aldrich), Ethylenediaminetetraacetic acid disodium salt dihydrate (EDTA-Na₂, 99%, Sigma-Aldrich), sterile filters 0.2 μm and 0.45 μm (Sartorius), Vivaspin T4 and T15 concentrator (Sartorius), Superdex 200PG (GE Healthcare), HSP27 BNA disulfide oligonucleotide (Biosynthesis), [O-(7-Azabenzotriazol-1-yl)-N,N,N,N-tetramethyluronium-hexafluorphosphat](HATU, 97%, Sigma-Aldrich), Dimethyl sulfoxide (DMSO, 99%, Sigma-Aldrich), N-(2-Aminoethyl)maleimide trifluoroacetate salt (AEM, 98%, Sigma-Aldrich), L-Cysteine (98.5%, Sigma-Aldrich), deionized water (DI) was freshly taken from Ultrapure Lab Water Systems (MilliQ, Merck), Nickel-nitrilotriacetic acid agarose (Ni-NTA agarose, Protino), Glycine (99.5%, VWR), 5,5-Dithiobis(2-nitrobenzoic acid (Ellman's reagent, DTNB, 98%, Sigma-Aldrich), Sodium bicarbonate (99.7%, Sigma-Aldrich), Sodium carbonate (99.9%, Sigma-Aldrich), PD MiniTrap desalting columns with Sephadex G-25 resin (GE Healthcare), PD10 G25 desalting column (GE Healthcare), Zeba Spin Desalting Columns in 0.5, 2, 5, and 10 mL (Thermo-Fisher), Vivaspin Centrifugal Filters T4 10 kDa MWCO, T4 100 kDa MWCO, and T15 (Sartorius), Biosep s3000 aSEC column (Phenomenex), Vivacell Ultrafiltration Units 10 and 30 kDa MWCO (Sartorius), Nalgene Rapid-Flow filter (Thermo-Fisher), Acrylamide (99.9%, Sigma-Aldrich), Sodium dodecyl sulfate (98%, Sigma-Aldrich), Ammonium persulfate (APS, 98%, Sigma-Aldrich), Glycerol (99%, Sigma-Aldrich), Bromophenol Blue (Sigma-Aldrich), Polyethylene glycol dodecyl ether (Brij-35, Sigma-Aldrich). All SO1861 derivates (SO1861-EMCH, SO1861-AEM, Dendron-[L-SO1861]n), all QS21 derivates (QS21-EMCH, QS21-AEM, Dendron-[L-QS21]n), and trifunctional linker derivatives were produced in house.

### Syntheses

1. V_{HH}-[S-Trifunctional linker-(L-SO1861)-(L-HSP27 BNA)]₄
HER2-V_{HH}-[S-Tri-(L-SO1861)-(L-HSP27)]₄, HER2-V_{HH}-[S-Tri-(blocked)-(L-HSP27)]₄,
EGFR-V_{HH}-[S-Tri-(L-SO1861)-(L-HSP27)]₄, EGFR-V_{HH}-[S-Tri-(blocked)-(L-HSP27)]₄,
CD71-V_{HH}-[S-Tri-(L-SO1861)-(L-HSP27)]₄, CD71-V_{HH}-[S-Tri-(blocked)-(L-HSP27)]₄,

HER2-V_{HH}, EGFR-V_{HH}, and CD71-V_{HH} are referred hereafter as “Ab”. Ab was conjugated via Michael-type thiol-ene reaction to two different maleimide (Mal) bearing HSP27 BNA derivatives which are referred hereafter as “HSP27-Mal”. These HSP27-Mal derivatives were namely: 1) Mal-Trifunctional linker-(L-SO1861)-(L-HSP27 BNA), 2) Mal-Trifunctional linker-(blocked)-(L-HSP27 BNA). The procedure is exemplary described for HER2-V_{HH}-[S-Trifunctional linker-(L-SO1861)-(L-HSP27 BNA)]₄:

Ab was reconstituted to 21 mg/ml with deionized water (DI), then diluted to 5 mg/ml using histidine buffer pH 6. To a 20 mg (4.0 ml) aliquot was added 10 μl/ml each of Tris concentrate (127 mg/ml, 1.05M), Tris.HCl concentrate (623 mg/ml, 3.95 M) and EDTA-Na₂ concentrate (95 mg/ml, 0.26 M) to give a 50 mM TBS, 2.5 mM EDTA buffer pH 7.5.

To Ab (2.1 mg, 0.5 mg/ml, 0.14 μmol) was added an aliquot of freshly prepared TCEP solution (1.00 mg/ml, 2.35 mole equivalents, 0.32 μmol), the mixture vortexed briefly then incubated for 90 minutes at 20° C. with roller-mixing. After incubation (prior to addition of construct), a ca. 0.2 mg (0.044 ml) aliquot of Ab-SH was removed from each mixture and purified by gel filtration using a zeba spin desalting column into TBS pH 7.5. These aliquots were characterized by UV-vis analysis and Ellman's assay (thiol to ab ratio=4.0). The bulk Ab-SH was split into two aliquots (0.11 mg, 7.6 nmol and 0.12 mg, 8.3 nmol), and to each aliquot was added an aliquot of each of the HSP27 BNA-Mal derivatives 1-2 (freshly prepared in TBS pH 7.5, 2 mg/ml, 1.3 mole equivalents per ‘thiol’, 40 nmol and 43 nmol), the mixtures vortexed briefly then incubated for 120 minutes at 20° C. Besides the Ab-HSP27 BNA derivatives 2 conjugation reaction, two aliquots of desalted Ab-SH (50 μg, 3.3 nmol) were reacted with NEM (1.3 mole equivalents per ‘thiol’, 17.3 nmol, 6.7 μl of a 0.25 mg/ml solution) or TBS pH 7.5 buffer (6.7 μl) for 120 minutes at 20° C., as positive and negative controls, respectively. After incubation (prior to addition of NEM), a 0.100 ml aliquot of Ab-construct 2 mixture was removed and purified by gel filtration using zeba spin desalting column into TBS pH 7.5. This aliquot was characterized by UV-vis and alongside positive and negative controls was characterized by Ellman's assay to obtain HSP27 BNA derivatives 2 incorporation. To each bulk Ab-construct mixture was added an aliquot of freshly prepared NEM solution (0.25 mg/ml, 2.5 mole equivalents, 19 and 21 nmol) and the mixtures purified by gel filtration using a 1.6×30 cm Sephadex G50M eluting with DPBS pH 7.5 followed by repeated centrifugal filtration and washing using a 100 kDa MWCO concentrator to give purified Ab-construct 1-2 conjugates. The products were filtered to 0.2 μm prior to dispensing for biological evaluation.

2. V_{HH}-[S-Trifunctional linker-(S-SO1861)-(L-HSP27 BNA)]₄
HER2-V_{HH}-[S-Tri-(S-SO1861)-(L-HSP27)]₄, HER2-V_{HH}-[S-Tri-(blocked)-(L-HSP27)]₄,
EGFR-V_{HH}-[S-Tri-(S-SO1861)-(L-HSP27)]₄, EGFR-V_{HH}-[S-Tri-(blocked)-(L-HSP27)]₄,
CD71-V_{HH}-[S-Tri-(S-SO1861)-(L-HSP27)]₄, CD71-V_{HH}-[S-Tri-(blocked)-(L-HSP27)]₄,

HER2-V_{HH}, EGFR-V_{HH}, and CD71-V_{HH} are referred hereafter as “Ab”. Ab was conjugated via Michael-type thiol-ene reaction to two different maleimide (Mal) bearing HSP27 BNA derivatives which are referred hereafter as “HSP27-Mal”. These HSP27-Mal derivatives were namely: 1) Mal-Trifunctional linker-(S-SO1861)-(L-HSP27 BNA), 2) Mal-Trifunctional linker-(blocked)-(L-HSP27 BNA). The procedure is exemplary described for HER2-V_{HH}-[S-Trifunctional linker-(S-SO1861)-(L-HSP27 BNA)]₄:

Ab was reconstituted to 21 mg/ml with deionized water (DI), then diluted to 5 mg/ml using histidine buffer pH 6. To a 20 mg (4.0 ml) aliquot was added 10 μl/ml each of Tris concentrate (127 mg/ml, 1.05 M), Tris.HCl concentrate (623 mg/ml, 3.95 M) and EDTA-Na₂ concentrate (95 mg/ml, 0.26 M) to give a 50 mM TBS, 2.5 mM EDTA buffer pH 7.5.

To Ab (2.1 mg, 0.5 mg/ml, 0.14 μmol) was added an aliquot of freshly prepared TCEP solution (1.00 mg/ml, 2.35 mole equivalents, 0.32 μmol), the mixture vortexed briefly then incubated for 90 minutes at 20° C. with roller-mixing. After incubation (prior to addition of construct), a ca. 0.2 mg (0.044 ml) aliquot of Ab-SH was removed from each mixture and purified by gel filtration using a zeba spin desalting column into TBS pH 7.5. These aliquots were characterized by UV-vis analysis and Ellman's assay (thiol to ab ratio=4.0). The bulk Ab-SH was split into two aliquots (0.11 mg, 7.6 nmol and 0.12 mg, 8.3 nmol), and to each aliquot was added an aliquot of each of the HSP27 BNA-Mal derivatives 1-2 (freshly prepared in TBS pH 7.5, 2 mg/ml, 1.3 mole equivalents per ‘thiol’, 40 nmol and 43 nmol), the mixtures vortexed briefly then incubated for 120 minutes at 20° C. Besides the Ab-HSP27 BNA derivatives 2 conjugation reaction, two aliquots of desalted Ab-SH (50 μg, 3.3 nmol) were reacted with NEM (1.3 mole equivalents per ‘thiol’, 17.3 nmol, 6.7 μl of a 0.25 mg/ml solution) or TBS pH 7.5 buffer (6.7 μl) for 120 minutes at 20° C., as positive and negative controls, respectively. After incubation (prior to addition of NEM), a 0.100 ml aliquot of Ab-construct 2 mixture was removed and purified by gel filtration using zeba spin desalting column into TBS pH 7.5. This aliquot was characterized by UV-vis and alongside positive and negative controls was characterized by Ellman's assay to obtain HSP27 BNA derivatives 2 incorporation. To each bulk Ab-construct mixture was added an aliquot of freshly prepared NEM solution (0.25 mg/ml, 2.5 mole equivalents, 19 and 21 nmol) and the mixtures purified by gel filtration using a 1.6×30 cm Sephadex G50M eluting with DPBS pH 7.5 followed by repeated centrifugal filtration and washing using a 100 kDa MWCO concentrator to give purified Ab-construct 1-2 conjugates. The products were filtered to 0.2 μm prior to dispensing for biological evaluation.

3. V_{HH}-[S-Trifunctional linker-(S-dendron-(L-SO1861)ₙ)-(L-HSP27 BNA)]₄
HER2-V_{HH}-[S-Tri-(S-dendron-(L-SO1861)n)-(L-HSP27)]₄, HER2-V_{HH}-[S-Tri-(blocked)-(L-HSP27)]₄,
EGFR-V_{HH}-[S-Tri-(S-dendron-(L-SO1861)n)-(L-HSP27)]₄, EGFR-V_{HH}-[S-Tri-(blocked)-(L-HSP27)]₄,
CD71-V_{HH}-[S-Tri-(S-dendron-(L-SO1861)n)-(L-HSP27)]₄, CD71-V_{HH}-[S-Tri-(blocked)-(L-HSP27)]₄,

HER2-V_{HH}, EGFR-V_{HH}, and CD71-V_{HH} are referred hereafter as “Ab”. Ab was conjugated via Michael-type thiol-ene reaction to two different maleimide (Mal) bearing HSP27 BNA derivatives which are referred hereafter as “HSP27-Mal”. These HSP27-Mal derivatives were namely: 1) Mal-Trifunctional linker-(S-dendron-(L-SO1861)n)-(L-HSP27 BNA), 2) Mal-Trifunctional linker-(blocked)-(L-HSP27 BNA). “n” refers to the number of SO1861 molecules that is 4, 8, or higher than 8. The procedure is exemplary described for HER2-V_{HH}-[S-Trifunctional linker-(S-dendron-(L-SO1861)4)-(L-HSP27 BNA)]₄:

Ab was reconstituted to 21 mg/ml with deionized water (DI), then diluted to 5 mg/ml using histidine buffer pH 6. To a 20 mg (4.0 ml) aliquot was added 10 μl/ml each of Tris concentrate (127 mg/ml, 1.05M), Tris.HCl concentrate (623 mg/ml, 3.95 M) and EDTA-Na₂ concentrate (95 mg/ml, 0.26 M) to give a 50 mM TBS, 2.5 mM EDTA buffer pH 7.5.

To Ab (2.1 mg, 0.5 mg/ml, 0.14 μmol) was added an aliquot of freshly prepared TCEP solution (1.00 mg/ml, 2.35 mole equivalents, 0.32 μmol), the mixture vortexed briefly then incubated for 90 minutes at 20° C. with roller-mixing. After incubation (prior to addition of construct), a ca. 0.2 mg (0.044 ml) aliquot of Ab-SH was removed from each mixture and purified by gel filtration using a zeba spin desalting column into TBS pH 7.5. These aliquots were characterized by UV-vis analysis and Ellman's assay (thiol to ab ratio=4.0). The bulk Ab-SH was split into two aliquots (0.11 mg, 7.6 nmol and 0.12 mg, 8.3 nmol), and to each aliquot was added an aliquot of each of the HSP27 BNA-Mal derivatives 1-2 (freshly prepared in TBS pH 7.5, 2 mg/ml, 1.3 mole equivalents per ‘thiol’, 40 nmol and 43 nmol), the mixtures vortexed briefly then incubated for 120 minutes at 20° C. Besides the Ab-HSP27 BNA derivatives 2 conjugation reaction, two aliquots of desalted Ab-SH (50 μg, 3.3 nmol) were reacted with NEM (1.3 mole equivalents per ‘thiol’, 17.3 nmol, 6.7 μl of a 0.25 mg/ml solution) or TBS pH 7.5 buffer (6.7 μl) for 120 minutes at 20° C., as positive and negative controls, respectively. After incubation (prior to addition of NEM), a 0.100 ml aliquot of Ab-construct 2 mixture was removed and purified by gel filtration using zeba spin desalting column into TBS pH 7.5. This aliquot was characterized by UV-vis and alongside positive and negative controls was characterized by Ellman's assay to obtain HSP27 BNA derivatives 2 incorporation. To each bulk Ab-construct mixture was added an aliquot of freshly prepared NEM solution (0.25 mg/ml, 2.5 mole equivalents, 19 and 21 nmol) and the mixtures purified by gel filtration using a 1.6×30 cm Sephadex G50M eluting with DPBS pH 7.5 followed by repeated centrifugal filtration and washing using a 100 kDa MWCO concentrator to give purified Ab-construct 1-2 conjugates. The products were filtered to 0.2 μm prior to dispensing for biological evaluation.

4. V_{HH}-[S-Trifunctional linker-(L-QS21)-(L-HSP27 BNA)]₄
HER2-V_{HH}-[S-Tri-(L-QS21)-(L-HSP27)]₄, HER2-V_{HH}-[S-Tri-(blocked)-(L-HSP27)]₄,
EGFR-V_{HH}-[S-Tri-(L-QS21)-(L-HSP27)]₄, EGFR-V_{HH}-[S-Tri-(blocked)-(L-HSP27)]₄,
CD71-V_{HH}-[S-Tri-(L-QS21)-(L-HSP27)]₄, CD71-V_{HH}-[S-Tri-(blocked)-(L-HSP27)]₄,

HER2-V_{HH}, EGFR-V_{HH}, and CD71-V_{HH} are referred hereafter as “Ab”. Ab was conjugated via Michael-type thiol-ene reaction to two different maleimide (Mal) bearing HSP27 BNA derivatives which are referred hereafter as “HSP27-Mal”. These HSP27-Mal derivatives were namely: 1) Mal-Trifunctional linker-(L-QS21)-(L-HSP27 BNA), 2) Mal-Trifunctional linker-(blocked)-(L-HSP27 BNA). The procedure is exemplary described for HER2-V_{HH}-[S-Trifunctional linker-(L-QS21)-(L-HSP27 BNA)]₄:

Ab was reconstituted to 21 mg/ml with deionized water (DI), then diluted to 5 mg/ml using histidine buffer pH 6. To a 20 mg (4.0 ml) aliquot was added 10 μl/ml each of Tris concentrate (127 mg/ml, 1.05 M), Tris.HCl concentrate (623 mg/ml, 3.95 M) and EDTA-Na₂ concentrate (95 mg/ml, 0.26 M) to give a 50 mM TBS, 2.5 mM EDTA buffer pH 7.5.

To Ab (2.1 mg, 0.5 mg/ml, 0.14 μmol) was added an aliquot of freshly prepared TCEP solution (1.00 mg/ml, 2.35 mole equivalents, 0.32 μmol), the mixture vortexed briefly then incubated for 90 minutes at 20° C. with roller-mixing. After incubation (prior to addition of construct), a ca. 0.2 mg (0.044 ml) aliquot of Ab-SH was removed from each mixture and purified by gel filtration using a zeba spin desalting column into TBS pH 7.5. These aliquots were characterized by UV-vis analysis and Ellman's assay (thiol to ab ratio=4.0). The bulk Ab-SH was split into two aliquots (0.11 mg, 7.6 nmol and 0.12 mg, 8.3 nmol), and to each aliquot was added an aliquot of each of the HSP27 BNA-Mal derivatives 1-2 (freshly prepared in TBS pH 7.5, 2 mg/ml, 1.3 mole equivalents per ‘thiol’, 40 nmol and 43 nmol), the mixtures vortexed briefly then incubated for 120 minutes at 20° C. Besides the Ab-HSP27 BNA derivatives 2 conjugation reaction, two aliquots of desalted Ab-SH (50 μg, 3.3 nmol) were reacted with NEM (1.3 mole equivalents per ‘thiol’, 17.3 nmol, 6.7 μl of a 0.25 mg/ml solution) or TBS pH 7.5 buffer (6.7 μl) for 120 minutes at 20° C., as positive and negative controls, respectively. After incubation (prior to addition of NEM), a 0.100 ml aliquot of Ab-construct 2 mixture was removed and purified by gel filtration using zeba spin desalting column into TBS pH 7.5. This aliquot was characterized by UV-vis and alongside positive and negative controls was characterized by Ellman's assay to obtain HSP27 BNA derivatives 2 incorporation. To each bulk Ab-construct mixture was added an aliquot of freshly prepared NEM solution (0.25 mg/ml, 2.5 mole equivalents, 19 and 21 nmol) and the mixtures purified by gel filtration using a 1.6×30 cm Sephadex G50M eluting with DPBS pH 7.5 followed by repeated centrifugal filtration and washing using a 100 kDa MWCO concentrator to give purified Ab-construct 1-2 conjugates. The products were filtered to 0.2 μm prior to dispensing for biological evaluation.

5. V_{HH}-[S-Trifunctional linker-(S-QS21)-(L-HSP27 BNA)]₄
HER2-V_{HH}-[S-Tri-(S-QS21)-(L-HSP27)]₄, HER2-V_{HH}-[S-Tri-(blocked)-(L-HSP27)]₄,
EGFR-V_{HH}-[S-Tri-(S-QS21)-(L-HSP27)]₄, EGFR-V_{HH}-[S-Tri-(blocked)-(L-HSP27)]₄,
CD71-V_{HH}-[S-Tri-(S-QS21)-(L-HSP27)]₄, CD71-V_{HH}-[S-Tri-(blocked)-(L-HSP27)]₄,

HER2-V_{HH}, EGFR-V_{HH}, and CD71-V_{HH} are referred hereafter as “Ab”. Ab was conjugated via Michael-type thiol-ene reaction to two different maleimide (Mal) bearing HSP27 BNA derivatives which are referred hereafter as “HSP27-Mal”. These HSP27-Mal derivatives were namely: 1) Mal-Trifunctional linker-(S-QS21)-(L-HSP27 BNA), 2) Mal-Trifunctional linker-(blocked)-(L-HSP27 BNA). The procedure is exemplary described for HER2-V_{HH}-[S-Trifunctional linker-(S-QS21)-(L-HSP27 BNA)]₄:

Ab was reconstituted to 21 mg/ml with deionized water (DI), then diluted to 5 mg/ml using histidine buffer pH 6. To a 20 mg (4.0 ml) aliquot was added 10 μl/ml each of Tris concentrate (127 mg/ml, 1.05 M), Tris.HCl concentrate (623 mg/ml, 3.95M) and EDTA-Na₂ concentrate (95 mg/ml, 0.26 M) to give a 50 mM TBS, 2.5 mM EDTA buffer pH 7.5.

To Ab (2.1 mg, 0.5 mg/ml, 0.14 μmol) was added an aliquot of freshly prepared TCEP solution (1.00 mg/ml, 2.35 mole equivalents, 0.32 μmol), the mixture vortexed briefly then incubated for 90 minutes at 20° C. with roller-mixing. After incubation (prior to addition of construct), a ca. 0.2 mg (0.044 ml) aliquot of Ab-SH was removed from each mixture and purified by gel filtration using a zeba spin desalting column into TBS pH 7.5. These aliquots were characterized by UV-vis analysis and Ellman's assay (thiol to ab ratio=4.0). The bulk Ab-SH was split into two aliquots (0.11 mg, 7.6 nmol and 0.12 mg, 8.3 nmol), and to each aliquot was added an aliquot of each of the HSP27 BNA-Mal derivatives 1-2 (freshly prepared in TBS pH 7.5, 2 mg/ml, 1.3 mole equivalents per ‘thiol’, 40 nmol and 43 nmol), the mixtures vortexed briefly then incubated for 120 minutes at 20° C. Besides the Ab-HSP27 BNA derivatives 2 conjugation reaction, two aliquots of desalted Ab-SH (50 μg, 3.3 nmol) were reacted with NEM (1.3 mole equivalents per ‘thiol’, 17.3 nmol, 6.7 μl of a 0.25 mg/ml solution) or TBS pH 7.5 buffer (6.7 μl) for 120 minutes at 20° C., as positive and negative controls, respectively. After incubation (prior to addition of NEM), a 0.100 ml aliquot of Ab-construct 2 mixture was removed and purified by gel filtration using zeba spin desalting column into TBS pH 7.5. This aliquot was characterized by UV-vis and alongside positive and negative controls was characterized by Ellman's assay to obtain HSP27 BNA derivatives 2 incorporation. To each bulk Ab-construct mixture was added an aliquot of freshly prepared NEM solution (0.25 mg/ml, 2.5 mole equivalents, 19 and 21 nmol) and the mixtures purified by gel filtration using a 1.6×30 cm Sephadex G50M eluting with DPBS pH 7.5 followed by repeated centrifugal filtration and washing using a 100 kDa MWCO concentrator to give purified Ab-construct 1-2 conjugates. The products were filtered to 0.2 μm prior to dispensing for biological evaluation.

6. V_{HH}-[S-Trifunctional linker-(S-Dendron-(L-QS21)n)-(L-HSP27 BNA)]₄
HER2-V_{HH}-[S-Tri-(S-dendron-(L-QS21)n)-(L-HSP27)]₄, HER2-V_{HH}-[S-Tri-(blocked)-(L-HSP27)]₄,
EGFR-V_{HH}-[S-Tri-(S-dendron-(L-QS21)n)-(L-HSP27)]₄, EGFR-V_{HH}-[S-Tri-(blocked)-(L-HSP27)]₄,
CD71-V_{HH}-[S-Tri-(S-dendron-(L-QS21)n)-(L-HSP27)]₄, CD71-V_{HH}-[S-Tri-(blocked)-(L-HSP27)]₄,

HER2-V_{HH}, EGFR-V_{HH}, and CD71-V_{HH} are referred hereafter as “Ab”. Ab was conjugated via Michael-type thiol-ene reaction to two different maleimide (Mal) bearing HSP27 BNA derivatives which are referred hereafter as “HSP27-Mal”. These HSP27-Mal derivatives were namely: 1) Mal-Trifunctional linker-(S-dendron-(L-QS21)n)-(L-HSP27 BNA), 2) Mal-Trifunctional linker-(blocked)-(L-HSP27 BNA). “n” refers to the number of QS21 molecules that is 4, 8, or higher than 8. The procedure is exemplary described for HER2-V_{HH}-[S-Trifunctional linker-(S-dendron-(L-QS21)4)-(L-HSP27 BNA)]₄:

Ab was reconstituted to 21 mg/ml with deionized water (DI), then diluted to 5 mg/ml using histidine buffer pH 6. To a 20 mg (4.0 ml) aliquot was added 10 μl/ml each of Tris concentrate (127 mg/ml, 1.05 M), Tris.HCl concentrate (623 mg/ml, 3.95M) and EDTA-Na₂ concentrate (95 mg/ml, 0.26 M) to give a 50 mM TBS, 2.5 mM EDTA buffer pH 7.5.

To Ab (2.1 mg, 0.5 mg/ml, 0.14 μmol) was added an aliquot of freshly prepared TCEP solution (1.00 mg/ml, 2.35 mole equivalents, 0.32 μmol), the mixture vortexed briefly then incubated for 90 minutes at 20° C. with roller-mixing. After incubation (prior to addition of construct), a ca. 0.2 mg (0.044 ml) aliquot of Ab-SH was removed from each mixture and purified by gel filtration using a zeba spin desalting column into TBS pH 7.5. These aliquots were characterized by UV-vis analysis and Ellman's assay (thiol to ab ratio=4.0). The bulk Ab-SH was split into two aliquots (0.11 mg, 7.6 nmol and 0.12 mg, 8.3 nmol), and to each aliquot was added an aliquot of each of the HSP27 BNA-Mal derivatives 1-2 (freshly prepared in TBS pH 7.5, 2 mg/ml, 1.3 mole equivalents per ‘thiol’, 40 nmol and 43 nmol), the mixtures vortexed briefly then incubated for 120 minutes at 20° C. Besides the Ab-HSP27 BNA derivatives 2 conjugation reaction, two aliquots of desalted Ab-SH (50 μg, 3.3 nmol) were reacted with NEM (1.3 mole equivalents per ‘thiol’, 17.3 nmol, 6.7 μl of a 0.25 mg/ml solution) or TBS pH 7.5 buffer (6.7 μl) for 120 minutes at 20° C., as positive and negative controls, respectively. After incubation (prior to addition of NEM), a 0.100 ml aliquot of Ab-construct 2 mixture was removed and purified by gel filtration using zeba spin desalting column into TBS pH 7.5. This aliquot was characterized by UV-vis and alongside positive and negative controls was characterized by Ellman's assay to obtain HSP27 BNA derivatives 2 incorporation. To each bulk Ab-construct mixture was added an aliquot of freshly prepared NEM solution (0.25 mg/ml, 2.5 mole equivalents, 19 and 21 nmol) and the mixtures purified by gel filtration using a 1.6×30 cm Sephadex G50M eluting with DPBS pH 7.5 followed by repeated centrifugal filtration and washing using a 100 kDa MWCO concentrator to give purified Ab-construct 1-2 conjugates. The products were filtered to 0.2 μm prior to dispensing for biological evaluation.

7. V_{HH}-[S-Trifunctional linker-(L-SO1861)-(L-dianthin)]₄
HER2-V_{HH}-[S-Tri-(L-SO1861)-(L-dianthin)]₄, HER2-V_{HH}-[S-Tri-(blocked)-(L-dianthin)]₄,
EGFR-V_{HH}-[S-Tri-(L-SO1861)-(L-dianthin)]₄, EGFR-V_{HH}-[S-Tri-(blocked)-(L-dianthin)]₄,
CD71-V_{HH}-[S-Tri-(L-SO1861)-(L-dianthin)]₄, CD71-V_{HH}-[S-Tri-(blocked)-(L-dianthin)]₄,

HER2-V_{HH}, EGFR-V_{HH}, and CD71-V_{HH} are referred hereafter as “Ab”. Ab was conjugated via Michael-type thiol-ene reaction to two different maleimide (Mal) bearing dianthin derivatives which are referred hereafter as “dianthin -Mal”. These dianthin -Mal derivatives were namely: 1) Mal-Trifunctional linker-(L-SO1861)-(L-dianthin), 2) Mal-Trifunctional linker-(blocked)-(L-dianthin). The procedure is exemplary described for HER2-V_{HH}-[S-Trifunctional linker-(L-SO1861)-(L-dianthin)]₄:

Ab was reconstituted to 21 mg/ml with deionized water (DI), then diluted to 5 mg/ml using histidine buffer pH 6. To a 20 mg (4.0 ml) aliquot was added 10 μl/ml each of Tris concentrate (127 mg/ml, 1.05 M), Tris.HCl concentrate (623 mg/ml, 3.95 M) and EDTA-Na₂ concentrate (95 mg/ml, 0.26 M) to give a 50 mM TBS, 2.5 mM EDTA buffer pH 7.5.

To Ab (2.1 mg, 0.5 mg/ml, 0.14 μmol) was added an aliquot of freshly prepared TCEP solution (1.00 mg/ml, 2.35 mole equivalents, 0.32 μmol), the mixture vortexed briefly then incubated for 90 minutes at 20° C. with roller-mixing. After incubation (prior to addition of construct), a ca. 0.2 mg (0.044 ml) aliquot of Ab-SH was removed from each mixture and purified by gel filtration using a zeba spin desalting column into TBS pH 7.5. These aliquots were characterized by UV-vis analysis and Ellman's assay (thiol to ab ratio=4.0). The bulk Ab-SH was split into two aliquots (0.11 mg, 7.6 nmol and 0.12 mg, 8.3 nmol), and to each aliquot was added an aliquot of each of the dianthin -Mal derivatives 1-2 (freshly prepared in TBS pH 7.5, 2 mg/ml, 1.3 mole equivalents per ‘thiol’, 40 nmol and 43 nmol), the mixtures vortexed briefly then incubated for 120 minutes at 20° C. Besides the Ab-dianthin derivatives 2 conjugation reaction, two aliquots of desalted Ab-SH (50 μg, 3.3 nmol) were reacted with NEM (1.3 mole equivalents per ‘thiol’, 17.3 nmol, 6.7 μl of a 0.25 mg/ml solution) or TBS pH 7.5 buffer (6.7 μl) for 120 minutes at 20° C., as positive and negative controls, respectively. After incubation (prior to addition of NEM), a 0.100 ml aliquot of Ab-construct 2 mixture was removed and purified by gel filtration using zeba spin desalting column into TBS pH 7.5. This aliquot was characterized by UV-vis and alongside positive and negative controls was characterized by Ellman's assay to obtain dianthin derivatives 2 incorporation. To each bulk Ab-construct mixture was added an aliquot of freshly prepared NEM solution (0.25 mg/ml, 2.5 mole equivalents, 19 and 21 nmol) and the mixtures purified by gel filtration using a 1.6×30 cm Sephadex G50M eluting with DPBS pH 7.5 followed by repeated centrifugal filtration and washing using a 100 KDa MWCO concentrator to give purified Ab-construct 1-2 conjugates. The products were filtered to 0.2 μm prior to dispensing for biological evaluation.

8. V_{HH}-[S-Trifunctional linker-(S-SO1861)-(L-dianthin)]₄
HER2-V_{HH}-[S-Tri-(S-SO1861)-(L-dianthin)]₄, HER2-V_{HH}-[S-Tri-(blocked)-(L-dianthin)]₄,
EGFR-V_{HH}-[S-Tri-(S-SO1861)-(L-dianthin)]₄, EGFR-V_{HH}-[S-Tri-(blocked)-(L-dianthin)]₄,
CD71-V_{HH}-[S-Tri-(S-SO1861)-(L-dianthin)]₄, CD71-V_{HH}-[S-Tri-(blocked)-(L-dianthin)]₄,

HER2-V_{HH}, EGFR-V_{HH}, and CD71-V_{HH} are referred hereafter as “Ab”. Ab was conjugated via Michael-type thiol-ene reaction to two different maleimide (Mal) bearing dianthin derivatives which are referred hereafter as “dianthin -Mal”. These dianthin -Mal derivatives were namely: 1) Mal-Trifunctional linker-(S-SO1861)-(L-dianthin), 2) Mal-Trifunctional linker-(blocked)-(L-dianthin). The procedure is exemplary described for HER2-V_{HH}-[S-Trifunctional linker-(S-SO1861)-(L-dianthin)]₄:

Ab was reconstituted to 21 mg/ml with deionized water (DI), then diluted to 5 mg/ml using histidine buffer pH 6. To a 20 mg (4.0 ml) aliquot was added 10 μl/ml each of Tris concentrate (127 mg/ml, 1.05 M), Tris.HCl concentrate (623 mg/ml, 3.95M) and EDTA-Na₂ concentrate (95 mg/ml, 0.26 M) to give a 50 mM TBS, 2.5 mM EDTA buffer pH 7.5.

To Ab (2.1 mg, 0.5 mg/ml, 0.14 μmol) was added an aliquot of freshly prepared TCEP solution (1.00 mg/ml, 2.35 mole equivalents, 0.32 μmol), the mixture vortexed briefly then incubated for 90 minutes at 20° C. with roller-mixing. After incubation (prior to addition of construct), a ca. 0.2 mg (0.044 ml) aliquot of Ab-SH was removed from each mixture and purified by gel filtration using a zeba spin desalting column into TBS pH 7.5. These aliquots were characterized by UV-vis analysis and Ellman's assay (thiol to ab ratio=4.0). The bulk Ab-SH was split into two aliquots (0.11 mg, 7.6 nmol and 0.12 mg, 8.3 nmol), and to each aliquot was added an aliquot of each of the dianthin -Mal derivatives 1-2 (freshly prepared in TBS pH 7.5, 2 mg/ml, 1.3 mole equivalents per ‘thiol’, 40 nmol and 43 nmol), the mixtures vortexed briefly then incubated for 120 minutes at 20° C. Besides the Ab-dianthin derivatives 2 conjugation reaction, two aliquots of desalted Ab-SH (50 μg, 3.3 nmol) were reacted with NEM (1.3 mole equivalents per ‘thiol’, 17.3 nmol, 6.7 μl of a 0.25 mg/ml solution) or TBS pH 7.5 buffer (6.7 μl) for 120 minutes at 20° C., as positive and negative controls, respectively. After incubation (prior to addition of NEM), a 0.100 ml aliquot of Ab-construct 2 mixture was removed and purified by gel filtration using zeba spin desalting column into TBS pH 7.5. This aliquot was characterized by UV-vis and alongside positive and negative controls was characterized by Ellman's assay to obtain dianthin derivatives 2 incorporation. To each bulk Ab-construct mixture was added an aliquot of freshly prepared NEM solution (0.25 mg/ml, 2.5 mole equivalents, 19 and 21 nmol) and the mixtures purified by gel filtration using a 1.6×30 cm Sephadex G50M eluting with DPBS pH 7.5 followed by repeated centrifugal filtration and washing using a 100 kDa MWCO concentrator to give purified Ab-construct 1-2 conjugates. The products were filtered to 0.2 μm prior to dispensing for biological evaluation.

9. V_{HH}-[S-Trifunctional linker-(S-dendron-(L-SO1861)ₙ)-(L-dianthin)]₄
HER2-V_{HH}-[S-Tri-(S-dendron-(L-SO1861)n)-(L-dianthin)]₄, HER2-V_{HH}-[S-Tri-(blocked)-(L-dianthin)]₄,
EGFR-V_{HH}-[S-Tri-(S-dendron-(L-SO1861)n)-(L-dianthin)]₄, EGFR-V_{HH}-[S-Tri-(blocked)-(L-dianthin)]₄,
CD71-V_{HH}-[S-Tri-(S-dendron-(L-SO1861)n)-(L-dianthin)]₄, CD71-V_{HH}-[S-Tri-(blocked)-(L-dianthin)]₄,

HER2-V_{HH}, EGFR-V_{HH}, and CD71-V_{HH} are referred hereafter as “Ab”. Ab was conjugated via Michael-type thiol-ene reaction to two different maleimide (Mal) bearing dianthin derivatives which are referred hereafter as “dianthin -Mal”. These dianthin -Mal derivatives were namely: 1) Mal-Trifunctional linker-(S-dendron-(L-SO1861)n)-(L-dianthin), 2) Mal-Trifunctional linker-(blocked)-(L-dianthin). “n” refers to the number of SO1861 molecules that is 4, 8, or higher than 8. The procedure is exemplary described for HER2-V_{HH}-[S-Trifunctional linker-(S-dendron-(L-SO1861)4)-(L-dianthin)]₄:

Ab was reconstituted to 21 mg/ml with deionized water (DI), then diluted to 5 mg/ml using histidine buffer pH 6. To a 20 mg (4.0 ml) aliquot was added 10 μl/ml each of Tris concentrate (127 mg/ml, 1.05 M), Tris.HCl concentrate (623 mg/ml, 3.95 M) and EDTA-Na₂ concentrate (95 mg/ml, 0.26 M) to give a 50 mM TBS, 2.5 mM EDTA buffer pH 7.5.

To Ab (2.1 mg, 0.5 mg/ml, 0.14 μmol) was added an aliquot of freshly prepared TCEP solution (1.00 mg/ml, 2.35 mole equivalents, 0.32 μmol), the mixture vortexed briefly then incubated for 90 minutes at 20° C. with roller-mixing. After incubation (prior to addition of construct), a ca. 0.2 mg (0.044 ml) aliquot of Ab-SH was removed from each mixture and purified by gel filtration using a zeba spin desalting column into TBS pH 7.5. These aliquots were characterized by UV-vis analysis and Ellman's assay (thiol to ab ratio=4.0). The bulk Ab-SH was split into two aliquots (0.11 mg, 7.6 nmol and 0.12 mg, 8.3 nmol), and to each aliquot was added an aliquot of each of the dianthin -Mal derivatives 1-2 (freshly prepared in TBS pH 7.5, 2 mg/ml, 1.3 mole equivalents per ‘thiol’, 40 nmol and 43 nmol), the mixtures vortexed briefly then incubated for 120 minutes at 20° C. Besides the Ab-dianthin derivatives 2 conjugation reaction, two aliquots of desalted Ab-SH (50 μg, 3.3 nmol) were reacted with NEM (1.3 mole equivalents per ‘thiol’, 17.3 nmol, 6.7 μl of a 0.25 mg/ml solution) or TBS pH 7.5 buffer (6.7 μl) for 120 minutes at 20° C., as positive and negative controls, respectively. After incubation (prior to addition of NEM), a 0.100 ml aliquot of Ab-construct 2 mixture was removed and purified by gel filtration using zeba spin desalting column into TBS pH 7.5. This aliquot was characterized by UV-vis and alongside positive and negative controls was characterized by Ellman's assay to obtain dianthin derivatives 2 incorporation. To each bulk Ab-construct mixture was added an aliquot of freshly prepared NEM solution (0.25 mg/ml, 2.5 mole equivalents, 19 and 21 nmol) and the mixtures purified by gel filtration using a 1.6×30 cm Sephadex G50M eluting with DPBS pH 7.5 followed by repeated centrifugal filtration and washing using a 100 kDa MWCO concentrator to give purified Ab-construct 1-2 conjugates. The products were filtered to 0.2 μm prior to dispensing for biological evaluation.

10. V_{HH}-[S-Trifunctional linker-(L-QS21)-(L-dianthin)]₄
HER2-V_{HH}-[S-Tri-(L-QS21)-(L-dianthin)]₄, HER2-V_{HH}-[S-Tri-(blocked)-(L-dianthin)]₄,
EGFR-V_{HH}-[S-Tri-(L-QS21)-(L-dianthin)]₄, EGFR-V_{HH}-[S-Tri-(blocked)-(L-dianthin)]₄,
CD71-V_{HH}-[S-Tri-(L-QS21)-(L-dianthin)]₄, CD71-V_{HH}-[S-Tri-(blocked)-(L-dianthin)]₄,

HER2-V_{HH}, EGFR-V_{HH}, and CD71-V_{HH} are referred hereafter as “Ab”. Ab was conjugated via Michael-type thiol-ene reaction to two different maleimide (Mal) bearing dianthin derivatives which are referred hereafter as “dianthin Mal”. These dianthin -Mal derivatives were namely: 1) Mal-Trifunctional linker-(L-QS21)-(L-dianthin), 2) Mal-Trifunctional linker-(blocked)-(L-dianthin). The procedure is exemplary described for HER2-V_{HH}-[S-Trifunctional linker-(L-QS21)-(L-dianthin)]₄:

Ab was reconstituted to 21 mg/ml with deionized water (DI), then diluted to 5 mg/ml using histidine buffer pH 6. To a 20 mg (4.0 ml) aliquot was added 10 μl/ml each of Tris concentrate (127 mg/ml, 1.05 M), Tris.HCl concentrate (623 mg/ml, 3.95 M) and EDTA-Na₂ concentrate (95 mg/ml, 0.26 M) to give a 50 mM TBS, 2.5 mM EDTA buffer pH 7.5.

To Ab (2.1 mg, 0.5 mg/ml, 0.14 μmol) was added an aliquot of freshly prepared TCEP solution (1.00 mg/ml, 2.35 mole equivalents, 0.32 μmol), the mixture vortexed briefly then incubated for 90 minutes at 20° C. with roller-mixing. After incubation (prior to addition of construct), a ca. 0.2 mg (0.044 ml) aliquot of Ab-SH was removed from each mixture and purified by gel filtration using a zeba spin desalting column into TBS pH 7.5. These aliquots were characterized by UV-vis analysis and Ellman's assay (thiol to ab ratio=4.0). The bulk Ab-SH was split into two aliquots (0.11 mg, 7.6 nmol and 0.12 mg, 8.3 nmol), and to each aliquot was added an aliquot of each of the dianthin -Mal derivatives 1-2 (freshly prepared in TBS pH 7.5, 2 mg/ml, 1.3 mole equivalents per ‘thiol’, 40 nmol and 43 nmol), the mixtures vortexed briefly then incubated for 120 minutes at 20° C. Besides the Ab-dianthin derivatives 2 conjugation reaction, two aliquots of desalted Ab-SH (50 μg, 3.3 nmol) were reacted with NEM (1.3 mole equivalents per ‘thiol’, 17.3 nmol, 6.7 μl of a 0.25 mg/ml solution) or TBS pH 7.5 buffer (6.7 μl) for 120 minutes at 20° C., as positive and negative controls, respectively. After incubation (prior to addition of NEM), a 0.100 ml aliquot of Ab-construct 2 mixture was removed and purified by gel filtration using zeba spin desalting column into TBS pH 7.5. This aliquot was characterized by UV-vis and alongside positive and negative controls was characterized by Ellman's assay to obtain dianthin derivatives 2 incorporation. To each bulk Ab-construct mixture was added an aliquot of freshly prepared NEM solution (0.25 mg/ml, 2.5 mole equivalents, 19 and 21 nmol) and the mixtures purified by gel filtration using a 1.6×30 cm Sephadex G50M eluting with DPBS pH 7.5 followed by repeated centrifugal filtration and washing using a 100 KDa MWCO concentrator to give purified Ab-construct 1-2 conjugates. The products were filtered to 0.2 μm prior to dispensing for biological evaluation.

11. V_{HH}-[S-Trifunctional linker-(S-QS21)-(L-dianthin)]₄
HER2-V_{HH}-[S-Tri-(S-QS21)-(L-dianthin)]₄, HER2-V_{HH}-[S-Tri-(blocked)-(L-dianthin)]₄,
EGFR-V_{HH}-[S-Tri-(S-QS21)-(L-dianthin)]₄, EGFR-V_{HH}-[S-Tri-(blocked)-(L-dianthin)]₄,
CD71-V_{HH}-[S-Tri-(S-QS21)-(L-dianthin)]₄, CD71-V_{HH}-[S-Tri-(blocked)-(L-dianthin)]₄,

HER2-V_{HH}, EGFR-V_{HH}, and CD71-V_{HH} are referred hereafter as “Ab”. Ab was conjugated via Michael-type thiol-ene reaction to two different maleimide (Mal) bearing dianthin derivatives which are referred hereafter as “dianthin -Mal”. These dianthin -Mal derivatives were namely: 1) Mal-Trifunctional linker-(S-QS21)-(L-dianthin), 2) Mal-Trifunctional linker-(blocked)-(L-dianthin). The procedure is exemplary described for HER2-V_{HH}-[S-Trifunctional linker-(S-QS21)-(L-dianthin)]₄:

Ab was reconstituted to 21 mg/ml with deionized water (DI), then diluted to 5 mg/ml using histidine buffer pH 6. To a 20 mg (4.0 ml) aliquot was added 10 μl/ml each of Tris concentrate (127 mg/ml, 1.05 M), Tris.HCl concentrate (623 mg/ml, 3.95M) and EDTA-Na₂ concentrate (95 mg/ml, 0.26 M) to give a 50 mM TBS, 2.5 mM EDTA buffer pH 7.5.

To Ab (2.1 mg, 0.5 mg/ml, 0.14 μmol) was added an aliquot of freshly prepared TCEP solution (1.00 mg/ml, 2.35 mole equivalents, 0.32 μmol), the mixture vortexed briefly then incubated for 90 minutes at 20° C. with roller-mixing. After incubation (prior to addition of construct), a ca. 0.2 mg (0.044 ml) aliquot of Ab-SH was removed from each mixture and purified by gel filtration using a zeba spin desalting column into TBS pH 7.5. These aliquots were characterized by UV-vis analysis and Ellman's assay (thiol to ab ratio=4.0). The bulk Ab-SH was split into two aliquots (0.11 mg, 7.6 nmol and 0.12 mg, 8.3 nmol), and to each aliquot was added an aliquot of each of the dianthin -Mal derivatives 1-2 (freshly prepared in TBS pH 7.5, 2 mg/ml, 1.3 mole equivalents per ‘thiol’, 40 nmol and 43 nmol), the mixtures vortexed briefly then incubated for 120 minutes at 20° C. Besides the Ab-dianthin derivatives 2 conjugation reaction two aliquots of desalted Ab-SH (50 μg, 3.3 nmol) were reacted with NEM (1.3 mole equivalents per ‘thiol’, 17.3 nmol, 6.7 μl of a 0.25 mg/ml solution) or TBS pH 7.5 buffer (6.7 μl) for 120 minutes at 20° C., as positive and negative controls, respectively. After incubation (prior to addition of NEM), a 0.100 ml aliquot of Ab-construct 2 mixture was removed and purified by gel filtration using zeba spin desalting column into TBS pH 7.5. This aliquot was characterized by UV-vis and alongside positive and negative controls was characterized by Ellman's assay to obtain dianthin derivatives 2 incorporation. To each bulk Ab-construct mixture was added an aliquot of freshly prepared NEM solution (0.25 mg/ml, 2.5 mole equivalents, 19 and 21 nmol) and the mixtures purified by gel filtration using a 1.6×30 cm Sephadex G50M eluting with DPBS pH 7.5 followed by repeated centrifugal filtration and washing using a 100 KDa MWCO concentrator to give purified Ab-construct 1-2 conjugates. The products were filtered to 0.2 μm prior to dispensing for biological evaluation.

12. V_{HH}-[S-Trifunctional linker-(S-dendron-(L-QS21)ₙ)-(L-dianthin)]₄
HER2-V_{HH}-[S-Tri-(S-dendron-(L-QS21)n)-(L-dianthin)]₄, HER2-V_{HH}-[S-Tri-(blocked)-(L-dianthin)]₄,
EGFR-V_{HH}-[S-Tri-(S-dendron-(L-QS21)n)-(L-dianthin)]₄, EGFR-V_{HH}-[S-Tri-(blocked)-(L-dianthin)]₄,
CD71-V_{HH}-[S-Tri-(S-dendron-(L-QS21)n)-(L-dianthin)]₄, CD71-V_{HH}-[S-Tri-(blocked)-(L-dianthin)]₄,

HER2-V_{HH}, EGFR-V_{HH}, and CD71-V_{HH} are referred hereafter as “Ab”. Ab was conjugated via Michael-type thiol-ene reaction to two different maleimide (Mal) bearing dianthin derivatives which are referred hereafter as “dianthin-Mal”. These dianthin-Mal derivatives were namely: 1) Mal-Trifunctional linker-(S-dendron-(L-QS21)n)-(L-dianthin-Mal), 2) Mal-Trifunctional linker-(blocked)-(L-dianthin-Mal). “n” refers to the number of QS21 molecules that is 4, 8, or higher than 8. The procedure is exemplary described for HER2-V_{HH}-[S-Trifunctional linker-(S-dendron-(L-QS21)4)-(L-dianthin-Mal)]₄:

Ab was reconstituted to 21 mg/ml with deionized water (DI), then diluted to 5 mg/ml using histidine buffer pH 6. To a 20 mg (4.0 ml) aliquot was added 10 μl/ml each of Tris concentrate (127 mg/ml, 1.05 M), Tris.HCl concentrate (623 mg/ml, 3.95 M) and EDTA-Na₂ concentrate (95 mg/ml, 0.26 M) to give a 50 mM TBS, 2.5 mM EDTA buffer pH 7.5.

To Ab (2.1 mg, 0.5 mg/ml, 0.14 μmol) was added an aliquot of freshly prepared TCEP solution (1.00 mg/ml, 2.35 mole equivalents, 0.32 μmol), the mixture vortexed briefly then incubated for 90 minutes at 20° C. with roller-mixing. After incubation (prior to addition of construct), a ca. 0.2 mg (0.044 ml) aliquot of Ab-SH was removed from each mixture and purified by gel filtration using a zeba spin desalting column into TBS pH 7.5. These aliquots were characterized by UV-vis analysis and Ellman's assay (thiol to ab ratio=4.0). The bulk Ab-SH was split into two aliquots (0.11 mg, 7.6 nmol and 0.12 mg, 8.3 nmol), and to each aliquot was added an aliquot of each of the dianthin-Mal derivatives 1-2 (freshly prepared in TBS pH 7.5, 2 mg/ml, 1.3 mole equivalents per ‘thiol’, 40 nmol and 43 nmol), the mixtures vortexed briefly then incubated for 120 minutes at 20° C. Besides the Ab-dianthin derivatives 2 conjugation reaction, two aliquots of desalted Ab-SH (50 μg, 3.3 nmol) were reacted with NEM (1.3 mole equivalents per ‘thiol’, 17.3 nmol, 6.7 μl of a 0.25 mg/ml solution) or TBS pH 7.5 buffer (6.7 μl) for 120 minutes at 20° C., as positive and negative controls, respectively. After incubation (prior to addition of NEM), a 0.100 ml aliquot of Ab-construct 2 mixture was removed and purified by gel filtration using zeba spin desalting column into TBS pH 7.5. This aliquot was characterized by UV-vis and alongside positive and negative controls was characterized by Ellman's assay to obtain dianthin derivatives 2 incorporation. To each bulk Ab-construct mixture was added an aliquot of freshly prepared NEM solution (0.25 mg/ml, 2.5 mole equivalents, 19 and 21 nmol) and the mixtures purified by gel filtration using a 1.6×30 cm Sephadex G50M eluting with DPBS pH 7.5 followed by repeated centrifugal filtration and washing using a 100 KDa MWCO concentrator to give purified Ab-construct 1-2 conjugates. The products were filtered to 0.2 μm prior to dispensing for biological evaluation.

### Example 3

Conjugates of the invention. FIG. 1F-I display four typical molecular assemblies or conjugates (covalent complexes) of the invention. These conjugates are manufactured and purified, for testing in cell-based bioassays, in vivo animal models, etc.

FIG. 1F is a cartoon representing an endosomal/lysosomal escape enhancing conjugate according to the invention, comprising at least one saponin moiety ‘S’ complexed with (covalently bound to) a targeting ligand such as an IgG (or an sdAb in some embodiments), wherein the saponin is linked directly to the antibody, or is bound to the antibody via a (cleavable) linker, the antibody further complexed with (covalently bound to) at least one effector moiety ‘E’ via (cleavable) bond(s). The saponins are typically linked to the —SH groups of the cysteines in the ligand, here an antibody. The effector moiety/moieties is/are typically linked to the —SH groups of the cysteines in the ligand, here an antibody. Typically, the at least one saponin is selected from SA1641, SO1861, GE1741, QS-21, QS-7, or derivatives thereof, and combinations thereof, and the saponin SO1861 (derivative) is preferred. Typical cell-surface molecule targeting ligands selected for incorporation in the conjugate of the invention are immunoglobulins specific for (tumor) cell-surface receptors such as trastuzumab, cetuximab, anti-CD71 monoclonal antibody, or EGF for binding to EGFR. In FIG. 1F the cell-targeting ligand is an antibody specific for a cell-surface receptor. Typical targeted cell-surface molecules are HER2, EGFR, CD20, CD22, Folate receptor 1, CD146, CD56, CD19, CD138, CD27L, PSMA, CanAg, integrin-alphaV, CA6, CD33, mesothelin, Cripto, CD3, CD30, CD33, CD239, CD70, CD123, CD352, DLL3, CD25, ephrinA4, MUC1, Trop2, CD38, FGFR3, CD123, DLL3, CEACAM5, HER3, CD74, PTK7, Notch3, FGF2, C4.4A, FLT3, CD71. Also the known tumor-targeting antibodies are preferred for manufacturing a conjugate of the invention according to FIG. 1F. Typically the effector moiety/moieties is/are selected from a (protein) toxin such as dianthin, saporin, ribosomal inactivating protein, or is/are an oligonucleotide such as an RNA, an siRNA, mRNA, BNA, or an enzyme. The saponin and the payload (effector moiety) are covalently coupled directly to the antibody or are linked to the antibody via a linker such as a cleavable linker, cleavable under acidic conditions, such as at a pH of 4.5-5.5.

Examples of endosomal/lysosomal escape enhancing conjugates of FIG. 1F that are manufactured and tested for activity by the current inventors are at least cetuximab, anti-CD71 monoclonal antibody, and trastuzumab coupled to (terminal) SO1861 and coupled to a payload such as HSP27 silencing ASO (BNA), dianthin, the enzyme Cre-recombinase. The term “terminal” in the context of the invention is to be understood as a molecule which is covalently linked to a single further molecule in the conjugates of the inventions. For example in the conjugate saponin-sdAb-effector moiety, it is to be understood that both the saponin and the effector moiety are terminal moieties in the conjugate, whereas the sdAb is the central moiety bearing the two terminal moieties.

FIG. 1G is a cartoon representing the endosomal/lysosomal escape enhancing conjugate according to the invention, comprising at least one saponin moiety ‘S’ complexed with a targeting ligand such as an IgG via a scaffold moiety such as a Dendron or PAMAM, wherein the saponin is linked directly to the dendron, or via a (cleavable) linker. The dendron moiety/moieties is/are typically linked to the —SH groups of the cysteines in the ligand (the antibody). Typically, the saponins are selected from SA1641, SO1861, GE1741, QS-21, QS-7 and combinations thereof and derivatives thereof, and the saponin SO1861 (derivative) is preferred. Typical cell-surface molecule targeting ligands selected for incorporation in the conjugate of the invention are immunoglobulins specific for (tumor) cell-surface receptors such as trastuzumab, anti-CD71 monoclonal antibody, cetuximab. Also the anti-tumor monoclonal antibodies known in the art are preferred for manufacturing a conjugate of the invention according to FIG. 1G. The conjugates comprise the antibody which is further complexed with at least one effector moiety ‘E’ wherein the effector moiety/moieties is/are linked to the same scaffold such as a dendron to which the at least saponin moiety is coupled, the effector moiety coupled to the dendron via (cleavable) bond(s) such as via a linker. Typically the antibody binds to any of cell-surface molecules HER2, EGFR, CD20, CD22, Folate receptor 1, CD146, CD56, CD19, CD138, CD27L, PSMA, CanAg, integrin-alphaV, CA6, CD33, mesothelin, Cripto, CD3, CD30, CD33, CD239, CD70, CD123, CD352, DLL3, CD25, ephrinA4, MUC1, Trop2, CD38, FGFR3, CD123, DLL3, CEACAM5, HER3, CD74, PTK7, Notch3, FGF2, C4.4A, FLT3, CD71.

Examples of endosomal/lysosomal escape enhancing conjugates of FIG. 1G that are manufactured and tested for activity by the current inventors are at least trastuzumab provided with at least a dendron, the at least one dendron bound to (terminal) saponin moiety/moieties and (terminal) payload moiety/moieties (effector moiety/moieties). The saponin is typically SO1861, the payload is typically BNA capable of silencing HSP27 (ASO (BNA)) or ApoB, or a (protein) toxin or an siRNA. The SO1861 (derivative) is coupled to the dendron via a cleavable hydrazone linkage (covalent bond).

### Example 4—Saponins Mixture of Quillaja saponaria Comprising QS-21, with Endosomal/Lysosomal Escape Enhancing Activity

Scheme Q displays the common molecular structure of a series of QS-21 saponins (in part adapted from: Conrado Pedebos, Laercio Pol-Fachin, Ramon Pons, Cilaine V. Teixeira Hugo Verli, Atomic Model and Micelle Dynamics of QS-21 Saponin, Molecules 2014, 19, 3744-3760; four isoforms, wherein each of the depicted glycans can be bound as the R group). A mixture of water-soluble saponins obtained from Quillaja saponaria (Sigma-Aldrich, product No. S4521; Roth, Item No. 6857; InvivoGen, product ‘Quil-A’) may be applied in an endosomal/lysosomal escape enhancing conjugate, composition and combination of the invention, based on endosomal/lysosomal escape enhancing properties of at least one individual saponin present in the mixture, e.g. QS-21, or based on a combination of two or more of the saponins comprised by the mixture, such as QS-21 and QS-7.

The inventors demonstrated that the mixture of saponins from Quillaja saponaria at 2.5 microgram/ml dose was capable of enhancing endosomal escape of dianthin, as tested with mammalian tumor cells in a cell-based bioassay. The effector molecule exposed to the cells was dianthin covalently coupled to the ligand EGF: EGF-dianthin. Cells tested were tumor cell lines HeLa for free saponins, and A431, MDA-MB-468, CaSki and A2058 for testing the saponins when covalently coupled to cetuximab.

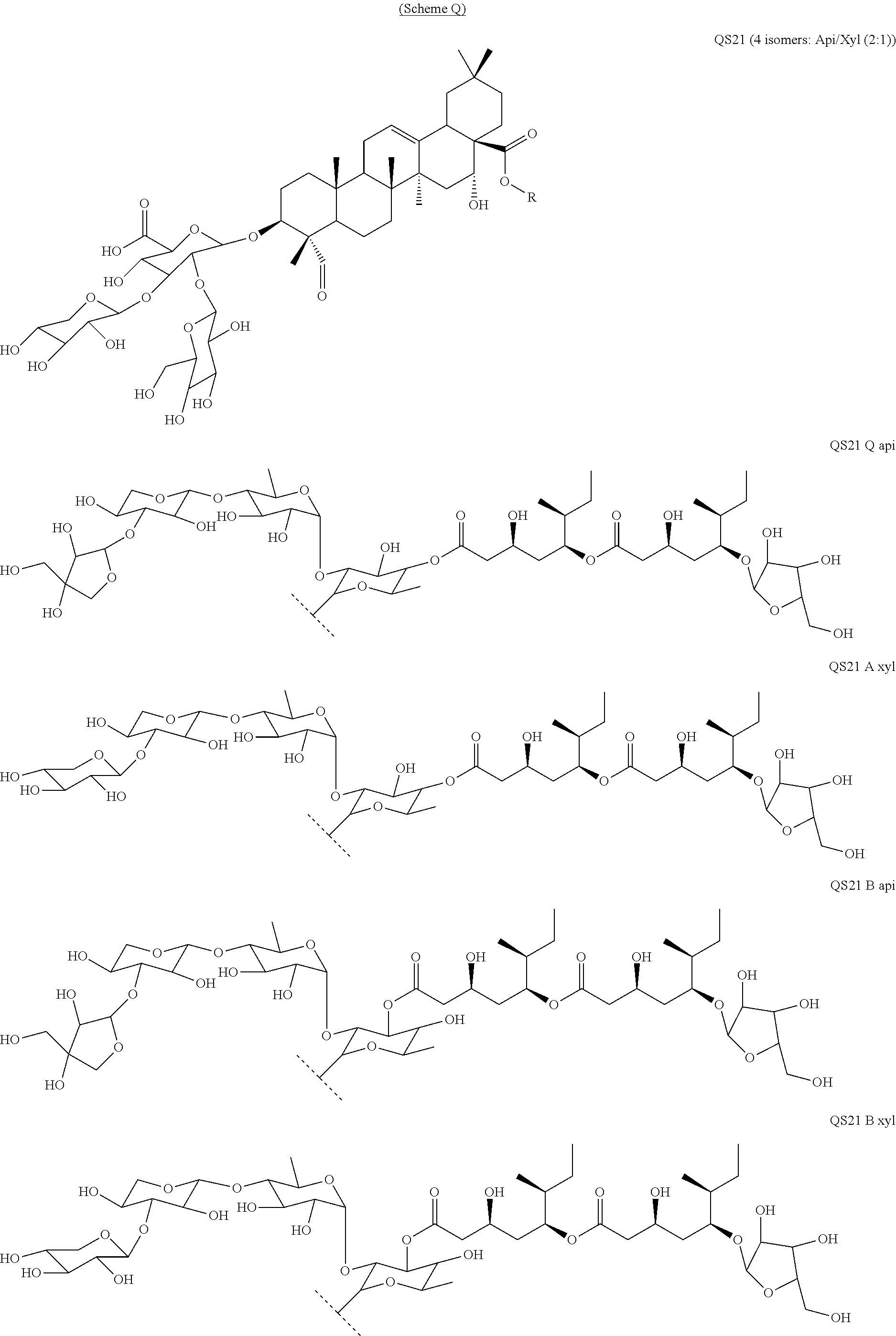

### Example 19. SO1861+EGFR/HER2/CD71 Targeted V_{HH}-Protein Toxin

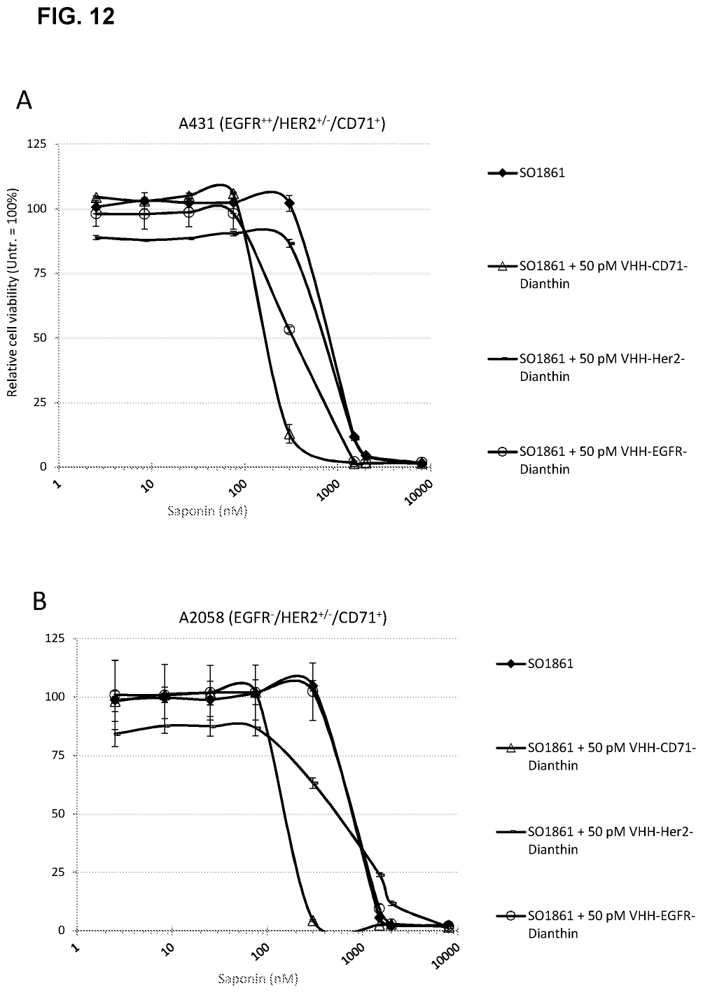

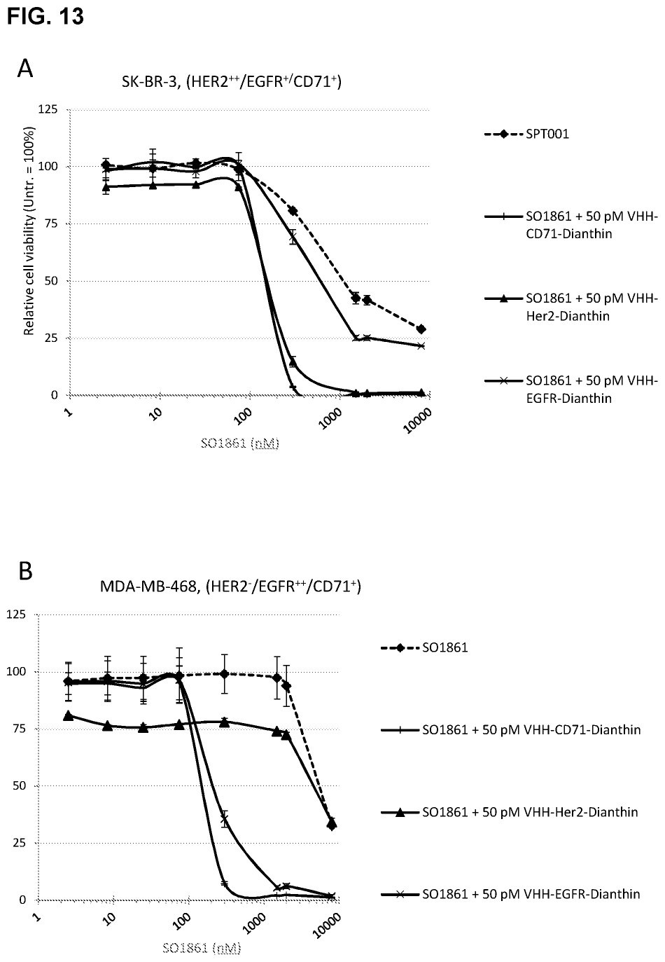

SO1861 was titrated on a fixed concentration of 50 pM V_{HH}-CD71-dianthin, 50 pM V_{HH}-HER2-dianthin or 50 pM V_{HH}-EGFR-dianthin. Targeted protein toxin-mediated cell killing on A431 (EGFR⁺⁺/HER2^{+/-}/CD71⁺), A2058 (EGFR⁻/HER2^{+/-}/CD71⁺), SK-BR-3 (HER2⁺⁺/EGFR⁺/CD71⁺) and MDA-MB-468 (HER2⁻/EGFR⁺⁺/CD71⁺) was determined. This revealed that SO1861+50 pM V_{HH}CD71-dianthin showed activity at IC50=200 nM in A431 (CD71⁺) and A2058 (CD71⁺) cells, whereas SO1861+50 pM V_{HH}EGFR-dianthin showed activity at IC50=400 nM in A431 (EGFR⁺⁺), but no activity in A2058 (EGFR⁻) (FIG. 12). SO1861+50 pM V_{HH}-HER2-dianthin showed no activity with any of the tolerated SO1861 concentrations (SO1861 tolerated concentration IC50=1000 nM) in A431 (HER2^{+/-}), whereas in A2058 (HER2^{+/-}), only slight activity could be detected at IC50=700 nM SO1861 (FIG. 12). Furthermore, SO1861+50 pM V_{HH}-CD71-dianthin or SO1861+50 pM V_{HH}-HER2-dianthin showed activity at IC50=200 nM in SK-BR-3 (HER2⁺⁺/CD71⁺), whereas in MDA-MB-468 (HER2⁻/CD71⁺) cells, SO1861+50 pM V_{HH}-CD71-dianthin showed activity at IC50=200 nM and SO1861+50 pM V_{HH}-HER2-dianthin showed no enhanced activity compared to SO1861 alone (FIG. 13). SO1861+50 pM V_{HH}-EGFR-dianthin showed activity at IC50=700 nM in SK-BR-3 (EGFR+) and strong activity (SO1861 IC50=300 nM) in MDA-MB-468 (EGFR⁺⁺) (FIG. 13).

### Example 20 V_{HH}-EGFR-dianthin+SO1861-EMCH or mAb-SO1861

The 1 target 2-components system (1T2C) is the combination treatment of mAb-SO1861 and V_{HH}-protein toxin, where mAb and V_{HH} recognize and bind the same cell surface receptor (FIG. 1E).

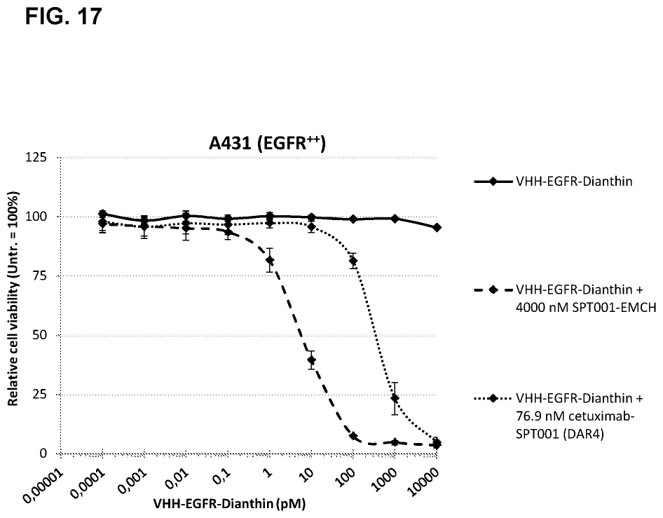

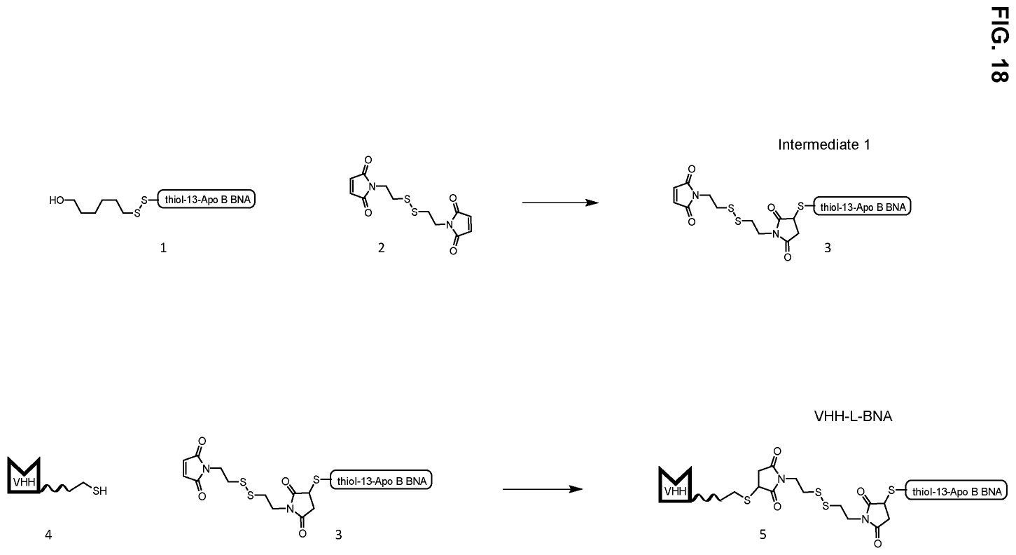

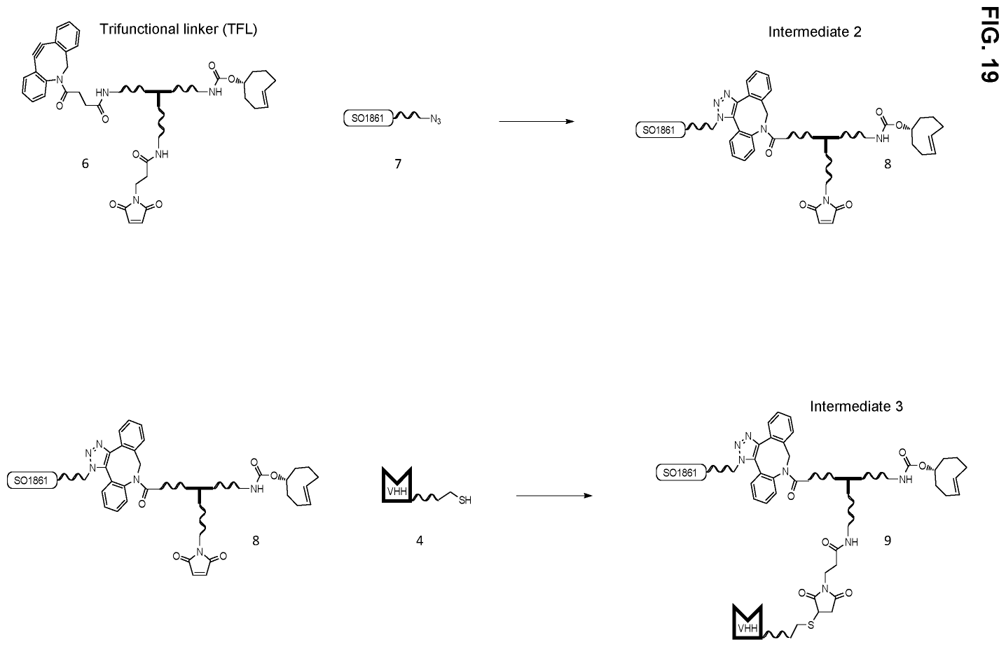

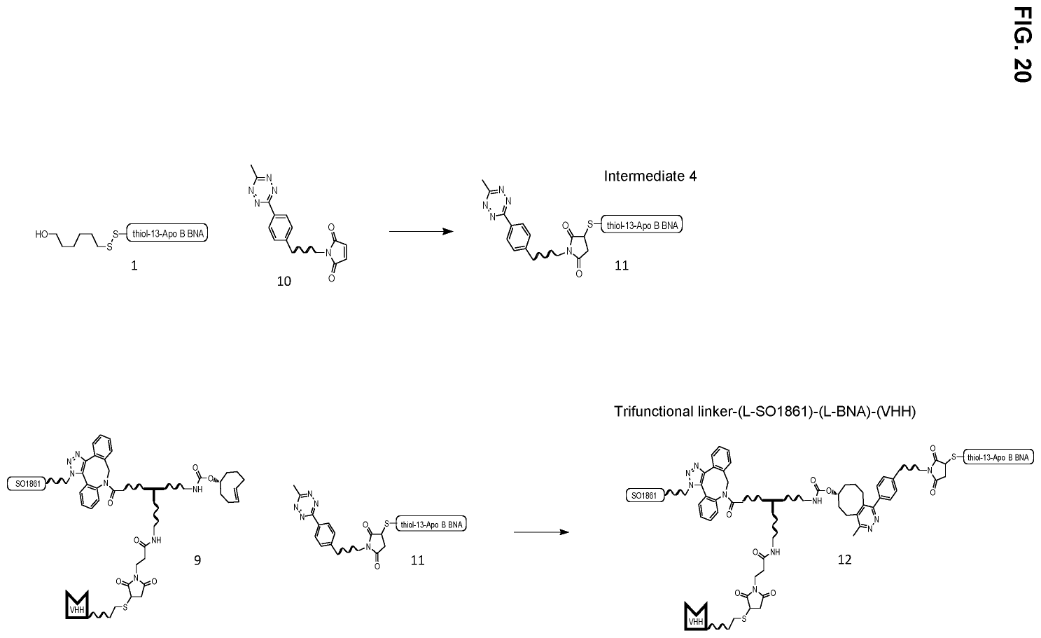

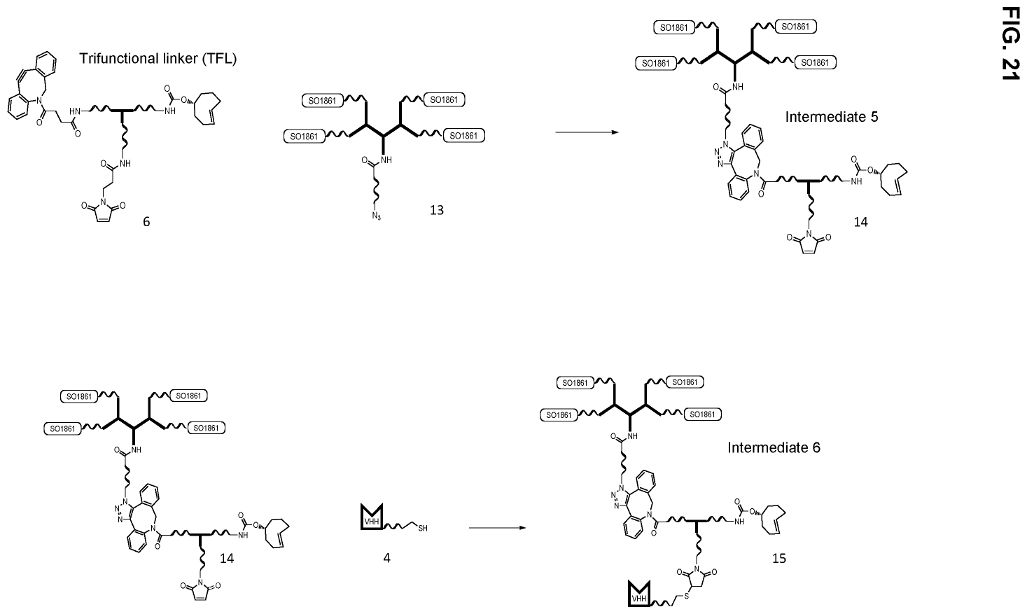

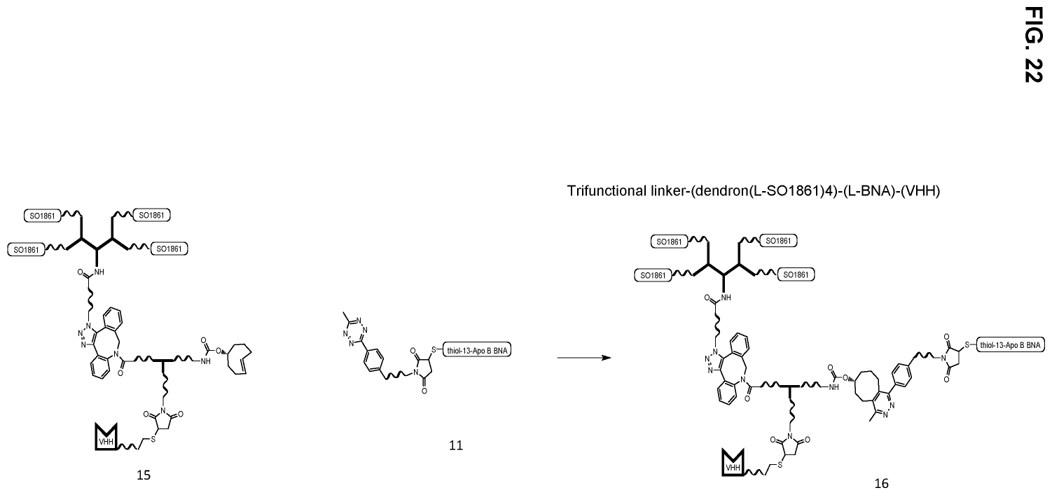

V_{HH}-EGFR-dianthin (conjugate) was titrated alone or on a fixed concentration of 4000 nM SO1861-EMCH or 76.9 nM cetuximab-SO1861 (DAR4) and targeted protein toxin mediated cell killing on A431 (EGFR⁺⁺) was determined. In A431 cells (EGFR⁺⁺) the combination of V_{HH}-EGFR-dianthin+4000 nM SO1861-EMCH revealed an IC50=4 pM compared to V_{HH}-EGFR-dianthin alone (IC50>10.000 pM), whereas tV_{HH}-EGFR-dianthin+76.9 nM cetuximab-SO1861 (DAR4) revealed an IC50=400 pM (FIG. 17).

### Example 21. Bivalent-V_{HH}-EGFR-dianthin or mAb-toxin+SO1861-EMCH or mAb-SO1861

The 1 target 2-components system (1T2C) is the combination treatment of mAb-SO1861 and V_{HH}-protein toxin, where mAb and V_{HH} recognize and bind the same cell surface receptor (FIG. 1E).

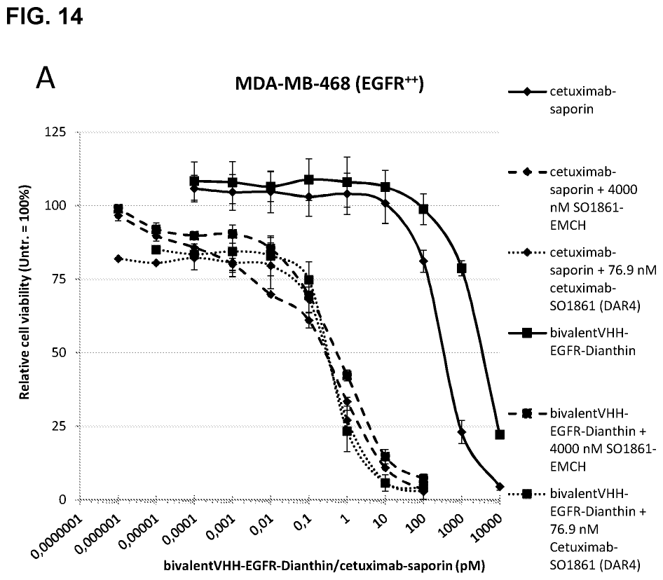

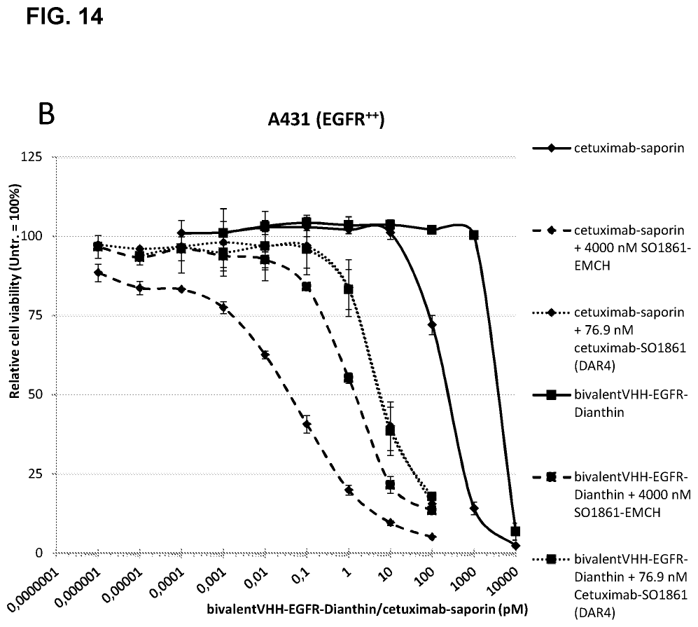

Cetuximab-saporin (conjugate) or bivalentV_{HH}-EGFR-dianthin (recombinant fusion protein) was titrated alone or on a fixed concentration of 4000 nM SO1861-EMCH or 76.9 nM cetuximab-SO1861 (DAR4) and targeted protein toxin mediated cell killing on MDA-MB-468 (EGFR⁺⁺) and A431 (EGFR⁺⁺) was determined. In MDA-MB-468 cells (EGFR⁺⁺) the combination of bivalentV_{HH}-EGFR-dianthin+4000 nM SO1861-EMCH, cetuximab-saporin+4000 nM SO1861-EMCH, bivalentV_{HH}-EGFR-dianthin+76.9 nM cetuximab-SO1861 (DAR4) or cetuximab-saporin+76.9 nM cetuximab-SO1861 (DAR4) revealed a strong increase in EGFR targeted toxin-mediated the cell killing activity (IC50=0.5 pM) compared to bivalentV_{HH}-EGFR-dianthin or cetuximab-saporin alone (IC50=4000 pM or IC50=300 pM, respectively) (FIG. 14A). In A431 cells (EGFR⁺⁺) the combination of bivalentV_{HH}-EGFR-dianthin+4000 nM SO1861-EMCH revealed an IC50=0.05 pM compared to bivalentV_{HH}-EGFR-dianthin alone (IC50=300 pM), cetuximab-saporin+4000 nM SO1861-EMCH revealed IC50=3 pM compared to cetuximab-saporin alone (IC50=4000 pM), bivalentV_{HH}-EGFR-dianthin+76.9 nM cetuximab-SO1861 (DAR4) or cetuximab-saporin+76.9 nM cetuximab-SO1861 (DAR4) revealed both an IC50=10 pM (FIG. 14B).

### Example 22. SO1861+Bivalent-V_{HH}-EGFR-dianthin

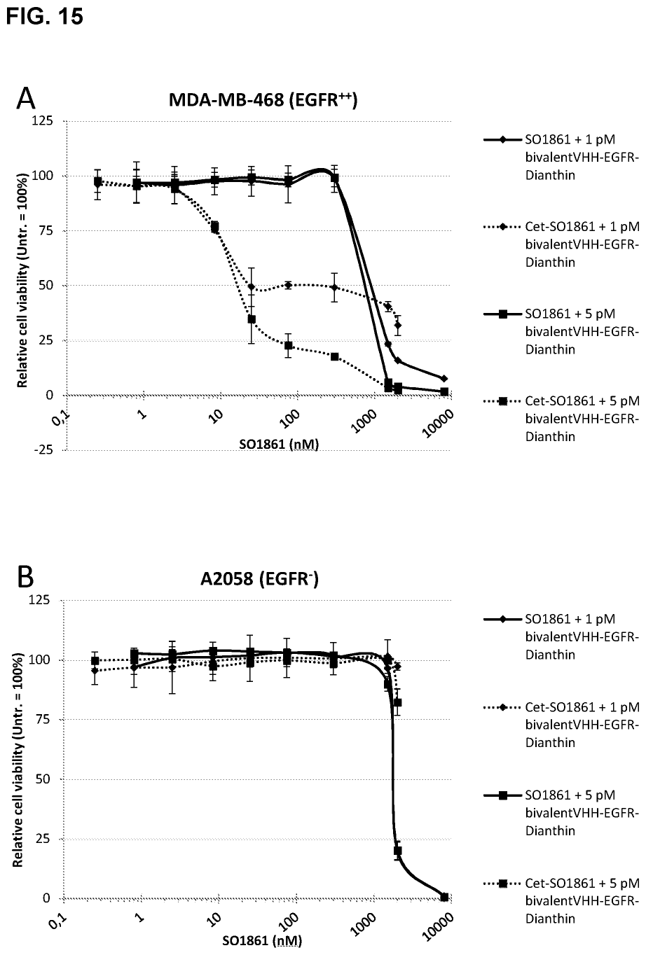

SO1861 or Cetuximab-SO1861 (DAR4) was titrated on a fixed concentration of 1 pM or 5 pM bivalentV_{HH}-EGFR-dianthin and targeted protein toxin mediated cell killing on MDA-MB-468 cells (EGFR⁺⁺) and A2058 cells (EGFR⁻) was determined. In MDA-MB-468 cells (EGFR⁺⁺) this revealed that very low concentrations cetuximab conjugated SO1861 in combination with 1 pM or 5 pM bivalentV_{HH}-EGFR-dianthin induced efficient cell killing (IC50=20 nM), whereas unconjugated SO1861+1 pM or 5 pM bivalentV_{HH}-EGFR-dianthin showed activity at IC50=1000 nM (FIG. 15A). In A2058 (EGFR⁻) activity was only observed at IC50>1000 nM (FIG. 15B).

### Example 23. mAb-SO1861+bivalentV_{HH}-EGFR-dianthin (1T2C and 2T2C)

The 1 target 2-components system (1T2C) is the combination treatment of mAb-SO1861 and V_{HH}-protein toxin, where mAb and V_{HH} recognize and bind the same cell surface receptor (FIG. 1E). The 2 target 2-components system (2T2C) is also the combination treatment of mAb-SO1861 and V_{HH}-protein toxin, where the mAb and V_{HH} recognize another cell surface receptor (FIG. 1D).

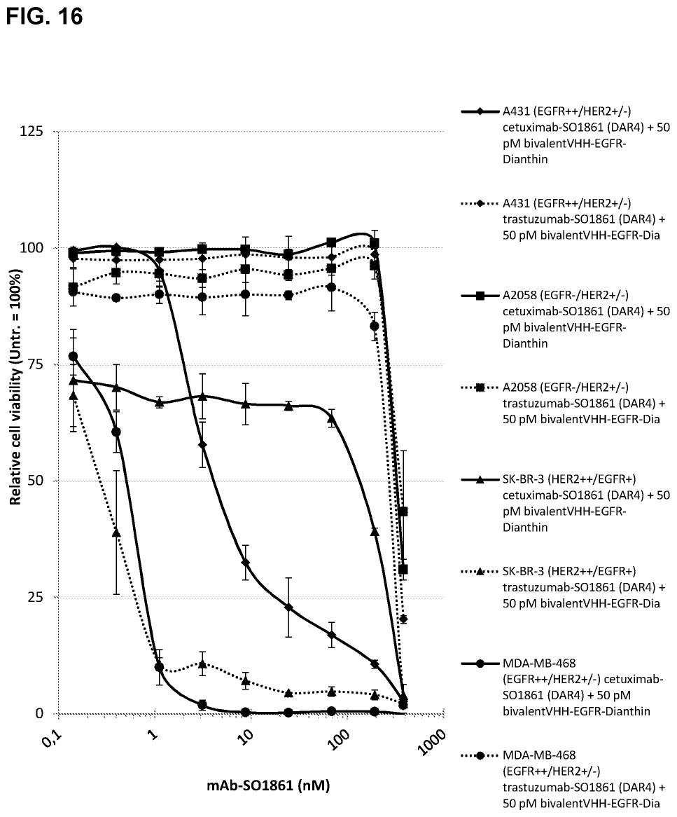

BivalentV_{HH}-EGFR-dianthin (with the amino-acid sequence depicted as the sequence of SEQ ID NO: 73) was produced as recombinant fusion protein. Cetuximab-SO1861 (DAR4) was titrated on a fixed (non-effective) concentration of 50 pM bivalentV_{HH}-EGFR-dianthin and targeted protein toxin mediated cell killing on A431 (EGFR⁺⁺/HER2^{+/-}), MDA-MB-468 (EGFR⁺⁺/HER2^{+/-}), SK-BR-3 (HER2⁺⁺/EGFR⁺) and A2058 (EGFR⁻/HER2^{+/-}) was determined. This revealed that very low concentrations cetuximab-SO1861 (DAR4) in combination with 50 pM bivalentV_{HH}-EGFR-dianthin induced efficient cell killing of MDA-MB-468 (EGFR⁺⁺) cells (IC50=0.5 nM) and A431 (EGFR⁺⁺) cells (IC50=4 nM) whereas on SK-BR-3 (EGFR⁺) or A2058 (EGFR⁻) cells activity was detected only with very high concentrations of cetuximab-SO1861 (IC50=200 nM and IC50=400 nM respectively) (FIG. 16).

This shows that low concentrations of cetuximab-SO1861 (DAR4) can efficiently induce endosomal escape of a bivalentV_{HH}-EGFR-dianthin only in high EGFR expressing cells, thereby inducing enhanced cell killing.

Next, trastuzumab-SO1861 (DAR4) was titrated on a fixed (non-effective) concentration of 50 pM bivalentV_{HH}-EGFR-dianthin and targeted protein toxin-mediated cell killing on A431 (EGFR⁺⁺/HER2^{+/-}), A2058 (EGFR⁻/HER2^{+/-}), SK-BR-3 (HER2⁺⁺/EGFR⁺) and MDA-MB-468 cells (HER2-/EGFR⁺⁺) was determined. This revealed that very low concentrations trastuzumab-SO1861 (DAR4) in combination with 50 pM bivalentV_{HH}-EGFR-dianthin induced efficient cell killing of SK-BR-3 cells (IC50=0.3 nM), whereas the combination showed only at very high concentrations trastuzumab-SO1861, cell killing activity in A431 (HER2^{+/-}), A2058 (HER2^{+/-}) and MDA-MB-468 (HER2⁻) cells (IC50=400 nM) (FIG. 16). This shows that low concentrations of trastuzumab-SO1861 (DAR4) can efficiently induce endosomal escape of a bivalentV_{HH}-EGFR-dianthin only in high HER2 expressing cells, thereby inducing enhanced cell killing.

### Materials and Methods

### Materials

SO1861 was isolated and purified by Analyticon Discovery GmbH from raw plant extract obtained from Saponaria officinalis. Trastuzumab (Tras, Herceptin®, Roche), Cetuximab (Cet, Erbitux®, Merck KGaA) were purchased from the pharmacy (Charite, Berlin). Bivalent-V_{HH}-EGFR-dianthin fusion (SEQ ID NO: 73) was produced as recombinant protein in E. coli according to standard procedures at GenScript (Leiden, The Netherlands). Monovalent V_{HH} were purchased from QVQ, Utrecht, The Netherlands (V_{HH}—HER2: clone name: Q17c; V_{HH}-CD71: clone name: Q52c V_{HH}-EGFR: clone name: Q86c). Cetuximab-saporin conjugates were produced and purchased from Advanced Targeting Systems (San Diego, CA). Dianthin-cys was produced and purchased from Proteogenix, France.

Tris(2-carboxyethyl)phosphine hydrochloride (TCEP, 98%, Sigma-Aldrich), 5,5-Dithiobis(2-nitrobenzoic acid) (DTNB, Ellman's reagent, 99%, Sigma-Aldrich), Zeba™ Spin Desalting Columns (2 mL, Thermo-Fisher), NuPAGE™ 4-12% Bis-Tris Protein Gels (Thermo-Fisher), NuPAGE™ MES SDS Running Buffer (Thermo-Fisher), Novex™ Sharp Pre-stained Protein Standard (Thermo-Fisher), PageBlue™ Protein Staining Solution (Thermo-Fischer), Pierce™ BCA Protein Assay Kit (Thermo-Fisher), N-Ethylmaleimide (NEM, 98%, Sigma-Aldrich), 1,4-Dithiothreitol (DTT, 98%, Sigma-Aldrich), Sephadex G25 (GE Healthcare), Sephadex G50 M (GE Healthcare), Superdex 200P (GE Healthcare), Isopropyl alcohol (IPA, 99.6%, VWR), Tris(hydroxymethyl)aminomethane (Tris, 99%, Sigma-Aldrich), Tris(hydroxymethyl)aminomethane hydrochloride (Tris.HCL, Sigma-Aldrich), L-Histidine (99%, Sigma-Aldrich), D-(+)-Trehalose dehydrate (99%, Sigma-Aldrich), Polyethylene glycol sorbitan monolaurate (TWEEN 20, Sigma-Aldrich), Dulbecco's Phosphate-Buffered Saline (DPBS, Thermo-Fisher), Guanidine hydrochloride (99%, Sigma-Aldrich), Ethylenediaminetetraacetic acid disodium salt dihydrate (EDTA-Na₂, 99%, Sigma-Aldrich), sterile filters 0.2 μm and 0.45 μm (Sartorius), Vivaspin T4 and T15 concentrator (Sartorius), Superdex 200PG (GE Healthcare), Tetra(ethylene glycol), Dimethyl sulfoxide (DMSO, 99%, Sigma-Aldrich), N-(2-Aminoethyl)maleimide trifluoroacetate salt (AEM, 98%, Sigma-Aldrich), L-Cysteine (98.5%, Sigma-Aldrich), deionized water (DI) was freshly taken from Ultrapure Lab Water Systems (MilliQ, Merck), Nickel-nitrilotriacetic acid agarose (Ni-NTA agarose, Protino), Glycine (99.5%, VWR), 5,5-Dithiobis(2-nitrobenzoic acid (Ellman's reagent, DTNB, 98%, Sigma-Aldrich), S-Acetylmercaptosuccinic anhydride Fluorescein (SAMSA reagent, Invitrogen) Sodium bicarbonate (99.7%, Sigma-Aldrich), Sodium carbonate (99.9%, Sigma-Aldrich), PD MiniTrap desalting columns with Sephadex G-25 resin (GE Healthcare), PD10 G25 desalting column (GE Healthcare), Zeba Spin Desalting Columns in 0.5, 2, 5, and 10 mL (Thermo-Fisher), Vivaspin Centrifugal Filters T4 10 kDa MWCO, T4 100 kDa MWCO, and T15 (Sartorius), Biosep s3000 aSEC column (Phenomenex), Vivacell Ultrafiltration Units 10 and 30 kDa MWCO (Sartorius), Nalgene Rapid-Flow filter (Thermo-Fisher),

### Methods

### SO1861-EMCH Synthesis

To SO1861 (121 mg, 0.065 mmol) and EMCH.TFA (110 mg, 0.325 mmol) was added methanol (extra dry, 3.00 mL) and TFA (0.020 mL, 0.260 mmol). The reaction mixture stirred at room temperature. After 1.5 hours the reaction mixture was subjected to preparative MP-LC.¹ Fractions corresponding to the product were immediately pooled together, frozen and lyophilized overnight to give the title compound (120 mg, 90%) as a white fluffy solid. Purity based on LC-MS 96%.

LRMS (m/z): 2069 [M−1]¹⁻

LC-MS r.t. (min): 1.08⁴

Cell viability assay

After treatment the cells were incubated for 72 hr at 37° C. before the cell viability was determined by a MTS-assay, performed according to the manufacturer's instruction (CellTiter 96@AQueous One Solution Cell Proliferation Assay, Promega). Briefly, the MTS solution was diluted 20× in DMEM without phenol red (PAN-Biotech GmbH) supplemented with 10% FBS. The cells were washed once with 200 μL/PBS well, after which 100 μL diluted MTS solution was added/well. The plate was incubated for approximately 20-30 minutes at 37° C. Subsequently, the OD at 492 nm was measured on a Thermo Scientific Multiskan FC plate reader (Thermo Scientific). For quantification the background signal of ‘medium only’ wells was subtracted from all other wells, before the cell viability percentage of treated/untreated cells was calculated, by dividing the background corrected signal of treated wells over the background corrected signal of the untreated wells (×100).

### FACS Analysis

Cells were seeded in DMEM (PAN-Biotech GmbH) supplemented with 10% fetal calf serum (PAN-Biotech GmbH) and 1% penicillin/streptomycin (PAN-Biotech GmbH), at 500,000 c/plate in 10 cm dishes and incubated for 48 hrs (5% CO₂, 37° C.), until a confluency of 90% was reached. Next, the cells were trypsinized (TryplE Express, Gibco Thermo Scientific) to single cells. 0.75×10⁶ Cells were transferred to a 15 mL falcon tube and centrifuged (1,400 rpm, 3 min). The supernatant was discarded while leaving the cell pellet submerged. The pellet was dissociated by gentle tapping the falcon tube on a vortex shaker and the cells were washed with 4 mL cold PBS (Mg²⁺ and Ca²⁺ free, 2% FBS). After washing the cells were resuspended in 3 mL cold PBS (Mg²⁺ and Ca²⁺ free, 2% FBS) and divided equally over 3 round bottom FACS tubes (1 mL/tube). The cells were centrifuged again and resuspended in 200 μL cold PBS (Mg²⁺ and Ca²⁺ free, 2% FBS) or 200 μL antibody solution; containing 5 μL antibody in 195 μL cold PBS (Mg²⁺ and Ca²⁺ free, 2% FBS). APC Mouse IgG1, K APC anti-human EGFR (#352906, Biolegend) was used to stain the EGFR receptor. PE anti-human HER2 APC anti-human CD340 (erbB2/HER-2) (#324408 Biolegend) was used to stain the HER2 receptor, PE Mouse IgG2a, κ Isotype Ctrl FC (#400212, Biolegend) was used as its matched isotype control. PE anti-human CD71 (#334106, Biolegend) was used to stain the CD71 receptor, PE Mouse IgG2a, K Isotype Ctrl FC (#400212, Biolegend) was used as its matched isotype control. Samples were incubated for 30 min at 4° C. on a tube roller mixer. Afterwards, the cells were washed 3× with cold PBS (Mg²⁺ and Ca²⁺ free, 2% FBS) and fixated for 20 min at room temperature using a 2% PFA solution in PBS. Cells were washed 2× with cold PBS, and resuspended in 250-350 μL cold PBS for FACS analysis. Samples were analyzed with a BD FACSCanto II flow cytometry system (BD Biosciences) and FlowJo software. FACS data per cell lines are in Table A2

### Procedure for the Conjugation of V_{HH}-Dianthin

Dianthin-Cys was concentrated by ultrafiltration using a vivaspin T15 10 KDa MWCO centrifugal filter and buffer exchanged into TBS pH 7.5. To the concentrated Dianthin-Cys was added an aliquot of freshly prepared TCEP solution (10.0 mg/ml), the mixture vortexed briefly then incubated for 60 minutes at 20° C. with roller-mixing. After incubation, the resulting Dianthin-SH was purified by gel filtration using a zeba spin desalting column then repeated centrifugal-wash cycles using a vivaspin T15 10 KDa MWCO centrifugal filter into TBS pH 7.5. The resulting Dianthin-SH was reacted with freshly prepared DTME solution (10 mg/ml) in DMSO, the mixture vortexed briefly then incubated for 60 minutes at 20° C. After, the Dianthin-DTME was obtained following purification by gel filtration using a zeba spin desalting column into TBS pH 7.5. The Dianthin-DTME was stored at 20° C. until conjugated. At the same time, an aliquot of V_{HH} was concentrated by ultrafiltration using a vivaspin T15 10 KDa MWCO centrifugal filter and buffer exchanged into TBS pH 7.5. To the concentrated V_{HH} was added an aliquot of freshly prepared TCEP solution (10.0 mg/ml), the mixture vortexed briefly then incubated for 60 minutes at 37° C. with roller-mixing. After incubation, the resulting V_{HH} was purified by gel filtration using a zeba spin desalting column then repeated centrifugal-wash cycles using a vivaspin T4 5 KDa MWCO centrifugal filter into TBS pH 7.5. An aliquot of the resulting V_{HH}-SH was reacted with Dianthin-DTME, the mixture vortexed briefly then incubated overnight at 20° C. After, the reaction mixture was concentrated using a vivaspin T4 10 KDa MWCO centrifuge tube and purified by gel filtration using a 1.6×35 cm Superdex 200PG column eluting into DPBS pH 7.5.

### Antibody-(L-SO1861)⁴

Trastuzumab, Cetuximab, are referred hereafter as “Ab”. Ab was conjugated to the saponin SO18161-EMCH via Michael-type thiol-ene conjugation reaction at DARs of 1, 2, 3, 4, 5, and 6. The SO1861-EMCH molecule obtains a labil (L) hydrazone bond between its structure and its maleimide function generating a labil bond between the saponin and Ab. The procedure is exemplary described for Trastuzumab-(L-SO1861)4 (See Table A3):

To a solution of Cetuximab (40 mg, 8.0 ml) was added 10 μl/ml each of Tris concentrate (127 mg/ml, 1.05 M), Tris.HCl concentrate (623 mg/ml, 3.95 M) and EDTA-Na₂ concentrate (95 mg/ml, 0.26 M) to give a 50 mM TBS, 2.5 mM EDTA buffer pH 7.5.

To Cetuximab divided into four portions (each of 9.73 mg, 4.864 mg/ml, 65 nmol) was added an aliquot of freshly prepared TCEP solution (0.5-2.0 mg/ml, 1.15-7.02 mole equivalents, 75-455 nmol), the mixtures vortexed briefly then incubated for 300 minutes at 20° C. with roller-mixing. After incubation (prior to addition of SO1861-EMCH), a ca. 1 mg (0.210 ml) aliquot of Ab-SH was removed from each mixture and purified by gel filtration using a zeba spin desalting column into TBS pH 7.5. These aliquots were characterized by UV-vis analysis and Ellman's assay (thiol to Ab ratio=2.0, 4.2, 5.9 and 6.8 respectively). To each of the bulk Ab-SH was added an aliquot of freshly prepared SO1861-EMCH solution (2 mg/ml, 1.3 mole equivalents per ‘thiol’, 0.15-0.61 μmol, 0.16-0.63 ml), the mixtures vortexed briefly then incubated for 120 minutes at 20° C. Besides each conjugation reaction, two aliquots of desalted Ab-SH (0.25 mg, 1.67 nmol) were reacted with NEM (1.3 mole equivalents per ‘thiol’, 4.3-17.4 nmol, 2.2-8.7 μl of a 0.25 mg/ml solution) or TBS pH 7.5 buffer (2.2-8.7 μl) for 120 minutes at 20° C., as positive and negative controls, respectively. After incubation (prior to addition of NEM), a 0.200 ml aliquot of Ab-SO1861-EMCH mixture was removed and purified by gel filtration using zeba spin desalting column into TBS pH 7.5. This aliquot was characterized by UV-vis and alongside positive and negative controls were characterized by Ellman's assay to obtain SO1861-EMCH incorporations. To the bulk Ab-SO1861-EMCH mixture was added an aliquot of freshly prepared NEM solution (2.5 mg/ml, 2.5-10 mole equivalents, 0.15-0.58 μmol) and the mixtures purified by zeba spin desalting columns eluting with DPBS pH 7.5 to give purified Cetuximab-(L-SO1861) conjugates (See Table A4). The products were normalized to 2.5 mg/ml and filtered to 0.2 μm prior to dispensing for biological evaluation.

### Materials and methods

### Abbreviations

AEM N-(2-Aminoethyl)maleimide trifluoroacetate salt
AMPD 2-Amino-2-methyl-1,3-propanediol
BOP (Benzotriazol-1-yloxy)tris(dimethylamino)phosphonium hexafluorophosphate
DIPEA N,N-diisopropylethylamine
DMF N,N-dimethylformamide
DTME Dithiobismaleimidoethane
DTT Dithiothreitol
EDCI·HCl 3-((Ethylimino)methyleneamino)-N,N-dimethylpropan-1-aminium chloride
EDTA Ethylenediaminetetraacetic acid
EMCH.TFA N-(ε-maleimidocaproic acid) hydrazide, trifluoroacetic acid salt
HATU 1-[Bis(dimethylamino)methylene]-1H-1,2,3-triazolo[4,5-b]pyridinium 3-oxid hexafluorophosphate
min minutes
NMM 4-Methylmorpholine
r.t. retention time
SEC Size exclusion chromatography
TBEU (Tris-(hydroxymethyl)-aminomethan)-Borat-EDTA-Urea
TCEP tris(2-carboxyethyl)phosphine hydrochloride
Temp temperature
TFA trifluoroacetic acid

### Analytical Methods

### LC-MS Method 1

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product:

neg or neg/pos within in a range of 1500-2400 or 2000-3000; ELSD: gas pressure 40 psi, drift tube temp: 50° C.; column: Acquity C18, 50×2.1 mm, 1.7 μm Temp: 60° C., Flow: 0.6 mL/min, lin. Gradient depending on the polarity of the product:
^{A}t₀=2% A, t_{5.0 min}=50% A, t_{6.0 min}=98% A
^{B}t₀=2% A, t_{5.0 min}=98% A, t_{6.0 min}=98% A

Posttime: 1.0 min, Eluent A: acetonitrile, Eluent B: 10 mM ammonium bicarbonate in water (pH=9.5).

### LC-MS Method 2

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product: pos/neg 100-800 or neg 2000-3000; ELSD: gas pressure 40 psi, drift tube temp: 50° C.; column: Waters XSelect™ CSH C18, 50×2.1 mm, 2.5 μm, Temp: 25° C., Flow: 0.5 mL/min, Gradient: t_{0 min}=5% A, t_{2.0 min}=98% A, t_{2.7 min}=98% A, Posttime: 0.3 min, Eluent A: acetonitrile, Eluent B: 10 mM ammonium bicarbonate in water (pH=9.5).

### LC-MS Method 3

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product pos/neg 105-800, 500-1200 or 1500-2500; ELSD: gas pressure 40 psi, drift tube temp: 50° C.; column: Waters XSelect™ CSH C18, 50×2.1 mm, 2.5 μm, Temp: 40° C., Flow: 0.5 mL/min, Gradient: t_{0 min}=5% A, t_{2.0 min}=98% A, t_{2.7 min}=98% A, Posttime: 0.3 min, Eluent A: 0.1% formic acid in acetonitrile, Eluent B: 0.1% formic acid in water.

### LC-MS Method 4

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product: pos/neg 100-800 or neg 2000-3000; ELSD: gas pressure 40 psi, drift tube temp: 50° C. column: Waters Acquity Shield RP18, 50×2.1 mm, 1.7 μm, Temp: 25° C., Flow: 0.5 mL/min, Gradient: t_{0 min}=5% A, t_{2.0 min}=98% A, t_{2.7 min}=98% A, Posttime: 0.3 min, Eluent A: acetonitrile, Eluent B: 10 mM ammonium bicarbonate in water (pH=9.5).

### Preparative Methods

### Preparative MP-LC Method 1

Instrument type: Reveleris™ prep MPLC; column: Waters XSelect™ CSH C18 (145×25 mm, 10 μm); Flow: 40 mL/min; Column temp: room temperature; Eluent A: 10 mM ammoniumbicarbonate in water pH=9.0); Eluent B: 99% acetonitrile+1% 10 mM ammoniumbicarbonate in water; Gradient:
^{A}t_{0 min}=5% B, t_{1 min}=5% B, t_{2 min}=10% B, t_{17 min}=50% B, t_{18 min}=100% B, t_{23 min}=100% B
^{A}t_{0 min}=5% B, t_{1 min}=5% B, t_{2 min}=20% B, t_{17 min}=60% B, t_{18 min}=100% B, t_{23 min}=100% B;

Detection UV: 210, 235, 254 nm and ELSD.

Preparative MP-LC method 2

Instrument type: Reveleris™ prep MPLC; Column: Phenomenex LUNA C18(3) (150×25 mm, 10 μm); Flow: 40 mL/min; Column temp: room temperature; Eluent A: 0.1% (v/v) Formic acid in water, Eluent B: 0.1% (v/v) Formic acid in acetonitrile; Gradient:
^{A}t_{0 min}=5% B, t_{1 min}=5% B, t_{2 min}=20% B, t_{17 min}=60% B, t_{18 min}=100% B, t_{23 min}=100% B
^{B}t_{0 min}=2% B, t_{1 min}=2% B, t_{2 min}=2% B, t_{17 min}=30% B, t_{18 min}=100% B, t_{23 min}=100% B
^{C}t_{0 min}=5% B, t_{1 min}=5% B, t_{2 min}=10% B, t_{17 min}=50% B, t_{18 min}=100% B, t_{23 min}=100% B
^{D}t_{0 min}=5% B, t_{1 min}=5% B, t_{2 min}=5% B, t_{17 min}=40% B, t_{18 min}=100% B, t_{23 min}=100% B;

Detection UV: 210, 235, 254 nm and ELSD.

### Preparative LC-MS Method 3

MS instrument type: Agilent Technologies G6130B Quadrupole; HPLC instrument type: Agilent Technologies 1290 preparative LC; Column: Waters XSelect™ CSH (C18, 150×19 mm, 10 μm); Flow: 25 ml/min; Column temp: room temperature; Eluent A: 100% acetonitrile; Eluent B: 10 mM ammonium bicarbonate in water pH=9.0; Gradient:
^{A}t₀=20% A, t_{2.5 min}=20% A, t_{11 min}=60% A, t_{13 min}=100% A, t_{17 min}=100% A
^{B}t₀=5% A, t_{2.5 min}=5% A, t_{11 min}=40% A, t_{13 min}=100% A, t_{17 min}=100% A;

Detection: DAD (210 nm); Detection: MSD (ESI pos/neg) mass range: 100-800; Fraction collection based on DAD.

### Preparative LC-MS Method 4

MS instrument type: Agilent Technologies G6130B Quadrupole; HPLC instrument type: Agilent Technologies 1290 preparative LC; Column: Waters XBridge Protein (C4, 150×19 mm, 10 μm); Flow: 25 ml/min; Column temp: room temperature; Eluent A: 100% acetonitrile; Eluent B: 10 mM ammonium bicarbonate in water pH=9.0; Gradient:
^{A}t₀=2% A, t_{2.5 min}=2% A, t_{11 min}=30% A, t_{13 min}=100% A, t_{17 min}=100% A
^{B}t₀=10% A, t_{2.5 min}=10% A, t_{11 min}=50% A, t_{13 min}=100% A, t_{17 min}=100% A
^{C}t₀=5% A, t_{2.5 min}=5% A, t_{11 min}=40% A, t_{13 min}=100% A, t_{17 min}=100% A;

Detection: DAD (210 nm); Detection: MSD (ESI pos/neg) mass range: 100-800; Fraction collection based on DAD

### Flash Chromatography

Grace Reveleris X2® C-815 Flash; Solvent delivery system: 3-piston pump with auto-priming, 4 independent channels with up to 4 solvents in a single run, auto-switches lines when solvent depletes; maximum pump flow rate 250 mL/min; maximum pressure 50 bar (725 psi); Detection: UV 200-400 nm, combination of up to 4 UV signals and scan of entire UV range, ELSD; Column sizes: 4-330 g on instrument, luer type, 750 g up to 3000 g with optional holder.

### UV-Vis Spectrophotometry

Protein concentrations were determined using a Thermo Nanodrop 2000 spectrometer and the following mass ε280 values ((mg/ml)-1 cm-1); Q8c (1.772), Q52c, (1.769), Q17c (1.802) and Q86(c) (2.246). Oligo concentrations were determined using a molar ε260 value of 153,000 M-1 cm-1.

Ellman's assay was carried out using a Perkin Elmer Lambda 25 Spectrophotometer and a literature molar E412 value of 14150 M-1 cm-1 for TNB. Experimentally determined molar E495=58,700 M-1 cm-1 and Rz280:495=0.428 were used for SAMSA-fluorescein.

### SEC

The conjugates were analysed by SEC using an Akta purifier 10 system and Biosep SEC-s3000 column eluting with DPBS:IPA (85:15). Conjugate purity was determined by integration of the Conjugate peak with respect to impurities/aggregate forms.

### SDS-PAGE and Western Blotting

Native proteins and conjugates were analysed under heat denaturing non-reducing and reducing conditions by SDS-PAGE against a protein ladder using a 4-12% bis-tris gel and MES as running buffer (200V, ˜40 minutes). Samples were prepared to 0.5 mg/ml, comprising LDS sample buffer and MOPS running buffer as diluent. For reducing samples, DTT was added to a final concentration of 50 mM. Samples were heat treated for 2 minutes at 90-95° C. and 5 μg (10 μl) added to each well. Protein ladder (10 μl) was loaded without pre-treatment. Empty lines were filled with 1×LDS sample buffer (10 μl). After the gel was run, it was washed thrice with DI water (100 ml) with shaking (15 minutes, 200 rpm). Coomassie staining was performed by shaker-incubating the gel with PAGEBlue protein stain (30 ml) (60 minutes, 200 rpm). Excess staining solution was removed, rinsed twice with DI water (100 ml) and destained with DI water (100 ml) (60 minutes, 200 rpm). The resulting gel was imaged and processed using imageJ.

### TBEU-PAGE

Native protein, conjugates and BNA standard were analysed under heat denaturing non-reducing and reducing conditions by TBEU-PAGE against an oligo ladder using a 15% TBE-Urea gel and TBE as running buffer (180V, ˜60 minutes). Samples were prepared to 0.5 mg/ml, and BNA standard was prepared to 20 μg/ml, respectively, all comprising TBE Urea sample buffer and purified H₂O as diluent. Samples and standards were heat treated for 3 minutes at 70° C. and 10 μl added to each well, equating to 5 μg of protein and conjugate samples, and 0.2 μg of BNA, per lane. Oligo ladder reconstituted to 0.1 μg/band/ml in TE pH 7.5 (2 μl) was loaded without pre-treatment. After the gel was run, it was stained with freshly prepared ethidium bromide solution (1 μg/ml) with shaking (40 minutes, 200 rpm). The resulting gel was visualised by UV epi-illumination (254 nm), imaged and processed using imageJ.

### MALDI-TOF-MS

MALDI-TOF spectra were recorded on a MALDI-Mass Spectrometer (Bruker Ultraflex III). Typically, the sample dissolved in MilliQ water in nanomolar to micromolar range was spotted on the target (MTP 384 target plate polished steel T F, Bruker Daltons) using either super-DHB (99%, Fluka) or sinapinic acid (SA, 99%, Sigma-Aldrich) as the matrix dissolved in acetonitrile (MADLI-TOF-MS tested, Sigma)/0.1% TFA (7:3 v/v) via the dried-droplet-method. PepMix (Peptide Calibration Standard, Bruker Daltons) or ProteMass (Protein Calibration Standard, Sigma-Aldrich) served as calibration standards.

### V_{HH}-L-BNA Synthesis (FIG. 18)

### Intermediate 1:

### BNA-DTME (Molecule 3)

To an aliquot of ApoB BNA disulfide (9.4 mg, 1.6 μmol, 1.00 ml) prepared by reconstitution of lyophilised BNA in TBS pH 7.5 to 10 mg/ml, was added an aliquot of freshly prepared DTT solution (50.0 mg/ml, 10 mole equivalents, 13.0 μmol), the mixture vortexed briefly then incubated for 60 minutes at 37° C. with roller-mixing. After incubation, the resulting BNA-SH was concentrated to <1 ml and purified by gel filtration using a PD10 G25 desalting column eluting with TBS pH 7.5. The resulting BNA-SH (6.4 mg, 1.1 μmol, 2.00 ml) was reacted with freshly prepared DTME solution (20 mg/ml, 10 mole equivalents, 11.0 μmol, 0.171 ml) in DMSO, the mixture vortexed briefly then incubated for 60 minutes at 2⁰° C. After, the BNA-DTME (5.4 mg, 0.92 μmol, 1.22 mg/ml) was obtained following purification by gel filtration using a PD10 G25 desalting column into TBS pH 7.5. The BNA-DTME was stored at 20° C. until conjugated.

### V_{HH}-L-BNA (Molecule 5)

An aliquot of V_{HH} (3.0 mg, 0.2 μmol) was concentrated by ultrafiltration using a vivaspin T4 5 kDa MWCO centrifugal filter and buffer exchanged into TBS pH 7.5. To the concentrated V_{HH} (2.8 mg, 0.19 μmol, 2.83 mg/ml) was added an aliquot of freshly prepared TCEP solution (5.0 mg/ml, 4 mole equivalents, 0.77 μmol), the mixture vortexed briefly then incubated for 60 minutes at 37° C. with roller-mixing. After incubation, the resulting V_{HH}-SH was purified by gel filtration using a zeba spin desalting column then repeated centrifugal-wash cycles using a vivaspin T4 5 kDa MWCO centrifugal filter into TBS pH 7.5. The resulting V_{HH}-SH (2.0 mg, 0.13 μmol, 0.90 mg/ml, V_{HH}:SH=1.0) was reacted with BNA-DTME (1.1 mole equivalents, 0.83 mg, 0.14 μmol), the mixture vortexed briefly then incubated overnight at 20° C. After, the reaction mixture was concentrated using a vivaspin T4 10 kDa MWCO centrifuge tube and purified by gel filtration using a 1.6×35 cm Superdex 200PG column eluting into DPBS pH 7.5. The product (FIG. 18) fractions were collected, pooled and concentrated to <1 ml using a vivaspin T4 10 KDa MWCO centrifuge tubes. Yield: 0.81 mg, 0.40 mg/ml, 29%.

Trifunctional linker-(L-SO1861)-(L-BNA)-(V_{HH}) (FIG. 19, 20)

Referring to FIG. 19-23, the conjugate comprising V_{HH}, saponin SO1861 and ApoB BNA was synthesized, using the trifunctional linker TFL with molecular structure:

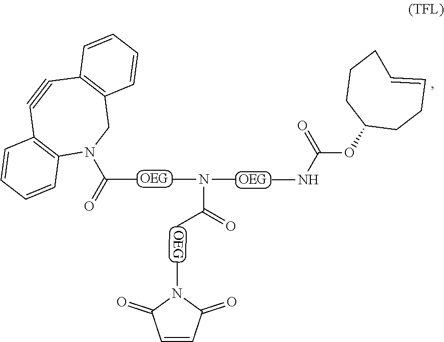

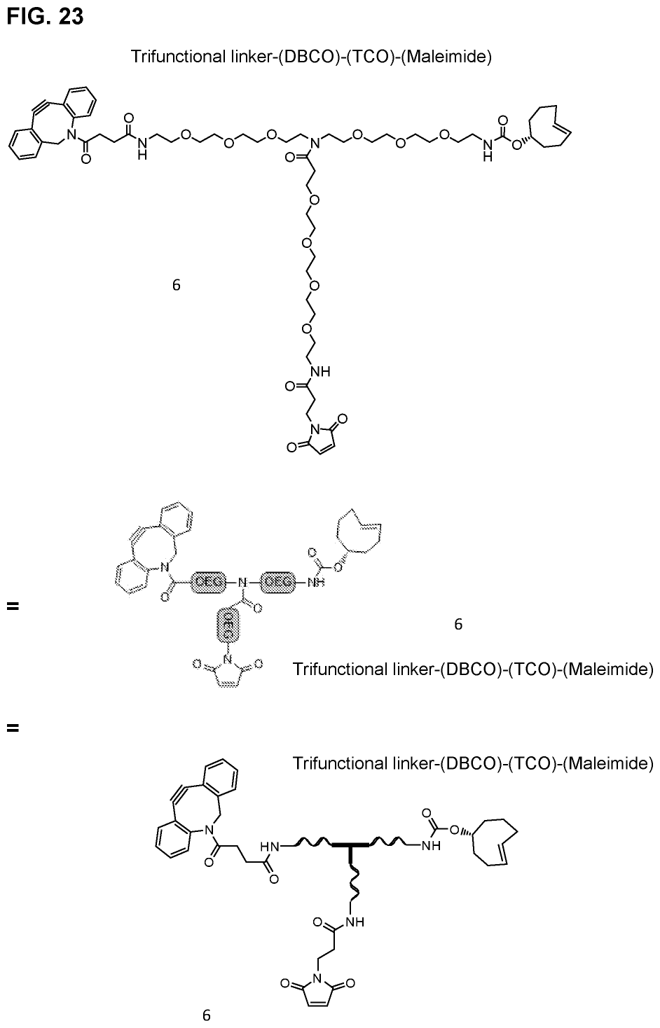

The molecular structure of the TFL (also referred to as (molecule 6) and as Trifunctional linker-(DBCO)-(TCO)-(Maleimide), is also depicted in FIG. 23. For conciseness reasons, the molecule 6 is also depicted as displayed here above and as given in FIG. 23.

The conjugate SO1861-L-ApoB BNA-V_{HH} Synthesis (V_{HH}-ApOB BNA-SO1861 conjugate).

### SO1861-L-azide (Molecule 7)

To SO1861 60 mg, 0.032 mmol)) and 1-azido-3,6,9,12-tetraoxapentadecane-15-hydrazide (39.3 mg, 0.129 mmol) was added methanol (extra dry, 1.00 mL) and TFA (9.86 μl, 0.129 mmol) and the reaction mixture was shaken for 1 min and left standing at room temperature. After 2 hours the reaction mixture was subjected to preparative MP-LC.¹ Fractions corresponding to the product (molecule 7) were immediately pooled together, frozen and lyophilized overnight to give the title compound (58.4 mg, 84%) as a white fluffy solid. Purity based on LC-MS 100%.
LRMS (m/z): 2150 [M−1]¹⁻
LC-MS r.t. (min): 1.10^{3B}

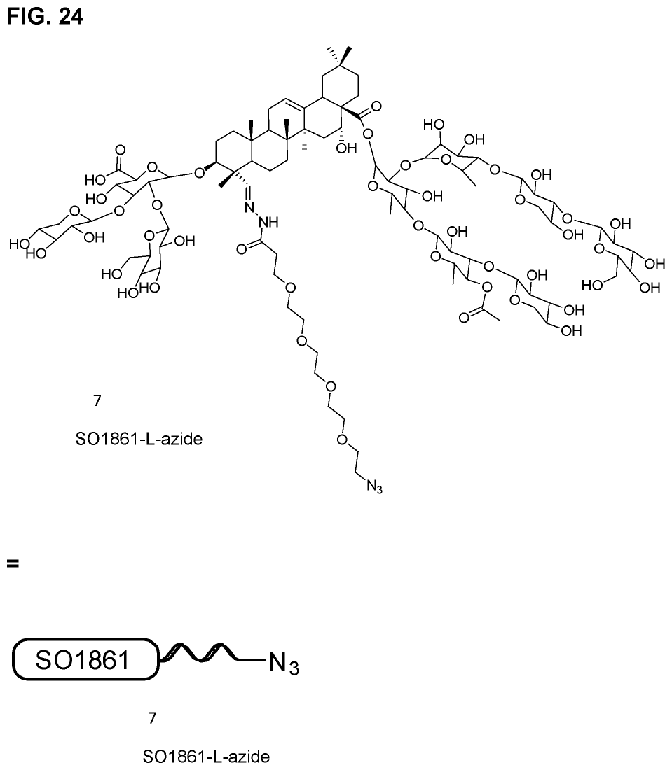

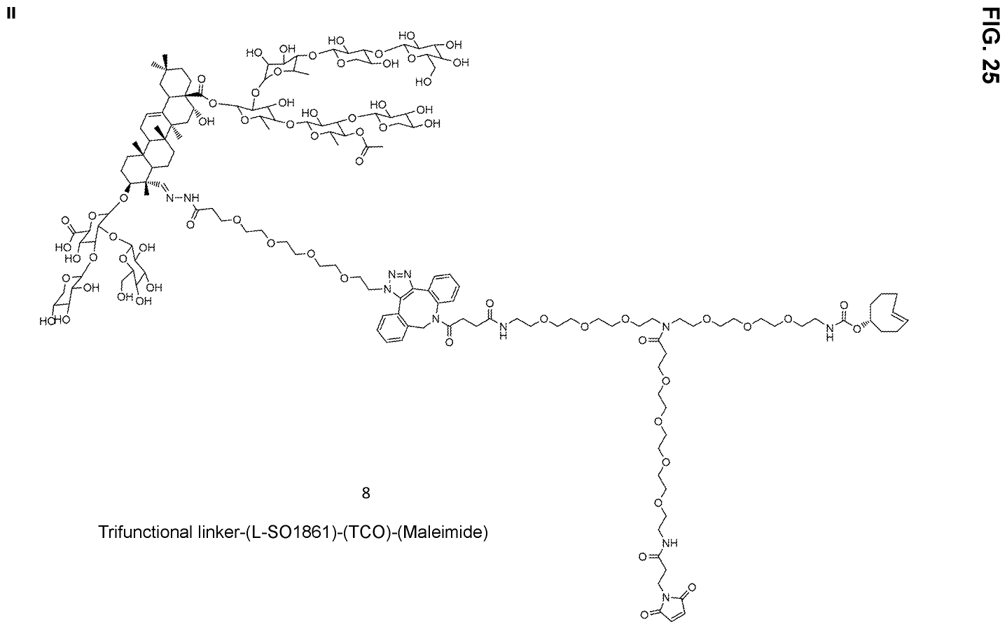

The molecular structure of molecule 7 is also depicted in FIG. 24, showing both the ‘all-atom’ representation and an abbreviated cartoon of the SO1861-L-azide, i.e. molecule 7.

### Intermediate 2:

Trifunctional linker-(L-SO1861)-(TCO)-(Maleimide) (Molecule 8)

A solution of TFL-(DBCO)-(TCO)-(Maleimide) (15 mg, 12.5 μmol) in DMF (2.0 mL) was added to SO1861-L-azide (26.8 mg, 12.5 μmol). The reaction mixture was shaken for 30 min and left standing at room temperature. After 30 min the reaction mixture was submitted to preparative MP-LC.^{1C} Fractions corresponding to the product were immediately pooled together, frozen and lyophilized overnight to give the title compound (31.4 mg, 75%) as a white fluffy solid. Purity based on LC-MS 96%.

MS (m/z): 3354 [M−1]¹⁻

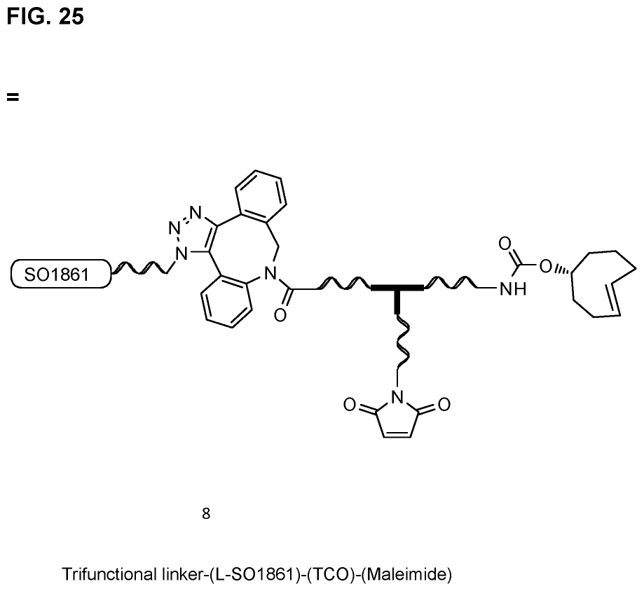

Molecule 8 is displayed in FIG. 25, in ‘all-atom’ representation and the Trifunctional linker-(L-SO1861)-(TCO)-(Maleimide) is also given as an abbreviated representation of molecule 8.

### Intermediate 3:

Trifunctional linker-(L-SO1861)-(TCO)-(V_{HH}) (Molecule 9)

An aliquot of V_{HH} (3.0 mg, 0.2 μmol) was concentrated by ultrafiltration using a vivaspin T4 5 kDa MWCO centrifugal filter and buffer exchanged into TBS pH 7.5. To the concentrated V_{HH} (2.8 mg, 0.19 μmol, 2.83 mg/ml) was added an aliquot of freshly prepared TCEP solution (5.0 mg/ml, 4 mole equivalents, 0.77 μmol), the mixture vortexed briefly then incubated for 60 minutes at 37° C. with roller-mixing. After incubation, the resulting V_{HH} was purified by gel filtration using a zeba spin desalting column then repeated centrifugal-wash cycles using a vivaspin T4 5 kDa MWCO centrifugal filter into TBS pH 7.5. The resulting V_{HH}-SH (molecule 4) (2.0 mg, 0.13 μmol, 0.90 mg/ml, V_{HH} Q8c:SH=1.0) was reacted with TFL-(L-SO1861)-(TCO)-(Maleimide) (1.1 mole equivalents, 0.5 mg, 0.15 μmol), the mixture vortexed briefly then incubated overnight at 20° C. After, the reaction mixture was concentrated using a vivaspin T4 10 kDa MWCO centrifuge tube and purified by gel filtration using a 1.6×35 cm Superdex 200PG column eluting into DPBS pH 7.5. The product fractions were collected, pooled and concentrated to <1 ml using a vivaspin T4 10 KDa MWCO centrifuge tubes. Yield: 1.3 mg, 0.40 mg/ml, 54%.

MS (m/z): 18062 [M+Na]¹⁺

### Intermediate 4:

methyltetrazine-L-ApoB BNA (Molecule 11)

To ApoB BNA-disulfide (molecule 1) (5.00 mg, 0.686 μmol) was added a solution of 20 mM ammonium bicarbonate with 2.5 mM TCEP (1.00 mL, 2.5 μmol). The reaction mixture was shaken for 1 min and left standing at room temperature. After 1 hour the reaction mixture was filtered by using a centrifugal filter with a molecular weight cut-off of 3000 Da (5000×g for 30 min, 2×0.50 mL). Next, the residue solution was washed twice with a solution of 20 mM ammonium bicarbonate with 2.5 mM TCEP (0.50 mL), each time filtered under the same conditions described above. As next, the residue solution was diluted with 20 mM ammonium bicarbonate (1.50 mL) and the resulting mixture was directly added to a solution of (E)-1-(4-((2-(6-(2,5-dioxo-2,5-dihydro-1H-pyrrol-1-yl)hexanoyl)hydrazineylidene)methyl)benzamido)-N-(4-(6-methyl-1,2,4,5-tetrazin-3-yl)benzyl)-3,6,9,12-tetraoxapentadecan-15-amide (molecule 10) (1.36 mg, 1.73 μmol) in acetonitrile (0.5 mL). The reaction mixture was shaken for 1 min and left standing at room temperature. After 30 min the reaction mixture was frozen and lyophilized overnight to yield the crude title product as a pink fluffy solid. To the crude product was added a solution of 20 mM ammonium bicarbonate (1.50 mL) and the resulting suspension was filtered over a 0.45 μm syringe filter. The filtrate was lyophilized overnight to yield the title product (molecule 11) (5.44 mg, quant) as a pink fluffy solid. Purity based on LC-MS 90%.

LRMS (m/z): 2648 [M−3]³⁻

LC-MS r.t. (min): 0.624

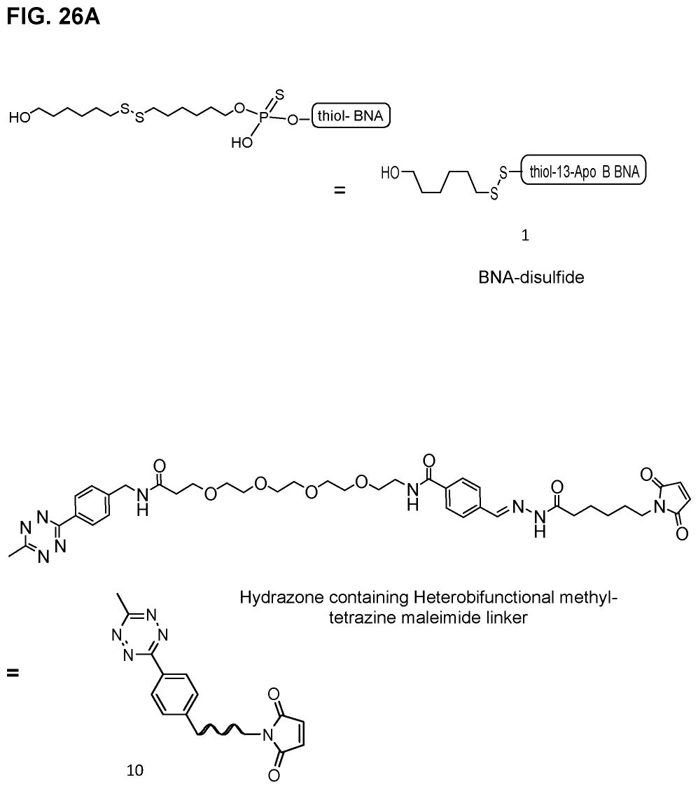

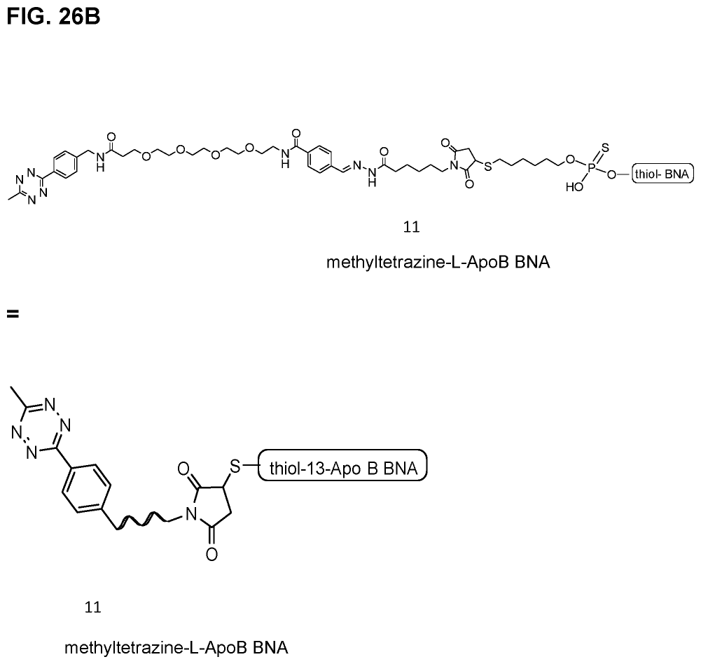

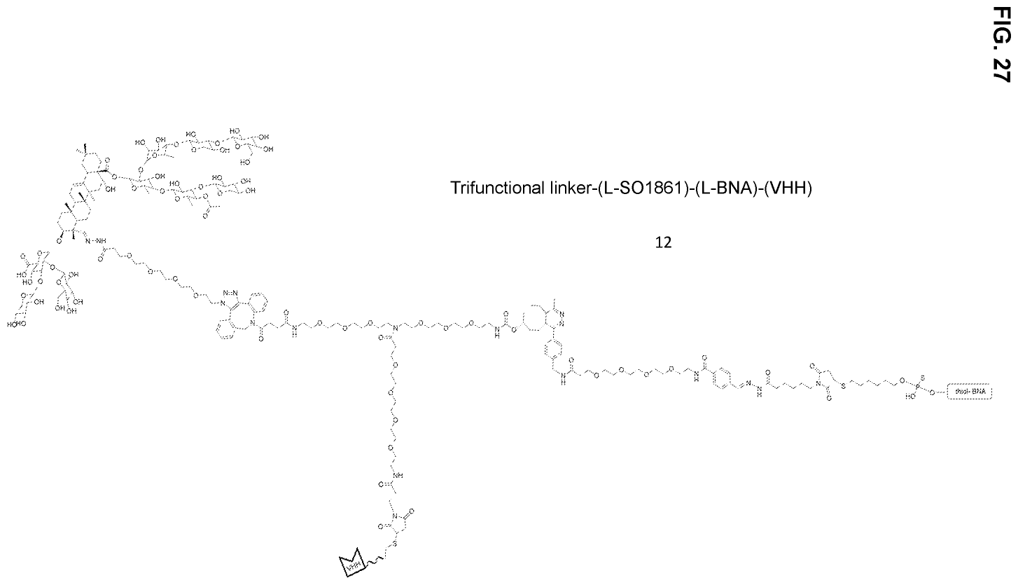

Molecule 11 (methyltetrazine-L-ApoB BNA) is depicted in FIG. 26B, molecule 1 (ApoB BNA-disulfide) and molecule 10 ((E)-1-(4-((2-(6-(2,5-dioxo-2,5-dihydro-1H-pyrrol-1-yl)hexanoyl)hydrazineylidene)methyl)benzamido)-N-(4-(6-methyl-1,2,4,5-tetrazin-3-yl)benzyl)-3,6,9,12-tetraoxapentadecan-15-amide) are depicted in FIG. 26A.

Trifunctional linker-(L-SO1861)-(L-BNA)-(V_{HH}) (Molecule 12)

To a solution of trifunctional linker-(L-SO1861)-(TCO)-(V_{HH}) (molecule 9) (3 mg, 0.16 μmol, 0.40 mg/ml) dissolved in DPBS pH 7.5 Methyltetrazine-BNA oligo (molecule 11) (0.63 mg, 96 nmol) was added. The reaction mixture was shaken for 1 min and was incubated overnight. After, the reaction mixture was concentrated using a vivaspin T4 10 kDa MWCO centrifuge tube and purified by gel filtration using a 1.6×35 cm Superdex 200PG column eluting into DPBS pH 7.5. The product fractions were collected, pooled and concentrated to <1 ml using a vivaspin T4 10 KDa MWCO centrifuge tubes. Yield: 2.4 mg, 0.40 mg/ml, 61%.

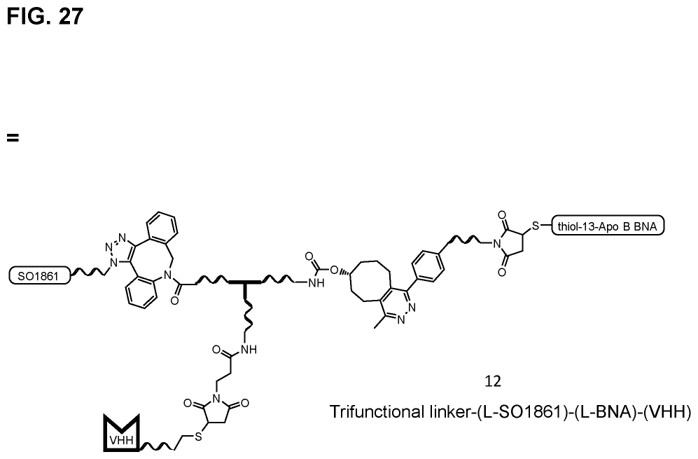

Trifunctional linker-(L-SO1861)-(L-BNA)-(V_{HH}) (molecule 12) is displayed in FIG. 27, in two representations.

Trifunctional linker-(dendron(-L-SO1861)⁴)-(L-BNA)-(V_{HH}) (FIGS. 21 and 22)

Referring to FIG. 19-22, the conjugate comprising V_{HH}, dendron(-L-SO1861)⁴ and ApoB BNA was synthesized, using the trifunctional linker (TFL; molecule 6) with molecular structure:

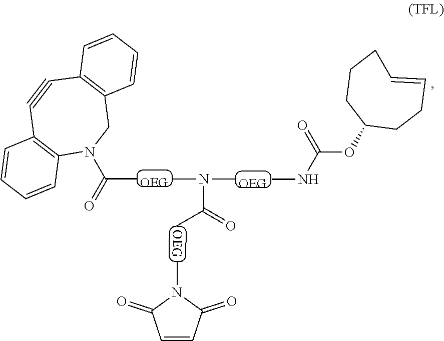

The conjugate dendron(-L-SO1861)4-L-BNA-V_{HH} synthesis (V_{HH}-BNA-dendron(-L-SO1861)4 conjugate) was formed.

dendron(-L-SO1861)⁴-azide (Molecule 13)
Dendron(SO1861)⁴-amine (6.81 mg, 0.748 μmol) and 2,5-dioxopyrrolidin-1-yl 1-azido-3,6,9,12-tetraoxapentadecan-15-oate (2.90 mg, 7.48 μmol) were dissolved in DMF(1.00 mL). Next, DIPEA (1.302 μL, 7.48 μmol) was added and the mixture was shaken for 1 min and left standing at room temperature. After 2 hours the reaction mixture was subjected to preparative LC-MS.^{3C} Fractions corresponding to the product were immediately pooled together, frozen and lyophilized overnight to give the title compound (5.86 mg, 84%) as a white fluffy solid. Purity based on LC-MS 90%.

LRMS (m/z): 2344 [M−4]⁴⁻

LC-MS r.t. (min): 4.78^{5B}

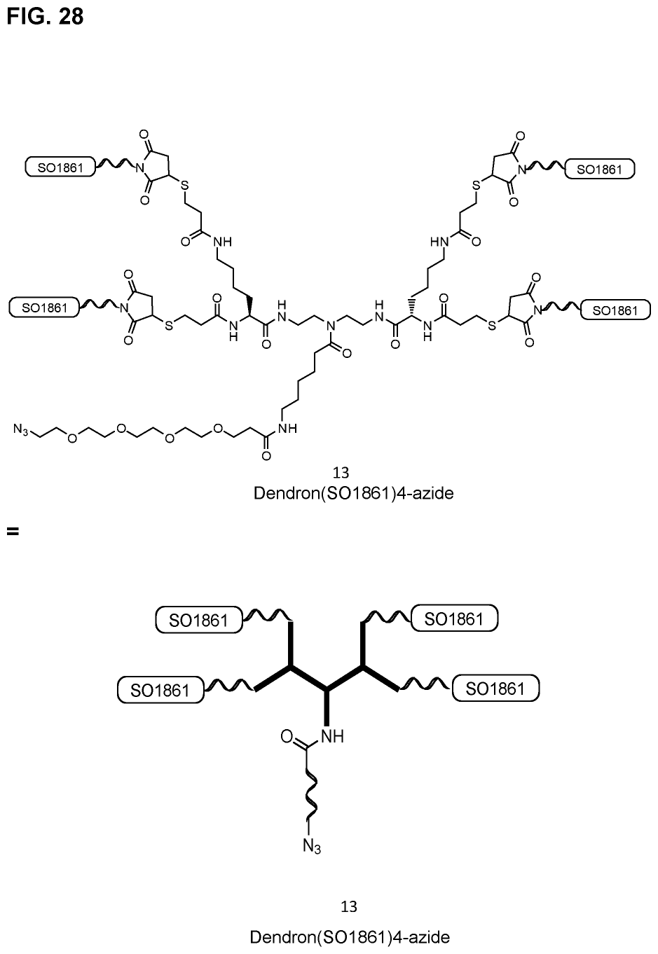

The molecular structure of molecule 13 is depicted in FIG. 28, as well as a simplified representation of the dendron(-L-SO1861)⁴-azide (also referred to as dendron(SO1861)4-azide).

### Intermediate 5:

Trifunctional linker-(dendron(-L-SO1861)⁴)-(TCO)-(Maleimide) (molecule 14) A solution of TFL-(DBCO)-(TCO)-(Maleimide) (molecule 6) (5 mg, 4.1 μmol) in DMF (2.0 mL) was added to dendron(-L-SO1861)₄-azide (molecule 13) (39.4 mg, 4.2 μmol). The reaction mixture was shaken for 90 min and left standing at room temperature. After 90 min the reaction mixture was submitted to preparative MP-LC.^{1C} Fractions corresponding to the product (molecule 14) were immediately pooled together, frozen and lyophilized overnight to give the title compound (31.2 mg, 72%) as a white fluffy solid. Purity based on LC-MS 96%.

MS (m/z): 10611 [M+Na]¹⁺

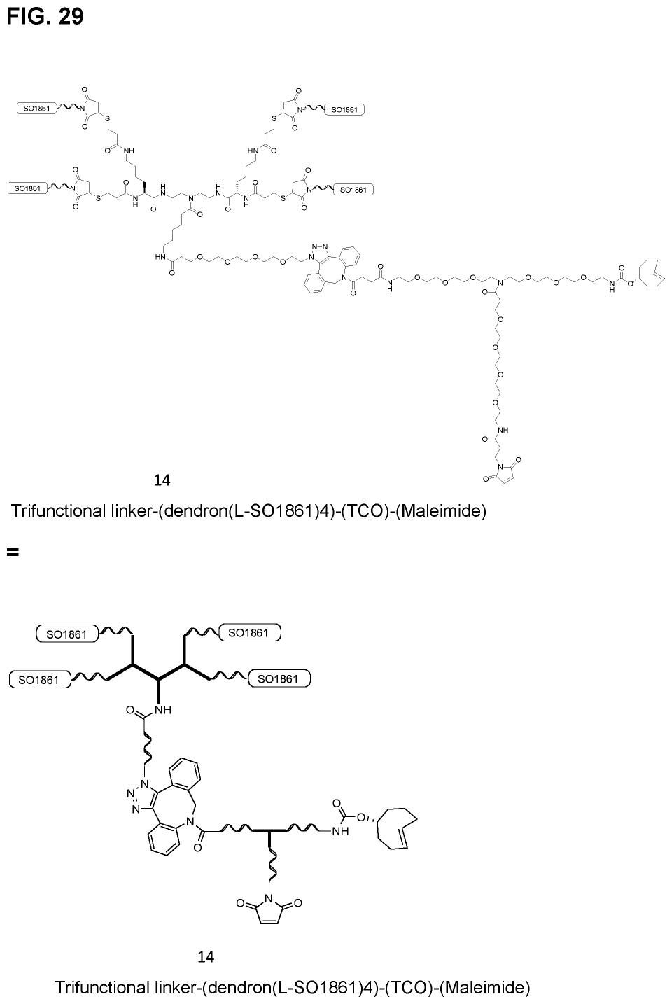

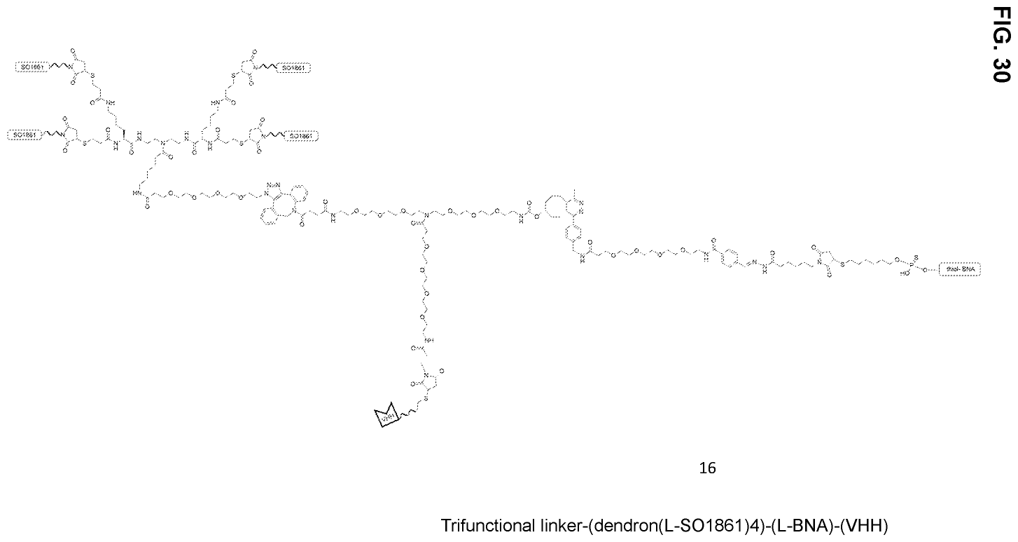

FIG. 29 displays the chemical structure of the molecule 14 (Trifunctional linker-(dendron(-L-SO1861)⁴)-(TCO)-(Maleimide)) and a simplified drawing of the same molecule.

### Intermediate 6:

Trifunctional linker-(dendron(-L-SO1861)⁴)-(TCO)-(V_{HH}) (Molecule 15)

An aliquot of V_{HH} (3.0 mg, 0.2 μmol) was concentrated by ultrafiltration using a vivaspin T4 5 kDa MWCO centrifugal filter and buffer exchanged into TBS pH 7.5. To the concentrated V_{HH} (2.8 mg, 0.19 μmol, 2.83 mg/ml) was added an aliquot of freshly prepared TCEP solution (5.0 mg/ml, 4 mole equivalents, 0.77 μmol), the mixture vortexed briefly then incubated for 60 minutes at 37° C. with roller-mixing. After incubation, the resulting V_{HH}-SH (molecule 4) was purified by gel filtration using a zeba spin desalting column then repeated centrifugal-wash cycles using a vivaspin T4 5 kDa MWCO centrifugal filter into TBS pH 7.5. The resulting V_{HH}-SH (2.0 mg, 0.13 μmol, 0.90 mg/ml, V_{HH}:SH=1.0) was reacted with TFL-(dendron(-L-SO1861)⁴)-(TCO)-(Maleimide) (molecule 14) (1.1 mole equivalents, 1.5 mg, 0.14 μmol), the mixture vortexed briefly then incubated overnight at 20° C. After, the reaction mixture was concentrated using a vivaspin T4 10 kDa MWCO centrifuge tube and purified by gel filtration using a 1.6×35 cm Superdex 200PG column eluting into DPBS pH 7.5. The product (molecule 15) fractions were collected, pooled and concentrated to <1 ml using a vivaspin T4 10 KDa MWCO centrifuge tubes. Yield: 0.95 mg, 0.40 mg/ml, 29%. The V_{HH} is for example V_{HH} Q8c:SH which is an sdAb for binding to HIVgp41 and produced by clone anti-HIVgp41 Q8C-tag, or the V_{HH} is for example V_{HH} 7D12 (SEQ ID NO: 75) or V_{HH} 9G8 (SEQ ID NO: 76), or the tandem of two V_{HH}'s 7D12-9G8 (SEQ ID NO: 74).

MS (m/z): 25295 [M+Na]¹⁺

### Intermediate 4:

methyltetrazine-L-ApoB BNA (Molecule 11)

To ApoB BNA disulfide (5.00 mg, 0.686 μmol) was added a solution of 20 mM ammonium bicarbonate with 2.5 mM TCEP (1.00 mL, 2.5 μmol). The reaction mixture was shaken for 1 min and left standing at room temperature. After 1 hour the reaction mixture was filtered by using a centrifugal filter with a molecular weight cut-off of 3000 Da (5000×g for 30 min, 2×0.50 mL). Next, the residue solution was washed twice with a solution of 20 mM ammonium bicarbonate with 2.5 mM TCEP (0.50 mL), each time filtered under the same conditions described above. As next, the residue solution was diluted with 20 mM ammonium bicarbonate (1.50 mL) and the resulting mixture was directly added to a solution of (E)-1-(4-((2-(6-(2,5-dioxo-2,5-dihydro-1H-pyrrol-1-yl)hexanoyl)hydrazineylidene)methyl)benzamido)-N-(4-(6-methyl-1,2,4,5-tetrazin-3-yl)benzyl)-3,6,9,12-tetraoxapentadecan-15-amide (1.36 mg, 1.73 μmol) in acetonitrile (0.5 mL). The reaction mixture was shaken for 1 min and left standing at room temperature. After 30 min the reaction mixture was frozen and lyophilized overnight to yield the crude title product as a pink fluffy solid. To the crude product was added a solution of 20 mM ammonium bicarbonate (1.50 mL) and the resulting suspension was filtered over a 0.45 μm syringe filter. The filtrate was lyophilized overnight to yield the title product (5.44 mg, quant) as a pink fluffy solid. Purity based on LC-MS 90%.

LRMS (m/z): 2648 [M−3]³⁻

LC-MS r.t. (min): 0.624

Trifunctional linker-(dendron(-L-SO1861)⁴)-(L-BNA)-(V_{HH}) (Molecule 16)

To a solution of TFL-(dendron(-L-SO1861)⁴)-(TCO)-(V_{HH}) (molecule 15) (2.2 mg, 87 nmol, 0.40 mg/ml) dissolved in DPBS pH 7.5 Methyltetrazine-BNA (molecule 11) (0.4 mg, 60 nmol) was added. The reaction mixture was shaken for 1 min and was incubated overnight. After, the reaction mixture was concentrated using a vivaspin T4 10 kDa MWCO centrifuge tube and purified by gel filtration using a 1.6×35 cm Superdex 200PG column eluting into DPBS pH 7.5. The product (molecule 16) fractions were collected, pooled and concentrated to <1 ml using a vivaspin T4 10 KDa MWCO centrifuge tubes. Yield: 2.0 mg, 0.40 mg/ml, 73%.

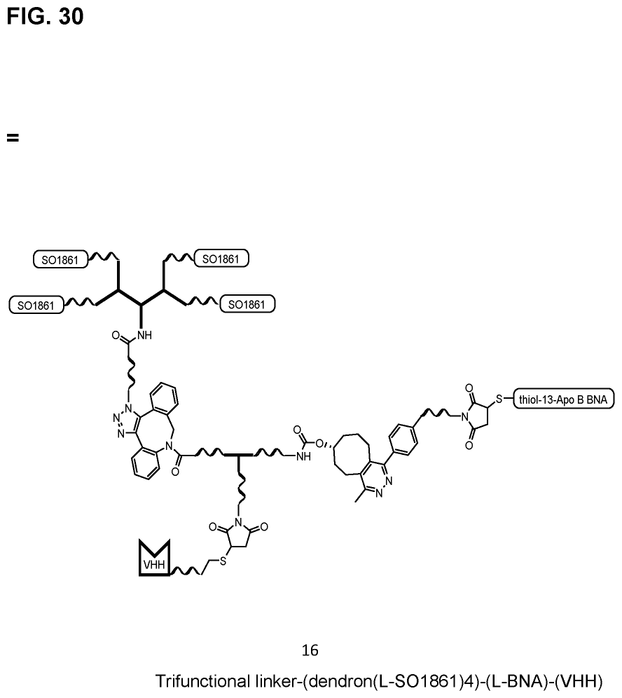

The molecular structure of Trifunctional linker-(dendron(-L-SO1861)⁴)-(L-BNA)-(V_{HH}) (molecule 16) is depicted in FIG. 30, also in abbreviated representation.

### Dendron for Coupling 8 SO1861 Moieties

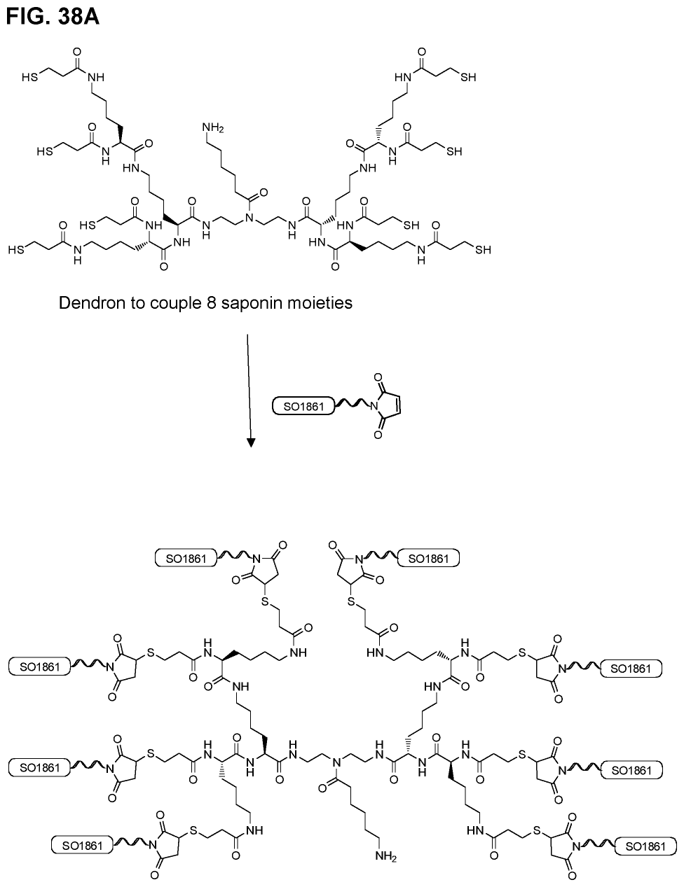

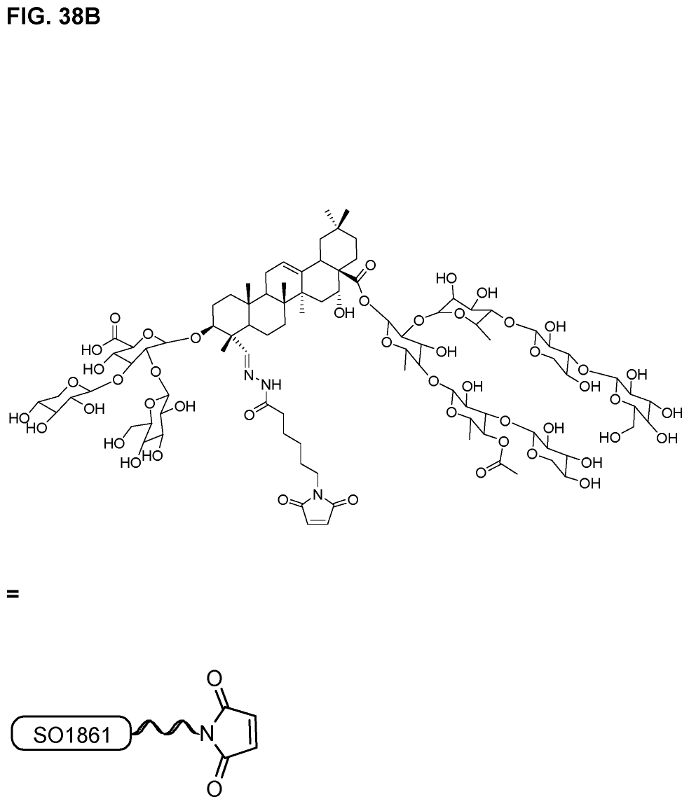

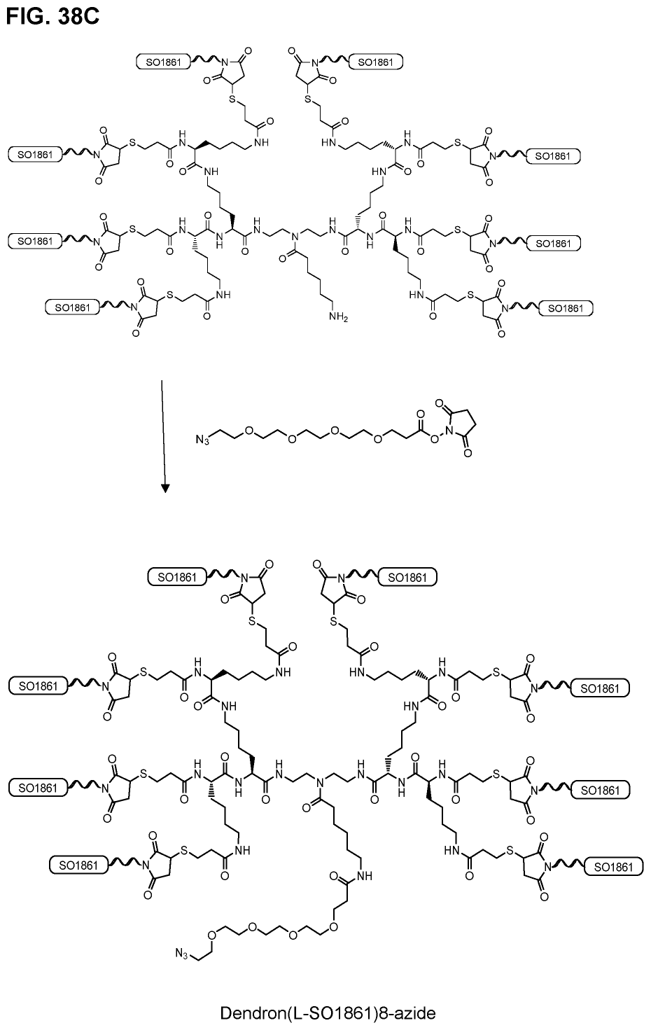

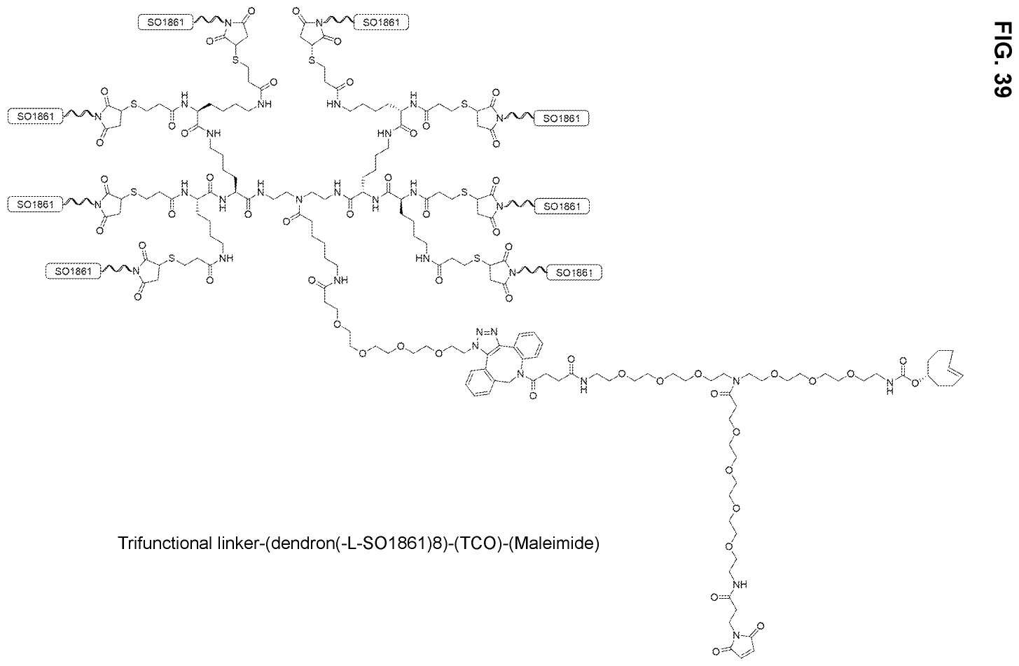

FIG. 38A displays the G3 dendron for covalently coupling of maximally 8 saponin moieties, here in the example SO1861 coupled to EMCH through a hydrazone bond, providing the maleimide group (displayed in FIG. 38B) for covalent coupling to the free —SH thiol groups of the dendron. FIG. 38A also displays the resulting dendron with 8 saponin moieties covalently bound thereto. FIG. 38C displays the subsequent step of providing the dendron(-L-SO1861)8 conjugate with the azide group through coupling as depicted, therewith providing dendron(-L-SO1861)8-azide.

Trifunctional linker-(dendron(-L-SO1861)⁸)

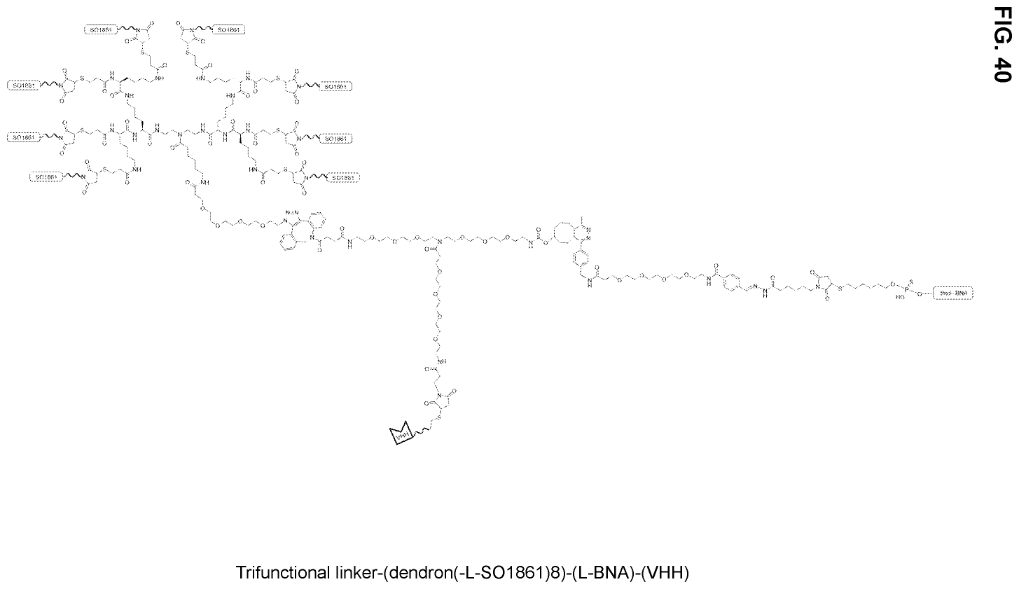

FIG. 39 displays the conjugation product Trifunctional linker-(dendron(-L-SO1861)8)-(TCO)-(Maleimide) of the dendron(-L-SO1861)8-azide (FIG. 38C) with the trifunctional linker (molecule 6), also referred to as TFL.

Trifunctional linker-(dendron(-L-SO1861)⁸)-(L-BNA)-(V_{HH})

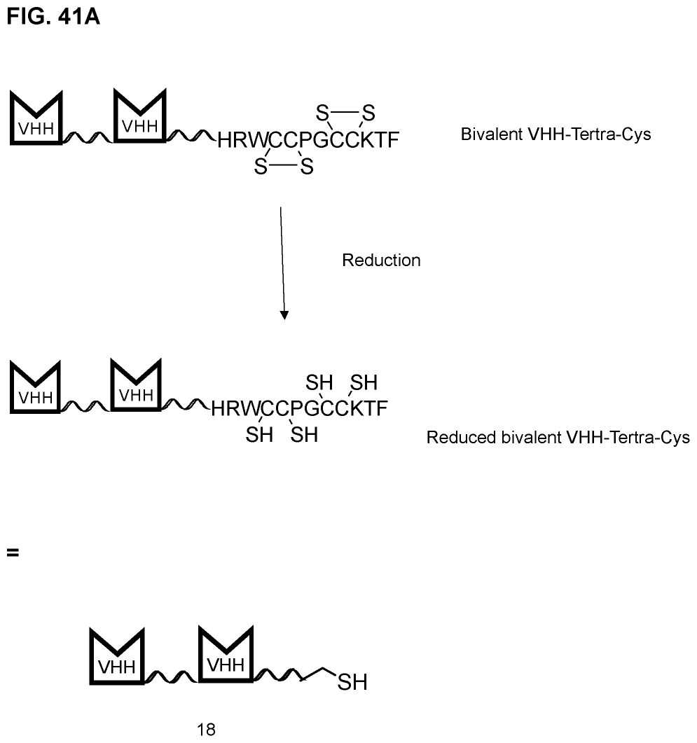

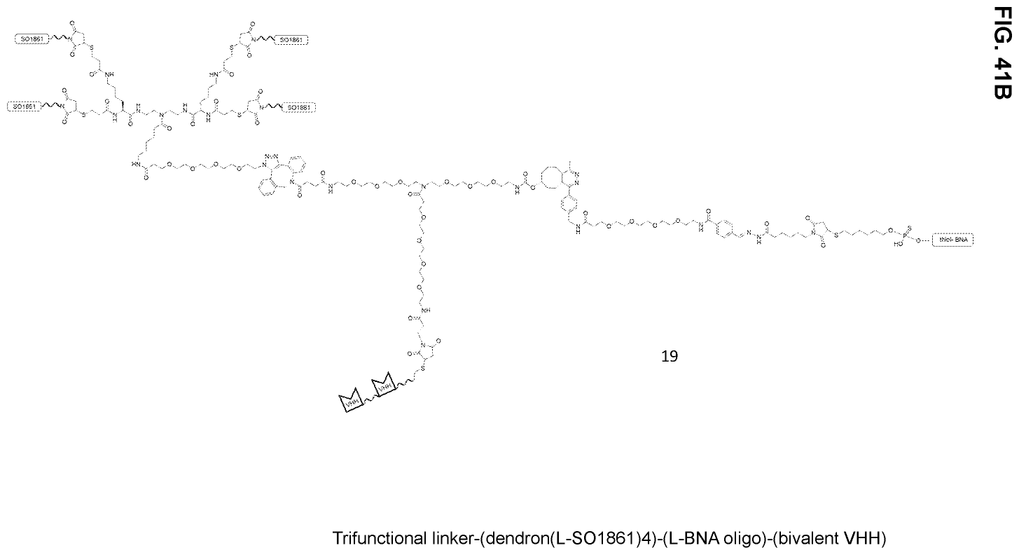

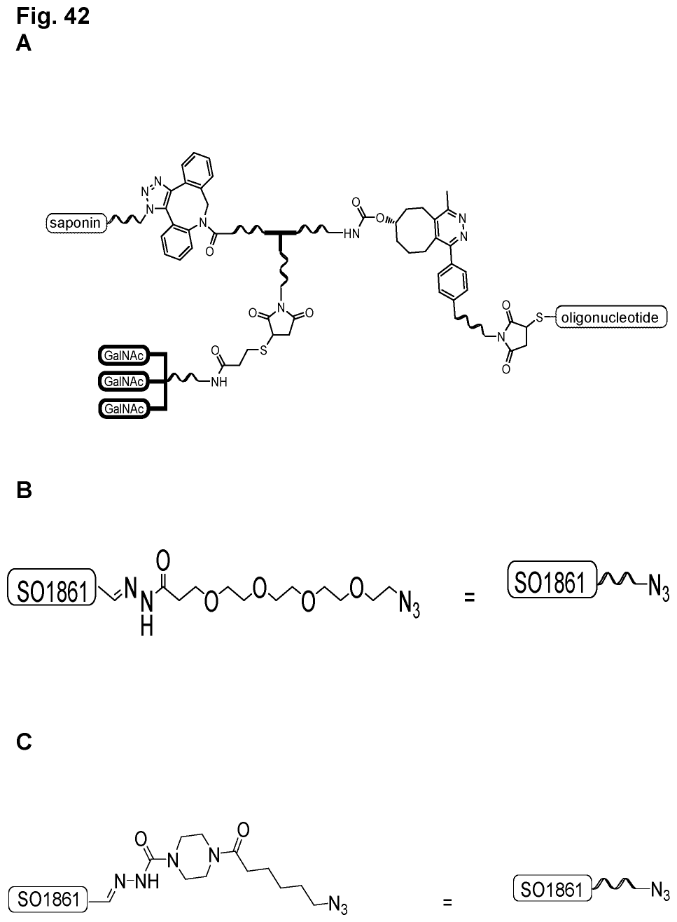

FIG. 40 displays the reaction product Trifunctional linker-(dendron(-L-SO1861)3)-(L-BNA)-(V_{HH}) of the conjugation of Trifunctional linker-(dendron(-L-SO1861)8)-(TCO)-(Maleimide) (FIG. 39) with the oligonucleotide as displayed in FIG. 36 and the V_{HH} domain which is coupled via a thiol group of a cysteine residue in the amino-acid sequence of the domain (see also FIG. 41A).

Trifunctional Linker-(Dendron(-L-SO1861)⁴)-(L-BNA)-(Bivalent V_{HH} with Tetra-Cysteine Linker HRWCCPGCCKTF)

FIG. 41A displays a bivalent V_{HH} with a C-terminal linker sequence comprising a tetra-Cys repeat for covalent coupling (SEQ ID NO: 77: Amino-acid sequence of tetra-Cys artificial linker). The disulphide bonds are reduced as depicted, providing the bivalent V_{HH} molecule with the thiol groups available for covalent bonding, for example with the maleimide group of the trifunctional linker displayed as molecule 6. FIG. 41B displays the 1-component bivalent V_{HH} conjugate Trifunctional linker-(dendron(L-SO1861)4)-(L-BNA oligo)-(bivalent V_{HH}) Comprising four saponin moieties and a single oligonucleotide moiety.

### Example B: Critical Micellar Concentration (CMC) of Saponins

### Materials and Methods

The critical micellar concentration (CMC) of saponin SO1861 derived from Saponaria officinalis (SO) and QS saponins derived from Quillaja saponaria (QS) (Table A5) was determined by the method of DeVendittis et al. (A fluorimetric method for the estimation of the critical micelle concentration of surfactants, Analytical Biochemistry, Volume 115, Issue 2, August 1981, Pages 278-286) as follows:

The emission spectrum of 8-Anilinonaphthalene-1-sulfonic acid (ANS) in either purified water (MQ) or PBS (Dulbecco's PBS+/+) was determined at dry weight concentrations of saponins ranging from 1 to 1400 pM to cover the range below and above the CMC. Above the CMC, the fluorescence yield of ANS increases and the wavelength of maximum emission decreases due to portioning of the fluorescent dye into micelles. Fluorescence yields were recorded on a Fluoroskan Ascent FL (Thermo Scientific) at an excitation wavelength of 355 nm, and an emission wavelength of 460 nm. 6 μg at a concentration of 75.86 μM of ANS were used per sample and measurement.

### Results

### SO1861 Saponin

The CMC value as obtained for the native SO1861 in PBS is 185 μM.

### QS Saponins

For the saponins derived from Quillaja saponaria (QS), QS7, QS17, QS18, QS21, CMC values have been determined which are displayed in Table A5.

TABLE A5
CMC values of QS saponins

| Saponin | CMC (μM) |
|---|---|
| QS7 | 230 ± 25 |
| QS21 | 75 ± 8 |
| QS17 | 70 ± 7 |
| QS18 | 68 ± 7 |

### Example C—Potency of 1-Component Conjugates Comprising a Single Copy of a Saponin or Comprising Multiple Copies of the Saponin, Wherein the Saponin is Covalently Linked Via an Acid-Labile Hydrazone Bond or an Acid-Labile Semicarbazone Bond

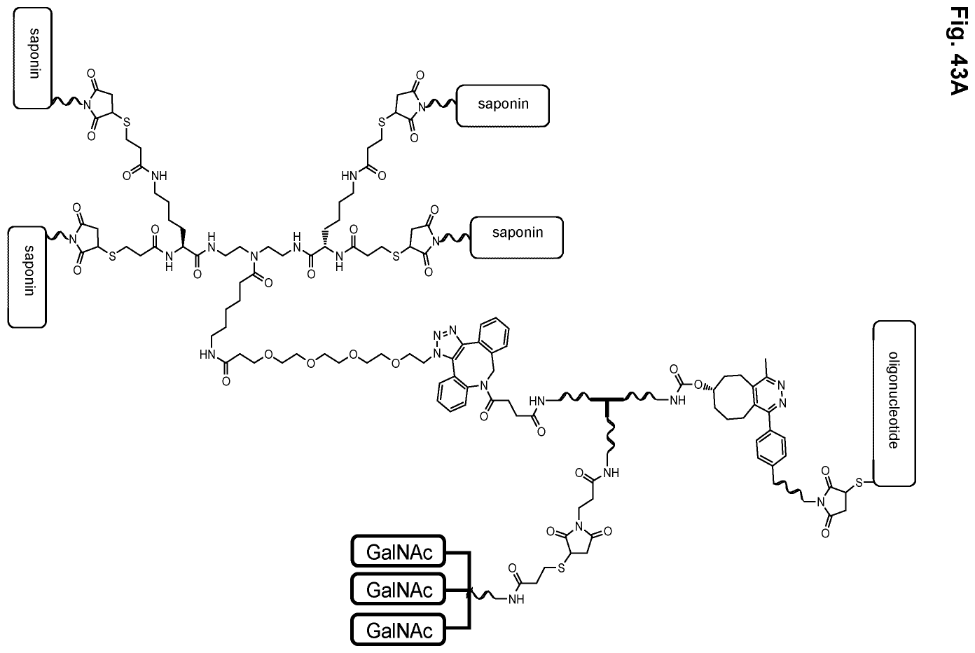

The effect of 1-component conjugates comprising ApoB BNA and 1, 4 or 8 copies of the saponin SO1861 and comprising the liver cell receptor asialoglycoprotein receptor (ASGPR) ligand tri-GalNAc, on silencing of the ApoB gene in mouse liver cells is assessed, as well as the ApoB protein expression under influence of the conjugates. Mice are treated with a series of conjugates, displayed in FIG. 42-44. The saponin(s) is/are covalently coupled to the trifunctional linker (molecule 6; TFL) through either a hydrazone bond, or a semicarbazone bond.

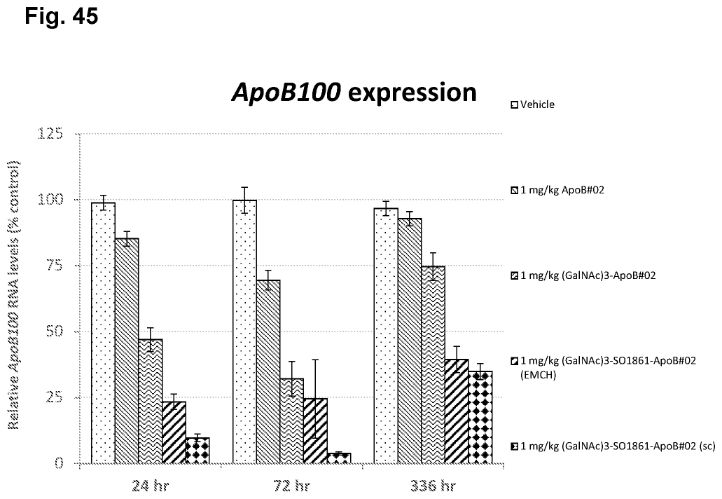

As FIG. 45 shows, after 24 hrs, the apoB RNA levels, i.e. gene expression, was ca 15% reduced in groups dosed with 1 mg/kg ApoB #02 BNA (n=3 animals per group), compared to the vehicle-dosed group (n=5 animals). Likewise, (GalNAc)₃-ApoB #02, at a dose of 1 mg/kg (corresponding to 0.54 mg/kg ApoB #02 BNA) reduced ApoB expression by ca 53% (n=3 animals) compared to the vehicle control after 24 hrs, while 0.1 mg/kg (GalNAc)₃-ApoB #02 alone did not show a noticeable reduction (n=3) compared to vehicle. Most notably, treatment with 1 mg/kg (GalNAc)3-SO1861-ApoB #02 BNA (corresponding to 0.44 mg/kg ApoB #02 BNA) resulted in and ca 77%% reduction for (GalNAc)3-SO1861-ApoB #02 BNA (EMCH) (see FIG. 42A, B; saponin coupled via an acid-labile hydrazone bond in the 1-component conjugate) and 90% reduction for (GalNAc)3-SO1861-ApoB #02 BNA (sc) (see FIG. 42A, C; saponin coupled via an acid-labile semicarbazone bond in the 1-component conjugate), compared to control already 24 hrs post dosing (n=3 animals in both groups each). The apoB RNA levels, i.e. gene expression, was not noticeably reduced in animals treated with 0.1 mg/kg ApoB #02 BNA (ApoB #02). The effect was durable, and after 336 hrs, apoB expression levels were reduced by 30% for 2 mg/kg ApoB #2 BNA (n=2 animals) and 25% for 1 mg/kg (GalNAc)3-ApoB #02. Treatment with different 1 mg/kg (GalNAc)₃-SO1861-ApoB #02 BNA conjugates resulted in ca 61% reduction for (GalNAc)3-SO1861-ApoB #02 BNA (EMCH) and 65% reduction for (GalNAc)3-SO1861-ApoB #02 BNA (sc) (n=3 animals in both groups each). In conclusion, conjugation of SO1861 to the payload markedly improves potency. In addition, the apoB RNA down-modulation in the liver also translated to apoB protein reduction in serum.

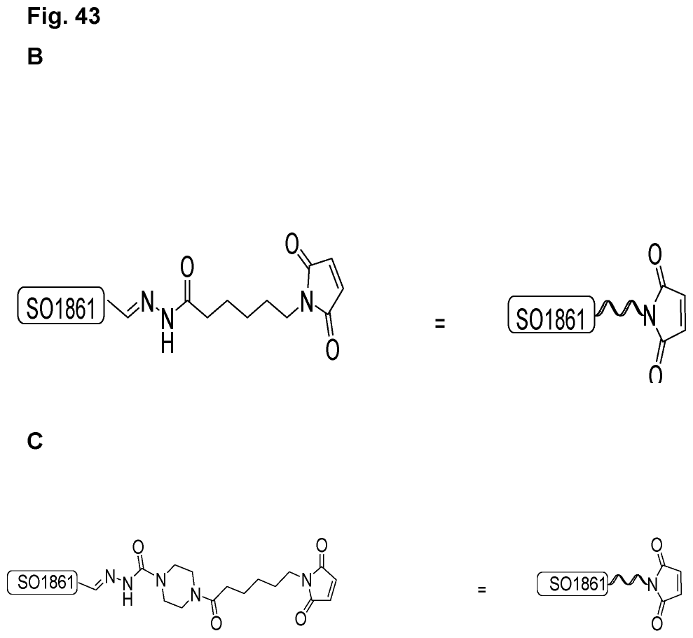

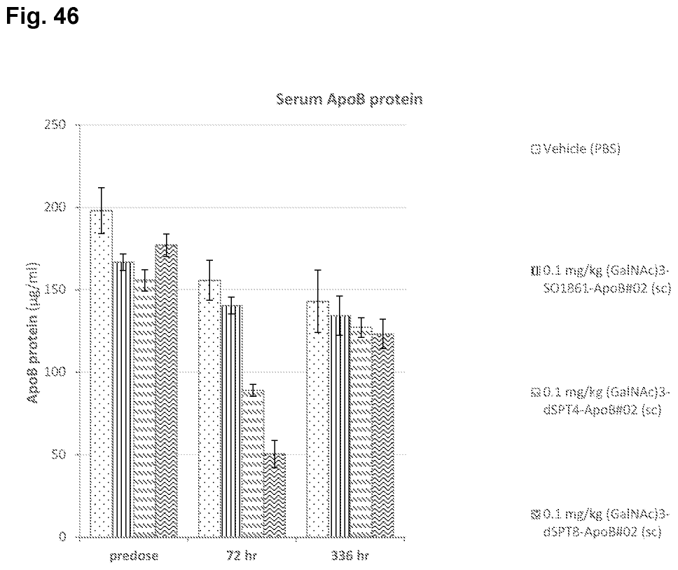

As FIG. 46 shows, observed apoB RNA down-modulation in the liver (see for example FIG. 45) also translated to apoB protein reduction in serum. Predose levels ranged from 156-198 μg/ml over the groups (n=8 per group). At 72 hrs post-dose, the apoB protein levels were reduced in animals treated with the benchmark 2 mg/kg ApoB #02 BNA (57 μg/ml average compared to 156 μg/ml of the vehicle control), and was even more reduced (to 18 μg/ml and 21 μg/ml of vehicle control, respectively) in the groups dosed with 1 mg/kg (GalNAc)3-SO1861-ApoB #02 (sc) (see FIG. 42A, C; saponin coupled via an acid-labile semicarbazone bond to the trifunctional linker in the 1-component conjugate), respectively, showing the markedly improved potency when adding SO1861 to the conjugate (n=8 animals per group). As seen with the RNA levels (not shown), when increasing the saponin-per-payload, by dosing 0.173 mg/kg (GalNAc)3-d(SO1861)4-ApoB #02 (sc) (see FIG. 43A, C; four saponins coupled via an acid-labile semicarbazone bond to the G2 dendron in the 1-component conjugate) or 0.265 mg/kg (GalNAc)₃-d(SO1861)8-ApoB #02 (sc) (see FIG. 43C and FIG. 44; eight saponins coupled via an acid-labile semicarbazone bond to the G3 dendron in the 1-component conjugate), respectively, serum apoB protein was reduced to 89 μg/ml and 50 μg/ml, respectively, while it was 141 μg/ml for 0.1 mg/kg (GalNAc)3-SO1861-ApoB #02 (sc), showing that an increase of saponin copies in the conjugate markedly increases potency of the conjugate in reducing apoB protein. After 336 hrs, the effect of protein reduction was still preserved in the 1 mg/kg (GalNAc)3-SO1861-ApoB #02 (sc) dose group and was 59 μg/ml, compared to 143 μg/ml in the vehicle control (n=5 mice in all groups) (not shown). The observed serum apoB protein reduction (shown in FIG. 46) translated well to serum LDL-cholesterol (LDL-C) levels.

### Example D—Release Kinetics of Saponin Linked Via a Hydrazone Bond or a Semicarbazone Bond in a Conjugate, Under Influence of Several pH Values

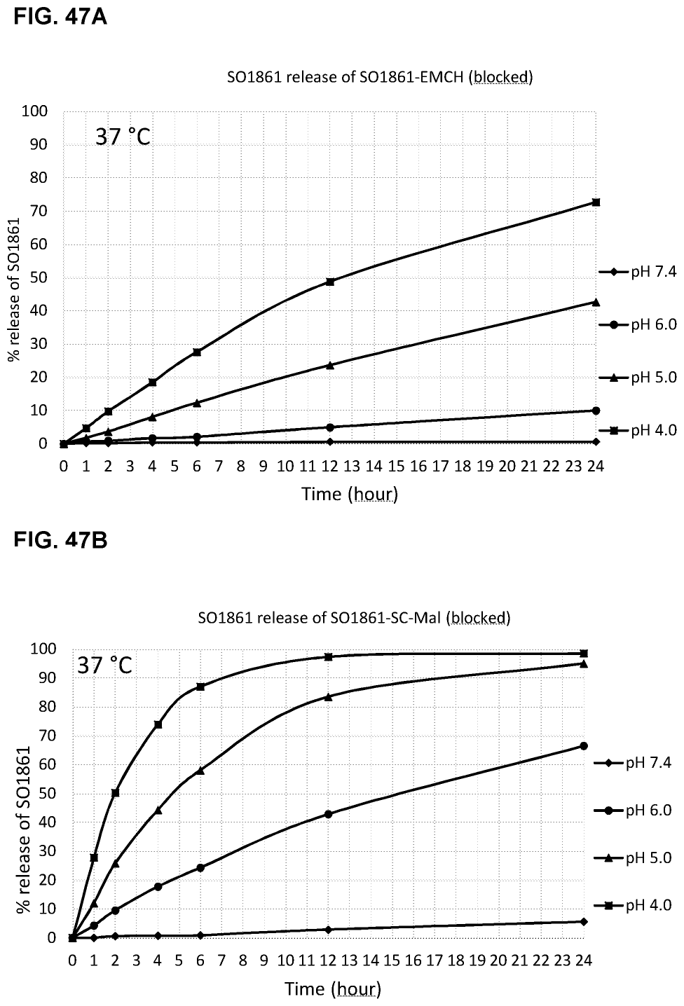

SO1861-SC-Mal (blocked) (saponin covalently coupled to the linker through an acid-labile semicarbazone (sc) bond) was tested for efficient pH sensitive release with a release kinetic assay and compared with release kinetics for SO1861-EMCH (blocked) (saponin covalently coupled to the linker through an acid-labile hydrazone bond). The assay was performed in buffers with pH 7.4, pH 6.0 pH, pH 5.0, pH 4.0 at 37° C. for 24 hours. This revealed that the saponin of the SO1861-SC-Mal (blocked) conjugate shows a 6-fold faster release compared to the release of the saponin from the SO1861-EMCH (blocked) conjugate (50% release at pH 4.0=2 hours (SO1861-SC-Mal (Blocked)) vs 50% release in 12 hours (SO1861-EMCH (Blocked)) (FIG. 47A, B). “Blocked” here refers to inactivating the maleimide group of the EMCH linker bound to the aldehyde group of the saponin (see FIG. 43B) or to inactivating the maleimide group of the linker tert-butyl 2-(4-(6-(2,5-dioxo-2,5-dihydro-1H-pyrrol-1-yl)hexanoyl)piperazine-1-carbonyl)hydrazine-1-carboxylate bound to the aldehyde group of the saponin (see FIG. 43C), upon reaction with 2-mercaptoethanol.

### LITERATURE REFERENCES

Wilton, E. E. et al. (2018) “sdAb-DB: The Single Domain Antibody Database”, ACS Synthetic Biology 7(11): 2480-2484. DOI: 10.1021/acssynbio.8b00407
Marta Kijanka & Frank-Jan Warnders & Mohamed El Khattabi & Marjolijn Lub-de Hooge & Gooitzen M. van Dam & Vasilis Ntziachristos & Liesbeth de Vries & Sabrina Oliveira & Paul M. P. van Bergen en Henegouwen, “Rapid optical imaging of human breast tumour xenografts using anti-HER2 V_{HH}S site-directly conjugated to IRDye 800CW for image-guided surgery”, Eur J Nucl Med Mol Imaging (2013) 40:1718-1729 DOI 10.1007/s00259-013-2471-2
Karen Mercier, Raimond Heukers and Chiraz Frydman, “Surface Plasmon Resonance imaging (SPRi)—Production of a single domain antibody Q17c directed against recombinant HER2 protein and its binding study by Surface Plasmon Resonance imaging technology”, Horiba Application Note Pharmaceuticals SPRi 42, 2019

SEQ ID NOS
SEQ ID NO: 1: Amino-acid coding DNA sequence of Anti-HER2 sdAb
2Rb17c from camelid
gaagttcagctgcaggaatctggtggtggtctggttcagccgggtggttctctgcgtctgtc
ttgcgcggcgtctggtttcatcttctctaacgacgcgatgacctgggttcgtcaggcgccgg
gtaaaggtctggaatgggtttcttctatcaactggtctggtacccacaccaactacgcggac
tctgttaaaggtcgtttcaccatctctcgtgacaacgcgaaacgtaccctgtacctgcagat
gaactctctgaaagacgaagacaccgcgctgtactactgcgttaccggttacggtgttacca
aaaccccgaccggtcagggtacccaggttaccgtttcttctcaccaccaccaccaccactct
ccgtctaccccgccgaccccgtctccgtctaccccgccgtgc
SEQ ID NO: 2: Amino-acid sequence of Anti-HER2 sdAb 2Rb17c
from camelid
EVQLQESGGGLVQPGGSLRLSCAASGFIFSNDAMTWVRQAPGKGLEWVSSINWSGTHTNYAD
SVKGRFTISRDNAKRTLYLQMNSLKDEDTALYYCVTGYGVTKTPTGQGTQVTVSSHHHHHHS
PSTPPTPSPSTPPC
SEQ ID NO: 3: Amino-acid coding DNA sequence of Anti-HER2 sdAb
NB2 from Camelus dromedarius
atggaagttcagctggttgaatctggtggtggtctggttcaggcgggtggttctctgcgtct
gtcttgcgcggcgtctggtatcaccttctctatcaacaccatgggttggtaccgtcaggcgc
cgggtaaacagcgtgaactggttgcgctgatctcttctatcggtgacacctactacgcggac
tctgttaaaggtcgtttcaccatctctcgtgacaacgcgaaaaacaccgtttacctgcagat
gaactctctgaaaccggaagacaccgcggtttactactgcaaacgtttccgtaccgcggcgc
agggtaccgactactggggtcagggtacccaggttaccgtttcttctcaccaccaccaccac
cac
SEQ ID NO: 4: Amino-acid sequence of Anti-HER2 sdAb NB2 from
Camelus dromedarius
MEVQLVESGGGLVQAGGSLRLSCAASGITFSINTMGWYRQAPGKQRELVALISSIGDTYYAD
SVKGRFTISRDNAKNTVYLQMNSLKPEDTAVYYCKRFRTAAQGTDYWGQGTQVTVSSHHHHH
H
SEQ ID NO: 5: Amino-acid coding DNA sequence of Anti-HER2 sdAb
pcNB2, a synthetic construct
atggaagttcagctggttgaaaaaggtggtggtcgtgttcaggcgggtggttctctgcgtct
gcgttgcgcggcgtctggtatcaccttctctatcaacaccatgggttggtaccgtcaggcgc
cgggtaaacagcgtgaactggttgcgctgatctcttctatcggtgacacctactacgcggac
tctgttaaaggtcgtttccgtatccgtcgtgacaacgcgaaaaacaccgtttacctgcgtat
gcgtcgtctgaaaccggaagacaccgcggtttactactgcaaacgtttccgtaccgcggcgc
agggtaccgactactggggtcagggtacccgtgttaccgtttctaaacaccaccaccaccac
cac
SEQ ID NO: 6: Amino-acid sequence of Anti-HER2 sdAb pcNB2, a
synthetic construct
MEVQLVEKGGGRVQAGGSLRLRCAASGITFSINTMGWYRQAPGKQRELVALISSIGDTYYAD
SVKGRFRIRRDNAKNTVYLRMRRLKPEDTAVYYCKRFRTAAQGTDYWGQGTRVTVSKHHHHH
H
SEQ ID NO: 7: amino-acid coding DNA sequence of Anti-HER1 sdAb
7D12 from camelid
gcggcgcaggttaaactggaagaatctggtggtggttctgttcagaccggtggttctctgcg
tctgacctgcgcggcgtctggtcgtacctctcgttcttacggtatgggttggttccgtcagg
cgccgggtaaagaacgtgaattcgtttctggtatctcttggcgtggtgactctaccggttac
gcggactctgttaaaggtcgtttcaccatctctcgtgacaacgcgaaaaacaccgttgacct
gcagatgaactctctgaaaccggaagacaccgcgatctactactgcgcggcggcggcgggtt
ctgcgtggtacggtaccctgtacgaatacgactactggggtcagggtacccaggttaccgtt
tcttct
SEQ ID NO: 8: amino-acid sequence of Anti-HER1 sdAb 7D12 from
camelid
AAQVKLEESGGGSVQTGGSLRLTCAASGRTSRSYGMGWFRQAPGKEREFVSGISWRGDSTGY
ADSVKGRFTISRDNAKNTVDLQMNSLKPEDTAIYYCAAAAGSAWYGTLYEYDYWGQGTQVTV
SS
SEQ ID NO: 9: amino-acid coding DNA sequence of Anti-HER1 sdAb
9G8 from camelid
gaagttcagctggttgaatctggtggtggtctggttcaggcgggtggttctctgcgtctgtc
ttgcgcggcgtctggtcgtaccttctcttcttacgcgatgggttggttccgtcaggcgccgg
gtaaagaacgtgaattcgttgttgcgatcaactggtcttctggttctacctactacgcggac
tctgttaaaggtcgtttcaccatctctcgtgacaacgcgaaaaacaccatgtacctgcagat
gaactctctgaaaccggaagacaccgcggtttactactgcgcggcgggttaccagatcaact
ctggtaactacaacttcaaagactacgaatacgactactggggtcagggtacccaggttacc
gtttcttct
SEQ ID NO: 10: amino-acid sequence of Anti-HER1 sdAb 9G8 from
camelid
EVQLVESGGGLVQAGGSLRLSCAASGRTFSSYAMGWFRQAPGKEREFVVAINWSSGSTYYAD
SVKGRFTISRDNAKNTMYLQMNSLKPEDTAVYYCAAGYQINSGNYNFKDYEYDYWGQGTQVT
VSS
SEQ ID NO: 11: Amino-acid coding DNA sequence of Anti-VGFR2
sdAb NTV1, a synthetic construct
atggcgcaggttcagctgctggaatctggtggtggtctggttcagccgggtggttctctgcg
tctgtcttgcgcggcgtctggttactctgttatcaacgacttcatgacctgggttcgtcagg
cgccgggtaaaggtctggaatgggtttcttctatctctgttgcggacggttctacctactac
gcggactctgttaaaggtcgtttcaccatctctcgtgacaactctaaaaacaccctgtacct
gcagatgaactctctgcgtgcggaagacaccgcggtttactactgcgcggcgcgtgttggtg
gtcgtgacctgggttggccgtacgaactggactactggggtcagggtaccctggttaccgtt
tcttct
SEQ ID NO: 12: Amino-acid sequence of Anti-VGFR2 sdAb NTV1,
a synthetic construct
MAQVQLLESGGGLVQPGGSLRLSCAASGYSVINDFMTWVRQAPGKGLEWVSSISVADGSTYY
ADSVKGRFTISRDNSKNTLYLQMNSLRAEDTAVYYCAARVGGRDLGWPYELDYWGQGTLVTV
SS
SEQ ID NO: 13: Amino-acid coding DNA sequence of Anti-VGFR2
sdAb NTV2, a synthetic construct
atggcgcaggttcagctgctggaatctggtggtggtctggttcagccgggtggttctctgcg
tctgtcttgcgcggcgtctggtttcaaaatcaccaacaaaaccatggcgtgggttcgtcagg
cgccgggtaaaggtctggaatgggtttcttctatcggttcttcttctggttctacctactac
gcggactctgttaaaggtcgtttcaccatctctcgtgacaactctaaaaacaccctgtacct
gcagatgaactctctgcgtgcggaagacaccgcggtttactactgcgcgcgtcgtaaaggta
accgtctgggtccggcggcgctgcgttcttggggtcagggtaccctggttaccgtttcttct
SEQ ID NO: 14: Amino-acid sequence of Anti-VGFR2 sdAb NTV2,
a synthetic construct
MAQVQLLESGGGLVQPGGSLRLSCAASGFKITNKTMAWVRQAPGKGLEWVSSIGSSSGSTYY
ADSVKGRFTISRDNSKNTLYLQMNSLRAEDTAVYYCARRKGNRLGPAALRSWGQGTLVTVSS
SEQ ID NO: 15: Amino-acid coding DNA sequence of Anti-VGFR2
sdAb NTV3, a synthetic construct
atggcgcaggttcagctgctggaatctggtggtggtctggttcagccgggtggttctctgcg
tctgtcttgcgcggcgtctggtgttcgtgttaactacaaatctatgtcttgggttcgtcagg
cgccgggtaaaggtctggaatgggtttctaccatcacctctcgtaacggttctacctactac
gcggactctgttaaaggtcgtttcaccatctctcgtgacaactctaaaaacaccctgtacct
gcagatgaactctctgcgtgcggaagacaccgcggtttactactgcgcgaccggtcgtgcgc
accacgcgccggttcgttactggggtcagggtaccctggttaccgtttcttct
SEQ ID NO: 16: Amino-acid sequence of Anti-VGFR2 sdAb NTV3,
a synthetic construct
MAQVQLLESGGGLVQPGGSLRLSCAASGVRVNYKSMSWVRQAPGKGLEWVSTITSRNGSTYY
ADSVKGRFTISRDNSKNTLYLQMNSLRAEDTAVYYCATGRAHHAPVRYWGQGTLVTVSS
SEQ ID NO: 17: Amino-acid coding DNA sequence of Anti-VGFR2
sdAb NTV4, a synthetic construct
atggcgcaggttcagctgctggaatctggtggtggtctggttcagccgggtggttctctgcg
tctgtcttgcgcggcgtctggtgttaccatcaccgacgaagacatgacccgtgttcgtcagg
cgccgggtaaaggtctggaatgggtttcttctatcctgaacaccggtggttctacctactac
gcggactctgttaaaggtcgtttcaccatctctcgtgacaactctaaaaacaccctgtacct
gcagatgaactctctgcgtgcggaagacaccgcggtttactactgcgcggcggttcacgaaa
aagcggcggacatgaacttctggggtcagggtaccctggttaccgtttcttct
SEQ ID NO: 18: Amino-acid sequence of Anti-VGFR2 sdAb NTV4,
a synthetic construct
AQVQLLESGGGLVQPGGSLRLSCAASGVTITDEDMTRVRQAPGKGLEWVSSILNTGGSTYYA
DSVKGRFTISRDNSKNTLYLQMNSLRAEDTAVYYCAAVHEKAADMNFWGQGTLVTVSS
SEQ ID NO: 19: amino-acid coding DNA sequence of Anti-human
CD19 sdAb SRB-85 from Bactrian camel
gaagttcagctgctggaatctggtggtggtctggttcagccgggggttctctgcgttcttgc
gaagcgtctggtttcaacgcgatgacctctatcgactcttggaccgacgcggttaaaggttg
ggttcgtcagccgccgggtaaaggtctggaatgggtttctcgtttcgcgatctctcaggaca
acgcgaaaaacaccgtttacctgcagatgaactctctgaaaccggaagacaccgcgatgtac
tactgcgcgctgtctaaatgctacacccgtgtttacgactactggggtcagggtacccaggt
taccgtttcttctggt
SEQ ID NO: 20: amino-acid sequence of Anti-human CD19 sdAb
SRB-85 from Bactrian camel
EVQLLESGGGLVQPGGSLRSCEASGFNAMTSIDSWTDAVKGWVRQPPGKGLEWVSRFAISQD
NAKNTVYLQMNSLKPEDTAMYYCALSKCYTRVYDYWGQGTQVTVSS
SEQ ID NO: 21: amino-acid coding DNA sequence of Anti-human
CD19 sdAb SRB-37 from Bactrian camel
gaagttcagctgcaggaatctggtggtggtctggttcagccgggtggttctctgcgtctgtc
ttgcgcggcgtctggtttcatctacatggttggtatcaaaaccgaacgtgacggtgttaaag
gttgggttcgtcaggcgccgggtaaaggtctggaatggctgtctcgtttcaccatcccgcgt
gacaacgcgaaaaacaccctgtacctgcagatgaacaacctgaaatctgaagacaccgcgct
gtactactgcgcgaccgaagaaaacgactggggtcagggtacccaggttaccgtttcttctg
gt
SEQ ID NO: 22: amino-acid sequence of Anti-human CD19 sdAb
SRB-37 from Bactrian camel
EVQLQESGGGLVQPGGSLRLSCAASGFIYMVGIKTERDGVKGWVRQAPGKGLEWLSRFTIPR
DNAKNTLYLQMNNLKSEDTALYYCATEENDWGQGTQVTVSS
SEQ ID NO: 23: Amino-acid coding DNA sequence of Anti-CTLA-4
sdAb NB16 from Camelus dromedarius
caggttcagctgcaggaatctggtggtggttctgttcaggcgggtggttctctgcgtctgtc
ttgcaccgcgtctggtttcggtgttgacggtaccgacatgggttggtaccgtcaggcgccgg
gtaacgaatgcgaactggtttcttctatctcttctatcggtatcggttactactctgaatct
gttaaaggtcgtttaccatctctcgtgacaacgcgaaaaacaccgtttacctgccagatgaa
ctctctgcgtccggacgacaccgcggtttactactgcggtcgtcgttggatcggttaccgtt
gcggtaactggggtcgtggtacccaggttaccgtttcttct
SEQ ID NO: 24: Amino-acid sequence of Anti-CTLA-4 sdAb NB16
from Camelus dromedarius
QVQLQESGGGSVQAGGSLRLSCTASGFGVDGTDMGWYRQAPGNECELVSSISSIGIGYYSES
VKGRFTISRDNAKNTVYLQMNSLRPDDTAVYYCGRRWIGYRCGNWGRGTQVTVSS
SEQ ID NO: 25: Amino-acid coding DNA sequence of Anti-CTLA-4
sdAb NB36 from Camelus dromedarius
caggttcagctgcaggaatctggtggtggttctgttcaggcgggtggttctctgcgtctgtc
ttgcaccggttctcgttacacctacaccatgggttggttccgtcaggcgccgggtaaagaac
gtgaaggtgttgttgcgatcaccgcgttcggttctccgttctacgcggactctgttaaaggt
cgtttcaccatctctcgtgacaacgcgaacaacaccatcttcctgcagatgaactctctgaa
accggaagactctgcgatgtactactgcgcggcgcgtggttcttctggtacctcttacaaat
ggaacgaatacggttcttacaactactggggtcagggtacccaggttaccgtttcttct
SEQ ID NO: 26: Amino-acid sequence of Anti-CTLA-4 sdAb NB36
from Camelus dromedarius
QVQLQESGGGSVQAGGSLRLSCTGSRYTYTMGWFRQAPGKEREGVVAITAFGSPFYADSVKG
RFTISRDNANNTIFLQMNSLKPEDSAMYYCAARGSSGTSYKWNEYGSYNYWGQGTQVTVSS
SEQ ID NO: 27: Amino-acid coding DNA sequence of Anti-CTLA-4
sdAb NB91 from Camelus dromedarius
caggttcagctgcaggaatctggtggtggttctgttcaggcgggtggttctctgcgtctgtc
ttgcgcggcgtctaaatacacctcttgcatgggttggttccgtcaggcgccgggtaaagaac
gtgaagttgttgcgcacatcgactctggtccgcgtaccctgtacgcggactctgttaaaggt
cgtttcaccatctctaaagacaacgcgaaaaacaccctgtacctggaaatgtctaccctgaa
accggacgacaccgcgatgtactactgcgcggcgggtccgatgtactctggttcttgcaact
acaactactggggtcagggtacccaggttaccgtttcttct
SEQ ID NO: 28: Amino-acid sequence of Anti-CTLA-4 sdAb NB91
from Camelus dromedarius
QVQLQESGGGSVQAGGSLRLSCAASKYTSCMGWFRQAPGKEREVVAHIDSGPRTLYADSVKG
RFTISKDNAKNTLYLEMSTLKPDDTAMYYCAAGPMYSGSCNYNYWGQGTQVTVSS
SEQ ID NO: 29: Amino-acid coding DNA sequence of Anti-human
PD-L1 sdAb A1 from Camelus dromedarius
Caggttcagctgcaggaatctggtggtggtctggttcagccgggtggttctctgcgtctgtc
ttgcgcggcgtctggtttcaccctggactactacgcgatcggttggttccgtcaggcgccgg
gtaaagaacgtgaaggtgtttcttgcatctcttcttctgacggttctacctactacgcggac
tctgttaaaggtcgtttcaccatctctcgtgacaacgcgaaaaacaccgtttacctgcagat
gtcttctctgaaaccggaagacaccgcggtttactactgcggtatctctggttcttgcctgc
tggaagactacggtatggactactggggtaaaggtacccaggttaccgtttcttct
SEQ ID NO: 30: Amino-acid sequence of Anti-human PD-L1 sdAb
A1 from Camelus dromedarius
QVQLQESGGGLVQPGGSLRLSCAASGFTLDYYAIGWFRQAPGKEREGVSCISSSDGSTYYAD
SVKGRFTISRDNAKNTVYLQMSSLKPEDTAVYYCGISGSCLLEDYGMDYWGKGTQVTVSS
SEQ ID NO: 31: Amino-acid coding DNA sequence of Anti-human
PD-L1 sdAb B1 from Camelus dromedarius
caggttcagctgcaggaatctggtggtggtctggttcacccgggtggttctctgcgtctgtc
ttgcgcggcgtctggtttctctctggacaactacgcgatcggttggttccgtcaggcgccgg
gtaaagaacgtgaaggtgtttcttgcatctcttctggttctgaaggtcgtcgttactacgcg
gacttcgttaaaggtcgtttcaccatctctcgtgacaacgcgaaaaacaccgcgttcctgca
gatgaactctctgaaaccggaagacaccgcggactactactgcgcgaccgttggtttctgct
cttctcagtacggtatggaattcgttggtgactactggggtcagggtacccaggttaccgtt
tcttct
SEQ ID NO: 32: Amino-acid sequence of Anti-human PD-L1 sdAb
B1 from Camelus dromedaries
QVQLQESGGGLVHPGGSLRLSCAASGFSLDNYAIGWFRQAPGKEREGVSCISSGSEGRRYYA
DFVKGRFTISRDNAKNTAFLQMNSLKPEDTADYYCATVGFCSSQYGMEFVGDYWGQGTQVTV
SS
SEQ ID NO: 33: Amino-acid sequence of Anti-mouse serum
albumin sdAb MSA21 (organism: artificial sequence)
QVQLQESGGGLVQPGGSLRLSCEASGFTFSRFGMTWVRQAPGKGVEWVSGISSLGDSTLYAD
SVKGRFTISRDNAKNTLYLQMNSLKPEDTAVYYCTIGGSLNPGGQGTQVTVSS
SEQ ID NO: 34: Amino-acid sequence of Anti-human serum
albumin sdAb Alb-1 (organism: artificial sequence)
AVQLVESGGGLVQPGNSLRLSCAASGFTFRSFGMSWVRQAPGKEPEWVSSISGSGSDTLYAD
SVKGRFTISRDNAKTTLYLQMNSLKPEDTAVYYCTIGGSLSRSSQGTQVTVSS
SEQ ID NO: 35: Amino-acid sequence of Anti-human serum
albumin sdAb Alb23 (Humanized, optimized Alb1) (organism:
artificial sequence)
EVQLLESGGGLVQPGGSLRLSCAASGFTFRSFGMSWVRQAPGKGPEWVSSISGSGSDTLYAD
SVKGRFTISRDNSKTLYLQMNSLRPEDTAVYYCTIGGSLSRSSQGTLVTVSS
SEQ ID NO: 36: Amino-acid sequence of Anti-EGFR V_{HH} 7A5
(organism: artificial; recombinant peptide)
EVQLVESGGGLVQAGGSLRLSCAASDRTFSSNNMGWFRQAPGKEREFVAAIGWGGLETHYSD
SVKGRFTISRDNAKNTVYLQMNSLKPEDTARYYCAVSSTRTVIYTLPRMYNYWGQGTQVTVS
S
SEQ ID NO: 37: Amino-acid sequence of Anti-EGFR V_{HH} 7D12
(organism: artificial; recombinant peptide)
QVKLEESGGGSVQTGGSLRLTCAASGRTSRSYGMGWFRQAPGKEREFVSGISWRGDSTGYAD
SVKGRFTISRDNAKNTVDLQMNSLKPEDTAIYYCAAAAGSAWYGTLYEYDYWGQGTQVTVSS
SEQ ID NO: 38: Amino-acid sequence of Anti-EGFR V_{HH} 7C12
(organism: artificial; recombinant peptide)
AVQLVESGGGSVQAGGSLRLTCAASGRTSRSYGMGWFRQAPGKEREFVSGISWRGDSTGYAD
SVKGRFTISRDNAKNTVDLQMNSLKPEDTAIYYCAAAAGSTWYGTLYEYDYWGQGTQVTVSS
SEQ ID NO: 39: Amino-acid sequence of Anti-insulin-like
growth factor 1 receptor V_{HH} 4B11 (organism: artificial;
recombinant peptide)
EVQLVESGGGLVQPGGSLRLSCAASGSIFTFNAMGWYRQAPGKQRELVAVIISGGSTHYVDS
VKGRFTISRDNAKKMVYLQMNSLKPEDTAVYYCNVKKFGDYWGQGTQVTVSS
SEQ ID NO: 40: Amino-acid sequence of Anti-insulin-like
growth factor 1 receptor V_{HH} 3G7 (organism: artificial;
recombinant peptide)
DVQLVESGGGLVQAGGSLRLSCAASESISTINVMAWYRQAPGKQRELVAEITRSGRTNYVDS
VKGRFTISRDNAKNTMYLQMNSLNLEDTAVYYCRTIDGSWREYWGQGTQVTVSS
SEQ ID NO: 41: Amino-acid sequence of Anti-insulin-like
growth factor 1 receptor V_{HH} 2C7 (organism: artificial;
recombinant peptide)
QVKLEESGGGLVQPGGSLRLSCVASGRTFSNYAIVIGWFRQAPGQEREFVAAINWNSRSTYY
ADSVKGRFTISRLNARNTVYLQMNRLKPEDTAVYDCAASHDSDYGGTNANLYDYWGQGTQVT
VSS
SEQ ID NO: 42: Amino-acid sequence of Anti-insulin-like
growth factor 1 receptor V_{HH} 1C7 (organism: artificial;
recombinant peptide)
QVKLEESGGGLVQAGGSLRLSCVASGRTFSRTANAWFRQAPGKEREFVATITWNSGTTRYAD
SVKGRFFISKDSAKNTIYLEMNSLEPEDTAVYYCAATAAAVITPTRGYYNYWGQGTQVTVSS
SEQ ID NO: 43: Amino-acid sequence of Anti-CEACAM V_{HH} NbCEA5
(organism: artificial; recombinant peptide)
EVQLVESGGGSVQAGGSLRLSCAASGDTYGSYWMGWFRQAPGKEREGVAAINRGGGYTVYAD
SVKGRFTISRDTAKNTVYLQMNSLRPDDTADYYCAASGVLGGLHEDWFNYWGQGTLVTVSS
SEQ ID NO: 44: Amino-acid sequence of Anti-CEACAM V_{HH} CEA5
(organism: artificial; recombinant peptide)
EVQLVESGGGSVQAGGSLRLSCAASGDTYGSYWMGWFRQAPGQEREAVAAINRGGGYTVYAD
SVKGRFTISRDNAKNTLYLQMNSLRPDDTADYYCAASGVLGGLHEDWFNYWGQGTLVTVSS
SEQ ID NO: 45: Amino-acid sequence of Anti-CD123 V_{HH} 57A07
(organism: artificial; recombinant peptide)
EVQLVESGGGLVQAGGSLRLSCAASGSIFSGNVMGWYRRQAPGKEREWVAAIASGGSIYYRD
SVKGRFTISRDNAKNTVYLQMNSLKPEDTAVYYCNSHPPTLPYWGLGTQVTVSS
SEQ ID NO: 46: Amino-acid sequence of Anti-CD123 V_{HH} 57B04
(organism: artificial; recombinant peptide)
EVQLVESGGGLVQPGGSLRLSCAASGINFRFNSMGWWRRRAPGKEREWVAAITSGDITNYRD
SVRGRFTISRDNVKNTVYLQMNTLKLEDTAVYYCNTFPPIADYWGLGTQVTVSS
SEQ ID NO: 47: Amino-acid sequence of Anti-CD123 V_{HH} 51D09
(organism: artificial; recombinant peptide)
EVQLVESGGGLVQPGGSLRLSCAASGSIFSGNTMGWYRQAPGKQRELVAAISSGGSTDYADS
VKGRFTISRDNSKNTVYLQMNSLRPEDTAVYYCNAAILLYRLYGYEEGDYWGLGTLVTVSS
SEQ ID NO: 48: Amino-acid sequence of Anti-CD123 V_{HH} 55C05
(organism: artificial; recombinant peptide)
EVQLVESGGGLVPAGDSLRLSCVASGRSLNTYTMGWFRQAPGKECEEVAAINWNGVYRDYAD
SAKGRETASRDNAMNTVFLQMNSLKPEDTAVYFCATATQGWDRHTEPSDFGSWGLGTQVTVS
S
SEQ ID NO: 49: Amino-acid sequence of Anti-CD123 V_{HH} 50F07
(organism: artificial; recombinant peptide)
EVQLVESGGGLVQPGGSLRLSCTGSGSTFSINAMGWYRQAPGKQRELVAAITSGGRTNYADS
VKGRFTISRDNSKNTVYLQMNSLRPEDTAVYYCNAAILLYRLYGYEEGDYWGLGTLVTVSS
SEQ ID NO: 50: Amino-acid sequence of Anti-CD123 V_{HH} 55F03
(organism: artificial; recombinant peptide)
EVQLVESGGGLVQAGGPLRLSCAASGRTFSSYVMGWFRQAPGKEREFVAAIYWSNGKTQYTD
SVKGRFTISGDNAKNTVYLQMNSLNPEDTAVYYCVADKDETGFRTLPIAYDYWGLGTQVTVS
S
SEQ ID NO: 51: Amino-acid sequence of Anti-CD123 V_{HH} 55A01
(organism: artificial; recombinant peptide)
EVQLVESGGGSVQAGGSLRLSCTTSGRALNMYVMGWFRQAPGNEREFVAATSSSGGSTSYPD
SVKGRFTISRDNAKNTVYLQMNSLKPEDTAAYRCAASPYVSTPTMNILEEYRYWGLGTQVTV
SS
SEQ ID NO: 52: Amino-acid sequence of Anti-c-MET V_{HH} 04E09
(organism: artificial sequence)
EVQLVESGGGLVQPGGSLRLSCAASGFILDYYAIGWFRQAPGKEREGVLCIDASDDITYYAD
SVKGRFTISRDNAKNTVYLQMNSLKPEDTGVYYCATPIGLSSSCLLEYDYDYWGQGTLVTVS
S
SEQ ID NO: 53: Amino-acid sequence of Anti-c-MET V_{HH} 06B08
(organism: artificial sequence)
EVQLVESGGGLVQAGGSLRLSCAASGRTISRYTMGWFRQAPGKEREFVAAISWSGDNTNYAD
SVKGRFTISRPNTKNTMYLQMNSLKPEDTAVYYCAADYRSGSYYQASEWTRPSGYDYWGQGT
LVTVSS
SEQ ID NO: 54: Amino-acid sequence of Anti-c-MET V_{HH} 06C12
(organism: artificial sequence)
EVQLVESGGGLVQPGGSLRLSCAASGFSLDYFAIGWFRQAPGKEREEISCISNSDGSTYYAN
SVKGRFTISIDNAKNTVYLQMTSLKPEDTAVYYCATPVGLGPFCKTTNDYDYSGQGTLVTVS
S
SEQ ID NO: 55: Amino-acid sequence of Anti-c-MET V_{HH} 06F10
(organism: artificial sequence)
EVQLVESGGGLVQPGGSLRLSCAASGFTLDYYAINWFRQAPGKEREGVSCISGGDGSTYYAD
SVKGRFTISRDNAKNTVYLQMNSLKPEDTAVYYCATALGLSSSCHGDGYDYWGQGTLVTVSS
SEQ ID NO: 56: Amino-acid sequence of Anti-Her3 V_{HH} 21F6
(organism: artificial sequence)
EVQLVESGGGLVQAGGSLRLSCAASGRTYYLNAMGWFRQGPGKDREFVAAIDWSDGNKDYAD
SVKGRFTISRDNAKNTVYLQMNSLKPEDTAVYYCAADTPPWGPMIYIESYDSWGQGTLVTVS
S
SEQ ID NO: 57: Amino-acid sequence of Anti-Her3 V_{HH} 4C7
(organism: artificial sequence)
EVQLVESGGGLVQAGGSLRLSCAASGFTFSSYPMSWVRQAPGKGPAWVSTVSPGGITTSYAD
SVKGRFTISRDNAKNTLYLQMNSLKPEDTAVYYCLRDLNNRGQGTLVTVSS
SEQ ID NO: 58: Amino-acid sequence of Anti-Her3 V_{HH} 17B5
(organism: artificial sequence)
EVQLVESGGGLVQPGGSLRLSCAASGSIGGLNAMAWYRQAPGKERELVAGIFGVGSTRYADS
VKGRFTISRDIAKNTVFLQMNSLNSEDTAVYYCRMSSVTRGSSDYWGQGTQVTVSS
SEQ ID NO: 59: Amino-acid sequence of Anti-Her3 V_{HH} 18G11
(organism: artificial sequence)
EVQLVESGGGLVQPGGSLRLSCAASGTLFKINAMGWYRQAPGKRRELVALITSSDTTDYAES
VEGRFTISRDNTWNAVYLQMNSLKPEDTAVYYCHSDHYSMGVPEKRVIMYGQGTQVTVSS
SEQ ID NO: 60: Amino-acid sequence of Anti-Her3 V_{HH} 34C7
(organism: artificial sequence)
EVQLVESGGGLVQPGGSLGLSCVASGSIFRINAMAWYRQAPGKQRELVAEITAGGSTNYADS
VKGRFTISVDNAWNTLYLQMNSLKVEDTAVYYCNLDHYTTWDRRSAYWGQGTQVTVSS
SEQ ID NO: 61: Amino-acid sequence of Anti-Her2 V_{HH} 47D5
(organism: llama)
KVQLVESGGGLVQPGGSLRLSCAASGSIFGFNDMAWYRQAPGKQRELVALISRVGVTSSADS
VKGRFTISRVNAKDTVYLQMNSLKPEDTAVYYCYMDQRLDGSTLAYWGQGTQVTVSS
SEQ ID NO: 62: Amino-acid sequence of Anti-Her2 V_{HH} 2D3
(organism: llama)
EVQLVESGGSLVQPGGSLRLSCAASGFTFDDYAMSWVRQVPGKGLEWVSSINWSGTHTDYAD
SVKGRFTISRNNANNTLYLQMNSLKSEDTAVYYCAKNWRDAGTTWFEKSGSAGQGTQVTVSS
SEQ ID NO: 63: Amino-acid sequence of Anti-Her2 V_{HH} 5F7
(organism: llama)
EVQLVESGGGLVQPGGSLRLSCAASGFTFSINTMGWYRQAPGKQRELVALISSIGDTYYADS
VKGRFTISRDNAKNTVYLQMNSLKPEDTAVYYCKRFRTAAQGTDYWGQGTQVTVSS
SEQ ID NO: 64: Amino-acid sequence of Anti-Her2 V_{HH} 13D11
(organism: llama)
EVQLVESGGGLVHPGGSLRLSCVGSGFSLDDYGMTWVRRAPGKGLEWVSSINWSGTHTDYAD
SVKGRFTISRDNAKNTLFLQMNSLNPEDTAVYYCGQGWKIVPTNPRGHGTQVTVSS
SEQ ID NO: 65: Amino-acid sequence of Anti-Her2 V_{HH} 2B4
(organism: llama)
EVQLVESGGGLVQPGGSLRLSCVGSGFSLDDYAMTWVRQAPGKGLEWVSSINWSGTHTDYAD
SVKGRFTISRDNAKNTLFLQMNSLSPEDTAVYYCNQGWKIRPTIPMGHGTQVTVSS
SEQ ID NO: 66: Amino-acid sequence of Anti-Her2 V_{HH} 2G2
(organism: llama)
EVQLVESGGGLVQPGGSLRLSCVASGFSLDDYGMTWVRQAPGKGLEWVSSINWSGTHTDYTD
PVKGRFTISRDNAKNTLFLQMNNLTPEDTAVYYCNRGWKIVPTDLGGHGTQVTVSS
SEQ ID NO: 67: Amino-acid sequence of Anti-Her2 V_{HH} 13G11
(organism: llama)
EVQLVESGGGLVQAGGSLRLSCAASGRTFISNYAMGWFRQAPGKEREFVATINWSGSHSDYA
DSVKGRFTISRDNAKNTVYLQMNNLKSEDTAVYYCAPGWGTAPLSTSVYWGQGTQVTVSS
SEQ ID NO: 68: Amino-acid sequence of Anti-Her2 V_{HH} 12E33
(organism: llama)
EVQLVESGGGMVQAGGSLRLSCAASGLTLSNYGMGWFRQAPGKEREFVSSINWSGTHTYDAD
FVKGRFIISRDNAKNTVYLQINSLKPEDTAVYYCAAGGWGTGRYNYWGQGTQVTVSS
SEQ ID NO: 69: Amino-acid sequence of Anti-Her2 V_{HH} 13F21
(organism: llama)
EVQLVESGGGLVQSGGSLRLSCVASGTIVSINATSWYRQAPGNQRELVATIIGDGRTHYADS
VKDRFTISRDAAANLVYLQMNSLKPSDTAIYSCNANGIESYGWGNRHFNYWTVGTQVTVSS
SEQ ID NO: 70: Amino-acid sequence of Anti-Her2 V_{HH} 11A101
(organism: llama)
EVQLVESGGGLVQAGGSLRLSCAASGRTFNAMGWFRQAPGKEREFVAAISRSPGVTYYADSV
KGRFTTSRDNAKNTVYLQMNDLKPEDTAVYYCAADFYLATLAHEYDYWGQGTQVTVSS
SEQ ID NO: 71: Amino-acid sequence of Anti-Her2 V_{HH} 11A22
(organism: llama)
EVQLVESGGGLVQAGGSLRLSCAASGRTFSSYAMAWFRQAPGTEREFIAGIRWSDGSTYYAD
SVKGRFTISRDNAKNTVYLQMNSLKPEDTAVYYCAADFYVSTLAHEYDYWGQGTQVTVSS
SEQ ID NO: 72: Amino-acid sequence of Anti-Her2 V_{HH} 12D44
(organism: llama)
KVQLVESGGGLVQAGGSLRLSCAASGRTFSSYAMAWFRQAPGTEREFIAGIRWSDGSTYYAD
SVKGRFTISRANAKNTVYLQMNGLKPEDTAVYYCAADFYVSTLAHEYDYWGQGTQVTVSS
SEQ ID NO: 73: Amino acid sequence of bivalent VHHEGFR-
dianthin-Cys
QVKLEESGGGSVQTGGSLRLTCAASGRTSRSYGMGWFRQAPGKEREFVSGISWRGDSTGYAD
SVKGRFTISRDNAKNTVDLQMNSLKPEDTAIYYCAAAAGSAWYGTLYEYDYWGQGTQVTVSS
GGGGSGGGGSEVQLVESGGGLVQAGGSLRLSCAASGRTFSSYAMGWFRQAPGKEREFVVAIN
WSSGSTYYADSVKGRFTISRDNAKNTMYLQMNSLKPEDTAVYYCAAGYQINSGNYNFKDYEY
DYWGQGTQVTVSSGGGGSGGGGSAAATAYTLNLANPSASQYSSFLDQIRNNVRDTSLIYGGT
DVAVIGAPSTTDKFLRLNFQGPRGTVSLGLRRENLYVVAYLAMDNANVNRAYYFKNQITSAE
LTALFPEVVVANQKQLEYGEDYQAIEKNAKITTGDQSRKELGLGINLLITMIDGVNKKVRVV
KDEARFLLIAIQMTAEAARFRYIQNLVTKNFPNKFDSENKVIQFQVSWSKISTAIFGDCKNG
VFNKDYDFGFGKVRQAKDLQMGLLKYLGRPKGGGGSGGGGSHRWCCPGCCKTFGGGGSHHHH
HHHHHHHRWCCPGCCKTF
SEQ ID NO: 74: Amino acid sequence of bivalent VHHEGFR-Cys
QVKLEESGGGSVQTGGSLRLTCAASGRTSRSYGMGWFRQAPGKEREFVSGISWRGDSTGYAD
SVKGRFTISRDNAKNTVDLQMNSLKPEDTAIYYCAAAAGSAWYGTLYEYDYWGQGTQVTVSS
GGGGSGGGGSEVQLVESGGGLVQAGGSLRLSCAASGRTFSSYAMGWFRQAPGKEREFVVAIN
WSSGSTYYADSVKGRFTISRDNAKNTMYLQMNSLKPEDTAVYYCAAGYQINSGNYNFKDYEY
DYWGQGTQVTVSSHHHHHHHHHHHRWCCPGCCKTF
SEQ ID NO: 75: Amino acid sequence of VHH 7D12
QVKLEESGGGSVQTGGSLRLTCAASGRTSRSYGMGWFRQAPGKEREFVSGISWRGDSTGYAD
SVKGRFTISRDNAKNTVDLQMNSLKPEDTAIYYCAAAAGSAWYGTLYEYDYWGQGTQVTVSS
SEQ ID NO: 76: Amino acid sequence of VHH 9G8
EVQLVESGGGLVQAGGSLRLSCAASGRTFSSYAMGWFRQAPGKEREFVVAINWSSGSTYYAD
SVKGRFTISRDNAKNTMYLQMNSLKPEDTAVYYCAAGYQINSGNYNFKDYEYDYWGQGTQVT
VSS
SEQ ID NO: 77: Amino-acid sequence of tetra-Cys artificial
linker
HRWCCPGCCKTF
SEQ ID NO: 78: Amino-acid sequence of bivalent V_{HH}-EGFR-
Dianthin
QVKLEESGGGSVQTGGSLRLTCAASGRTSRSYGMGWFRQAPGKEREFVSGISWRGDSTGYAD
SVKGRFTISRDNAKNTVDLQMNSLKPEDTAIYYCAAAAGSAWYGTLYEYDYWGQGTQVTVSS
GGGGSGGGGSEVQLVESGGGLVQAGGSLRLSCAASGRTFSSYAMGWFRQAPGKEREFVVAIN
WSSGSTYYADSVKGRFTISRDNAKNTMYLQMNSLKPEDTAVYYCAAGYQINSGNYNFKDYEY
DYWGQGTQVTVSSGGGGSGGGGSAAATAYTLNLANPSASQYSSFLDQIRNNVRDTSLIYGGT
DVAVIGAPSTTDKFLRLNFQGPRGTVSLGLRRENLYVVAYLAMDNANVNRAYYFKNQITSAE
LTALFPEVVVANQKQLEYGEDYQAIEKNAKITTGDQSRKELGLGINLLITMIDGVNKKVRVV
KDEARFLLIAIQMTAEAARFRYIQNLVTKNFPNKFDSENKVIQFQVSWSKISTAIFGDCKNG
VFNKDYDFGFGKVRQAKDLQMGLLKYLGRPKGGGGSGGGGSHRWCCPGCCKTFGGGGSHHHH
HHHHHHHRWCCPGCCKTF

SEQ ID NO: 47: Amino-acid sequence of Anti-CD123 V_{HH} 51D09 (organism: artificial; recombinant peptide) EVQLVESGGGLVQPGGSLRLSCAASGSIFSGNTMGWYRQAPGKQRELVAAISSGGSTDYADSVKGR FTISRDNSKNTVYLQMNSLRPEDTAVYYCNAAILLYRLYGYEEGDYWGLGTLVTVSS

SEQ ID NO: 48: Amino-acid sequence of Anti-CD123 V_{HH} 55C05 (organism: artificial; recombinant peptide) EVQLVESGGGLVPAGDSLRLSCVASGRSLNTYTMGWFRQAPGKECEEVAAINWNGVYRDYADSAK GRETASRDNAMNTVFLQMNSLKPEDTAVYFCATATQGWDRHTEPSDFGSWGLGTQVTVSS

SEQ ID NO: 49: Amino-acid sequence of Anti-CD123 V_{HH} 50F07 (organism: artificial; recombinant peptide) EVQLVESGGGLVQPGGSLRLSCTGSGSTFSINAMGWYRQAPGKQRELVAAITSGGRTNYADSVKGR FTISRDNSKNTVYLQMNSLRPEDTAVYYCNARISAGTAFWLWSDYEYWGLGTLVTVSS

SEQ ID NO: 50: Amino-acid sequence of Anti-CD123 V_{HH} 55F03 (organism: artificial; recombinant peptide) EVQLVESGGGLVQAGGPLRLSCAASGRTFSSYVMGWFRQAPGKEREFVAAIYWSNGKTQYTDSVK GRFTISGDNAKNTVYLQMNSLNPEDTAVYYCVADKDETGFRTLPIAYDYWGLGTQVTVSS

SEQ ID NO: 51: Amino-acid sequence of Anti-CD123 V_{HH} 55A01 (organism: artificial; recombinant peptide) EVQLVESGGGSVQAGGSLRLSCTTSGRALNMYVMGWFRQAPGNEREFVAATSSSGGSTSYPDSVK GRFTISRDNAKNTVYLQMNSLKPEDTAAYRCAASPYVSTPTMNILEEYRYWGLGTQVTVSS

SEQ ID NO: 52: Amino-acid sequence of Anti-c-MET V_{HH} 04E09 (organism: artificial sequence) EVQLVESGGGLVQPGGSLRLSCAASGFILDYYAIGWFRQAPGKEREGVLCIDASDDITYYADSVKGRF TISRDNAKNTVYLQMNSLKPEDTGVYYCATPIGLSSSCLLEYDYDYWGQGTLVTVSS

SEQ ID NO: 53: Amino-acid sequence of Anti-c-MET V_{HH} 06B08 (organism: artificial sequence) EVQLVESGGGLVQAGGSLRLSCAASGRTISRYTMGWFRQAPGKEREFVAAISWSGDNTNYADSVKG RFTISRPNTKNTMYLQMNSLKPEDTAVYYCAADYRSGSYYQASEWTRPSGYDYWGQGTLVTVSS

SEQ ID NO: 54: Amino-acid sequence of Anti-c-MET V_{HH} 06012 (organism: artificial sequence) EVQLVESGGGLVQPGGSLRLSCAASGFSLDYFAIGWFRQAPGKEREEISCISNSDGSTYYANSVKGR FTISIDNAKNTVYLQMTSLKPEDTAVYYCATPVGLGPFCKTTNDYDYSGQGTLVTVSS

SEQ ID NO: 55: Amino-acid sequence of Anti-c-MET V_{HH} 06F10 (organism: artificial sequence) EVQLVESGGGLVQPGGSLRLSCAASGFTLDYYAINWFRQAPGKEREGVSCISGGDGSTYYADSVKG RFTISRDNAKNTVYLQMNSLKPEDTAVYYCATALGLSSSCHGDGYDYWGQGTLVTVSS

1. A conjugate for delivery of an effector molecule from outside a first cell into the cytosol of said first cell, the conjugate comprising at least one saponin, at least one effector molecule, and at least one single domain antibody (sdAb),
wherein the saponin, the effector molecule, and the at least one sdAb are covalently bound together;
wherein the at least one saponin is a triterpenoid saponin of 12,13-dehydrooleanane type comprising an aglycone core structure selected from:
2alpha-hydroxy oleanolic acid,
16alpha-hydroxy oleanolic acid,
hederagenin (23-hydroxy oleanolic acid),
16alpha,23-dihydroxy oleanolic acid,
gypsogenin,
quillaic acid,
protoaescigenin-21(2-methylbut-2-enoate)-22-acetate,
23-oxo-barringtogenol C-21,22-bis(2-methylbut-2-enoate),
23-oxo-barringtogenol C-21(2-methylbut-2-enoate)-16,22-diacetate,
3,16,28-trihydroxy oleanan-12-en,
gypsogenic acid,
wherein the at least one effector molecule is selected from: a pharmaceutically active substance, a toxin, an oligonucleotide, a peptide, and a protein; and wherein the at least one sdAb targets a first cell surface molecule that is present on the first cell.

2. The conjugate according to claim 1, wherein the conjugate comprises at least one multivalent nanobody, preferably at least one bivalent nanobody comprising two single domain antibodies (sdAbs), and/or
wherein the effector molecule has a molecular weight of less than 200 kDa, preferably less than 150 kDa, more preferably less than 100 kDa, more preferably less than 50 kDa and/or, when the at least one effector molecule is an oligonucleotide, wherein the oligonucleotide has a size of 150 nt or less, preferably 5-150 nt, more preferably 8-100 nt, even more preferably 10-50 nt.

3. (canceled)

4. The conjugate of claim 1, wherein the cell is:
an aberrant cell selected from a tumor cell, an auto-immune cell, an infected cell such as a virally infected cell, and a cell comprising a gene defect or an enzyme defect, preferably wherein the cell is a tumor cell; or
a liver cell or an aberrant liver cell such as a tumor cell; and/or
wherein the sdAb(s) is/are selected from:
a V_{H} domain derived from a heavy chain of an antibody, preferably of immunoglobulin G origin, and/or preferably of human origin,
a V_{L} domain derived from a light chain of an antibody, preferably of immunoglobulin G origin, and/or preferably of human origin, and
a V_{HH} domain derived from a heavy-chain only antibody (HCAb), preferably from Camelidae origin or Ig-NAR origin such as a variable heavy chain new antigen receptor (V_{NAR}) domain, more preferably the HCAb is from Camelidae origin,
preferably wherein the sdAb(s) is/are selected from (a) V_{HH} domain(s) derived from an HCAb from Camelidae origin (camelid V_{H}) such as derived from an HCAb from camel, lama, alpaca, dromedary, vicuna, guanaco and Bactrian camel; and/or
wherein the conjugate comprises 1-20 sdAbs, preferably at least one multivalent nanobody such as any of a divalent-hexavalent, preferably trivalent-pentavalent, nanobody, preferably at least one bivalent nanobody, preferably 1-8, more preferably 1-6, even more preferably 1-4 sdAb's or 1-4 bivalent nanobodies, preferably 1, 2, 3 or 4 sdAb's or 1 or 2 tetravalent, trivalent and/or bivalent nanobodies, preferably 1 bivalent nanobody, or 1 bivalent nanobody and at least 1, preferably 1, further sdAb; and/or
wherein the cell surface molecule is a cell surface receptor, preferably an endocytic cell-surface receptor, preferably a tumor-cell specific receptor, more preferably the cell-surface molecule is selected from any one or more of: CD71, CA125, EpCAM(17-1A), CD52, CEA, CD44v6, FAP, EGF-IR, integrin, syndecan-1, vascular integrin alpha-V beta-3, HER2, EGFR, CD20, CD22, Folate receptor 1, CD146, CD56, CD19, CD138, CD27L receptor, prostate specific membrane antigen (PSMA), CanAg, integrin-alphaV, CA6, CD33, mesothelin, Cripto, CD3, CD30, CD239, CD70, CD123, CD352, DLL3, CD25, ephrinA4, MUC-1, Trop2, CEACAM5, CEACAM6, HER3, CD74, PTK7, Notch3, FGF2, C4.4A, FLT3, CD38, FGFR3, CD7, PD-Li, CTLA-4, CD52, PDGFRA, VEGFR1, VEGFR2, c-Met (HGFR), EGFR1, RANKL, ADAMTS5, CD16, CXCR7 (ACKR3), glucocorticoid-induced TNFR-related protein (GITR), even more preferably the cell surface molecule is selected from: HER2, c-Met, VEGFR2, CXCR7, CD71, EGFR and EGFR1, even more preferably the cell surface molecule is EGFR; and/or
wherein the sdAb(s), preferably a multivalent nanobody, more preferably a single bivalent nanobody or one bivalent nanobody and one further sdAb, are at least selected from: anti-CD71 sdAb(s), anti-HER2 sdAb(s), anti-CD20 sdAb(s), anti-CA125 sdAb(s), anti-EpCAM (17-1A) sdAb(s), anti-EGFR sdAb(s), anti-CD30 sdAb(s), anti-CD33 sdAb(s), anti-vascular integrin alpha-v beta-3 sdAb(s), anti-CD52 sdAb(s), anti-CD22 sdAb(s), anti-CEA sdAb(s), anti-CD44v6 sdAb(s), anti-FAP sdAb(s), anti-CD19 sdAb(s), anti-CanAg sdAb(s), anti-CD56 sdAb(s), anti-CD38 sdAb(s), anti-CA6 sdAb(s), anti-IGF-1R sdAb(s), anti-integrin sdAb(s), anti-syndecan-1 sdAb(s), anti-CD79b sdAb, anti-c-Met sdAb(s), anti-EGFR1 sdAb(s), anti-VEGFR2 sdAb(s), anti-CXCR7 sdAb(s) and anti-HIVgp41 sdAb(s), and optionally also anti-albumin sdAb(s), wherein the sdAbs are preferably V_{HH}(s), more preferably camelid V_{H}(s).

5-8. (canceled)

9. The conjugate of claim 1, wherein the sdAbs are at least derived from or based on any one or more of immunoglobulins selected from:
an anti-CD71 antibody such as IgG type OKT-9,
an anti-HER2 antibody such as trastuzumab (Herceptin), pertuzumab,
an anti-CD20 antibody such as rituximab, ofatumumab, tositumomab, obinutuzumab ibritumomab,
an anti-CA125 antibody such as oregovomab,
an anti-EpCAM (17-1A) antibody such as edrecolomab,
an anti-EGFR antibody such as cetuximab, matuzumab, panitumumab, nimotuzumab,
an anti-CD30 antibody such as brentuximab,
an anti-CD33 antibody such as gemtuzumab, huMy9-6,
an anti-vascular integrin alpha-v beta-3 antibody such as etaracizumab,
an anti-CD52 antibody such as alemtuzumab,
an anti-CD22 antibody such as epratuzumab, pinatuzumab, binding fragment (Fv) of anti-CD22 antibody moxetumomab, humanized monoclonal antibody inotuzumab,
an anti-CEA antibody such as labetuzumab,
an anti-CD44v6 antibody such as bivatuzumab,
an anti-FAP antibody such as sibrotuzumab,
an anti-CD19 antibody such as huB4,
an anti-CanAg antibody such as huC242,
an anti-CD56 antibody such as huN901,
an anti-CD38 antibody such as daratumumab, OKT-10 anti-CD38 monoclonal antibody,
an anti-CA6 antibody such as DS6,
an anti-IGF-1R antibody such as cixutumumab, 3B7,
an anti-integrin antibody such as CNTO 95,
an anti-syndecan-1 antibody such as B-B4,
an anti-CD79b such as polatuzumab,
an anti-HIVgp41 antibody, preferably any one of an anti-HIVgp41 antibody,
an anti-CD71 antibody,
an anti-HER2 antibody, and
an anti-EGFR antibody;
wherein more preferably the sdAbs are at least derived from or based on any one or more of: trastuzumab, pertuzumab, cetuximab, matuzumab, an anti-CD71 antibody, and OKT-9, even more preferably trastuzumab, cetuximab, and the anti-CD71 antibody OKT-9.

10. The conjugate of claim 1, wherein at least one of the sdAbs competes with binding of any one of the immunoglobulins according to claim 9, to the cell surface molecule, and/or wherein the binding site on the first cell-surface molecule for the at least one of the sdAbs is the same or overlaps with the binding site on the first cell-surface molecule for any one of the immunoglobulins according to claim 9.

11. The conjugate of claim 1, wherein the sdAbs are capable of binding to at least HER2, CD71, HIVgp41 or EGFR, preferably EGFR; and
wherein the sdAbs preferably are a V_{HH}, more preferably a camelid V_{H}.

12. The conjugate of claim 2, wherein the conjugate comprises at least one multivalent nanobody, preferably at least one bivalent nanobody comprising two sdAbs, of which nanobody at least one sdAb binds to the first cell surface molecule that is present on the first cell; and/or
wherein the at least one multivalent nanobody, preferably at least one bivalent nanobody comprising two sdAbs, comprises two sdAbs which are the same sdAbs or which are two different sdAbs; and/or
wherein one of the sdAbs of the at least one multivalent nanobody, preferably the at least one bivalent nanobody comprising two sdAbs, binds to the first cell surface molecule and at least one sdAb binds to albumin; and/or
wherein the conjugate further comprises an albumin binding protein and/or albumin; and/or
wherein at least one multivalent nanobody, preferably at least one bivalent nanobody comprising two sdAbs, is multiparatopic such as biparatopic, and/or multi-specific such as bi-specific for the first cell-surface molecule and for a second cell-surface molecule also present at the first cell, or the second cell-surface molecule present at a second cell.

13-16. (canceled)

17. The conjugate of claim 11,
wherein the sdAbs capable of binding to HER2 are selected from: sdAb produced by clone 11A4, clone 18C3, clone 22G12, clone Q17, and clone Q17-C-tag;
wherein the sdAbs capable of binding to EGFR is produced by clone anti-EGFR Q86-C-tag,
wherein the sdAbs capable of binding to CD71 is produced by clone anti-CD71 Q52-C-tag; and
wherein the sdAbs capable of binding to HIVgp41 is produced by clone anti-HIVgp41 Q8C-tag;
preferably wherein the sdAbs are encoded by a cDNA of any one of the SEQ ID NO: 1, 3, 5, 7, 9, 11, 13, 15, 17, 19, 21, 23, 25, 27, 29 and 31, or wherein the sdAbs have an amino-acid sequence according to any one or more of SEQ ID NO: 2, 4, 6, 8, 10, 12, 14, 16, 18, 20, 22, 24, 26, 28, 30, 32, 36-71 or 72, or an amino-acid sequence with at least 95% sequence identity with an amino-acid sequence according to any one or more of SEQ ID NO: 2, 4, 6, 8, 10, 12, 14, 16, 18, 20, 22, 24, 26, 28, 30, 32, 36-71 or 72, preferably at least 96%, more preferably at least 97%, even more preferably at least 98%, even more preferably at least 99%; and/or
wherein the bivalent nanobody is a hetero-bivalent nanobody, consisting of a first and second sdAb; and/or
wherein the conjugate comprises at least one bivalent nanobody, preferably a single bivalent nanobody, comprising a first and second sdAb, wherein the first sdAb has an amino-acid sequence of SEQ ID NO: 75 or an amino-acid sequence with at least 95% sequence identity with SEQ ID NO: 75, preferably at least 96%, more preferably at least 97%, even more preferably at least 98%, even more preferably at least 99%, and the second sdAb has an amino-acid sequence of SEQ ID NO: 76 or an amino-acid sequence with at least 95% sequence identity with SEQ ID NO: 76, preferably at least 96%, more preferably at least 97%, even more preferably at least 98%, even more preferably at least 99%; and/or
wherein the hetero-bivalent nanobody is a biparatopic nanobody, preferably a biparatopic nanobody with amino-acid sequence of SEQ ID NO: 74 or an amino-acid sequence with at least 95% sequence identity with SEQ ID NO: 74, preferably at least 96%, more preferably at least 97%, even more preferably at least 98%, even more preferably at least 99%.

18-20. (canceled)

21. The conjugate of claim 1, wherein the at least one saponin is a triterpenoid saponin of the 12,13-dehydrooleanane type comprising an aldehyde group at position C-23; and/or
wherein the at least one saponin comprises an aglycone core structure selected from quillaic acid and gypsogenin, more preferably the at least one saponin comprises the aglycone core structure quillaic acid; and/or
wherein the at least one saponin comprises one or both of: a first saccharide chain bound to the C3 atom or to the C28 atom of the aglycone core structure of the at least one saponin, preferably bound to the C3 atom, and a second saccharide chain bound to the C28 atom of the aglycone core structure of the at least one saponin, and preferably the at least one saponin comprises the first and the second saccharide chain;
wherein preferably the at least one saponin comprises the first saccharide chain that is selected from:
GlcA-,
Glc-,
Rha-(1→2)-Ara-,
Gal-(1→2)-[Xyl-(1→3)]-GlcA-,
Glc-(1→2)-[Glc-(1→4)]-GlcA-,
Glc-(1→2)-Ara-(1→3)-[Gal-(1→2)]-GlcA-,
Xyl-(1→2)-Ara-(1→3)-[Gal-(1→2)]-GlcA-,
Glc-(1→3)-Gal-(1→2)-[Xyl-(1→3)]-Glc-(1→4)-Gal-,
Rha-(1→2)-Gal-(1-3)-[Glc-(1→2)]-GlcA-,
Ara-(1→4)-Rha-(1→2)-Glc-(1→2)-Rha-(1→2)-GlcA-,
Ara-(1→4)-Fuc-(1→2)-Glc-(1→2)-Rha-(1→2)-GlcA-,
Ara-(1→4)-Rha-(1→2)-Gal-(1→2)-Rha-(1→2)-GlcA-,
Ara-(1→4)-Fuc-(1→2)-Gal-(1→2)-Rha-(1→2)-GlcA-,
Ara-(1→4)-Rha-(1→2)-Glc-(1→2)-Fuc-(1→2)-GlcA-,
Ara-(1→4)-Fuc-(1→2)-Glc-(1→2)-Fuc-(1→2)-GlcA-,
Ara-(1→4)-Rha-(1→2)-Gal-(1→2)-Fuc-(1→2)-GlcA-,
Ara-(1→4)-Fuc-(1→2)-Gal-(1→2)-Fuc-(1→2)-GlcA-,
Xyl-(1→4)-Rha-(1→2)-Glc-(1→2)-Rha-(1→2)-GlcA-,
Xyl-(1→4)-Fuc-(1→2)-Glc-(1→2)-Rha-(1→2)-GlcA-,
Xyl-(1→4)-Rha-(1→2)-Gal-(1→2)-Rha-(1→2)-GlcA-,
Xyl-(1→4)-Fuc-(1→2)-Gal-(1→2)-Rha-(1→2)-GlcA-,
Xyl-(1→4)-Rha-(1→2)-Glc-(1→2)-Fuc-(1→2)-GlcA-,
Xyl-(1→4)-Fuc-(1→2)-Glc-(1→2)-Fuc-(1→2)-GlcA-,
Xyl-(1→4)-Rha-(1→2)-Gal-(1→2)-Fuc-(1→2)-GlcA-, and
Xyl-(1→4)-Fuc-(1→2)-Gal-(1→2)-Fuc-(1→2)-GlcA-; and/or
wherein the at least one saponin comprises the second saccharide chain selected from:
Glc-,
Gal-,
Rha-(1→2)-[Xyl-(1-4)]-Rha-,
Rha-(1→2)-[Ara-(1→3)-Xyl-(1→4)]-Rha-,
Ara-,
Xyl-,
Xyl-(1→4)-Rha-(1→2)-[R1-(→4)]-Fuc- wherein R1 is 4E-Methoxycinnamic acid,
Xyl-(1→4)-Rha-(1→2)-[R2-(→4)]-Fuc- wherein R2 is 4Z-Methoxycinnamic acid,
Xyl-(1→4)-[Gal-(1→3)]-Rha-(1→2)-4-OAc-Fuc-,
Xyl-(1→4)-[Glc-(1→3)]-Rha-(1→2)-3,4-di-OAc-Fuc-,
Xyl-(1→4)-[Glc-(1→3)]-Rha-(1→2)-[R3-(→4)]-3-OAc-Fuc- wherein R3 is 4E-Methoxycinnamic acid,
Glc-(1→3)-Xyl-(1→4)-[Glc-(1→3)]-Rha-(1→2)-4-OAc-Fuc-,
Glc-(1→3)-Xyl-(1→4)-Rha-(1→2)-4-OAc-Fuc-,
(Ara- or Xyl-)(1→3)-(Ara- or Xyl-)(1→4)-(Rha- or Fuc-)(1→2)-[4-OAc-(Rha- or Fuc-)(1→4)]-(Rha- or Fuc-),
Xyl-(1→3)-Xyl-(1→4)-Rha-(1→2)-[Qui-(1→4)]-Fuc-,
Api-(1→3)-Xyl-(1→4)-[Glc-(1→3)]-Rha-(1→2)-Fuc-,
Xyl-(1→4)-[Gal-(1→3)]-Rha-(1→2)-Fuc-,
Xyl-(1→4)-[Glc-(1→3)]-Rha-(1→2)-Fuc-,
Ara/Xyl-(1→4)-Rha/Fuc-(1→4)-[Glc/Gal-(1-2)]-Fuc-,
Api-(1→3)-Xyl-(1→4)-[Glc-(1→3)]-Rha-(1→2)-[R4-(→4)]-Fuc- wherein R4 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R5-(→4)]-Fuc- wherein R5 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[Rha-(1→3)]-4-OAc-Fuc-,
Api-(1→3)-Xyl-(1→4)-[Glc-(1→3)]-Rha-(1→2)-[Rha-(1→3)]-4-OAc-Fuc-,
6-OAc-Glc-(1→3)-Xyl-(1→4)-Rha-(1→2)-[3-OAc-Rha-(1→3)]-Fuc-,
[4,6-di-OAc-Glc-(1→3)]-[Xyl-(1-4)]-Rha-(1→2)-[3,4-di-OAc-Qui-(1→4)]-Fuc-
Glc-(1→3)-Xyl-(1→4)-Rha-(1→2)-[3-OAc-Rha-(1→3)]-Fuc-,
Xyl-(1→3)-Xyl-(1→4)-Rha-(1→2)-[Qui-(1→4)]-Fuc-,
Glc-(1→3)-[Xyl-(1→4)]-Rha-(1-2)-[Qui(1→4)]-Fuc-,
Glc-(1→3)-Xyl-(1→4)-Rha-(1→2)-[Xyl-(1→3)-4-OAc-Qui-(1→4)]-Fuc-,
Xyl-(1→3)-Xyl-(1→4)-Rha-(1→2)-[3,4-di-OAc-Qui-(1→4)]-Fuc-,
Glc-(1→3)-[Xyl-(1-4)]-Rha-(1→2)-Fuc-,
6-OAc-Glc-(1→3)-[Xyl-(1→4)]-Rha-(1→2)-Fuc-,
Glc-(1→3)-[Xyl-(1→3)-Xyl-(1→4)]-Rha-(1→2)-Fuc-,
Xyl-(1→3)-Xyl-(1→4)-Rha-(1→2)-[Xyl-(1→3)-4-OAc-Qui-(1→4)]-Fuc-,
Api/Xyl-(1→3)-Xyl-(1→4)-[Glc-(1→3)]-Rha-(1→2)-[Rha-(1→3)]-4OAc-Fuc-,
Api-(1→3)-Xyl-(1→4)-[Glc-(1→3)]-Rha-(1→2)-[Rha-(1→3)]-4OAc-Fuc-,
Api/Xyl-(1→3)-Xyl-(1→4)-[Glc-(1→3)]-Rha-(1→2)-[R6-(→4)-Fuc- wherein R6 is 5-O-[5-O-Rha-(1→2)-Ara/Api-3,5-dihydroxy-6-methyl-octanoyl]-3,5-dihydroxy-6-methyl-octanoic acid),
Api/Xyl-(1→3)-Xyl-(1→4)-[Glc-(1→3)]-Rha-(1→2)-[R7-(→4)]-Fuc- wherein R7 is 5-O-[5-O-Ara/Api-3,5-dihydroxy-6-methyl-octanoyl]-3,5-dihydroxy-6-methyl-octanoic acid),
Api/Xyl-(1→3)-Xyl-(1→4)-[Glc-(1→3)]-Rha-(1→2)-[R8-(→4)]-Fuc- wherein R8 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R9-(→4)]-Fuc- wherein R9 is 5-O-[5-O-Ara/Api-3,5-dihydroxy-6-methyl-octanoyl]-3,5-dihydroxy-6-methyl-octanoic acid),
Xyl-(1→3)-Xyl-(1→4)-Rha-(1→2)-[R10-(→4)]-Fuc- wherein R10 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R11-(→3)]-Fuc- wherein R11 is 5-O-[5-O-Ara/Api-3,5-dihydroxy-6-methyl-octanoyl]-3,5-dihydroxy-6-methyl-octanoic acid),
Xyl-(1→3)-Xyl-(1→4)-Rha-(1→2)-[R12-(→3)]-Fuc- wherein R12 is 5-O-[5-O-Ara/Api-3,5-dihydroxy-6-methyl-octanoyl]-3,5-dihydroxy-6-methyl-octanoic acid), and
Glc-(1→3)-[Glc-(1→6)]-Gal-.

22-24. (canceled)

25. The conjugate of claim 1, wherein the at least one saponin comprises a first saccharide chain and a second saccharide chain of claim 21,
wherein preferably the first saccharide chain comprises more than one saccharide moiety and the second saccharide chain comprises more than one saccharide moiety; and
wherein the aglycone core structure preferably is quillaic acid or gypsogenin, more preferably quillaic acid; and/or
wherein the at least one saponin comprises a first saccharide chain bound to the C3 atom of the aglycone core structure of the at least one saponin;
wherein the first saccharide chain is Gal-(1→2)-[Xyl-(1→3)]-GlcA; and
wherein preferably the aglycone core structure is quillaic acid or gypsogenin, more preferably quillaic acid; and/or
wherein one, two or three, preferably one or two, more preferably one, of:
an aldehyde group in the aglycone core structure of the at least one saponin has been derivatised when present,
a carboxyl group of a glucuronic acid moiety in a first saccharide chain of the at least one saponin has been derivatised when present in the at least one saponin, and
at least one acetoxy (Me(CO)O—) group in a second saccharide chain of the at least one saponin has been derivatised if present, and/or
wherein the at least one saponin comprises:
(i) an aglycone core structure comprising an aldehyde group which has been derivatised by:
reduction to an alcohol;
transformation into a hydrazone bond through reaction with N-g-maleimidocaproic acid hydrazide (EMCH), wherein the maleimide group of the EMCH is optionally derivatised by formation of a thioether bond with mercaptoethanol;
transformation into a hydrazone bond through reaction with N-[β-maleimidopropionic acid]hydrazide (BMPH) wherein the maleimide group of the BMPH is optionally derivatised by formation of a thioether bond with mercaptoethanol; or
transformation into a hydrazone bond through reaction with N-[κ-maleimidoundecanoic acid]hydrazide (KMUH), wherein the maleimide group of the KMUH is optionally derivatised by formation of a thioether bond with mercaptoethanol; or
(ii) a first saccharide chain comprising a carboxyl group, preferably a carboxyl group of a glucuronic acid moiety, which has been derivatised by transformation into an amide bond through reaction with 2-amino-2-methyl-1,3-propanediol (AMPD) or N-(2-aminoethyl)maleimide (AEM); or
(iii) a second saccharide chain comprising an acetoxy group (Me(CO)O—) which has been derivatised by transformation into a hydroxyl group (HO—) by deacetylation: or
(iv) any combination of two or three derivatisations (i), (ii) and/or (iii), preferably any combination of two derivatisations of (i), (ii) and (iii); and/or
wherein the at least one saponin is any one or more of:
a) saponin selected from list A:
Quillaja saponaria saponin mixture, or a saponin isolated from Quillaja saponaria, for example Quil-A, QS-17-api, QS-17-xyl, QS-21, QS-21A, QS-21B, QS-7-xyl;
Saponinum album saponin mixture, or a saponin isolated from Saponinum album;
Saponaria officinalis saponin mixture, or a saponin isolated from Saponaria officinalis; and
Quillaja bark saponin mixture, or a saponin isolated from Quillaja bark, for example Quil-A, QS-17-api, QS-17-xyl, QS-21, QS-21A, QS-21B, QS-7-xyl; or
b) a saponin comprising a gypsogenin aglycone core structure, selected from list B:
SA1641, gypsoside A, NP-017772, NP-017774, NP-017777, NP-017778, NP-018109, NP-017888, NP-017889, NP-018108, SO1658 and Phytolaccagenin; or
c) a saponin comprising a quillaic acid aglycone core structure, selected from list C;
AG1856, AG1, AG2, Agrostemmoside E, GE1741, Gypsophila saponin 1 (Gyp1), NP-017674, NP-017810, NP-003881, NP-017676, NP-017677, NP-017705, NP-017706, NP-017773, NP-017775, SA1657, Saponarioside B, SO1542, SO1584, SO1674, SO1700, SO1730, SO1772, SO1832, SO1861, SO1862, SO1904, QS-7, QS-7 api, QS-17, QS-18, QS-21A-apio, QS-21A-xylo, QS-21 B-apio and QS-21 B-xylo; or
d) a saponin comprising a 12, 13-dehydrooleanane type aglycone core structure without an aldehyde group at the C-23 position of the aglycone, selected from list D:
Aescin Ia, aescinate, alpha-Hederin, AMA-1, AMR, AS6.2, AS64R, Assamsaponin F, dipsacoside B, esculentoside A, macranthoidin A, NP-005236, NP-012672, Primula acid 1, saikosaponin A, saikosaponin D, Teaseed saponin I and Teaseedsaponin J,
wherein preferably the at least one saponin is any one or more of a saponin selected from list A, B or C, more preferably from list B or C, most preferably a saponin selected from list C; and/or
wherein the at least one saponin is any one or more of:
AG1856, GE1741, a saponin isolated from Quillaja saponaria, Quil-A, QS-17, QS-21, QS-7, SA1641, a saponin isolated from Saponaria officinalis, Saponarioside B, SO1542, SO1584, SO1658, SO1674, SO1700, SO1730, SO1772, SO1832, SO1861, SO1862 and SO1904, preferably the at least one saponin is any one or more of QS-21, SO1832, SO1861, SA1641 and GE1741, more preferably the at least one saponin is QS-21, SO1832, SO1861 or AG1856, even more preferably the at least one saponin is SO1832, SO1861 or AG1856, most preferably, the at least one saponin is SO1832 or SO1861, or is SO1861; and/or
wherein the at least one saponin is a saponin isolated from Saponaria officinalis, preferably the at least one saponin is any one or more of Saponarioside B, SO1542, SO1584, SO1658, SO1674, SO1700, SO1730, SO1772, SO1832, SO1861, SO1862 and SO1904, more preferably the at least one saponin is any one or more of SO1832, SO1861 and SO1862, even more preferably SO1832 and SO1861, even more preferably the at least one saponin is SO1861, and/or wherein the at least one saponin is originating from a mono-desmosidic or bi-desmosidic triterpene saponin, or derivative thereof, belonging to the type of a 12,13-dehydrooleanane saponin with an aldehyde function in position C23 and optionally comprising a glucuronic acid unit in a first saccharide chain bound at the C3beta-OH group of the aglycone core structure of the saponin, preferably at least one saponin originating from a bi-desmosidic triterpene saponin, belonging to the type of a 12,13-dehydrooleanane saponin with an aldehyde function in position C23 and comprising a glucuronic acid unit in a first saccharide chain bound at the C3beta-OH group of the aglycone core structure of the saponin, wherein the aglycone core structure is gypsogenin or quillaic acid, preferably quillaic acid, and/or wherein the conjugate comprising more than one copy of the saponin, preferably 1-64 copies of the saponin, more preferably 2-32 copies of the saponin, even more preferably 4-16 copies of the saponin, most preferably 4-8 copies of the saponin.

26-31. (canceled)

32. The conjugate of claim 1, wherein the oligonucleotide is selected from deoxyribonucleic acid (DNA) oligomer, ribonucleic acid (RNA) oligomer, anti-sense oligonucleotide (ASO, AON), short interfering RNA (siRNA), anti-microRNA (anti-miRNA), DNA aptamer, RNA aptamer, mRNA, mini-circle DNA, peptide nucleic acid (PNA), phosphoramidate morpholino oligomer (PMO), phosphorothioate-modified antisense oligonucleotide (PS-ASO), 2′-O-methyl (2′-OMe) phosphorothioate RNA, 2′-O-methoxyethyl (2′-O-MOE) RNA {2′-O-methoxyethyl-RNA (MOE)}, locked nucleic acid (LNA), bridged nucleic acid (BNA), 2′-deoxy-2′-fluoroarabino nucleic acid (FANA), 2′-O-methoxyethyl-RNA (MOE), 3′-fluoro hexitol nucleic acid (FHNA), glycol nucleic acid (GNA), xeno nucleic acid oligonucleotide, and threose nucleic acid (TNA), wherein preferably the oligonucleotide is a BNA, more preferably wherein the oligonucleotide is a BNA for silencing HSP27 protein expression or a BNA for silencing apolipoprotein B expression; and/or
wherein the oligonucleotide is selected from short interfering RNA (siRNA), short hairpin RNA (shRNA), anti-hairpin-shaped microRNA (miRNA), single-stranded RNA, aptamer RNA, double-stranded RNA (dsRNA), anti-microRNA (anti-miRNA, anti-miR), antisense oligonucleotide (ASO), mRNA, DNA, antisense DNA, locked nucleic acid (LNA), bridged nucleic acid (BNA), 2′-O,4′-aminoethylene bridged nucleic Acid (BNANC), BNA-based siRNA, and BNA-based antisense oligonucleotide (BNA-AON); and/or
wherein the at least one effector molecule is an oligonucleotide selected from an anti-miRNA, a BNA-AON, and an siRNA, such as BNA-based siRNA, preferably selected from chemically modified siRNA, metabolically stable siRNA and chemically modified, metabolically stable siRNA; and/or
wherein the oligonucleotide is an oligonucleotide that is capable of silencing a gene, when present in a cell comprising such gene, wherein the gene is selected from apolipoprotein B (apoB), HSP27, transthyretin (TTR), proprotein convertase subtilisin/kexin type 9 (PCSK9), delta-aminolevulinate synthase 1 (ALAS1), antithrombin 3 (AT3), glycolate oxidase (GO), complement component C5 (CC5), X gene of hepatitis B virus (HBV), S gene of HBV, alpha-1 antitrypsin (AAT), and lactate dehydrogenase (LDH), and/or wherein the gene is capable of targeting an aberrant miRNA when present in a cell comprising such aberrant miRNA; and/or
wherein the oligonucleotide is an oligonucleotide that is capable of targeting an mRNA, when present in a cell comprising such mRNA, wherein the mRNA is involved in expression of any one of proteins: apoB, HSP27, TTR, PCSK9, ALAS1, AT3, GO, CC5, expression product of X gene of HBV, expression product of S gene of HBV, AAT, and LDH; or wherein the mRNA is capable of antagonizing or restoring an miRNA function such as inhibiting an oncogenic miRNA (onco-miR) or suppressing of expression of an onco-miR, when present in a cell comprising such an miRNA.

33-40. (canceled)

41. The conjugate according to claim 1, wherein the at least one effector molecule is an oligonucleotide; and/or
wherein the conjugate comprises 1-16 effector molecules, preferably oligonucleotide(s), preferably 1-4 effector molecules, most preferably 1 or 2 effector molecule(s), wherein the effector molecule(s) is/are preferably covalently bound in the conjugate via a cleavable bond, selected from:
a bond subject to cleavage under acidic conditions such as a semi-carbazone bond, a hydrazone bond, an imine bond, an acetal bond including a 1,3-dioxolane bond, a ketal bond, an ester bond, and/or an oxime bond;
a bond susceptible to proteolysis, for example an amide bond or a peptide bond, preferably subject to proteolysis by Cathepsin B;
a red/ox-cleavable bond such as a disulfide bond, or a thiol-exchange reaction-susceptible bond such as a thio-ether bond, preferably a hydrazone bond or a semicarbazone bond, more preferably a hydrazone bond.

42-43. (canceled)

44. The conjugate according to claim 1, wherein the at least one effector molecule is a pharmaceutically active substance.

45. The conjugate of claim 1, wherein the conjugate comprises at least one first linker, wherein:
each of the at least one sdAb bound thereto, preferably at least one bivalent nanobody, more preferably a single bivalent nanobody;
the at least one saponin bound thereto; and
the at least one effector molecule covalently bound thereto,
wherein the at least one effector molecule is bound to said at least one first linker separately, either directly, or via a first, second and third additional linker for the at least one sdAb, the at least one saponin and the at least one effector molecule, respectively; and
wherein preferably, the conjugate comprises at least one of a first linker with one bivalent nanobody, at least one saponin and at least one, preferably one, effector molecule covalently bound to that first linker, separately, either directly, or via a first, second and third additional linker for conjugating the at least one bivalent nanobody, the at least one saponin and the at least one effector molecule; and/or
wherein the first linker is a trifunctional linker, preferably wherein the conjugate comprises 1-4 of said trifunctional linkers for every at least one sdAb or every multivalent nanobody, preferably bivalent nanobody, comprised by the conjugate, more preferably 1-2, even more preferably 1 trifunctional linker; or
wherein the first linker is a trifunctional linker, preferably wherein the conjugate comprises on average 1-4, preferably on average 1.2-1.8 of said trifunctional linkers;
preferably wherein the at least one sdAb or the multivalent nanobody such as the bivalent nanobody comprises a first additional linker comprising at least one cysteine residue, preferably 1-4 cysteine residues, preferably a tetracysteine repeat such as sequence HRWCCPGCCKTF (SEQ ID NO: 77), and wherein each of the trifunctional linkers, preferably one trifunctional linker, is bound to this first additional linker;
more preferably wherein the conjugate comprises a single multivalent nanobody, preferably a bivalent nanobody, comprising said first additional linker comprising at least one cysteine residue, preferably 1-4 cysteine residues, preferably a tetracysteine repeat such as sequence HRWCCPGCCKTF (SEQ ID NO: 77); and
wherein all of the one or more trifunctional linkers, preferably one trifunctional linker, are each/is separately bound to a cysteine residue of the tetracysteine repeat of the first additional linker.

46-47. (canceled)

48. The conjugate according to claim 45, wherein the conjugate comprises any one of one multivalent nanobody selected from a trivalent nanobody or a bivalent nanobody, 1-4 sdAb's, 1-2 sdAb's, and 1 bivalent nanobody, preferably one bivalent nanobody and/or 3 sdAbs preferably comprising a bivalent nanobody.

49-50. (canceled)

51. The conjugate of claim 45, wherein the at least one saponin is covalently bound directly to an amino-acid residue of the first linker, preferably to a cysteine and/or to a lysine, and/or is covalently bound via the first additional linker, wherein preferably said first additional linker is a cleavable linker; and/or
wherein the first additional linker to which the one or more saponins are covalently bound comprises a polymeric molecule or an oligomeric molecule to which the one or more saponins are covalently bound, the polymeric molecule or the oligomeric molecule selected from: a dendron, a poly-ethylene glycol such as any one of PEG3-PEG30, preferably any one of PEG4-PEG12, preferably the polymeric molecule or the oligomeric molecule of the conjugate is a dendron such as a poly-amidoamine (PAMAM) dendrimer: preferably:
wherein the first additional linker that covalently binds the one or more saponins to the first linker is a dendron to which the one or more saponins are covalently bound, preferably a G2 dendron, a G3 dendron, a G4 dendron or a G5 dendron or a poly-amidoamine (PAMAM) dendrimer, more preferably a G2 dendron or a G3 dendron or a poly-amidoamine (PAMAM) dendrimer, more preferably a G2 dendron or a G3 dendron.

52-53. (canceled)

54. The conjugate of claim 45, wherein the at least one saponin is covalently bound via a cleavable first additional linker to the first linker; or
wherein the cleavable first additional linker is subject to cleavage under acidic conditions, reductive conditions, enzymatic conditions and/or light-induced conditions, and preferably the cleavable first additional linker comprises a cleavable bond selected from:
a bond subject to cleavage under acidic conditions such as a semi-carbazone bond, a hydrazone bond, an imine bond, an acetal bond including a 1,3-dioxolane bond, a ketal bond, an ester bond, and/or an oxime bond;
a bond susceptible to proteolysis, for example an amide or a peptide bond, preferably subject to proteolysis by Cathepsin B; and
a red/ox-cleavable bond such as a disulfide bond, or a thiol-exchange reaction-susceptible bond such as a thio-ether bond.

55. (canceled)

56. The conjugate of claim 54, wherein the cleavable first additional linker is subject to cleavage in vivo under acidic conditions such as the acidic conditions present in endosomes and/or lysosomes of mammalian cells, preferably human cells such as tumor cells, preferably at pH 4.0-6.5, and more preferably at pH≤5.5; and/or
wherein the at least one saponin is covalently bound to the first additional linker or cleavable first additional linker via any one or more of: a semi-carbazone bond, a hydrazone bond, an imine bond, an acetal bond including a 1,3-dioxolane bond, a ketal bond, an ester bond, and/or an oxime bond, preferably a semicarbazone bond or a hydrazone bond, more preferably a hydrazone bond; and/or
wherein the conjugate is obtained by conjugating the at least one effector molecule, the at least one sdAb, or the at least one multivalent nanobody, preferably the at least one bivalent nanobody comprising two sdAbs, with at least one saponin wherein said at least one saponin comprises an aglycone core structure comprising an aldehyde function in position C23, which aldehyde function is involved in the covalent bonding to the first linker, the first additional linker or the cleavable first additional linker, preferably the cleavable first additional linker, and/or
wherein the first linker is a trifunctional linker that is in its non-conjugated form represented by Structure A: 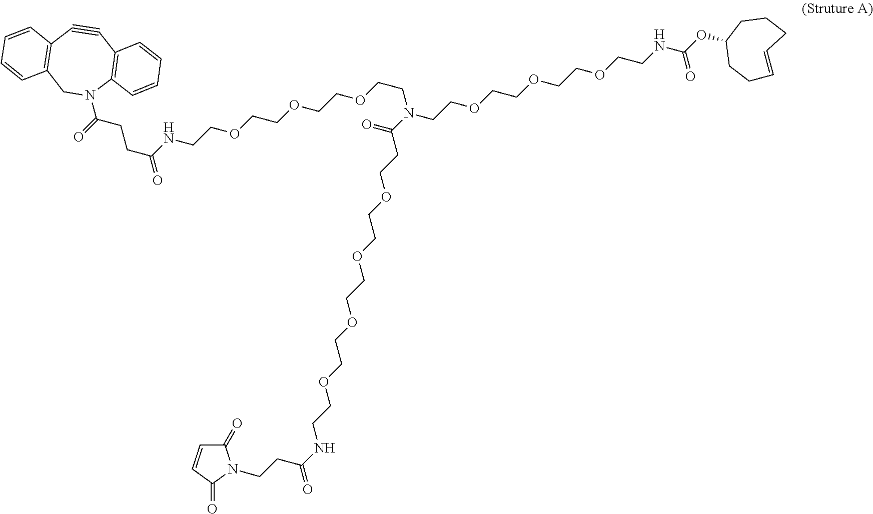
and/or
wherein the first linker of the conjugate is the trifunctional linker of Structure A and wherein the conjugate has a molecular structure represented by Structure B: 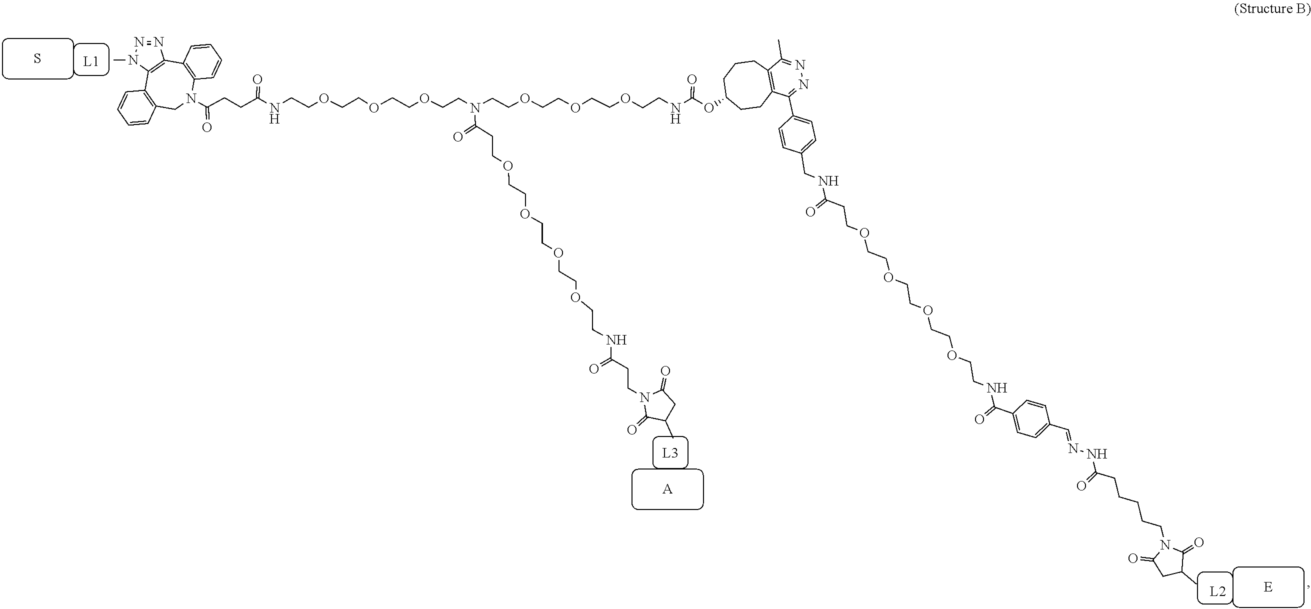
wherein:
S represents the at least one saponin, E is the at least one, preferably one, effector molecule,
A is the at least one sdAb such as a single sdAb, or the at least one multivalent nanobody, preferably the at least one bivalent nanobody comprising a first sdAb and a second sdAb,
L1 is the first additional linker to which the at least one saponin is covalently bound, L1 optionally comprising an oligomeric or polymeric molecule to which the at least one saponin is covalently bound,
L2 is the second additional linker to which the at least one, preferably one effector molecule is covalently bound, and
L3 is the third additional linker to which the at least one sdAb, the at least one multivalent nanobody, preferably the at least one bivalent nanobody, more preferably one bivalent nanobody, is covalently bound, wherein L1, L2 and L3 are the same or different, preferably different.

57-60. (canceled)

61. A pharmaceutical composition comprising the conjugate of claim 1, and optionally a pharmaceutically acceptable excipient and/or pharmaceutically acceptable diluent.

62. (canceled)

63. A method for the treatment or the prophylaxis of any one or more of: a cancer, an auto-immune disease such as rheumatoid arthritis, an enzyme deficiency, a disease related to an enzyme deficiency, a gene defect, a disease relating to a gene defect, an infection such as a viral infection, hypercholesterolemia, primary hyperoxaluria, haemophilia A, haemophilia B, alpha-1 antitrypsin related liver disease, acute hepatic porphyria, an amyloidosis and transthyretin-mediated amyloidosis, preferably a cancer such as bladder cancer such as metastatic bladder cancer, urothelial carcinoma, cancer of the urinary tract, urologic neoplasms, prostate cancer such as castration resistant prostate cancer, breast cancer, malignant neoplasm of pancreas, ovary cancer, lung cancer such as non-squamous non-small cell lung cancer and squamous cell carcinoma of lung in a subject in need thereof, wherein the method comprises administering the pharmaceutical composition of claim 61 or the conjugate of claim 1 to a subject; and/or
wherein the method is for:
the treatment or prevention of cancer in a human subject, preferably a cancer selected from the list above; and/or
the treatment or prophylaxis of cancer in a patient in need thereof, wherein the at least one sdAb, the at least one multivalent nanobody, preferably the at least one bivalent nanobody comprising two sdAbs, binds to the first cell-surface molecule of the first cell, preferably to a tumor-cell surface molecule of the cell, more preferably to a tumor cell-specific surface molecule of the cell, wherein preferably the cancer is a cancer selected from the list above; and/or
the pharmaceutical composition, preferably a therapeutically effective amount of the pharmaceutical composition is administered to a patient in need thereof, preferably a human patient.

64. (canceled)


SEQUENCE LISTING (78 sequences)

SEQ ID NO: 1 - DNA, 414 bp, Camelus dromedarius
  source 1..414: mol_type = other DNA
gaagttcagctgcaggaatctggtggtggtctggttcagccgggtggttctctgcgtctgtcttgcgcggcgtctggtttcatcttctctaacgacgcgatgacctgggttcgtcaggcgccgggtaaaggtctggaatgggtttcttctatcaactggtctggtacccacaccaactacgcggactctgttaaaggtcgtttcaccatctctcgtgacaacgcgaaacgtaccctgtacctgcagatgaactctctgaaagacgaagacaccgcgctgtactactgcgttaccggttacggtgttaccaaaaccccgaccggtcagggtacccaggttaccgtttcttctcaccaccaccaccaccactctccgtctaccccgccgaccccgtctccgtctaccccgccgtgc

SEQ ID NO: 2 - protein, 138 aa, Camelus dromedarius
EVQLQESGGGLVQPGGSLRLSCAASGFIFSNDAMTWVRQAPGKGLEWVSSINWSGTHTNYADSVKGRFTISRDNAKRTLYLQMNSLKDEDTALYYCVTGYGVTKTPTGQGTQVTVSSHHHHHHSPSTPPTPSPSTPPC

SEQ ID NO: 3 - DNA, 375 bp, Camelus dromedarius
  source 1..375: mol_type = other DNA
atggaagttcagctggttgaatctggtggtggtctggttcaggcgggtggttctctgcgtctgtcttgcgcggcgtctggtatcaccttctctatcaacaccatgggttggtaccgtcaggcgccgggtaaacagcgtgaactggttgcgctgatctcttctatcggtgacacctactacgcggactctgttaaaggtcgtttcaccatctctcgtgacaacgcgaaaaacaccgtttacctgcagatgaactctctgaaaccggaagacaccgcggtttactactgcaaacgtttccgtaccgcggcgcagggtaccgactactggggtcagggtacccaggttaccgtttcttctcaccaccaccaccaccac

SEQ ID NO: 4 - protein, 125 aa, Camelus dromedarius
MEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHH

SEQ ID NO: 5 - DNA, 375 bp, synthetic construct
  misc_feature 1..375: note = Amino-acid coding DNA sequence of Anti-HER2 dsAb pcNB2
  source 1..375: mol_type = other DNA
atggaagttcagctggttgaaaaaggtggtggtcgtgttcaggcgggtggttctctgcgtctgcgttgcgcggcgtctggtatcaccttctctatcaacaccatgggttggtaccgtcaggcgccgggtaaacagcgtgaactggttgcgctgatctcttctatcggtgacacctactacgcggactctgttaaaggtcgtttccgtatccgtcgtgacaacgcgaaaaacaccgtttacctgcgtatgcgtcgtctgaaaccggaagacaccgcggtttactactgcaaacgtttccgtaccgcggcgcagggtaccgactactggggtcagggtacccgtgttaccgtttctaaacaccaccaccaccaccac

SEQ ID NO: 6 - protein, 125 aa, synthetic construct
  REGION 1..125: note = Amino-acid sequence of Anti-HER2 dsAb pcNB2
MEVQLVEKGGGRVQAGGSLRLRCAASGITFSINTMGWYRQAPGKQRELVALISSIGDTYYADSVKGRFRIRRDNAKNTVYLRMRRLKPEDTAVYYCKRFRTAAQGTDYWGQGTRVTVSKHHHHHH

SEQ ID NO: 7 - DNA, 378 bp, Camelus dromedarius
  source 1..378: mol_type = other DNA
gcggcgcaggttaaactggaagaatctggtggtggttctgttcagaccggtggttctctgcgtctgacctgcgcggcgtctggtcgtacctctcgttcttacggtatgggttggttccgtcaggcgccgggtaaagaacgtgaattcgtttctggtatctcttggcgtggtgactctaccggttacgcggactctgttaaaggtcgtttcaccatctctcgtgacaacgcgaaaaacaccgttgacctgcagatgaactctctgaaaccggaagacaccgcgatctactactgcgcggcggcggcgggttctgcgtggtacggtaccctgtacgaatacgactactggggtcagggtacccaggttaccgtttcttct

SEQ ID NO: 8 - protein, 126 aa, Camelus dromedarius
AAQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 9 - DNA, 381 bp, Camelus dromedarius
  source 1..381: mol_type = other DNA
gaagttcagctggttgaatctggtggtggtctggttcaggcgggtggttctctgcgtctgtcttgcgcggcgtctggtcgtaccttctcttcttacgcgatgggttggttccgtcaggcgccgggtaaagaacgtgaattcgttgttgcgatcaactggtcttctggttctacctactacgcggactctgttaaaggtcgtttcaccatctctcgtgacaacgcgaaaaacaccatgtacctgcagatgaactctctgaaaccggaagacaccgcggtttactactgcgcggcgggttaccagatcaactctggtaactacaacttcaaagactacgaatacgactactggggtcagggtacccaggttaccgtttcttct

SEQ ID NO: 10 - protein, 127 aa, Camelus dromedarius
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 11 - DNA, 378 bp, synthetic construct
  misc_feature 1..378: note = Amino-acid coding DNA sequence of Anti-VGFR2 dsAb NTV1
  source 1..378: mol_type = other DNA
atggcgcaggttcagctgctggaatctggtggtggtctggttcagccgggtggttctctgcgtctgtcttgcgcggcgtctggttactctgttatcaacgacttcatgacctgggttcgtcaggcgccgggtaaaggtctggaatgggtttcttctatctctgttgcggacggttctacctactacgcggactctgttaaaggtcgtttcaccatctctcgtgacaactctaaaaacaccctgtacctgcagatgaactctctgcgtgcggaagacaccgcggtttactactgcgcggcgcgtgttggtggtcgtgacctgggttggccgtacgaactggactactggggtcagggtaccctggttaccgtttcttct

SEQ ID NO: 12 - protein, 126 aa, synthetic construct
  REGION 1..126: note = Amino-acid sequence of Anti-VGFR2 dsAb NTV1
MAQVQLLESGGGLVQPGGSLRLSCAASGYSVINDFMTWVRQAPGKGLEWVSSISVADGSTYYADSVKGRFTISRDNSKNTLYLQMNSLRAEDTAVYYCAARVGGRDLGWPYELDYWGQGTLVTVSS

SEQ ID NO: 13 - DNA, 372 bp, synthetic construct
  misc_feature 1..372: note = Amino-acid coding DNA sequence of Anti-VGFR2 dsAb NTV2
  source 1..372: mol_type = other DNA
atggcgcaggttcagctgctggaatctggtggtggtctggttcagccgggtggttctctgcgtctgtcttgcgcggcgtctggtttcaaaatcaccaacaaaaccatggcgtgggttcgtcaggcgccgggtaaaggtctggaatgggtttcttctatcggttcttcttctggttctacctactacgcggactctgttaaaggtcgtttcaccatctctcgtgacaactctaaaaacaccctgtacctgcagatgaactctctgcgtgcggaagacaccgcggtttactactgcgcgcgtcgtaaaggtaaccgtctgggtccggcggcgctgcgttcttggggtcagggtaccctggttaccgtttcttct

SEQ ID NO: 14 - protein, 124 aa, synthetic construct
  REGION 1..124: note = Amino-acid sequence of Anti-VGFR2 dsAb NTV2
MAQVQLLESGGGLVQPGGSLRLSCAASGFKITNKTMAWVRQAPGKGLEWVSSIGSSSGSTYYADSVKGRFTISRDNSKNTLYLQMNSLRAEDTAVYYCARRKGNRLGPAALRSWGQGTLVTVSS

SEQ ID NO: 15 - DNA, 363 bp, synthetic construct
  misc_feature 1..363: note = Amino-acid coding DNA sequence of Anti-VGFR2 dsAb NTV3
  source 1..363: mol_type = other DNA
atggcgcaggttcagctgctggaatctggtggtggtctggttcagccgggtggttctctgcgtctgtcttgcgcggcgtctggtgttcgtgttaactacaaatctatgtcttgggttcgtcaggcgccgggtaaaggtctggaatgggtttctaccatcacctctcgtaacggttctacctactacgcggactctgttaaaggtcgtttcaccatctctcgtgacaactctaaaaacaccctgtacctgcagatgaactctctgcgtgcggaagacaccgcggtttactactgcgcgaccggtcgtgcgcaccacgcgccggttcgttactggggtcagggtaccctggttaccgtttcttct

SEQ ID NO: 16 - protein, 121 aa, synthetic construct
  REGION 1..121: note = Amino-acid sequence of Anti-VGFR2 dsAb NTV3
MAQVQLLESGGGLVQPGGSLRLSCAASGVRVNYKSMSWVRQAPGKGLEWVSTITSRNGSTYYADSVKGRFTISRDNSKNTLYLQMNSLRAEDTAVYYCATGRAHHAPVRYWGQGTLVTVSS

SEQ ID NO: 17 - DNA, 363 bp, synthetic construct
  misc_feature 1..363: note = Amino-acid coding DNA sequence of Anti-VGFR2 dsAb NTV4
  source 1..363: mol_type = other DNA
atggcgcaggttcagctgctggaatctggtggtggtctggttcagccgggtggttctctgcgtctgtcttgcgcggcgtctggtgttaccatcaccgacgaagacatgacccgtgttcgtcaggcgccgggtaaaggtctggaatgggtttcttctatcctgaacaccggtggttctacctactacgcggactctgttaaaggtcgtttcaccatctctcgtgacaactctaaaaacaccctgtacctgcagatgaactctctgcgtgcggaagacaccgcggtttactactgcgcggcggttcacgaaaaagcggcggacatgaacttctggggtcagggtaccctggttaccgtttcttct

SEQ ID NO: 18 - protein, 120 aa, synthetic construct
  REGION 1..120: note = Amino-acid sequence of Anti-VGFR2 dsAb NTV4
AQVQLLESGGGLVQPGGSLRLSCAASGVTITDEDMTRVRQAPGKGLEWVSSILNTGGSTYYADSVKGRFTISRDNSKNTLYLQMNSLRAEDTAVYYCAAVHEKAADMNFWGQGTLVTVSS

SEQ ID NO: 19 - DNA, 327 bp, Camelus bactrianus
  source 1..327: mol_type = other DNA
gaagttcagctgctggaatctggtggtggtctggttcagccgggtggttctctgcgttcttgcgaagcgtctggtttcaacgcgatgacctctatcgactcttggaccgacgcggttaaaggttgggttcgtcagccgccgggtaaaggtctggaatgggtttctcgtttcgcgatctctcaggacaacgcgaaaaacaccgtttacctgcagatgaactctctgaaaccggaagacaccgcgatgtactactgcgcgctgtctaaatgctacacccgtgtttacgactactggggtcagggtacccaggttaccgtttcttctggt

SEQ ID NO: 20 - protein, 108 aa, Camelus bactrianus
EVQLLESGGGLVQPGGSLRSCEASGFNAMTSIDSWTDAVKGWVRQPPGKGLEWVSRFAISQDNAKNTVYLQMNSLKPEDTAMYYCALSKCYTRVYDYWGQGTQVTVSS

SEQ ID NO: 21 - DNA, 312 bp, Camelus bactrianus
  source 1..312: mol_type = other DNA
gaagttcagctgcaggaatctggtggtggtctggttcagccgggtggttctctgcgtctgtcttgcgcggcgtctggtttcatctacatggttggtatcaaaaccgaacgtgacggtgttaaaggttgggttcgtcaggcgccgggtaaaggtctggaatggctgtctcgtttcaccatcccgcgtgacaacgcgaaaaacaccctgtacctgcagatgaacaacctgaaatctgaagacaccgcgctgtactactgcgcgaccgaagaaaacgactggggtcagggtacccaggttaccgtttcttctggt

SEQ ID NO: 22 - protein, 103 aa, Camelus bactrianus
EVQLQESGGGLVQPGGSLRLSCAASGFIYMVGIKTERDGVKGWVRQAPGKGLEWLSRFTIPRDNAKNTLYLQMNNLKSEDTALYYCATEENDWGQGTQVTVSS

SEQ ID NO: 23 - DNA, 351 bp, Camelus dromedarius
  source 1..351: mol_type = other DNA
caggttcagctgcaggaatctggtggtggttctgttcaggcgggtggttctctgcgtctgtcttgcaccgcgtctggtttcggtgttgacggtaccgacatgggttggtaccgtcaggcgccgggtaacgaatgcgaactggtttcttctatctcttctatcggtatcggttactactctgaatctgttaaaggtcgtttcaccatctctcgtgacaacgcgaaaaacaccgtttacctgcagatgaactctctgcgtccggacgacaccgcggtttactactgcggtcgtcgttggatcggttaccgttgcggtaactggggtcgtggtacccaggttaccgtttcttct

SEQ ID NO: 24 - protein, 117 aa, Camelus dromedarius
QVQLQESGGGSVQAGGSLRLSCTASGFGVDGTDMGWYRQAPGNECELVSSISSIGIGYYSESVKGRFTISRDNAKNTVYLQMNSLRPDDTAVYYCGRRWIGYRCGNWGRGTQVTVSS

SEQ ID NO: 25 - DNA, 369 bp, Camelus dromedarius
  source 1..369: mol_type = other DNA
caggttcagctgcaggaatctggtggtggttctgttcaggcgggtggttctctgcgtctgtcttgcaccggttctcgttacacctacaccatgggttggttccgtcaggcgccgggtaaagaacgtgaaggtgttgttgcgatcaccgcgttcggttctccgttctacgcggactctgttaaaggtcgtttcaccatctctcgtgacaacgcgaacaacaccatcttcctgcagatgaactctctgaaaccggaagactctgcgatgtactactgcgcggcgcgtggttcttctggtacctcttacaaatggaacgaatacggttcttacaactactggggtcagggtacccaggttaccgtttcttct

SEQ ID NO: 26 - protein, 123 aa, Camelus dromedarius
QVQLQESGGGSVQAGGSLRLSCTGSRYTYTMGWFRQAPGKEREGVVAITAFGSPFYADSVKGRFTISRDNANNTIFLQMNSLKPEDSAMYYCAARGSSGTSYKWNEYGSYNYWGQGTQVTVSS

SEQ ID NO: 27 - DNA, 351 bp, Camelus dromedarius
  source 1..351: mol_type = other DNA
caggttcagctgcaggaatctggtggtggttctgttcaggcgggtggttctctgcgtctgtcttgcgcggcgtctaaatacacctcttgcatgggttggttccgtcaggcgccgggtaaagaacgtgaagttgttgcgcacatcgactctggtccgcgtaccctgtacgcggactctgttaaaggtcgtttcaccatctctaaagacaacgcgaaaaacaccctgtacctggaaatgtctaccctgaaaccggacgacaccgcgatgtactactgcgcggcgggtccgatgtactctggttcttgcaactacaactactggggtcagggtacccaggttaccgtttcttct

SEQ ID NO: 28 - protein, 117 aa, Camelus dromedarius
QVQLQESGGGSVQAGGSLRLSCAASKYTSCMGWFRQAPGKEREVVAHIDSGPRTLYADSVKGRFTISKDNAKNTLYLEMSTLKPDDTAMYYCAAGPMYSGSCNYNYWGQGTQVTVSS

SEQ ID NO: 29 - DNA, 366 bp, Camelus dromedarius
  source 1..366: mol_type = other DNA
caggttcagctgcaggaatctggtggtggtctggttcagccgggtggttctctgcgtctgtcttgcgcggcgtctggtttcaccctggactactacgcgatcggttggttccgtcaggcgccgggtaaagaacgtgaaggtgtttcttgcatctcttcttctgacggttctacctactacgcggactctgttaaaggtcgtttcaccatctctcgtgacaacgcgaaaaacaccgtttacctgcagatgtcttctctgaaaccggaagacaccgcggtttactactgcggtatctctggttcttgcctgctggaagactacggtatggactactggggtaaaggtacccaggttaccgtttcttct

SEQ ID NO: 30 - protein, 122 aa, Camelus dromedarius
QVQLQESGGGLVQPGGSLRLSCAASGFTLDYYAIGWFRQAPGKEREGVSCISSSDGSTYYADSVKGRFTISRDNAKNTVYLQMSSLKPEDTAVYYCGISGSCLLEDYGMDYWGKGTQVTVSS

SEQ ID NO: 31 - DNA, 378 bp, Camelus dromedarius
  source 1..378: mol_type = other DNA
caggttcagctgcaggaatctggtggtggtctggttcacccgggtggttctctgcgtctgtcttgcgcggcgtctggtttctctctggacaactacgcgatcggttggttccgtcaggcgccgggtaaagaacgtgaaggtgtttcttgcatctcttctggttctgaaggtcgtcgttactacgcggacttcgttaaaggtcgtttcaccatctctcgtgacaacgcgaaaaacaccgcgttcctgcagatgaactctctgaaaccggaagacaccgcggactactactgcgcgaccgttggtttctgctcttctcagtacggtatggaattcgttggtgactactggggtcagggtacccaggttaccgtttcttct

SEQ ID NO: 32 - protein, 126 aa, Camelus dromedarius
QVQLQESGGGLVHPGGSLRLSCAASGFSLDNYAIGWFRQAPGKEREGVSCISSGSEGRRYYADFVKGRFTISRDNAKNTAFLQMNSLKPEDTADYYCATVGFCSSQYGMEFVGDYWGQGTQVTVSS

SEQ ID NO: 33 - protein, 115 aa, synthetic construct
  REGION 1..115: note = Amino-acid sequence of Anti-mouse serum albumin dsAb MSA21
QVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 34 - protein, 115 aa, synthetic construct [sequence omitted]

SEQ ID NO: 35 - protein, 114 aa, synthetic construct
  REGION 1..114: note = Amino-acid sequence of Anti-human serum albumin dsAb Alb23
EVQLLESGGGLVQPGGSLRLSCAASGFTFRSFGMSWVRQAPGKGPEWVSSISGSGSDTLYADSVKGRFTISRDNSKTLYLQMNSLRPEDTAVYYCTIGGSLSRSSQGTLVTVSS

SEQ ID NO: 36 - protein, 125 aa, synthetic construct
  REGION 1..125: note = Amino-acid sequence of Anti-EGFR VHH 7A5
EVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSS

SEQ ID NO: 37 - protein, 124 aa, synthetic construct
  REGION 1..124: note = Amino-acid sequence of Anti-EGFR VHH 7D12
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 38 - protein, 124 aa, synthetic construct
  REGION 1..124: note = Amino-acid sequence of Anti-EGFR VHH 7C12
AVQLVESGGGSVQAGGSLRLTCAASGRTSRSYGMGWFRQAPGKEREFVSGISWRGDSTGYADSVKGRFTISRDNAKNTVDLQMNSLKPEDTAIYYCAAAAGSTWYGTLYEYDYWGQGTQVTVSS

SEQ ID NO: 39 - protein, 114 aa, synthetic construct
  REGION 1..114: note = Amino-acid sequence of Anti-insulin-like growth factor 1 receptor VHH 4B11
EVQLVESGGGLVQPGGSLRLSCAASGSIFTFNAMGWYRQAPGKQRELVAVIISGGSTHYVDSVKGRFTISRDNAKKMVYLQMNSLKPEDTAVYYCNVKKFGDYWGQGTQVTVSS

SEQ ID NO: 40 - protein, 116 aa, synthetic construct
  REGION 1..116: note = Amino-acid sequence of Anti-insulin-like growth factor 1 receptor VHH 3G7
DVQLVESGGGLVQAGGSLRLSCAASESISTINVMAWYRQAPGKQRELVAEITRSGRTNYVDSVKGRFTISRDNAKNTMYLQMNSLNLEDTAVYYCRTIDGSWREYWGQGTQVTVSS

SEQ ID NO: 41 - protein, 127 aa, synthetic construct
  REGION 1..127: note = Amino-acid sequence of Anti-insulin-like growth factor 1 receptor VHH 2C7
QVKLEESGGGLVQPGGSLRLSCVASGRTFSNYAIVIGWFRQAPGQEREFVAAINWNSRSTYYADSVKGRFTISRLNARNTVYLQMNRLKPEDTAVYDCAASHDSDYGGTNANLYDYWGQGTQVTVSS

SEQ ID NO: 42 - protein, 124 aa, synthetic construct
  REGION 1..124: note = Amino-acid sequence of Anti-insulin-like growth factor 1 receptor VHH 1C7
QVKLEESGGGLVQAGGSLRLSCVASGRTFSRTANAWFRQAPGKEREFVATITWNSGTTRYADSVKGRFFISKDSAKNTIYLEMNSLEPEDTAVYYCAATAAAVITPTRGYYNYWGQGTQVTVSS

SEQ ID NO: 43 - protein, 123 aa, synthetic construct
  REGION 1..123: note = Amino-acid sequence of Anti-CEACAM VHH NbCEA5
EVQLVESGGGSVQAGGSLRLSCAASGDTYGSYWMGWFRQAPGKEREGVAAINRGGGYTVYADSVKGRFTISRDTAKNTVYLQMNSLRPDDTADYYCAASGVLGGLHEDWFNYWGQGTLVTVSS

SEQ ID NO: 44 - protein, 123 aa, synthetic construct
  REGION 1..123: note = Amino-acid sequence of Anti-CEACAM VHH CEA5
EVQLVESGGGSVQAGGSLRLSCAASGDTYGSYWMGWFRQAPGQEREAVAAINRGGGYTVYADSVKGRFTISRDNAKNTLYLQMNSLRPDDTADYYCAASGVLGGLHEDWFNYWGQGTLVTVSS

SEQ ID NO: 45 - protein, 116 aa, synthetic construct
  REGION 1..116: note = Amino-acid sequence of Anti-CD123 VHH 57A07
EVQLVESGGGLVQAGGSLRLSCAASGSIFSGNVMGWYRRQAPGKEREWVAAIASGGSIYYRDSVKGRFTISRDNAKNTVYLQMNSLKPEDTAVYYCNSHPPTLPYWGLGTQVTVSS

SEQ ID NO: 46 - protein, 116 aa, synthetic construct
  REGION 1..116: note = Amino-acid sequence of Anti-CD123 VHH 57B04
EVQLVESGGGLVQPGGSLRLSCAASGINFRFNSMGWWRRRAPGKEREWVAAITSGDITNYRDSVRGRFTISRDNVKNTVYLQMNTLKLEDTAVYYCNTFPPIADYWGLGTQVTVSS

SEQ ID NO: 47 - protein, 123 aa, synthetic construct
  REGION 1..123: note = Amino-acid sequence of Anti-CD123 VHH 51D09
EVQLVESGGGLVQPGGSLRLSCAASGSIFSGNTMGWYRQAPGKQRELVAAISSGGSTDYADSVKGRFTISRDNSKNTVYLQMNSLRPEDTAVYYCNAAILLYRLYGYEEGDYWGLGTLVTVSS

SEQ ID NO: 48 - protein, 125 aa, synthetic construct
  REGION 1..125: note = Amino-acid sequence of Anti-CD123 VHH 55C05
EVQLVESGGGLVPAGDSLRLSCVASGRSLNTYTMGWFRQAPGKECEEVAAINWNGVYRDYADSAKGRETASRDNAMNTVFLQMNSLKPEDTAVYFCATATQGWDRHTEPSDFGSWGLGTQVTVSS

SEQ ID NO: 49 - protein, 124 aa, synthetic construct
  REGION 1..124: note = Amino-acid sequence of Anti-CD123 VHH 50F07
EVQLVESGGGLVQPGGSLRLSCTGSGSTFSINAMGWYRQAPGKQRELVAAITSGGRTNYADSVKGRFTISRDNSKNTVYLQMNSLRPEDTAVYYCNARISAGTAFWLWSDYEYWGLGTLVTVSS

SEQ ID NO: 50 - protein, 125 aa, synthetic construct
  REGION 1..125: note = Amino-acid sequence of Anti-CD123 VHH 55F03
EVQLVESGGGLVQAGGPLRLSCAASGRTFSSYVMGWFRQAPGKEREFVAAIYWSNGKTQYTDSVKGRFTISGDNAKNTVYLQMNSLNPEDTAVYYCVADKDETGFRTLPIAYDYWGLGTQVTVSS

SEQ ID NO: 51 - protein, 126 aa, synthetic construct
  REGION 1..126: note = Amino-acid sequence of Anti-CD123 VHH 55A01
EVQLVESGGGSVQAGGSLRLSCTTSGRALNMYVMGWFRQAPGNEREFVAATSSSGGSTSYPDSVKGRFTISRDNAKNTVYLQMNSLKPEDTAAYRCAASPYVSTPTMNILEEYRYWGLGTQVTVSS

SEQ ID NO: 52 - protein, 125 aa, synthetic construct
  REGION 1..125: note = Amino-acid sequence of Anti-c-MET VHH 04E09
EVQLVESGGGLVQPGGSLRLSCAASGFILDYYAIGWFRQAPGKEREGVLCIDASDDITYYADSVKGRFTISRDNAKNTVYLQMNSLKPEDTGVYYCATPIGLSSSCLLEYDYDYWGQGTLVTVSS

SEQ ID NO: 53 - protein, 130 aa, synthetic construct
  REGION 1..130: note = Amino-acid sequence of Anti-c-MET VHH 06B08
EVQLVESGGGLVQAGGSLRLSCAASGRTISRYTMGWFRQAPGKEREFVAAISWSGDNTNYADSVKGRFTISRPNTKNTMYLQMNSLKPEDTAVYYCAADYRSGSYYQASEWTRPSGYDYWGQGTLVTVSS

SEQ ID NO: 54 - protein, 125 aa, synthetic construct
  REGION 1..125: note = Amino-acid sequence of Anti-c-MET VHH 06C12
EVQLVESGGGLVQPGGSLRLSCAASGFSLDYFAIGWFRQAPGKEREEISCISNSDGSTYYANSVKGRFTISIDNAKNTVYLQMTSLKPEDTAVYYCATPVGLGPFCKTTNDYDYSGQGTLVTVSS

SEQ ID NO: 55 - protein, 124 aa, synthetic construct
  REGION 1..124: note = Amino-acid sequence of Anti-c-MET VHH 06F10
EVQLVESGGGLVQPGGSLRLSCAASGFTLDYYAINWFRQAPGKEREGVSCISGGDGSTYYADSVKGRFTISRDNAKNTVYLQMNSLKPEDTAVYYCATALGLSSSCHGDGYDYWGQGTLVTVSS

SEQ ID NO: 56 - protein, 125 aa, synthetic construct
  REGION 1..125: note = Amino-acid sequence of Anti-Her3 VHH 21F6
EVQLVESGGGLVQAGGSLRLSCAASGRTYYLNAMGWFRQGPGKDREFVAAIDWSDGNKDYADSVKGRFTISRDNAKNTVYLQMNSLKPEDTAVYYCAADTPPWGPMIYIESYDSWGQGTLVTVSS

SEQ ID NO: 57 - protein, 113 aa, synthetic construct
  REGION 1..113: note = Amino-acid sequence of Anti-Her3 VHH 4C7
EVQLVESGGGLVQAGGSLRLSCAASGFTFSSYPMSWVRQAPGKGPAWVSTVSPGGITTSYADSVKGRFTISRDNAKNTLYLQMNSLKPEDTAVYYCLRDLNNRGQGTLVTVSS

SEQ ID NO: 58 - protein, 118 aa, synthetic construct
  REGION 1..118: note = Amino-acid sequence of Anti-Her3 VHH 17B5
EVQLVESGGGLVQPGGSLRLSCAASGSIGGLNAMAWYRQAPGKERELVAGIFGVGSTRYADSVKGRFTISRDIAKNTVFLQMNSLNSEDTAVYYCRMSSVTRGSSDYWGQGTQVTVSS

SEQ ID NO: 59 - protein, 122 aa, synthetic construct
  REGION 1..122: note = Amino-acid sequence of Anti-Her3 VHH 18G11
EVQLVESGGGLVQPGGSLRLSCAASGTLFKINAMGWYRQAPGKRRELVALITSSDTTDYAESVEGRFTISRDNTWNAVYLQMNSLKPEDTAVYYCHSDHYSMGVPEKRVIMYGQGTQVTVSS

SEQ ID NO: 60 - protein, 120 aa, synthetic construct
  REGION 1..120: note = Amino-acid sequence of Anti-Her3 VHH 34C7
EVQLVESGGGLVQPGGSLGLSCVASGSIFRINAMAWYRQAPGKQRELVAEITAGGSTNYADSVKGRFTISVDNAWNTLYLQMNSLKVEDTAVYYCNLDHYTTWDRRSAYWGQGTQVTVSS

SEQ ID NO: 61 - protein, 119 aa, Lama glama
KVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 62 - protein, 124 aa, Lama glama
EVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSS

SEQ ID NO: 63 - protein, 118 aa, Lama glama
EVQLVESGGGLVQPGGSLRLSCAASGFTFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 64 - protein, 118 aa, Lama glama
EVQLVESGGGLVHPGGSLRLSCVGSGFSLDDYGMTWVRRAPGKGLEWVSSINWSGTHTDYADSVKGRFTISRDNAKNTLFLQMNSLNPEDTAVYYCGQGWKIVPTNPRGHGTQVTVSS

SEQ ID NO: 65 - protein, 118 aa, Lama glama
EVQLVESGGGLVQPGGSLRLSCVGSGFSLDDYAMTWVRQAPGKGLEWVSSINWSGTHTDYADSVKGRFTISRDNAKNTLFLQMNSLSPEDTAVYYCNQGWKIRPTIPMGHGTQVTVSS

SEQ ID NO: 66 - protein, 118 aa, Lama glama
EVQLVESGGGLVQPGGSLRLSCVASGFSLDDYGMTWVRQAPGKGLEWVSSINWSGTHTDYTDPVKGRFTISRDNAKNTLFLQMNNLTPEDTAVYYCNRGWKIVPTDLGGHGTQVTVSS

SEQ ID NO: 67 - protein, 122 aa, Lama glama
EVQLVESGGGLVQAGGSLRLSCAASGRTFISNYAMGWFRQAPGKEREFVATINWSGSHSDYADSVKGRFTISRDNAKNTVYLQMNNLKSEDTAVYYCAPGWGTAPLSTSVYWGQGTQVTVSS

SEQ ID NO: 68 - protein, 119 aa, Lama glama
EVQLVESGGGMVQAGGSLRLSCAASGLTLSNYGMGWFRQAPGKEREFVSSINWSGTHTYDADFVKGRFIISRDNAKNTVYLQINSLKPEDTAVYYCAAGGWGTGRYNYWGQGTQVTVSS

SEQ ID NO: 69 - protein, 119 aa, Lama glama; identical to SEQ ID NO: 68

SEQ ID NO: 70 - protein, 120 aa, Lama glama
EVQLVESGGGLVQAGGSLRLSCAASGRTFNAMGWFRQAPGKEREFVAAISRSPGVTYYADSVKGRFTTSRDNAKNTVYLQMNDLKPEDTAVYYCAADFYLATLAHEYDYWGQGTQVTVSS

SEQ ID NO: 71 - protein, 122 aa, Lama glama
EVQLVESGGGLVQAGGSLRLSCAASGRTFSSYAMAWFRQAPGTEREFIAGIRWSDGSTYYADSVKGRFTISRDNAKNTVYLQMNSLKPEDTAVYYCAADFYVSTLAHEYDYWGQGTQVTVSS

SEQ ID NO: 72 - protein, 122 aa, Lama glama
KVQLVESGGGLVQAGGSLRLSCAASGRTFSSYAMAWFRQAPGTEREFIAGIRWSDGSTYYADSVKGRFTISRANAKNTVYLQMNGLKPEDTAVYYCAADFYVSTLAHEYDYWGQGTQVTVSS

SEQ ID NO: 73 - protein, 576 aa, synthetic construct
  REGION 1..576: note = Bivalent VHHEGFR-dianthin-Cys
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGGSAAATAYTLNLANPSASQYSSFLDQIRNNVRDTSLIYGGTDVAVIGAPSTTDKFLRLNFQGPRGTVSLGLRRENLYVVAYLAMDNANVNRAYYFKNQITSAELTALFPEVVVANQKQLEYGEDYQAIEKNAKITTGDQSRKELGLGINLLITMIDGVNKKVRVVKDEARFLLIAIQMTAEAARFRYIQNLVTKNFPNKFDSENKVIQFQVSWSKISTAIFGDCKNGVFNKDYDFGFGKVRQAKDLQMGLLKYLGRPKGGGGSGGGGSHRWCCPGCCKTFGGGGSHHHHHHHHHHHRWCCPGCCKTF

SEQ ID NO: 74 - protein, 283 aa, synthetic construct
  REGION 1..283: note = Bivalent VHHEGFR-Cys
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSHHHHHHHHHHHRWCCPGCCKTF

SEQ ID NO: 75 - protein, 124 aa, synthetic construct; identical to SEQ ID NO: 37
  REGION 1..124: note = VHH 7D12

SEQ ID NO: 76 - protein, 127 aa, synthetic construct; identical to SEQ ID NO: 10
  REGION 1..127: note = VHH 9G8

SEQ ID NO: 77 - protein, 12 aa, synthetic construct
  REGION 1..12: note = Tetra-Cys linker
HRWCCPGCCKTF

SEQ ID NO: 78 - protein, 576 aa, synthetic construct; identical to SEQ ID NO: 73
  REGION 1..576: note = bivalent VHH-EGFR-Dianthin